(19) United States
(12) Patent Application Publication
(10) Pub. No.: US 2004/0023253 A1
(43) Pub. Date: Feb. 5, 2004

(54) Device structure for closely spaced electrodes

(76) Inventors: Sandeep Kunwar, Hillsborough, CA (US); George T. Mathai, Castro Valley, CA (US)

Correspondence Address: PENNIE AND EDMONDS, 1155 AVENUE OF THE AMERICAS, NEW YORK, NY, 100362711

(21) Appl. No.: 10/335,482

(22) Filed: Dec. 26, 2002

Related U.S. Application Data
(63) Continuation-in-part of application No. PCT/US02/18319, filed on Jun. 10, 2002.
(63) Continuation-in-part of application No. 09/970,087, filed on Oct. 2, 2001.
(60) Provisional application No. 60/297,583, filed on Jun. 11, 2001.
(60) Provisional application No. 60/378,938, filed on May 10, 2002.

(51) Int. Cl. C12Q 1/68; C12M 1/34

(52) U.S. Cl. 435/6; 435/287.2; 205/777.5

(57) ABSTRACT

A biosensor comprising a plurality of devices on a substrate. Each device in the plurality of devices occupying a different region on the substrate. Each device in the plurality of devices comprises a first electrically conducting material, a spacer, and a second electrically conducting material. The first electrically conducting material is overlaid on a first portion of the different region on the substrate occupied by a device and the spacer is overlaid on a second portion of the different region on the substrate that is occupied by the device. The first electrically conducting material and the spacer abut each other. The second electrically conducting material is overlaid on a portion of the spacer.

### 1. CROSS-REFERENCE TO RELATED APPLICATIONS

This application is a continuation-in-part of U.S. patent application Ser. No. 09/970,087, filed Oct. 2, 2001, which claims the benefit of U.S. Provisional Patent Application No. 60/297,583, filed on Jun. 11, 2001, each of which is incorporated herein by reference in their entireties. Furthermore, this application claims priority, under 35 U.S.C. § 119(e), of U.S. Provisional Patent Application No. 60/378,938 filed on May 10, 2002 which is incorporated herein, by reference, in its entirety.

### 2. FIELD OF THE INVENTION

This invention pertains to a biosensor for detecting and/or quantifying analytes. More particularly, this invention pertains to a biosensor based on a detection element that is a single macromolecule spanning two electrodes. The invention additionally pertains to methods of manufacturing such biosensors.

### 3. BACKGROUND OF THE INVENTION

Biosensors are devices that can detect and/or quantify analytes using known interactions between a targeted analyte and a binding agent that is typically a biological macromolecule, such as an enzyme, receptor, nucleic acid, protein, lectin, or antibody. Biosensors have applications in virtually all areas of human endeavor. For example, biosensors have utility in fields as diverse as blood glucose monitoring for diabetics, the recognition of poisonous gas and/or explosives, the detection of chemicals commonly associated with spoiled or contaminated food, genetic screening, environmental testing, and the like. Thus, the term “biosensor” refers to a sensor that uses a biological macromolecule (e.g. nucleic acid, carbohydrate, protein, antibody, etc.) to specifically recognize/bind to a target analyte. The term “molecular sensing apparatus” is used interchangeably with the term “biosensor”.

Biosensors are commonly categorized according to two features, namely, the type of macromolecule utilized in the device and the means for detecting the contact between the binding agent and the targeted analyte. Major classes of biosensors include enzyme (or catalytic) biosensors, immunosensors and DNA biosensors.

Enzyme (or catalytic) biosensors typically utilize one or more enzymes as the macromolecule and take advantage of the complimentary shape of the selected enzyme and the targeted analyte. Enzymes are proteins that perform most of the catalytic work in biological systems and are known for highly specific catalysis. The shape and reactivity of a given enzyme limits its catalytic activity to a very small number of possible substrates. Enzyme biosensors rely on the specific chemical changes related to the enzyme/analyte interaction as the means for recognizing contact with the targeted analyte. For example, upon interaction with an analyte, an enzyme biosensor may generate electrons, a colored chromophore or a change in pH as the result of the relevant enzymatic reaction. Alternatively, upon interaction with an analyte, an enzyme biosensor may cause a change in a fluorescent or chemiluminescent signal that can be recorded by an appropriate detection system.

Immunosensors utilize antibodies as binding agents. Antibodies are protein molecules that generally do not perform catalytic reactions, but specifically bind to particular “target” molecules (antigens). Antibodies are quite specific in their interactions and, unlike most enzymes, they are capable of recognizing and selectively binding to very large bodies such as single cells. Thus, in addition to detection of small analytes, antibody-based biosensors allow for the identification of certain pathogens such as dangerous bacterial strains.

DNA biosensors typically utilize the complementary nature of DNA double-strands. They are designed for the specific detection of particular nucleic acids. A DNA biosensor sensor generally uses a single-stranded DNA as the binding agent. The nucleic acid material in a given test sample is placed into contact with the binding agent under conditions where the biosensor DNA and the target nucleic acid analyte can form a hybrid duplex. If a nucleic acid in the test sample is complementary to a nucleic acid used in the biosensor, the two interact (e.g., the two bind to each other). The interaction can be monitored by various means such as a change in mass at the sensor surface or the presence of a fluorescent or radioactive signal. In alternative arrangements, the target nucleic acid(s) are bound to the sensor and contacted with labeled probes to allow for identification of the sequence(s) of interest.

When a single-stranded DNA binds to a complementary single-stranded DNA or RNA, the charge conducting characteristics of the DNA change. Charge transfer and transport in DNA is a function of many different phenomena, including the redox potential of the bases in the DNA, base-stacking characteristics, structural distortion, as well as the sequence of the DNA. See, for example, Cai et al., 2000, Applied Physics Letters 77, pp. 3105-3106; and Giese et al., 2001, Nature 412, pp. 318-320. Further, the studies of Fink & Schönenberger, Hjort & Stafström, and Kasumov et al. indicate that at least some DNA sequences are molecular conductors. See Fink & Schönenberger, 1999, Nature 398, pp. 407-410; Hjort & Stafström, 2001, Physical Review Letters 87, 228101-1228101-4; Kasumov et al., 2001, Science 291, pp. 280-282.

While biosensors have potential and while hundreds of biosensors have been described in patents and in the literature, actual commercial use of biosensors remains limited. Features of biosensors that have limited their commercial acceptance include a lack the sensitivity and/or speed of detection necessary to accomplish certain tasks, problems with long term stability, difficulty miniaturizing the sensor, and the like. In addition, a number of biosensors must be pre-treated with salts and/or enzyme cofactors, a practice that is inefficient and bothersome.

Thus, given the above background, what is needed in the art are improved biosensors.

### 4. SUMMARY OF THE INVENTION

This invention pertains to novel sensors (biosensors) that are useful for detecting a wide range of macromolecules as well as macromolecule binding events. In some embodiments, the biosensors of the present invention bind to one or more macromolecules. Then, the macromolecules are exposed to analytes. Binding events between the macromolecules and the analytes are detected as measured changes in electrical signals.

In preferred embodiments, each tested macromolecule spans a gap between two electrodes. Binding of a target analyte to the macromolecule changes conductivity, or other electrical properties, of the sensor thereby facilitating ready detection of the binding event and thus detection and/or quantification of the bound analyte. Because the biosensors of this invention provide a change in conductance or charge flow when bound by the target analyte, they are easily read using electronic/electrochemical means and do not require the use of detectable labels or external electron donors or acceptors.

A significant advantage of the present invention is that electrode pairs used to bind to test macromolecules are separated in the z dimension on a substrate. That is, each electrode pair is on a different layer on the substrate. Thus, using known semiconductor manufacturing techniques to precisely adjust layer thickness, the separation between the electrode pairs can be precisely and accurately manufactured at lengths that are optimal for the study of macromolecule binding events.

One aspect of the present invention provides a biosensor comprising a plurality of devices on a substrate. Each device in the plurality of devices occupies a different region on an insulator layer and each device in the plurality of devices is for binding to a macromolecule. The insulator layer is overlaid on the substrate. Each device in the plurality of devices comprises (i) a first electrically conducting material overlaid on a first portion of the different region of the insulator layer occupied by the device, (ii) a spacer overlaid on a second portion of the different region, and (iii) a second electrically conducting material. In each device in the plurality of devices, the first electrically conducting material and the spacer abut each other and the second electrically conducting material is overlaid on a portion of the spacer.

Another aspect of the present invention provides a biosensor comprising a plurality of devices on a substrate. Each device in the plurality of devices occupies a different region on the substrate and each device in the plurality of devices is for binding to a macromolecule. Each device in the plurality of devices comprises (i) a first electrically conducting material overlaid on a first portion of the different region on the substrate occupied by the device, (ii) a spacer overlaid on a second portion of the different region, and (iii) a second electrically conducting material. In each device in the plurality of devices, the first electrically conducting material and the spacer abut each other and the second electrically conducting material is overlaid on a portion of the spacer.

In some embodiments, the second electrically conducting material overlaps the first electrically conducting material of a device in the plurality of devices by a distance, thereby forming a cavity. In some embodiments, this distance is 150 Angstroms or less, 100 Angstroms or less, or 50 Angstroms or less.

In some embodiments, a passivation layer overlays the second electrically conducting material. In some embodiments, this passivation layer comprises silicon oxide, silicon dioxide, silicon nitride, silicon oxy-nitride, polyamide, oxidized aluminum, or photoresist.

In some embodiments, a first portion of the macromolecule binds to a top portion of the first electrically conducting material and a second portion of the macromolecule binds to a side-wall of the second electrically conducting material in a device in the plurality of devices.

In still other embodiments, a first passivation layer overlays a portion of the first electrically conducting material and a second passivation layer overlays the second electrically conducting material. Furthermore, a first portion of the macromolecule binds to a top portion of the first electrically conducting material that is not covered by the first passivation layer and a second portion of the macromolecule binds to a side portion of second electrically conducting material. In some embodiments, the first passivation layer and the second passivation layer each independently comprise silicon oxide, silicon dioxide, silicon nitride, silicon oxy-nitride, polyamide, oxidized aluminum, or photoresist.

Another aspect of the present invention provides a biosensor comprising a plurality of devices on a substrate. Each device in the plurality of devices occupies a different region on an insulator layer and each device in the plurality of devices is for binding to a macromolecule. The insulator layer is overlaid on the substrate. Each device in the plurality of devices is associated with a different cavity in the insulator layer. Each device in the plurality of devices comprises (i) a first electrically conducting material overlaid in the cavity associated with the device, (ii) a second electrically conducting material that is overlaid on the insulator outside of the cavity, and (iii) a passivation layer overlaid on the second electrically conducting material. In some embodiments, the passivation layer comprises silicon oxide, silicon dioxide, silicon nitride, silicon oxy-nitride, polyamide, oxidized aluminum, or photoresist. In some embodiments, a different cavity associated with a device in the plurality of devices has a width of between 900 Angstroms and 20,000 Angstroms or a width between 500 Angstroms and 900 Angstroms.

Another aspect of the present invention provides a biosensor comprising a plurality of devices on a substrate. Each device in the plurality of devices occupies a different region on an insulator layer and each device in the plurality of devices is for binding to a macromolecule. The insulator layer is overlaid on the substrate. Each device in the plurality of devices comprises: (i) a first electrically conducting material overlaid on a first portion of the different region of the insulator layer occupied by the device and (ii) a second electrically conducting material overlaid on a second portion of the region. Another embodiment of the invention provides a biosensor comprising a plurality of devices on a substrate. Each device in the plurality of devices occupies a different region on the substrate and each device in the plurality of devices is for binding to a macromolecule. Each device in the plurality of devices comprises (i) a first electrically conducting material overlaid on a first portion of the different region of the substrate occupied by the device and (ii) a second electrically conducting material overlaid on a second portion of the different region.

In some embodiments, the first electrically conducting material and the second electrically conducting material of a device in the plurality of devices are separated by a distance of 10 Angstroms or greater or 30 Angstroms or greater. In some embodiments, the passivation layer overlays a portion of the second electrically conducting material in a device in the plurality of devices. In some embodiments, the passivation layer comprises silicon oxide, silicon dioxide, silicon nitride, silicon oxy-nitride, polyamide, oxidized aluminum, or photoresist.

In some embodiments, a first portion of a macromolecule binds to a top portion of a first electrically conducting material and a second portion of the macromolecule binds to a side portion of a second electrically conducting material in a device in the plurality of devices. In other embodiments, a first portion of a macromolecule binds to a side portion of a first electrically conducting material and a second portion of a macromolecule binds to a side portion of a second electrically conducting material in a device in the plurality of devices. In yet other embodiments, a first portion of a macromolecule binds to a top portion of a first electrically conducting material and a second portion of the macromolecule binds to a top portion of the second electrically conducting material in a device in the plurality of devices.

In some embodiments, the second electrically conducting material is thicker than the first electrically conducting material in a device in the plurality of devices. In some embodiments, the second electrically conducting material and the first electrically conducting material in a device in said plurality of devices have the same thickness. In some embodiments, the first and second electrically conducting material in a device in the plurality of devices are separated by a distance and there is a gap in the insulator layer between the first electrically conducting material and the second electrically conducting material in the device. In some embodiments, this gap has a width between 60 Angstroms and 500 Angstroms, a width between 60 Angstroms and 1000 Angstroms, a width between 60 Angstroms and 10,000 Angstroms, a width between 60 Angstroms and 30,000 Angstroms, a width between 60 Angstroms and 100,000 Angstroms, or a width that exceeds a distance that separates the first electrically conducting material and the second electrically conducting material of the device.

Yet another aspect of the present invention provides a biosensor comprising a plurality of devices on a substrate. Each device in the plurality of devices occupies a different region on an insulator layer. Each device in the plurality of devices is for binding to a macromolecule. The insulator layer is overlaid on the substrate. Each device in the plurality of devices comprises (i) a first electrically conducting material overlaid on the different region of the insulator layer occupied by the device, (ii) a spacer overlaying the first electrically conducting material, (iii) a second electrically conducting material overlaid on the spacer, and (iv) a passivation layer overlaid on the second electrically conducting material. The spacer includes a thin segment and a thick segment and the thin segment of the spacer is not as thick as the thick segment of the spacer. In some embodiments, the thin segment of the spacer in a device in the plurality of devices includes a cavity, and a first portion of a macromolecule binds to a top portion of the first electrically conducting material and a second portion of the macromolecule binds to a bottom portion of the second electrically conducting material in the cavity.

In some embodiments, a distance between the first electrically conducting material and the second electrically conducting material in a device in the plurality of devices is between 25 Angstroms and 104 Angstroms, between 40 Angstroms and 102 Angstroms, or between 40 Angstroms and 80 Angstroms. In some embodiments, a first portion of the macromolecule binds to a side portion of the first electrically conducting material and a second portion of the macromolecule binds to a side portion of the second electrically conducting material in a device in the plurality of devices.

Another aspect of the present invention provides a biosensor comprising a plurality of devices on a substrate. Each device in the plurality of devices occupies a different region on an insulator layer. Each device in the plurality of devices is for binding to a macromolecule. The insulator layer is overlaid on the substrate. Each device in the plurality of devices comprises (i) a first electrically conducting material overlaid on the different region of the insulator layer occupied by the device, (ii) a spacer overlaying a portion of the first electrically conducting material, (iii) a second electrically conducting material overlaid on the spacer so that a cavity is formed, and (iv) a passivation layer overlaid on the second electrically conducting material. In some embodiments, the passivation layer comprises silicon oxide, silicon dioxide, silicon nitride, silicon oxy-nitride, polyamide, oxidized aluminum, or photoresist.

In some embodiments, a first portion of the macromolecule binds to a side portion of the first electrically conducting material and a second portion of the macromolecule binds to a side portion of the second electrically conducting material in a device in the plurality of devices. In some embodiments, a first portion of the macromolecule binds to a top portion of the first electrically conducting material and a second portion of the macromolecule binds to a bottom portion of the second electrically conducting material in the cavity in a device in the plurality of devices.

Another aspect of the present invention provides a biosensor comprising a plurality of devices on a substrate. Each device in the plurality of devices occupies a different region on an insulator layer and each device in the plurality of devices for binding to a macromolecule. The insulator layer is overlaid on the substrate. Each device in the plurality of devices comprises (i) a first electrically conducting material overlaid on a first portion of the different region of the insulator layer that is occupied by the device, (ii) a spacer overlaid on a second portion of the different region of the insulator layer that is occupied by the device, (iii) a second electrically conducting material that abuts a side-wall of the spacer facing the first electrically conducting material, (iv) and a first passivation layer that overlays the spacer and a portion of the second electrically conducting material. In some embodiments, there is no insulator layer and each device in the plurality of devices is overlaid on a different region of the substrate.

In some embodiments, the first passivation layer and/or the second passivation layer comprises silicon oxide, silicon dioxide, silicon nitride, silicon oxy-nitride, polyamide, oxidized aluminum, or photoresist. In some embodiments, a first portion of a macromolecule binds to a top portion of the first electrically conducting material and a second portion of the macromolecule binds to a side-wall of the second electrically conducting material in a device in the plurality of devices. In some embodiments, the second passivation layer overlays a portion of the first electrically conducting material and a first portion of the macromolecule binds to a top portion of the first electrically conducting material that is not covered by the second passivation layer and a second portion of the macromolecule binds to a side-wall of the second electrically conducting material. In some embodiments, the second passivation layer comprises silicon oxide, silicon dioxide, silicon nitride, silicon oxy-nitride, polyamide, oxidized aluminum, or photoresist. In some embodiments, the insulator includes a gap that is between the first electrically conducting material and the spacer. In some embodiments, the spacer includes a crevice that exposes a portion of the second electrically conducting material. Another embodiment of the present invention provides a biosensor comprising a plurality of devices on a substrate. Each device in the plurality of devices occupies a different region on an insulator layer and each device in the plurality of devices is for binding to a macromolecule. The insulator layer is overlaid on the substrate. Each device in the plurality of devices comprises (i) a first electrically conducting material overlaid on a first portion of the different region of the insulator layer occupied by the device, (ii) a spacer overlaid on a second portion of the different region of the insulator layer. The spacer includes a main body and an extended portion. The extended portion of the spacer abuts the first electrically conducting material the first portion of the insulator layer does not overlap with the second portion of the insulator layer. Each device in the plurality of devices further comprises a second electrically conducting material overlaid on the main body of the spacer and a first passivation layer overlaid on the second electrically conducting material.

In some embodiments, a first portion of the macromolecule binds to a top portion of the first electrically conducting material and a second portion of the macromolecule binds to a side-wall of the second electrically conducting material in a device in the plurality of devices. In some embodiments, a second passivation layer overlays a portion of the first electrically conducting material and a first portion of the macromolecule binds to a top portion of the first electrically conducting material that is not covered by the first passivation layer and a second portion of the macromolecule binds to a side portion of the second electrically conducting material in a device in the plurality of devices.

In some embodiments, the extended portion of the spacer has a width of more than 20 Angstroms, more than 50 Angstroms, or more than 100 Angstroms in a device in the plurality of devices. In some embodiments, the extended portion of the spacer comprises a gap in a device in the plurality of devices. In some embodiments, the main portion of the spacer includes a crevice that exposes a bottom portion of the second electrically conductive material. In still other embodiments, a first portion of the macromolecule binds to an upper surface of the first electrically conducting material and a second portion of the macromolecule binds to a side portion of the second electrically conducting material.

Another aspect of the present invention provides a biosensor for binding a macromolecule. The biosensor comprises a substrate and an insulator layer overlaid on the substrate. The insulator layer comprises a plurality of steps. A first step in the plurality of steps is at a different height, with respect to the substrate, than a second step in the plurality of steps. Each step in the plurality of steps is associated with a different electrically conducting layer that is overlaid on the step and each electrically conducting layer on each step in the plurality of steps is electrically insulated from all other electrically conducting layers in the biosensor. In some embodiments, each electrically conducting layer in the biosensor is addressable by an electrical source. In some embodiments, an electrically conducting layer associated with a step in the plurality of steps is electrically insulated from all other electrically conducting layers in the biosensor by a cavity in the step. In some embodiments, the difference in height, with respect to the substrate, between a first step in the plurality of steps and a second step in the plurality of steps is between 60 Angstroms and 200 Angstroms, less than 500 Angstroms, or less than 1000 Angstroms. In some embodiments, the first step and the second step are adjacent to each other and a first portion of a macromolecule binds to the first step in the plurality of steps and a second portion of the macromolecule binds to the second step.

In some embodiments, a portion of the first electrically conducting material and a portion of the second electrically conducting material are separated by a distance that is less than 200 Angstroms, less than 100 Angstroms, or between 40 Angstroms and 80 Angstroms in a device in the plurality of devices. In various embodiments, the plurality of devices comprises 1,000 devices to 250,000 devices or 10,000 devices to 60,000 devices. In some embodiments, the plurality of devices is arranged in an array having at least 200 rows and at least 200 columns on the substrate.

In some embodiments, the substrate is an insulator. In some embodiments, the substrate comprises silicon, silicon oxide, silicon dioxide, silicon nitride, Teflon, alumina, glass, sapphire, a selinide, or polyester. In some embodiments, the first electrically conducting material and/or the second electrically conducting material has a resistivity less than 10-6 ohm-meters in a device in the plurality of devices. In some embodiments, the first electrically conducting material and the second electrically conducting material are comprised of the same composition in a device in the plurality of devices. In some embodiments, the first electrically conducting material and/or the second electrically conducting material are comprised of different compositions in a device in the plurality of devices.

In some embodiments, the first electrically conducting material comprises aluminum, nickel, platinum, iron, copper, silver, gold, indium tin oxide, chromium, titanium, zinc, or tin, or an alloy of aluminum, nickel, platinum, iron, copper, silver, gold, chromium, titanium, zinc or tin in a device in the plurality of devices. In some embodiments, the first electrically conducting material comprises a metal carbide, a metal nitride, a metal boride, a conductive oxide, a metal silicide or a metal sulfide in a device in the plurality of devices. In some embodiments, the insulator comprises a material having a resistivity greater than 10⁻¹ ohmmeters in a device in the plurality of devices. In some embodiments, the insulator comprises TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene or a high resistivity plastic in a device in the plurality of devices.

In some embodiments the spacer comprises metal carbide, a metal nitride, a metal boride, a conductive oxide, a metal silicide or a metal sulfide in a device in the plurality of devices. In some embodiments, the spacer comprises a material having a resistivity greater than 10⁻¹ ohmmeters in a device in the plurality of devices. In some embodiments, the spacer comprises TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene or a high resistivity plastic in a device in the plurality of devices.

In some embodiments a biological macromolecule binds to a device in the plurality devices and the biological macromolecule comprises a nucleic acid, a protein, a polypeptide, a peptide, an antibody, a carbohydrate, a polysaccharide, a lipid, a fatty acid or a sugar.

Another aspect of the present invention provides a method of manufacturing a biosensor for binding a macromolecule. The method comprises depositing a first insulator layer onto a substrate. Next, a second insulator layer is deposited on the first insulator layer. The second insulator layer is patterned, thereby forming a spacer and exposing a portion of the first insulator layer. Electrically conducting material is deposited on the spacer and the portion of the first insulator layer that is exposed. The electrically conducting material deposited on the portion of the first insulator layer is patterned to form a first electrically conducting material. In addition, the electrically conducting material that is deposited on the spacer is patterned to form a second electrically conducting material.

In some embodiments, the depositing of the first insulator layer and/or the second insulator layer is performed by thermal oxidation of silicon, chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, sputter deposition, vacuum evaporation, laser ablated deposition, atomic layer deposition, molecular beam deposition, ion beam deposition, hot filament chemical vapor deposition or screen printing. In some embodiments, the depositing of the second insulator layer on the first insulator layer comprises chemical vapor deposition of silicon oxide or silicon nitride.

In some embodiments, the patterning of the second insulator layer comprises (i) application of a photolithographic photoresist coating to the second insulator layer, (ii) optical imaging of the photolithographic photoresist coating through an optical mask, (iii) developing the photolithographic photoresist coating, (iv) etching the spacer; and (v) removing the photolithographic photoresist coating. In some embodiments, the photolithographic photoresist coating is a negative resist or a positive resist. In some embodiments, the photolithographic photoresist coating is an azide/isoprene negative resist, polymethylmethacrylate (PMMA), polymethylisopropyl ketone (PMIPK), poly-butene-1-sulfone (PBS), poly-(trifluoroethyl chloroacrylate) TFECA, copolymer-α-cyano ethyl acrylate-α-amido ethyl acrylate) (COP), poly-(2-methyl pentene-1-sulfone) (PMPS), phenol-formaldehyde novolak resin, or LOR 3A. In some embodiments, the photolithographic photoresist coating is developed by exposing the photolithographic photoresist coating to xylene, Stoddart solvent, n-butyl acetate, sodium hydroxide, potassium hydroxide, or tetramethylammonium hydroxide.

In some embodiments, the etching of the spacer comprises wet etching, wet spray etching, vapor etching, plasma etching, ion beam etching or reactive ion etching. In some embodiments, the photolithographic photoresist coating comprises exposing the photolithographic photoresist coating to a strong acid, an acid-oxidant combination, an organic solvent stripper, or an alkaline stripper.

In some embodiments, the depositing the layer of electrically conducting material on the spacer and the portion of the first insulator layer that is exposed is performed by chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, direct current diode sputtering, radio frequency diode sputtering, direct current magnetron sputtering, radio frequency magnetron sputtering, vacuum evaporation, collimated sputtering, laser ablated deposition, atomic layer deposition, molecular beam deposition, ionized physical vapor deposition, ion beam deposition, atomic layer deposition, hot filament chemical vapor deposition, screen printing, electroless metal deposition, electroplating, or electroless/immersion gold.

In some embodiments, the patterning of the electrically conducting material deposited on the portion of the first insulator layer to form a first electrically conducting material and the patterning of the electrically conducting material deposited on the spacer to form a second electrically conducting material comprises (i) application of a photolithographic photoresist coating to the electrically conducting material, (ii) optical imaging of the photolithographic photoresist coating through an optical mask, (iii) developing the photolithographic photoresist coating, (iv) etching the electrically conducting material, and (v) and removing the photolithographic photoresist coating.

In some embodiments, the photolithographic photoresist coating is a negative resist or a positive resist. In some embodiments, the photolithographic photoresist coating is an azide/isoprene negative resist, polymethylmethacrylate (PMMA), polymethylisopropyl ketone (PMIPK), poly-butene-1-sulfone (PBS), poly-(trifluoroethyl chloroacrylate) TFECA, copolymer-(α-cyano ethyl acrylate-α-amido ethyl acrylate) (COP), poly-(2-methyl pentene-1-sulfone) (PMPS), phenol-formaldehyde novolak resin, or LOR 3A.

In some embodiments, the photolithographic photoresist coating is developed by exposure to xylene, Stoddart solvent, n-butyl acetate, sodium hydroxide, potassium hydroxide, or tetramethylammonium hydroxide. In some embodiments, the etching of the electrically conducting material comprises wet etching, wet spray etching, vapor etching, plasma etching, ion beam etching or reactive ion etching. In some embodiments, the removing of the photolithographic photoresist coating comprises exposing the photolithographic photoresist coating to a strong acid, an acid-oxidant combination, an organic solvent stripper, or an alkaline stripper. In some embodiments, the depositing of the layer of electrically conducting material on the spacer and the portion of the first insulator layer that is exposed is performed by chemical vapor deposition.

In some embodiments, the depositing of the electrically conducting material on the spacer and the portion of the first insulator layer that is exposed is deposited at an angle with respect to the substrate. In some embodiments, this angle is between 0 and 2π radians, π/2 radians, or π/4 radians.

Yet another aspect of the present invention provides a method of processing a biosensor for binding a macromolecule. The method comprises etching a stack. The stack comprises a substrate, a first insulator layer overlaid on the substrate, a first electrically conducting material overlaid on the first insulator layer, a passivation layer overlaid on the first electrically conducting material, and a sacrificial insulator layer overlaid on the passivation layer. The etching forms a cavity that extends through the sacrificial insulator layer, the passivation layer, the first electrically conducting material, and the first insulator layer. The method continues with the formation of a second insulator layer at a bottom of the cavity. A second electrically conducting material is deposited on the second insulator layer. Finally, the sacrificial insulator layer overlaid on the passivation layer is removed.

In some embodiments, the etching comprises a wet etching process, a wet spray etching technique, a vapor etching process, plasma etching, ion beam etching, or reactive ion etching. In some embodiments, the substrate is made out of silicon and the formation of the second insulator layer comprises growing silicon oxide on the substrate.

Still another aspect of the present invention provides a biosensor. The biosensor comprises a plurality of devices. Each device is for binding a macromolecule. The biosensor comprises a substrate, a first insulator layer overlaid on the substrate, a first electrically conducting material overlaid on the insulator, and a passivation layer overlaid on the first electrically conducting material. Each device in the plurality of devices comprises (i) a cavity that extends through the passivation layer, the first electrically conducting material, and the first insulator layer, (ii) a second insulator layer in the cavity, and (iii) a second electrically conducting material on the second insulator layer. In some embodiments, the first insulator layer has a thickness of between 10 Angstroms and 10,000 Angstroms or between 100 Angstroms and 200 Angstroms. In some embodiments, the first insulator layer has a thickness of that is between 400 Angstroms and 800 Angstroms and optionally comprises silicon oxide.

Another aspect of the present invention provides a biosensor comprising a plurality of devices on a substrate. Each device in the plurality of devices is for binding a macromolecule. The substrate comprises a plurality of upper steps and a plurality of lower steps. Each upper step in the plurality of upper steps is associated with a lower step in the plurality of lower steps. For each device in the plurality of devices, a first electrically conducting material in the device overlays an upper step in the plurality of upper steps and a second electrically conducting material in the device overlays the lower step associated with the upper step. In some embodiments, the substrate is sealed onto a die attach surface of a package body and the package body includes a plurality of leads. In some embodiments, this package body is enclosed with an upper piece in a package. In some embodiments, this upper piece is ceramic and/or has an access hole. In some embodiments, the package is a dual in-line package, a single in-line package, or a ball grid array package. In some embodiments, the package is attached to a printed circuit board. In some embodiments, the printed circuit board is interfaced with a data acquisition card. In some embodiments, the printed circuit board is interfaced with a digital multimeter. In some embodiments, the demultiplexer has complementary metal-oxide semiconductor architecture.

In some embodiments, the plurality of devices comprises at least 100 devices, at least 10,000 devices, 10,000 to 10⁵ devices, or 10⁷ to 10⁹ devices. In some embodiments, the biosensor further comprises a plurality of bonding pads and a plurality of interconnects on the substrate. An interconnect in the plurality of interconnects joins a bonding pad in the plurality of bonding pads to a first electrically conducting material or a second electrically conducting material in a device in the plurality of devices. In some embodiments, a bonding pad in the plurality of bonding pads is connected to a lead in the plurality of leads.

Some embodiments in accordance with this aspect of the invention further provide a demultiplexer. The demultiplexer selectively connects a first electrically conducting material or a second electrically conducting material in a device in the plurality of devices to a bonding pad in the plurality of bonding pads.

A final aspect of the present invention provides a method of manufacturing a packaged biosensor. The method comprises depositing an electrically conducting layer onto a substrate. The substrate comprises a plurality of upper steps and a plurality of lower steps. Each upper step in the plurality of upper steps is associated with a lower step in the plurality of lower steps. In the method, the electrically conducting layer is patterned to form a plurality of electrode pairs, a plurality of bonding pads, and a plurality of interconnects. An interconnect, in the plurality of interconnects, joins an electrode, in the plurality of electrode pairs, to a bonding pad, in the plurality of bonding pads. Each electrode pair comprises a first electrode and a second electrode. The first electrode is on an upper step in the plurality of upper steps and the second electrode is on the lower step in the plurality of lower steps that is associated with the upper step.

The method continues with the sealing of the substrate to a die attach surface of a package body. The package body includes a plurality of leads. A bonding pad in the plurality of bonding pads is attached to a lead in the plurality of leads. Finally, the package body is enclosed with an upper piece, thereby manufacturing the packaged biosensor. In some embodiments, the method further comprises curing the biosensor in, for example, a curing oven.

In some embodiments, patterning creates a plurality of die, each die comprising a plurality of electrode pairs, a plurality of bonding pads and a plurality of interconnects on the substrate. In such embodiments, the method further comprises separating a die from the plurality of die. Sawing may perform this separation.

In some embodiments, sealing of the substrate to the die-attach surface of the package body comprises an epoxy die attachment technique or a eutectic die attachment technique. In some embodiments, the attaching of a bonding pad in the plurality of bonding pads to a lead in the plurality of leads is repeated. In some embodiments, this attaching is performed using a wire bonding technique, a flip-chip technique, or a beam-lead technique.

In some embodiments, the package body is a dual in-line package, single-in-line package, or a ball grid array package. In some embodiments, the patterning of the electrically conducting layer also forms a demultiplexer and this demultiplexer selectively connects an electrode in the plurality of electrode pairs to a bonding pad in the plurality of bonding pads. In some embodiments, the demultiplexer has complementary metal-oxide semiconductor architecture.

Some embodiments of the present invention further comprise attaching the biosensor to a printed circuit board. Some embodiments of the present invention further comprise interfacing the printed circuit board with a data acquisition card. Some embodiments of the method further comprise interfacing the biosensor with a digital multimeter.

Another aspect of the invention provides a method of detecting an analyte with a biosensor. The biosensor has any of the structures described herein. For example, in one embodiment, the biosensor comprises a plurality of devices. Each device in the plurality of devices occupies a different region on an insulator layer. The insulator layer is overlaid on the substrate. Each device in the plurality of devices comprises: (i) a first electrically conducting material; (ii) a spacer overlaid on a second portion of the different region of the insulator layer that is occupied by the device; and (iii) a second electrically conducting material. The first electrically conducting material is overlaid on a first portion of the different region of the insulator layer occupied by the device. The first portion of the different region on the insulator does not overlap the second portion of the different region on the insulator. The second electrically conducting material is overlaid on at least a portion of the spacer. The method comprises (a) attaching a first portion of a macromolecule to a first electrically conducting material and a second portion of said macromolecule to a second electrically conducting material in a device in said plurality of devices, (b) detecting a connection between the first electrically conducting material and the second electrically conducting material, (c) contacting the macromolecule with the analyte such that the analyte binds to the macromolecule thereby forming a macromolecule/analyte complex that comprises the macromolecule and the analyte; and, (d) detecting a difference in the connection between the first electrically conducting material and the second electrically conducting material.

### 5. BRIEF DESCRIPTION OF THE DRAWINGS

FIG. 1 illustrates a side plan view of a biosensor in accordance with one embodiment of the present invention.

FIG. 2 illustrates a side plan view of another biosensor in accordance with one embodiment of the present invention.

FIG. 3 illustrates a side plan view of yet another biosensor in accordance with one embodiment of the present invention.

FIGS. 4A-4D illustrate side plan views of biosensors in accordance with various embodiments of the present invention.

FIGS. 5A-5D illustrate side plan views of biosensors in accordance with various embodiments of the present invention.

FIGS. 6A-6D illustrate side plan, views of biosensors in accordance with various embodiments of the present invention.

FIGS. 7A-7D illustrate side plan views of biosensors in accordance with various embodiments of the present invention.

FIGS. 8A-8D illustrate side plan views of biosensors in accordance with various embodiments of the present invention.

FIGS. 9A-9D illustrate side plan views of biosensors in accordance with various embodiments of the present invention.

FIGS. 10A-10D illustrate side plan views of biosensors in accordance with various embodiments of the present invention.

FIGS. 11A-11D illustrate side plan views of biosensors in accordance with various embodiments of the present invention.

FIGS. 12A-12D illustrate side plan views of biosensors in accordance with various embodiments of the present invention.

FIGS. 13A-13D illustrate side plan views of biosensors in accordance with various embodiments of the present invention.

FIGS. 14A-14D illustrate side plan views of biosensors in accordance with various embodiments of the present invention.

FIGS. 15A-15D illustrate side plan views of biosensors in accordance with various embodiments of the present invention.

FIGS. 16A-16D illustrate side plan views of biosensors in accordance with various embodiments of the present invention.

FIGS. 17A-17D illustrate side plan views of biosensors in accordance with various embodiments of the present invention.

FIGS. 18A-18D illustrate side plan views of biosensors in accordance with various embodiments of the present invention.

FIGS. 19A-19D illustrate side plan views of biosensors in accordance with various embodiments of the present invention.

FIGS. 20A-20D illustrate side plan views of biosensors in accordance with various embodiments of the present invention.

FIGS. 21A-21D illustrate side plan views of biosensors in accordance with various embodiments of the present invention.

FIGS. 22A-22D illustrate side plan views of biosensors in accordance with various embodiments of the present invention.

FIGS. 23A-23D illustrate side plan views of biosensors in accordance with various embodiments of the present invention.

FIGS. 24A-24D illustrate side plan views of biosensors in accordance with various embodiments of the present invention.

FIGS. 25A-25D illustrate side plan views of biosensors in accordance with various embodiments of the present invention.

FIGS. 26A-26D illustrate side plan views of biosensors in accordance with various embodiments of the present invention.

FIGS. 27A-27D illustrate side plan views of biosensors in accordance with various embodiments of the present invention.

FIGS. 28A-28D illustrate side plan views of biosensors in accordance with various embodiments of the present invention.

FIGS. 29A-29D illustrate side plan views of biosensors in accordance with various embodiments of the present invention.

FIGS. 30A-30D illustrate side plan views of biosensors in accordance with various embodiments of the present invention.

FIGS. 31A-31D illustrate side plan views of biosensors in accordance with various embodiments of the present invention.

FIGS. 32A-32D illustrate side plan views of biosensors in accordance with various embodiments of the present invention.

FIGS. 33A-33D illustrate side plan views of biosensors in accordance with various embodiments of the present invention.

FIGS. 34A-34D illustrate side plan views of biosensors in accordance with various embodiments of the present invention.

FIGS. 35A-35D illustrate side plan views of biosensors in accordance with various embodiments of the present invention.

FIGS. 36A-36D illustrate side plan views of biosensors in accordance with various embodiments of the present invention.

FIGS. 37A-37D illustrate side plan views of biosensors in accordance with various embodiments of the present invention.

FIGS. 38A-38C illustrate side plan views of biosensors in accordance with various embodiments of the present invention.

FIGS. 39A-39D illustrate side plan views of biosensors in accordance with various embodiments of the present invention.

FIGS. 40A-40D illustrate side plan views of biosensors in accordance with various embodiments of the present invention.

FIGS. 41A-41D illustrate side plan views of biosensors in accordance with various embodiments of the present invention.

FIGS. 42A-42D illustrate side plan views of biosensors in accordance with various embodiments of the present invention.

FIGS. 43A-43D illustrate side plan views of biosensors in accordance with various embodiments of the present invention.

FIGS. 44A-44D illustrate side plan views of biosensors in accordance with various embodiments of the present invention.

FIGS. 45A-45D illustrate side plan views of biosensors in accordance with various embodiments of the present invention.

FIGS. 46A-46D illustrate side plan views of biosensors in accordance with various embodiments of the present invention.

FIGS. 47A-47D illustrate side plan views of biosensors in accordance with various embodiments of the present invention.

FIGS. 48A-48D illustrate side plan views of biosensors in accordance with various embodiments of the present invention.

FIGS. 49A-49D illustrate side plan views of biosensors in accordance with various embodiments of the present invention.

FIGS. 50A-50D illustrate side plan views of biosensors in accordance with various embodiments of the present invention.

FIGS. 51A-51D illustrate side plan views of biosensors in accordance with various embodiments of the present invention.

FIGS. 52A-52D illustrate side plan views of biosensors in accordance with various embodiments of the present invention.

FIGS. 53A-53D illustrate side plan views of biosensors in accordance with various embodiments of the present invention.

FIGS. 54A-54D illustrate side plan views of biosensors in accordance with various embodiments of the present invention.

FIGS. 55A-55D illustrate side plan views of biosensors in accordance with various embodiments of the present invention.

FIGS. 56A-56D illustrate side plan views of biosensors in accordance with various embodiments of the present invention.

FIGS. 57A-57D illustrate side plan views of biosensors in accordance with various embodiments of the present invention.

FIGS. 58A-58D illustrate side plan views of biosensors in accordance with various embodiments of the present invention.

FIGS. 59A-59D illustrate side plan views of biosensors in accordance with various embodiments of the present invention.

FIGS. 60A-60D illustrate side plan views of biosensors in accordance with various embodiments of the present invention.

FIGS. 61A-61B illustrate side plan views of biosensors in accordance with various embodiments of the present invention.

FIGS. 62A-62D illustrate side plan views of biosensors in accordance with various embodiments of the present invention.

FIGS. 63A-63D illustrate side plan views of biosensors in accordance with various embodiments of the present invention.

FIGS. 64A-64D illustrate side plan views of biosensors in accordance with various embodiments of the present invention.

FIGS. 65A-65D illustrate side plan views of biosensors in accordance with various embodiments of the present invention.

FIGS. 66A-66D illustrate side plan views of biosensors in accordance with various embodiments of the present invention.

FIGS. 67A-67D illustrate side plan views of biosensors in accordance with various embodiments of the present invention.

FIGS. 68A-68D illustrate side plan views of biosensors in accordance with various embodiments of the present invention.

FIGS. 69A-69D illustrate side plan views of biosensors in accordance with various embodiments of the present invention.

FIGS. 70A-70D illustrate side plan views of biosensors in accordance with various embodiments of the present invention.

FIGS. 71A-71D illustrate side plan views of biosensors in accordance with various embodiments of the present invention.

FIGS. 72A-72D illustrate side plan views of biosensors in accordance with various embodiments of the present invention.

FIGS. 73A-73D illustrate side plan views of biosensors in accordance with various embodiments of the present invention.

FIGS. 74A-74D illustrate side plan views of biosensors in accordance with various embodiments of the present invention.

FIGS. 75A-75D illustrate side plan views of biosensors in accordance with various embodiments of the present invention.

FIGS. 76A-76D illustrate side plan views of biosensors in accordance with various embodiments of the present invention.

FIGS. 77A-77B illustrate side plan views of biosensors in accordance with various embodiments of the present invention.

FIG. 78 illustrates a top plan view of a biosensor array in accordance with one embodiment of the present invention.

FIG. 79 illustrates a side plan view of a device in accordance with one embodiment of the present invention.

FIGS. 80A-80I illustrate various processing stages in the manufacture of a biosensor in accordance with one embodiment of the present invention.

FIG. 81 illustrates a side plan view of a biosensor as well as the method for manufacturing the biosensor in accordance with one embodiment of the present invention.

FIG. 82 illustrates a side plan view of a biosensor as well as the method for manufacturing the biosensor in accordance with one embodiment of the present invention.

FIG. 83 illustrates a side plan view of a biosensor as well as the method for manufacturing the biosensor in accordance with one embodiment of the present invention.

FIG. 84 illustrates a side plan view of a biosensor as well as the method for manufacturing the biosensor in accordance with one embodiment of the present invention.

FIG. 85 illustrates a side plan view of a biosensor as well as the method for manufacturing the biosensor in accordance with one embodiment of the present invention.

FIG. 86 illustrates a side plan view of a biosensor as well as the method for manufacturing the biosensor in accordance with one embodiment of the present invention.

FIG. 87 illustrates a side plan view of a biosensor as well as the method for manufacturing the biosensor in accordance with one embodiment of the present invention.

FIG. 88 illustrates a side plan view of a biosensor as well as the method for manufacturing the biosensor in accordance with one embodiment of the present invention.

FIG. 89 illustrates a side plan view of a biosensor as well as the method for manufacturing the biosensor in accordance with one embodiment of the present invention.

FIG. 90 illustrates a side plan view of a biosensor as well as the method for manufacturing the biosensor in accordance with one embodiment of the present invention.

FIG. 91 illustrates a side plan view of an array of biosensors in accordance with one embodiment of the present invention.

FIGS. 92A-92F illustrate processing steps used to manufacture an illustrative device 144 in accordance with one embodiment of the invention.

FIGS. 93A-93E illustrate methods of packaging biosensors in accordance with one embodiment of the present invention.

FIG. 94 illustrates a packaged biosensor in accordance with one embodiment of the present invention.

FIG. 95 illustrates an array of biosensors in accordance with one embodiment of the present invention.

Like reference numerals refer to corresponding parts throughout the several views of the drawings.

### 6. DETAILED DESCRIPTION

The present invention provides stable biosensors that have numerous commercial applications. The biosensors of the present invention do not have to be pre-treated with salts and/or enzyme cofactors. An overview of the structure of the biosensors of the present invention is described in Section 6.1. Various features of the biosensors of the present invention are described in Sections 6.2 through 6.12. Sections 7.1 through 7.10 describe novel methods used to make the biosensors of the present invention. Section 7.11 describes planar arrays of biosensors in accordance with the present invention. Section 7.12 describes methods for analyte detection and quantification. Sections 7.13, 7.14, and 7.15 respectively describe cassettes, integrated assay devices, and kits in accordance with the present invention. Section 7.16 describes methods for monitoring electron transfer through bound macromolecule/analyte complexes using the present invention. Section 8 describes packages biosensors and methods for packaging biosensors in accordance with the present invention. Section 9 describes biosensors that comprise a very dense array of electrode pairs. Section 10 describes biosensors that include a plurality of arrays in a microwell plate format.

6.1 Biosensor Configurations in Accordance with Various Embodiments of the Present Invention

The present invention provides a number of different biosensor configurations. Each biosensor configuration provides unique advantages. For example, some biosensor configurations are advantageous because of their ease of manufacture. Other biosensor configurations of the present invention are advantageous because of the electrical isolation they provide between electrodes within the biosensor. This electrical isolation lowers leakage currents. Still other biosensors of the present invention are advantageous because of their enhanced assay sensitivity.

6.1.1 Illustrative Biosensor with Non-Overlapping Electrodes

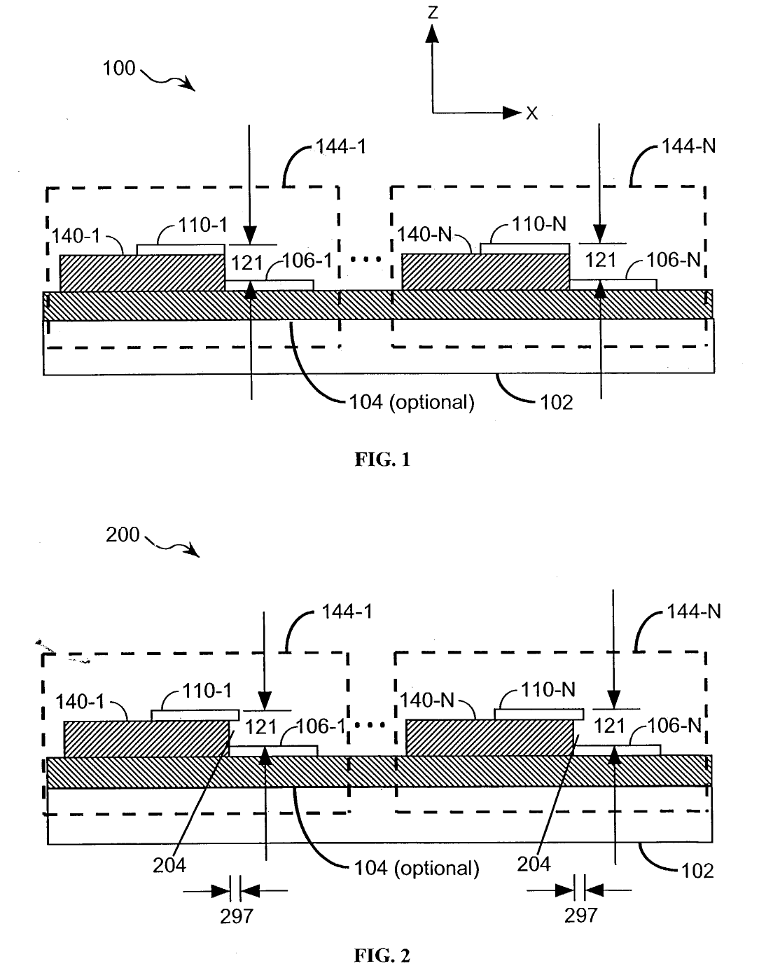

FIG. 1 illustrates a side plan view of a novel biosensor 100 in accordance with one embodiment of the present invention. Biosensor 100 includes non-overlapping materials 106 and 110. In some embodiments, a predetermined distance 121 in the z-dimension separates the top of material 106 and the top of material 110. In some embodiments, materials 106 and 110 are made of conductive, semi-conductive, or resistive materials. In some embodiments, predetermined distance 121 is achieved by overlaying material 110 on a spacer 140.

As illustrated in FIG. 1, spacer 140 and materials 106 and 110 comprise a discrete device 144. In instances where materials 106 and 110 are electrodes, each device 144 has an electrode-insulator-electrode configuration. It will be appreciated that each device 144 may serve as an independent sensor for a particular application.

An advantage of the present invention is that predetermined distance 121 can be precisely controlled by separating materials 106 and 110 in the z dimension (FIG. 1) rather than the x dimension or the y dimension (not shown, perpendicular to the plane of FIG. 1). Separation in the z dimension is controlled using precise semiconductor manufacturing techniques that are described in more detail in Section 6.2, below. The ability to precisely control the separation (distance 121) of closely spaced materials 106 and 110 has use in a broad range of fields. Examples include, but are not limited to, the construction of biosensors, the assembly of nanocircuits and other nanostructures, computer memory, electronic and computer switches, material science, construction, surface science, medical devices, medical therapeutics and more.

In one embodiment of the present invention, materials 106 and 110 are electrodes. A macromolecule 120, or pool of macromolecules 120, can be directly bound to electrodes 106 and 110. Alternatively, a macromolecule or pool of macromolecules can be coupled to the first electrode 106 and/or the second electrode 110 through one or more linkers or functional groups (e.g., thiols). Generally speaking, macromolecules 120 are attached to electrodes 106 and 110 in such a manner that sufficient area on the macromolecule 120 is left so that the macromolecule 120 can bind with its “cognate” target molecule. When macromolecule 120 binds its cognate target molecule, a binding agent/target molecule complex is formed whose conductivity is different than the conductive of macromolecule 120 alone. This change in conductivity is readily detected indicating the presence and/or concentration of macromolecule 120 on the biosensor (e.g., on biosensor 100).

In reference to FIG. 1, one embodiment of the present invention provides a biosensor 100 comprising a plurality of devices 144 on a substrate 102. Each device 144 in the plurality of devices 144 occupies a different region on an optional insulator layer 104. The optional insulator layer 144 is overlaid on substrate 102. Furthermore, each device 144 in the plurality of devices comprises (i) a first electrically conducting material 106 having a top surface, wherein the first electrically conducting material 106 is overlaid on a first portion of optional insulator layer 104, (ii) a spacer 140 overlaid on a second portion of the insulator layer 104, and (iii) a second electrically conducting material 110 overlaid on a portion of spacer 144. As illustrated in FIG. 1, the first electrically conducting material 106 and spacer 144 abut each other. Furthermore, for any given device 144 in the plurality of devices, the first portion of insulator layer 104 occupied by the device does not overlap with the second portion of insulator layer 104 occupied by the device. As used herein, a device 144 “occupies” that portion of insulator layer 104 which is overlaid by a component (e.g. material 106, spacer 140, etc.) of the device. In embodiments where insulator 104 is not used, each device 144 occupies a portion of substrate 102 and material 106 and spacer 140 each directly overlay a portion of substrate 102.

In some embodiments in accordance with FIG. 1, a distance between a plane including the top surface of the first electrically conducting material 106 and a plane including the top surface of the second electrically conducting material 110 is less than 500 Angstroms. In some embodiments of the present invention, the distance between a plane including the top surface of the first electrically conducting material 106 and a plane including the top surface of the second electrically conducting material 110 is less than 250 Angstroms. In still other embodiments, a distance between a plane including the top surface of the first electrically conducting material and a plane including the top surface of the second electrically conducting material is less than 100 Angstroms. In still other embodiments of the present invention, a distance between a plane including the top surface of the first electrically conducting material 106 and a plane including the top surface of the second electrically conducting material 110 is between 40 Angstroms and 60 Angstroms.

6.1.2 Illustrative Biosensor with Overlapping Electrodes

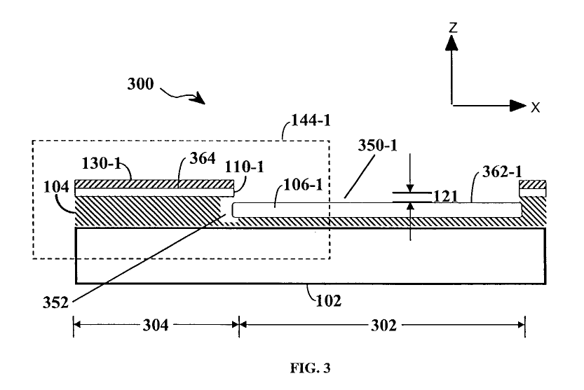

FIG. 2 illustrates a side plan view of a novel biosensor 200 in accordance with another embodiment of the present invention. Biosensor 200 is similar to biosensor 100 (FIG. 1) with the exception that materials 106 and 110 overlap each other. As illustrated in FIG. 2, materials 106 and 110 overlap, thereby creating a cavity 204. Furthermore, in the embodiment illustrated in FIG. 2, there is no composition, such as spacer 140 or insulator layer 104 in cavity 204.

The width 297 of cavity 204 defines the amount that materials 106 and 110 overlap in biosensor 200 (FIG. 2). In some embodiments of the present invention, cavity 204 has a width 297 that is 10,000 Angstroms or less, 5,000 Angstroms or less, 1000 Angstroms or less, 500 Angstroms or less, 300 Angstroms or less, 250 Angstroms or less, 200 Angstroms or less, 150 Angstroms or less, 100 Angstroms or less, 50 Angstroms or less, or 20 Angstroms or less.

6.1.3 Illustrative Biosensor with Cavity in the Insulator Layer

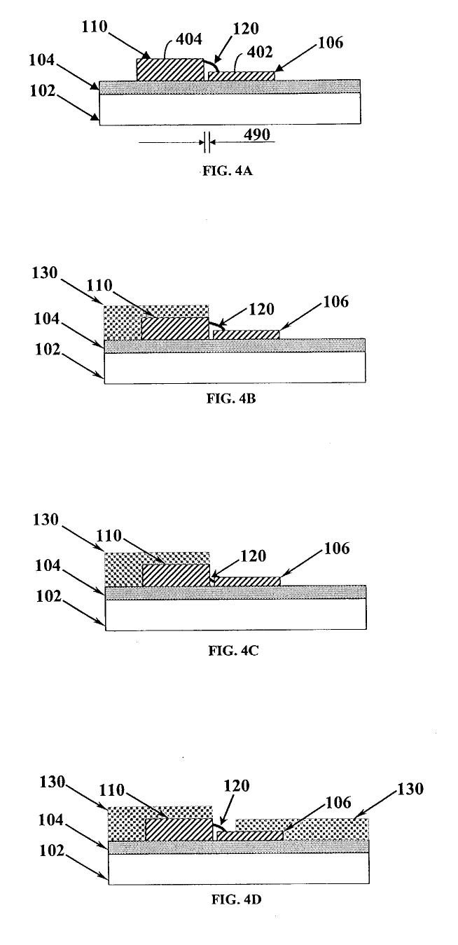

FIG. 3 illustrates a side plan view of yet another biosensor 300 in accordance with another embodiment of the present invention. Biosensor 300 includes substrate 102. Insulator layer 104 overlays substrate 102. As illustrated in FIG. 3, cavity 350 is introduced into portion 302 of insulator layer 104 and material 106 is deposited in cavity 350. Further, material 110 is overlaid on portion 304 of insulator layer (FIG. 3). In this way, insulator layer 104 is used to separate material 106 from material 110 in the z dimension. Finally, optional passivator 130 is overlaid on material 110 to complete device 144. Biosensor 300 (FIG. 3) differs from biosensors 100 and 200 in the sense that biosensor 300 does not use a spacer 140 to separate material 106 from material 110. Rather, in biosensor 300, desired separation between material 106 and 110 is achieved by the formation of cavity 350.

Some embodiments of the present invention provide a biosensor comprising a plurality of devices 144 on a substrate 102 (FIG. 3). Each device in the plurality of devices occupies a different region on an insulator layer 104. The insulator layer 104 is overlaid on substrate 102 and each device 144 in the plurality of devices 144 is associated with a different cavity 350 in the insulator layer. Only one such device 144 is shown in FIG. 3. However, biosensor 300 may have any number of devices 144 and each such device 144 includes a cavity 350. Each device 144 in the plurality of devices 144 of biosensor 300 comprise (i) a first electrically conducting material 106 having a top surface 362, wherein material 106 is overlaid in the different cavity 350 associated with the device 144, (ii) a second electrically conducting material 110 having a top surface 364, wherein material 110 is overlaid on insulator 104 in a region outside of the cavity 350 associated with the device 144; and (iii) a passivation layer 130 overlaid on material 110.

Referring again to FIG. 3, in some embodiments of the present invention, an additional cavity 352 is etched into insulator layer 104 to further isolate material 106 from material 110. In some embodiments of the present invention, each cavity 350 in biosensor 300 has a width 302 of between 900 Angstroms and 20,000 Angstroms, a width 302 between 500 Angstroms and 900 Angstroms, a width 302 between 10,000 Angstroms and 100,000 Angstroms, or a width 302 that is greater than 50,000 Angstroms.

6.1.4 Additional Biosensor Configurations

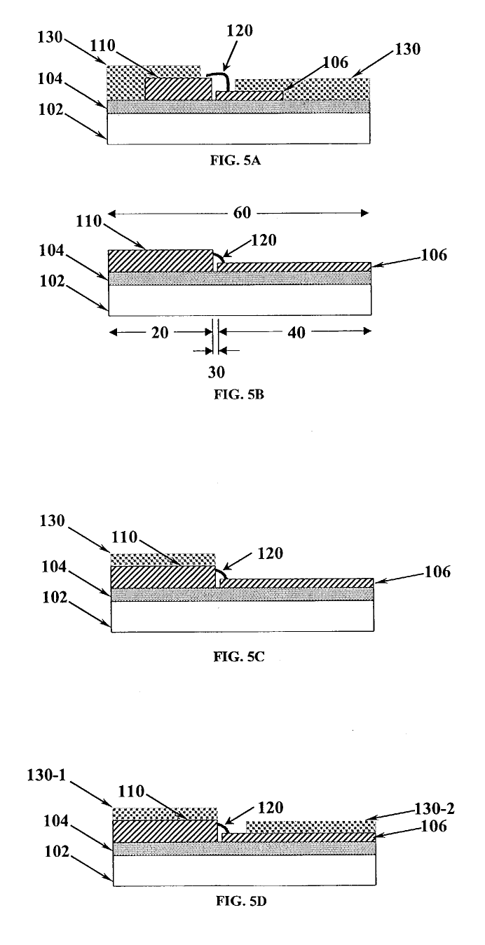

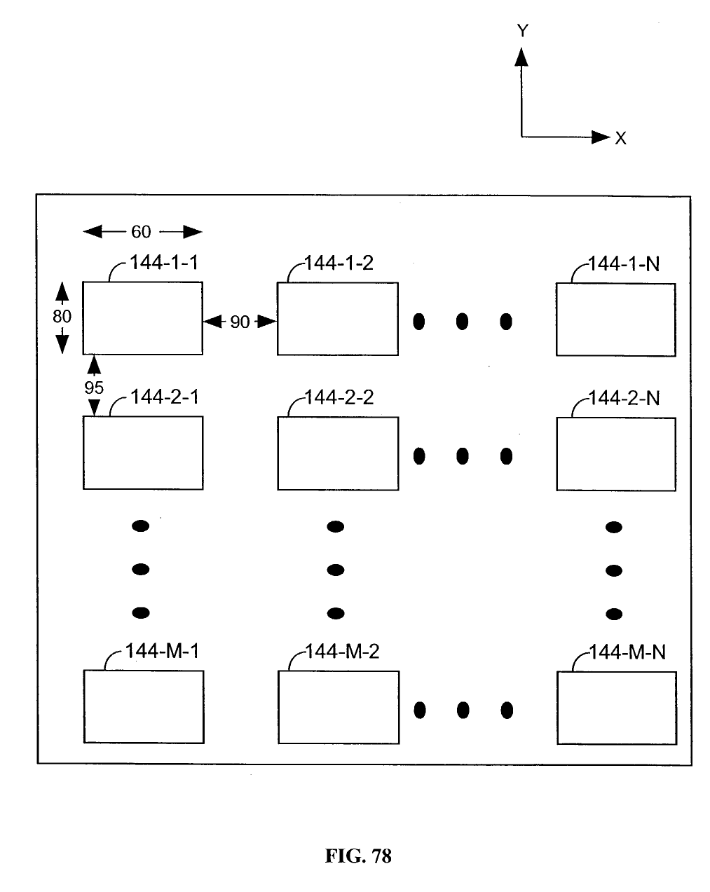

Some embodiments of biosensors (e.g. biosensors 100, 200 and 300) in accordance with the present invention have been described. Attention now turns to FIGS. 4A through 77B, which illustrate plan views of several biosensors in accordance with additional embodiments of the present invention. Although only a single device 144 is shown in the biosensor configurations illustrated in FIGS. 4A through 77B, it will be appreciated that any number of devices 144 may be found in the biosensors illustrated in FIGS. 4A through 77B.

Attaching specific entities (e.g. macromolecules) at locations on materials 106 and 110 in biosensors 100, 200, 300, or the biosensors illustrated in FIGS. 4A through 77B may be used either to bridge the entity between materials 106 and 110 or to localize the entities for further reactions. In the case where entities are bridged, one end of the entity may be attached to, for example, material 106-1 while another end of the entity may be attached to, for example, material 110-1 (FIG. 1). In the case where materials 106 and 110 are electrodes, such a bridging configuration can be used as a biosensor. That is, changes in the electrical conductivity of the entity can be precisely measured. Such measurements may be used to detect when a foreign object binds to the bridged entity. As such, the biosensors of the present invention may be used as a sensor of molecular events. Such sensors can be used in many fields including, but not limited to, biology, chemistry, physics, genomics and proteomics.

FIG. 4A illustrates a biosensor in which a spacer 140 (FIG. 1) is not used. Desired separation between materials 110 and 106 is achieved by the difference in the thickness of materials 110 and 106. In the biosensor illustrated in FIG. 4A, one portion (e.g., a first end) of macromolecule 120 binds to the upper surface of material 106 and another portion (e.g., a second end) of macromolecule 120 binds to a side of material 106. In some embodiments of the present invention, material 106 and material 110 are separated by a distance 490 that is 5 Angstroms or greater, 10 Angstroms or greater, 20 Angstroms or greater, 30 Angstroms or greater, or 100 Angstroms or greater (FIG. 4A).

Referring again to FIG. 4A, some embodiments of the present invention provide a biosensor comprising a plurality of devices 144 on a substrate 102. Each device 144 in the plurality of devices 144 occupies a different region on an insulator layer 104. Insulator layer 104, in turn, overlays substrate 102. Furthermore, each device 144 in the plurality of devices 144 comprises (i) a first electrically conducting material 106 having a top surface 402 and (ii) a second electrically conducting material 110 having a top surface 404. Material 106 is overlaid on a first portion of the region of insulator layer 104 occupied by device 144 and material 110 is overlaid on a second portion of the region of insulator layer 104 occupied by device 144. Furthermore, this first portion of insulator layer 104 does not overlap with the second portion of insulator layer 104.

The only difference between the biosensor illustrated in FIG. 4B and the biosensor illustrated in FIG. 4A is the presence of a passivation layer 130 in the biosensor illustrated in FIG. 4B. The materials used to form passivation layer 130 are described in more detail in Section 6.6, below. Passivation layer 130 helps to localize where a macromolecule 120 will bind to material 110. As shown in FIG. 4B, passivation layer 130 covers each exposed surface of material 110 except the right-hand side of material 110. Thus, macromolecule 120 can only bind to the exposed portion of material 110.

The distance between material 106 and material 110 must be optimized in order to achieve a measurable electrical conductivity signal using a macromolecule 120. Accordingly, the present invention provides a number of different configurations in order to provide a suitable distance between materials 106 and 110. One such configuration is illustrated in FIG. 4C. In the biosensor of FIG. 4C, the spacing between materials 106 and 110 is used provide the appropriate spacing between materials 106 and 110. In the biosensor illustrated in FIG. 4C, macromolecule 120 binds to opposing surfaces of material 106 and material 110. Therefore, the distance between material 106 and material 110 determines the distance that an electrical current must travel across a macromolecule 120.

In the biosensor illustrated in FIG. 4D, macromolecule 120 binds to a top surface of material 106 and a sidewall of material 110. This specific binding configuration is facilitated by the use of passivation layers 130 that cover portions of materials 106 and 110, thereby preventing macromolecule 120 from binding to the covered portions.

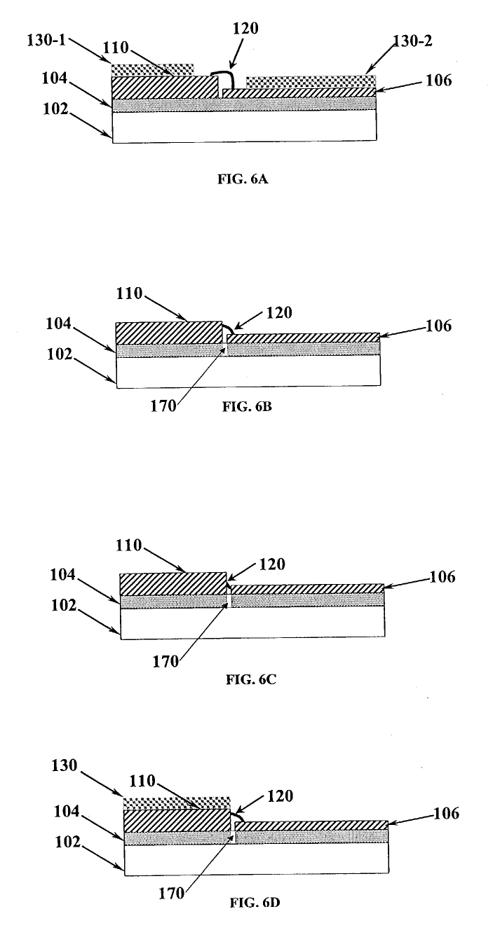

In the biosensor illustrated in FIG. 5A, macromolecule 120 binds to a top surface of material 106 and a top surface of material 110. Similar to the biosensor illustrated in FIG. 4D, the specific binding configuration between the biosensor and macromolecule 120 is facilitated by the use of passivation layers 130 that cover portions of materials 106 and 110, thereby preventing macromolecule 120 from binding to the covered portions of materials 106 and 110. In particular, unlike the case in FIG. 4D, the passivation layer 130 that covers material 110 does not completely cover the top portion of layer 110. This allows macromolecule 120 to bind to the exposed portion of material 110.

The biosensor of FIG. 5B is identical to the biosensor of FIG. 4A, with the exception that materials 106 and 110 extend out to the entire length of substrate 102. Thus, as illustrated in FIG. 5C, when a passivation layer 130 is used on a biosensor such as that illustrated in FIG. 5B, only the top surface of material 110 needs to be covered. Thus, in some instances the biosensor illustrated in FIG. 5C can be manufactured more quickly and/or more inexpensively then the biosensor illustrated in FIG. 4B.

In a variation of the biosensor illustrated in FIG. 5C, the biosensor illustrated in FIG. 5D uses two passivation layers 130. Passivation layer 130-1 completely overlays material 110 while passivation layer 130-2 overlays only a portion of material 106. Because of the passivation layers 130 in the biosensor illustrated in FIG. 5D, macromolecule 120 spans the top of material 106 and a sidewall of material 110.

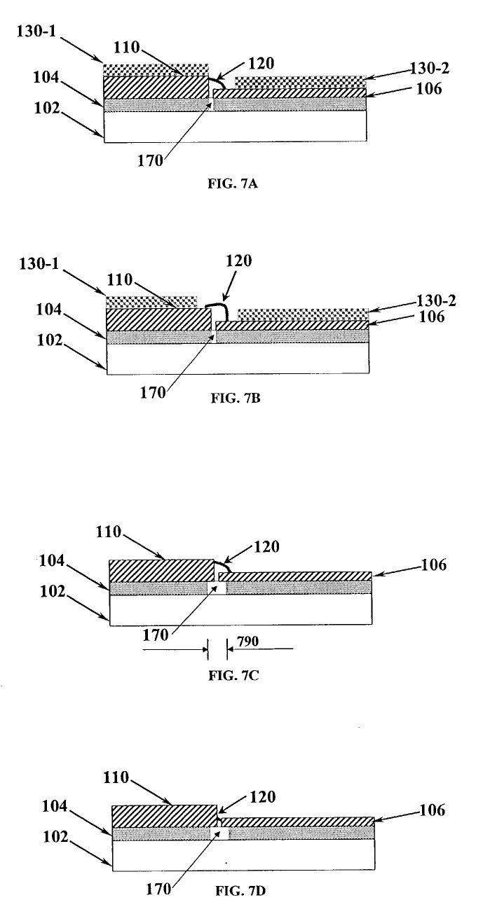

The biosensor illustrated in FIG. 6A employs two passivation layers 130. Passivation layer 130-1 overlays a portion of material 110 and passivation layer 130-1 overlays a portion of material 106. Thus, a top region of material 110 and material 106 remains exposed even after the passivation layers 130 are overlaid onto materials 106 and 100. In the biosensor illustrated in FIG. 6A, macromolecule 120 spans the top of material 106 and the top of material 110.

In some embodiments in accordance with the biosensor illustrated in FIG. 6B, material 110 is thicker than material 106. However, some biosensor embodiments of the present invention include devices 144 that have the configuration shown in FIGS. 4A, 4B, 4C, 4D 5A, 5B, 5C, 5D, 6A, 6B, 6C, 6D, 7A, 7B, 7C, 7D, 8A, 8B, or 8C wherein material 110 and material 106 have the same thickness. Therefore, in some embodiments in accordance with FIG. 6B, material 106 and material 110 have the same thickness (not shown). In some embodiments in accordance with FIG. 6B, macromolecule 120 spans from the top of material 106 to a sidewall of material 110. In addition, there is a gap 170 in insulator layer 104. Gap 170 coincides with the separation between materials 106 and 110. Gap 170 effectively increases the distance of the path from material 106 to material 110 (or vice versa) that undesirable leakage current must travel when macromolecule 120 is not used as an electrical conduit. Thus, the biosensor illustrated in FIG. 6B is advantageous because it has improved insulator properties that prevent the short circuiting of materials 106 and 110. The biosensor illustrated in FIG. 6C is identical to the biosensor illustrated in FIG. 6B with the exception that macromolecule 120 spans from a side-wall of material 106 to the opposing side-wall of material 110. The biosensor illustrated in FIG. 6D is identical to the biosensor illustrated in FIG. 6D with the exception that the biosensor illustrated in FIG. 6D includes a passivation layer 130 that overlays material 110. The use of passivation layer 130 in the biosensor illustrated in FIG. 6D helps force macromolecule 120 to span the top of material 106 and a sidewall of material 110.

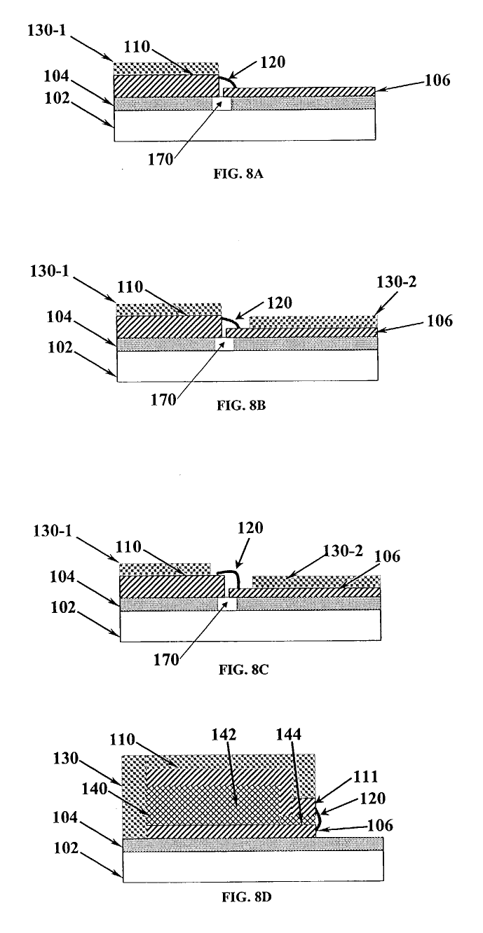

The biosensor illustrated in FIG. 7A is identical to the biosensor illustrated in FIG. 5D with the exception that a gap 170 in insulator layer 104 coincides with the separation between materials 106 and 110. Gap 170 effectively increases the distance of the path from material 106 to material 110 (or vice versa) that undesirable leakage current must travel when macromolecule 120 is not used as an electrical conduit. Thus, the biosensor illustrated in FIG. 7A is advantageous in some applications because it has improved insulator properties that prevent undesirable leakage currents.

The biosensor illustrated in FIG. 7B is identical to the biosensor illustrated in FIG. 6A with the exception that a gap 170 in insulator layer 104 coincides with the separation between materials 106 and 110. Thus, the biosensor illustrated in FIG. 7B is advantageous in some applications because it has improved insulator properties that prevent the short circuiting of materials 106 and 110.

The biosensor illustrated in FIG. 7C includes a gap 170 in insulator 104. Like previously illustrated biosensors, gap 170 coincides with the separation between materials 106 and 110. However, in the biosensor illustrated in FIG. 7C, gap 170 is, in fact, wider than the separation between materials 106 and 110. This wider gap 170 serves to further increase the distance of the path from material 106 to material 110 (or vice versa) that current must travel if macromolecule 120 is not used as an electrical conduit. Thus, the biosensor illustrated in FIG. 7C is advantageous because it has improved insulator properties that prevent the short circuiting of materials 106 and 110. In some embodiments of the present invention, gap 170 has a width 790 (FIG. 7C) that is between 60 Angstroms and 500 Angstroms, between 60 Angstroms and 10,000 Angstroms, between 60 Angstroms and 30,000 Angstroms, between 60 Angstroms and 100,000 Angstroms, or between 50 Angstroms and 1,000,000 Angstroms.

In the biosensor illustrated in FIG. 7C, macromolecule 120 spans from the top of material 106 to a sidewall of material 110. The biosensor illustrated in FIG. 7D is identical to the biosensor illustrated in FIG. 7C with the exception that macromolecule 120 spans from the side-wall of material 106 to the opposing side-wall of material 110.

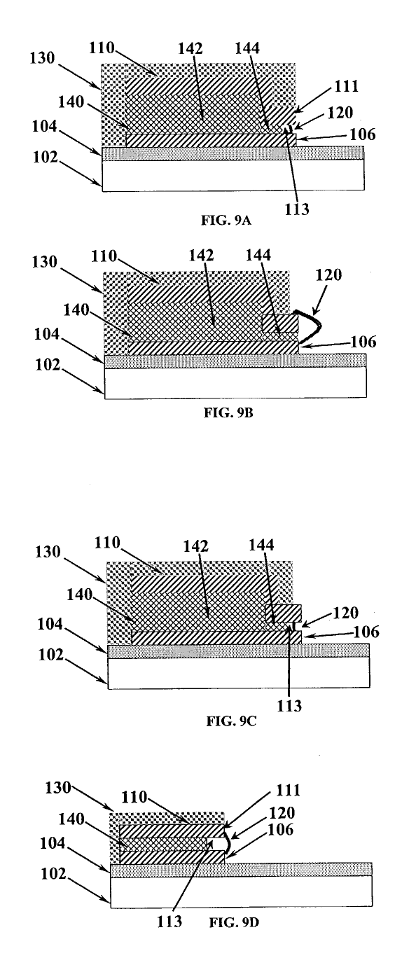

The biosensor illustrated in FIG. 8A is identical to the biosensor illustrated in FIG. 7C with the exception that a passivation layer 130 overlays material 110. Passivation layer 130 helps to force macromolecule 120 to span from the sidewall of material 10 to the top of material 106. The biosensor illustrated in FIG. 8B is identical to the biosensor illustrated in FIG. 8A with the exception that a second passivation layer 130-2 overlays a portion of layer 106. The use of passivation layers 130-1 and 130-2 in the biosensor illustrated in FIG. 8B helps to force macromolecule 120 to span from the top of material 106 to a sidewall of material 110. The biosensor illustrated in FIG. 8C is identical to the biosensor illustrated in FIG. 8B with the exception that passivation layer 130 only overlays a portion of material 110. Thus, an upper portion of material 110 and material 106 is exposed even after passivation layers 130-1 and 130-2 are overlaid on the biosensor. In the biosensor illustrated in FIG. 8C, macromolecule 120 spans the exposed portion of material 110 and the exposed portion of material 106.

The biosensor illustrated in FIG. 8D includes a substrate 102 and an optional insulator 104 that is overlaid on substrate 102. In the case where optional insulator 104 is not used, material 106 is overlaid on substrate 102 (not shown). In the case where optional insulator 104 is used, material 106 is overlaid onto a portion of insulator 104. Next, a spacer 140 is overlaid on material 106. Spacer 140 has two segments, a thick segment 142 and a thin segment 144. The thickness of thin segment 142 defines a separation distance between material 106 and material 110, which is overlaid on spacer 140. Further, passivation layer 130 overlays all exposed surfaces of material 110 except sidewall 111. Passivation layer 130 helps to cause macromolecule 120 to span from sidewall 111 of material 110 to the exposed sidewall of material 106. In some embodiments, a distance between electrically conducting material 106 and electrically conducting material 110 is between 60 Angstroms and 1 Angstroms, between 80 Angstroms and 300 Angstroms, or between 100 Angstroms and 200 Angstroms.

Referring again to FIG. 8D, one embodiment of the present invention provides a biosensor that includes a plurality of devices 144 on a substrate 102. Each device 144 in the plurality of devices 144 occupies a different region on an insulator layer 104. Furthermore, each device 144 in the plurality of devices 144 is capable of binding to a macromolecule 120. In this biosensor, the insulator layer 104 is overlaid on the substrate 102. At least one device 144 in the plurality of devices comprises (i) a first electrically conducting material 106, (ii) a spacer 140 overlaying the first electrically conducting material 106, (iii) a second electrically conducting material 110 overlaid on the spacer 140, and (iv) a passivation layer 130 overlaid on the second electrically conducting material 110. In this device, the first electrically conducting material 106 is overlaid on the different region of the insulator layer occupied by the device 144. Further, the spacer 140 includes a step region 144 and a main region 142 and the step region 144 of the spacer 140 is not as thick as the main region 142 of the spacer 140.

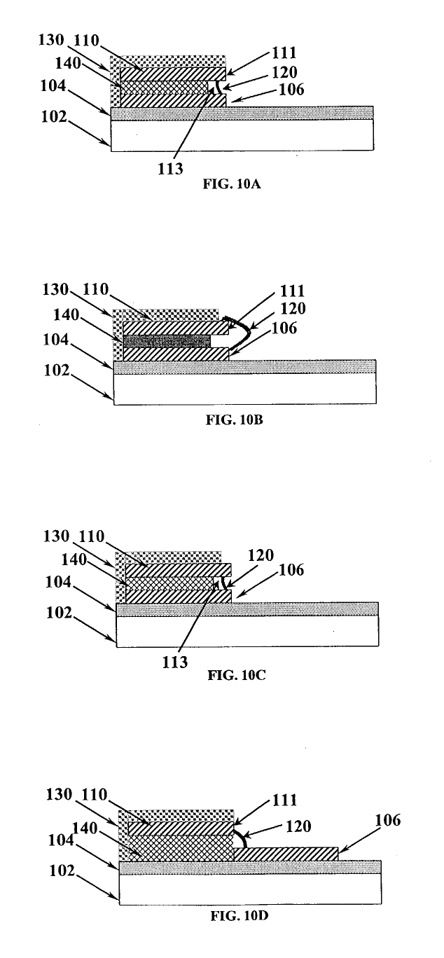

The biosensor illustrated in FIG. 9A is identical to the biosensor illustrated in FIG. 8D with the exception that thin segment 142 of spacer 140 does not extend all the way to edge 111 of material 110. Because of this, a cavity 113 forms in the biosensor illustrated in FIG. 9A. Macromolecule 120 spans from material 106 to material 110 in cavity 113 of the biosensor illustrated in FIG. 9A.

The biosensor illustrated in FIG. 9B is identical to the biosensor illustrated in FIG. 8D with the exception that passivation layer 130 does not cover the entire upper surface of material 110. In the biosensor illustrated in FIG. 9B, macromolecule 120 spans from the exposed sidewall of material 106 to the exposed upper portion of material 110.

The biosensor illustrated in FIG. 9C is identical to the biosensor illustrated in FIG. 9A with the exception that passivation layer 130 does not overlay the entire upper surface of material 110. Macromolecule 120 spans from material 106 to material 110 in cavity 113 of the biosensor illustrated in FIG. 9C.

The biosensor illustrated in FIG. 9D includes a substrate 102 and an optional insulator 104 overlaid on substrate 102. In the case where optional insulator 104 is not used, material 106 is overlaid on substrate 102 (not shown). In the case where optional insulator 104 is used, material 106 is overlaid onto a portion of insulator 104. Next, a spacer 140 is overlaid onto a portion of material 106. The thickness of spacer 140 defines a separation distance between material 106 and material 110, which is overlaid on spacer 140. Further, a passivation layer 130 overlays all exposed surfaces of material 110 except sidewall 111 of material 110. Passivation layer 130 causes macromolecule 120 to span from sidewall 111 of material 110 to the exposed sidewall of material 106.

Referring again to FIG. 9D, one embodiment of the present invention provides a biosensor comprising a plurality of devices 144 on a substrate 102. Each device 144 in the plurality of devices 144 occupies a different region on the substrate 102 and each device 144 in the plurality of devices 144 is capable of binding to a macromolecule 120. At least one device in the plurality of devices comprises (i) a first electrically conducting material, wherein the first electrically conducting material is overlaid on the different region of the substrate 102 occupied by the device 144, (ii) a spacer 140 overlaying the first electrically conducting material 106, (iii) a second electrically conducting material on the spacer 140 so that a cavity 113 is formed, and (iv) a passivation layer 130 overlaid on the second electrically conducting material.

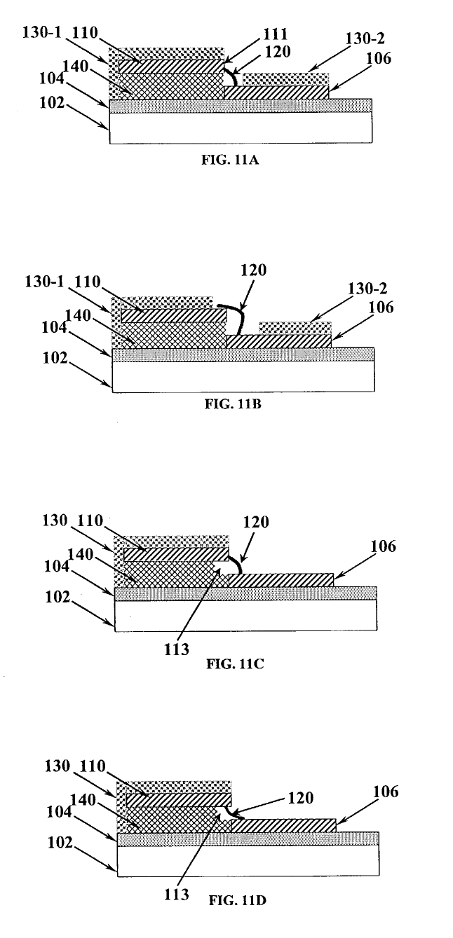

The biosensor illustrated in FIG. 10A is identical to the biosensor illustrated in FIG. 9D with the exception that macromolecule 120 contacts different portions of materials 106 and 110 in the biosensor. In the biosensor illustrated in FIG. 10A, a cavity 113 is formed between materials 106 and 110. Macromolecule 120 spans between the upper surface of material 106 and the lower surface of material 110 within cavity 113.

The biosensor illustrated in FIG. 10B is identical to the biosensor illustrated in FIG. 9D with the exception that passivation layer 130 covers only a portion of the upper surface of material 110. Thus, in the biosensor illustrated in FIG. 10B, a portion of the upper surface of material 110 is exposed. In the biosensor illustrated in FIG. 10B, macromolecule 120 spans between the exposed sidewall of material 106 and the exposed portion of the upper surface of material 110.

The biosensor illustrated in FIG. 10C is identical to the biosensor illustrated in FIG. 10B with the exception that macromolecule 120 spans between the lower surface of material 110 and the upper surface of material 106 in cavity 113. Cavity 113 forms because spacer 140 overlays only a portion of layer 106 and because material 110, which overlays spacer 140, overhangs spacer 140.

The biosensor illustrated in FIG. 10D includes a substrate 102 and an optional insulator 104 that is overlaid on substrate 102. In the case where optional insulator 104 is not used, material 106 is overlaid on a first portion of substrate 102 and spacer 140 is overlaid on a second portion of substrate 102 where the second portion of substrate 102 is adjacent to the first portion of substrate 102 (not shown). In the case where optional insulator 104 is used, material 106 is overlaid onto a first portion of insulator 104 and spacer 140 is overlaid on a second portion of insulator 104 where the second portion of insulator 104 is adjacent to the first portion of insulator 104. Next, material 110 is overlaid on spacer 140 and passivation layer 130 is overlaid on all exposed portions of material 110 except sidewall 111. Macromolecule 120 spans between sidewall 111 of material 110 and the upper surface of material 106.

Referring again to FIG. 10D, one embodiment of the present invention provides a biosensor comprising a plurality of devices 144 on a substrate 102. Each device 144 in the plurality of devices 144 occupy a different region on an insulator layer 104 and each device 144 in the plurality of devices 144 is capable of binding to a macromolecule 120. The insulator layer 104 is overlaid on the substrate 102. Each device 144 in the plurality of devices (i) comprises a first electrically conducting material 106, having a top surface, that is overlaid on a first portion of the different region of the insulator layer 104 occupied by the device 144, (ii) a spacer 140 overlaid on a second portion of the different region of the insulator layer 104, and (iii) a second electrically conducting material 110 having a top surface that is overlaid on a portion of spacer 140. Furthermore, in each device in the plurality of devices, first electrically conducting material 106 and spacer 140 abut each other and the first and second portions of insulator layer 104 do not overlap with each other. In some embodiments in accordance with FIG. 10D, insulator layer 104 is not used. In such instances, each device 144 is overlaid onto substrate 102. In some embodiments, a passivation layer 130 overlays the second electrically conducting material 110. In the biosensor illustrated in FIG. 10D, a first portion of a macromolecule 120 binds to a top portion of the first electrically conducting material 106 and a second portion of the macromolecule 120 binds to a side portion of the second electrically conducting material 110 in the illustrated device 144.

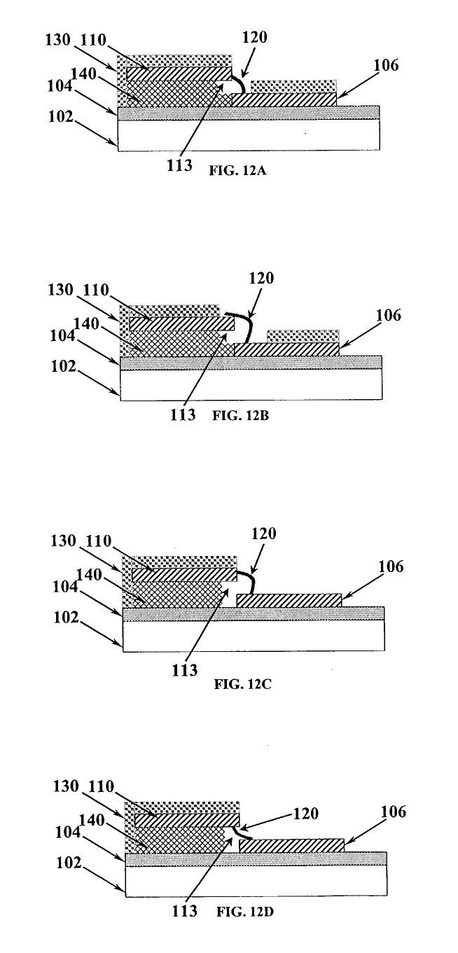

The biosensor illustrated in FIG. 11A is identical to the biosensor illustrated in FIG. 10D with the exception that the biosensor illustrated in FIG. 11A includes a second passivation layer 130-2 that overlays a portion of material 106. The second passivation layer 130-2 facilitates the localization of macromolecule 120. In some embodiments, for example, passivation layer 130-2 prevents nonspecific binding of macromolecule 120 to undesired regions for material 106. In the biosensor illustrated in FIG. 11A, a first passivation layer 130-2 overlays a portion of the first electrically conducting material 106 and a second passivation layer 130-1 overlays the second electrically conducting material 110. Furthermore, a first portion of the macromolecule 120 binds to a top portion of the first electrically conducting material 106 that is not covered by the first passivation layer 130-2 and a second portion of the macromolecule 120 binds to a side portion of the second electrically conducting material 110.

The biosensor illustrated in FIG. 11B is identical to the biosensor illustrated in FIG. 11A with the exception that passivation layer 130-1 only covers a portion of material 110. Therefore, a portion of the upper side of material 110 remains exposed. Furthermore, macromolecule 120 spans the exposed portion of the upper side of material 110 and the exposed portion of the upper side of material 106. Referring to FIG. 1B, in one embodiment of the present invention a first passivation layer 130-2 overlays a portion of electrically conducting material 106 and a second passivation layer 130-1 overlays a portion of electrically conducting material 110. Furthermore, a first portion of macromolecule 120 binds to a top portion of electrically conducting material 106 that is not covered by passivation layer 130-2 and a second portion of macromolecule 120 binds to a top portion of the electrically conducting material 110 that is not covered by passivation layer 130-1.

The biosensor illustrated in FIG. 11C is identical to the biosensor illustrated in FIG. 10D with the exception that a portion of spacer 140 is cut away, exposing a cavity 113. Cavity 113 effectively increases the distance of the path from material 106 to material 110 (or vice versa) that current must travel if macromolecule 120 is not used as an electrical conduit. Thus, the biosensor illustrated in FIG. 11C is advantageous because it has improved insulator properties that prevent the short circuiting of materials 106 and 110. To manufacture the biosensor illustrated in FIG. 11C, spacer 140 may be deposited and then cavity 113 may be formed by etching spacer 140. Alternatively, spacer 140 may be formed with cavity 113 using known microfabrication techniques. Referring to FIG. 11C, a passivation layer 130 overlays electrically conducting material 110 and spacer 140 includes a gap 113 exposing a portion of the bottom of electrically conducting material 110. Furthermore, a first portion of a macromolecule 120 binds to a top portion of electrically conducting material 106 and a second portion of a macromolecule 120 binds to a side portion of electrically conducting material 110.

The biosensor illustrated in FIG. 11D is identical to the biosensor illustrated in FIG. 11C with the exception that macromolecule 120 spans between the upper surface of material 106 and the lower surface of material 110 as illustrated. Referring to the biosensor illustrated in FIG. 11D, passivation layer 130 overlays electrically conducting material 110 and spacer 140 includes a gap 113 exposing a portion of the bottom of electrically conducting material 110. Furthermore, a first portion of macromolecule 120 binds to a top portion of electrically conducting material 106 and a second portion of the macromolecule 120 binds to the portion of the bottom of electrically conducting material 110 that is exposed by gap 113.

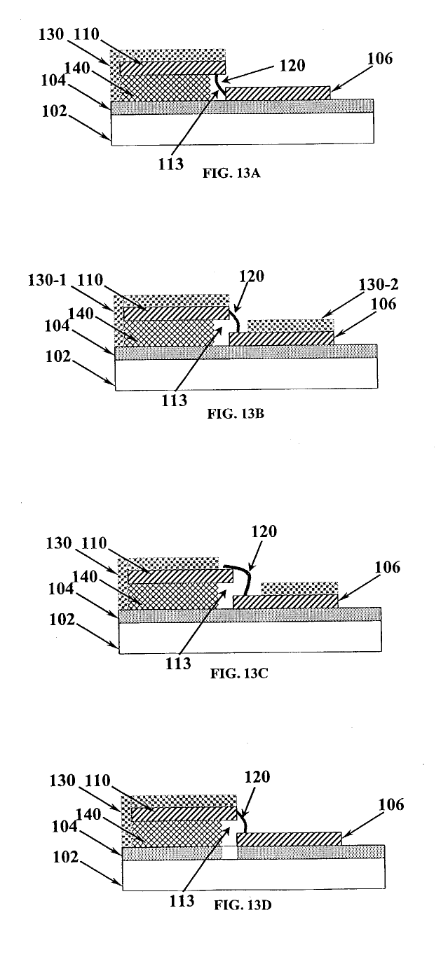

The biosensor illustrated in FIG. 12A is identical to the biosensor illustrated in FIG. 11A with the exception that a portion of spacer 140 is cut away, exposing a cavity 113. Cavity 113 effectively increases the distance of the path from material 106 to material 110 (or vice versa) that current must travel if macromolecule 120 is not used as an electrical conduit. Thus, the biosensor illustrated in FIG. 12A is advantageous because it has improved insulator properties that prevent the short circuiting of materials 106 and 110.

The biosensor illustrated in FIG. 12B is identical to the biosensor illustrated in FIG. 11B with the exception that a portion of spacer 140 is cut away, exposing a cavity 113. Cavity 113 effectively increases the distance of the path from material 106 to material 110 (or vice versa) that current must travel if macromolecule 120 is not used as an electrical conduit. Thus, the biosensor illustrated in FIG. 12B is advantageous because it has improved insulator properties that prevent the short circuiting of materials 106 and 110.

The biosensor illustrated in FIG. 12C is identical to the biosensor illustrated in FIG. 11C with the exception that cavity 113 extends all the way to insulator layer 104 in the biosensor illustrated in FIG. 12C. This extension increases the distance of the path from material 106 to material 110 (or vice versa) that undesirable leakage current must travel if macromolecule 120 is not used as an electrical conduit. Referring again to FIG. 12C, some embodiments of the present invention provide a biosensor in which a passivation layer 130 overlays electrically conducting material 110 and spacer 140 includes a gap 113 exposing a portion of the bottom of electrically conducting material 110. In this embodiment, gap 113 extends to insulation layer 104.

The biosensor illustrated in FIG. 12D is identical to the biosensor illustrated in FIG. 11D with the exception that cavity 113 extends all the way to insulator layer 104 in the biosensor illustrated in FIG. 12D. This extension increases the distance of the path from material 106 to material 110 (or vice versa) that current must travel if macromolecule 120 is not used as an electrical conduit.

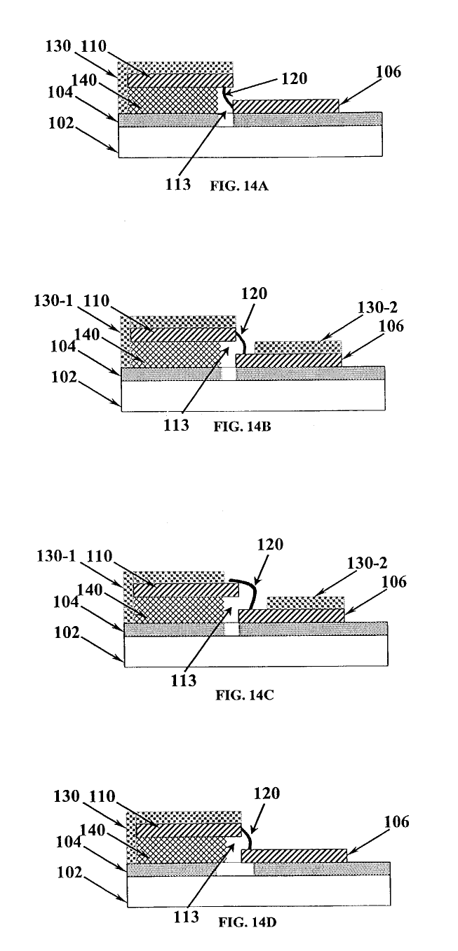

The biosensor illustrated in FIG. 13A is identical to the biosensor illustrated in FIG. 12D with the exception that macromolecule 120 spans from a side-wall of material 106 to the lower exposed surface of material 110. Further, the biosensor illustrated in FIG. 13B is identical to the biosensor illustrated in FIG. 12A with the exception that cavity 113 extends all the way to insulator layer 104 in the biosensor illustrated in FIG. 13B. The biosensor illustrated in FIG. 13C is identical to the biosensor illustrated in FIG. 12B with the exception that cavity 113 extends all the way to insulator layer 104 in the biosensor illustrated in FIG. 13C. The configuration of the biosensor illustrated in FIG. 13D is identical to the biosensor illustrated in FIG. 12C with the exception that cavity 113 extends all the way to substrate 102 in the biosensor illustrated in FIG. 13D. Referring again to FIG. 13D, in one embodiment of the present invention passivation layer 130-1 overlays electrically conducting material 110 and spacer 140 includes a gap 113 exposing a portion of the bottom of electrically conducting material 110. In this embodiment, gap 133 extends to substrate 102 through insulation layer 104. The extension of cavity 113 in the biosensors illustrated in FIGS. 13A, 13B, 13C, and 13D increase the distance of the path from material 106 to material 110 (or vice versa) that undesirable leakage current must travel if macromolecule 120 is not used as an electrical conduit.

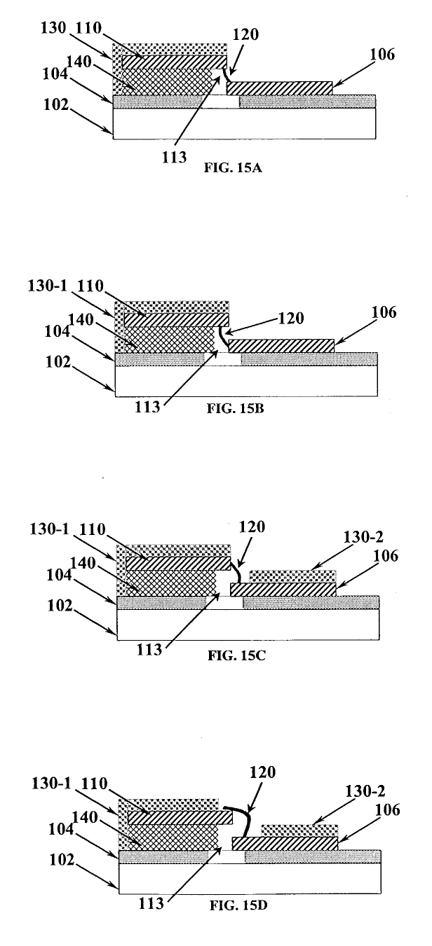

The biosensor illustrated in FIG. 14A is identical to the biosensor illustrated in FIG. 13A with the exception that cavity 113 extends all the way to substrate 102 in the biosensor illustrated in FIG. 14A. The biosensor illustrated in FIG. 14B is identical to the biosensor illustrated in FIG. 13B with the exception that cavity 113 extends all the way to substrate 102 in the biosensor illustrated in FIG. 14B. The biosensor illustrated in FIG. 14C is identical to the biosensor illustrated in FIG. 13C with the exception that cavity 113 extends all the way to substrate 102 in the biosensor illustrated in FIG. 14C. The biosensor illustrated in FIG. 14D is identical to the biosensor illustrated in FIG. 13D with the exception that cavity 113 is extended. In particular, a portion of insulator 104 is removed such that spacer 140 and material 106 overhang into cavity 113. The extension of cavity 113 in the biosensors illustrated in FIGS. 14A, 14B, 14C, and 14D increase the distance of the path from material 106 to material 110 (or vice versa) that current must travel if macromolecule 120 is not used as an electrical conduit.

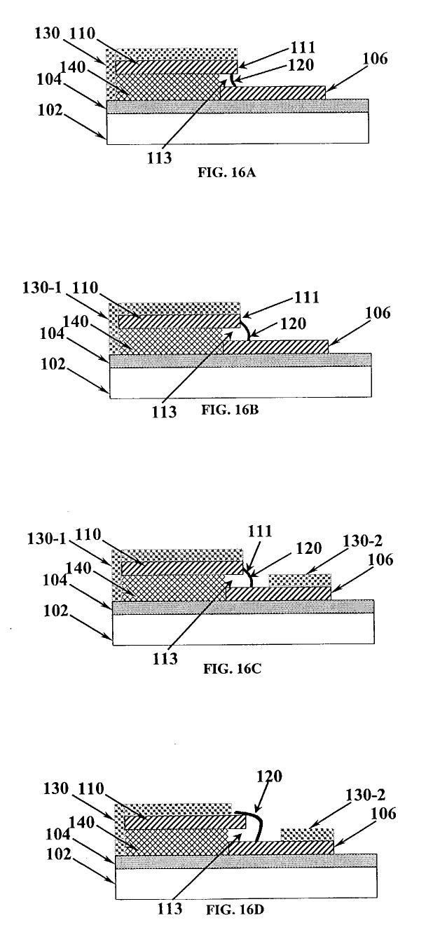

The configuration of the biosensor illustrated in FIG. 15A is identical to the configuration of the biosensor illustrated in FIG. 14D with the exception that macromolecule extends from the top of material 106 to the exposed bottom surface of material 110. The configuration of the biosensor illustrated in FIG. 15B is identical to the configuration of the biosensor illustrated in FIG. 15A with the exception that macromolecule 120 extends from the side of material 106 to the exposed bottom surface of material 110. The configuration of the biosensor illustrated in FIG. 15C is identical to the configuration of the biosensor illustrated in FIG. 14D with the exception that a second passivation layer 130 overlays a portion of material 106. In the biosensor illustrated in FIG. 15C, macromolecule 120 spans between the exposed portion of the topside of material 106 and the side portion of material 110. The biosensor illustrated in FIG. 15D is identical to the biosensor illustrated in FIG. 14C with the exception that cavity 113 is extended. In particular, a portion of insulator 104 is removed such that spacer 140 and material 106 overhang into cavity 113 in the biosensor illustrated in FIG. 15D.

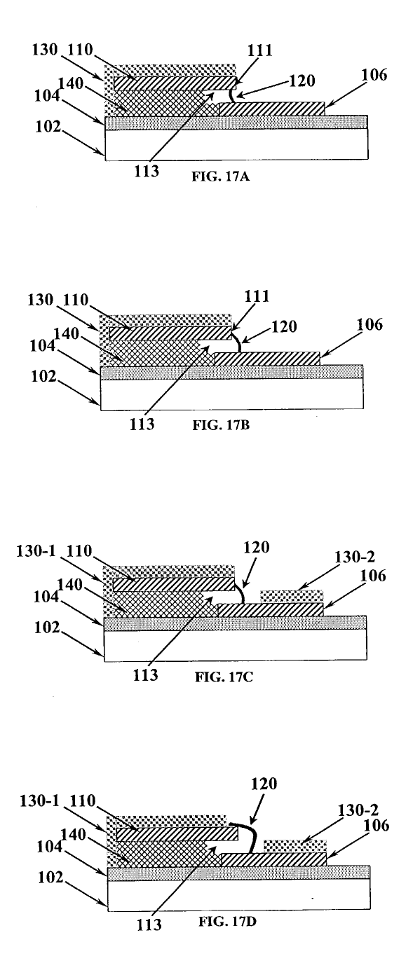

The biosensor illustrated in FIG. 16A includes a substrate 102 and an optional insulator 104 that is overlaid on substrate 102. In the case where optional insulator 104 is not used, material 106 is overlaid on a first portion of substrate 102 and spacer 140 is overlaid on a second portion of substrate 102 where the second portion of substrate 102 is adjacent to the first portion of substrate 102 (not shown). In the case where optional insulator 104 is used, material 106 is overlaid onto a first portion of insulator 104 and spacer 140 is overlaid onto a second portion of insulator 104 where the second portion of insulator 104 is adjacent to the first portion of insulator 104. Next, material 110 is overlaid on spacer 140 in such a manner that material 110 extends past spacer 140 thereby forming cavity 113. Passivation layer 130 is overlaid on all exposed portions of material 110 except sidewall 111. Macromolecule 120 spans between the exposed bottom of material 110 and the top of material 106 in cavity 113. The configuration of the biosensor illustrated in FIG. 16B is identical to the configuration of the biosensor illustrated in FIG. 16A with the exception that macromolecule 120 spans from the top of material 106 to side-wall 111 of material 110. The configuration of the biosensor illustrated in FIG. 16C is identical to the configuration of the biosensor illustrated in FIG. 16B with the exception that a passivation layer 130-2 overlays a portion of material 106. In some embodiments, passivation layer 130-2 in the biosensor illustrated in FIG. 16C prevents macromolecule 120 from binding to undesirable regions of material 106. The configuration of the biosensor illustrated in FIG. 16D is identical to the configuration of the biosensor illustrated in FIG. 16C except that passivation layer 130-1 only covers a portion of material 110. Thus, a portion of the topside of material 110 is exposed even after passivation layer 130-1 is overlaid on material 110. Furthermore, in the biosensor illustrated in FIG. 16D, the macromolecule spans from the exposed upper surface of material 106 to the exposed upper surface of material 110.

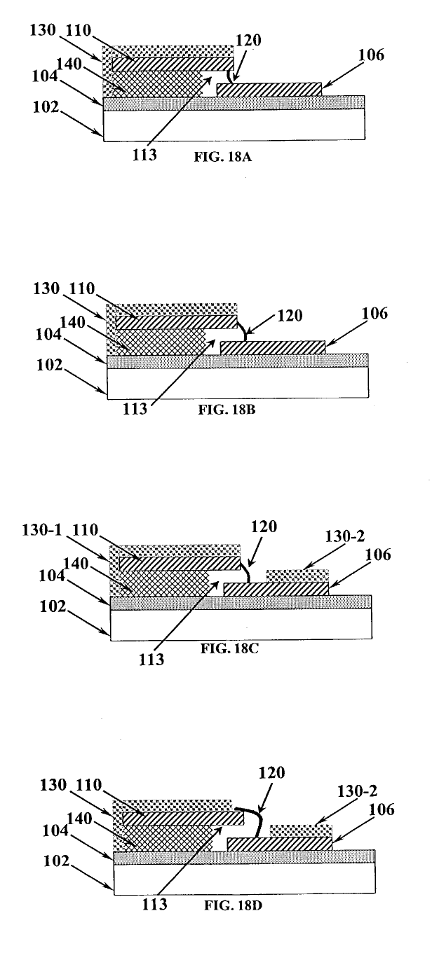

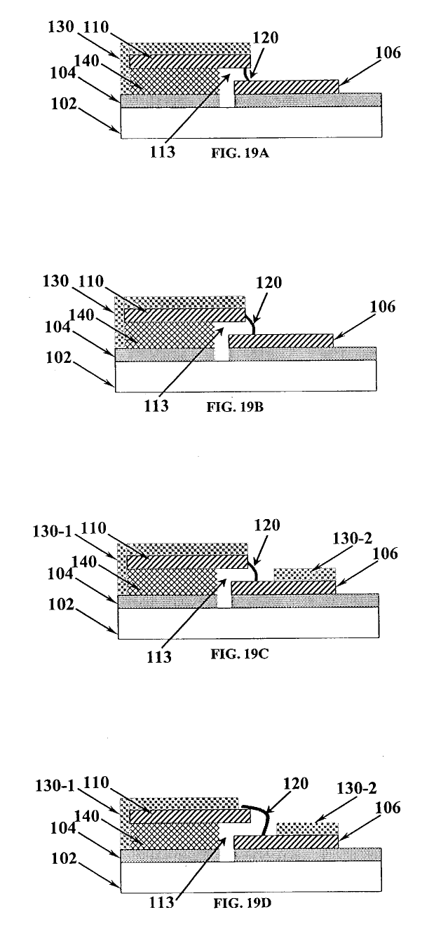

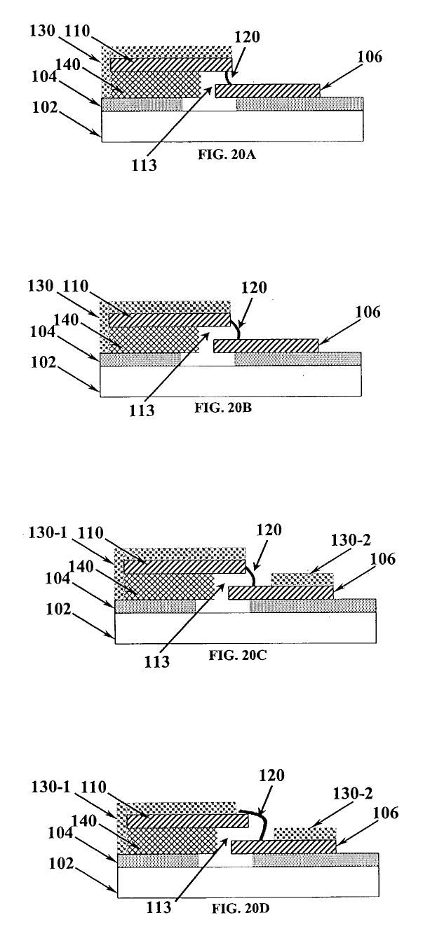

The configuration of the biosensors illustrated in FIGS. 17A, 17B, 17C, and 17D are identical to the configuration of the biosensors respectively illustrated in FIGS. 16A, 16B, 16C, and 16D with the exception that cavity 113 extends into spacer 140. The extension of cavity 113 in the biosensors illustrated in FIGS. 17A, 17B, 17C, and 17D increases the distance of the path from material 106 to material 110 (or vice versa) that current must travel if macromolecule 120 is bypassed. The configuration of the biosensors illustrated in FIGS. 18A, 18B, 18C, and 18D are identical to the configuration of the biosensors respectively illustrated in FIGS. 17A, 17B, 17C, and 17D with the exception that cavity 113 extends into spacer 140 all the way down to insulator layer 104. That is, in the biosensors illustrated in FIGS. 18A, 18B, 18C, and 18D, cavity 113 includes a gap that has the thickness as spacer 140. The extension of cavity 113 in the biosensors illustrated in FIGS. 18A, 18B, 18C, and 18D increases the distance of the path from material 106 to material 110 (or vice versa) that current must travel if macromolecule 120 is bypassed. The configuration of the biosensors illustrated in FIGS. 19A, 19B, 19C, and 19D are identical to the configuration of the biosensors respectively illustrated in FIGS. 18A, 18B, 18C, and 18D with the exception that cavity 113 extends into spacer 140 all the way down to substrate 102. That is, in the biosensors illustrated in FIGS. 18A, 18B, 18C, and 18D, cavity 113 includes a gap that has the same height as the combined thickness of spacer 140 and insulator 104. The extension of cavity 113 in the biosensors illustrated in FIGS. 19A, 19B, 19C, and 19D increases the distance of the path from material 106 to material 110 (or vice versa) that current must travel if macromolecule 120 is bypassed.

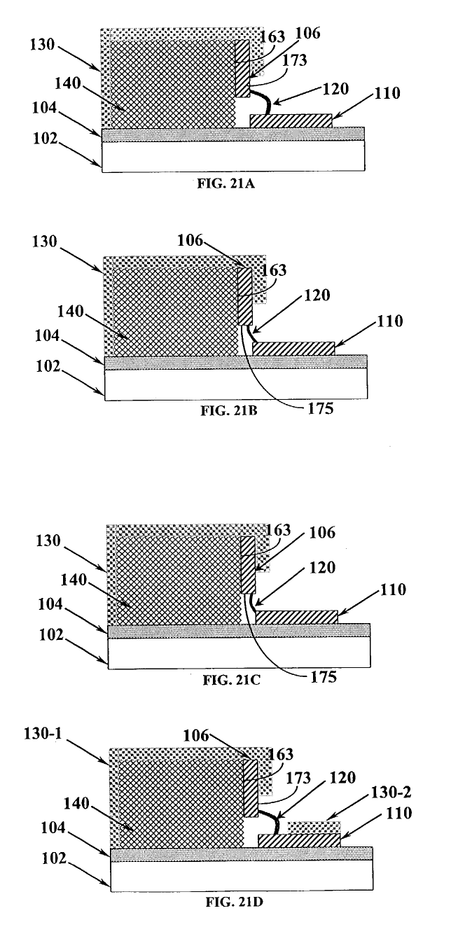

The configuration of the biosensors illustrated in FIGS. 20A, 20B, 20C, and 20D are identical to the configuration of the biosensors respectively illustrated in FIGS. 19A, 19B, 19C, and 19D with the exception that cavity 113 extends into spacer 140 all the way down to substrate 102. In fact, a portion of insulator 104 is absent in cavity 113 of the biosensors illustrated in FIG. 20 such that spacer 140 and material 106 overhang into cavity 113. The extension of cavity 113 in the biosensors illustrated in FIGS. 20A, 20B, 20C, and 20D increases the distance of the path from material 106 to material 110 (or vice versa) that current must travel if macromolecule 120 is bypassed.

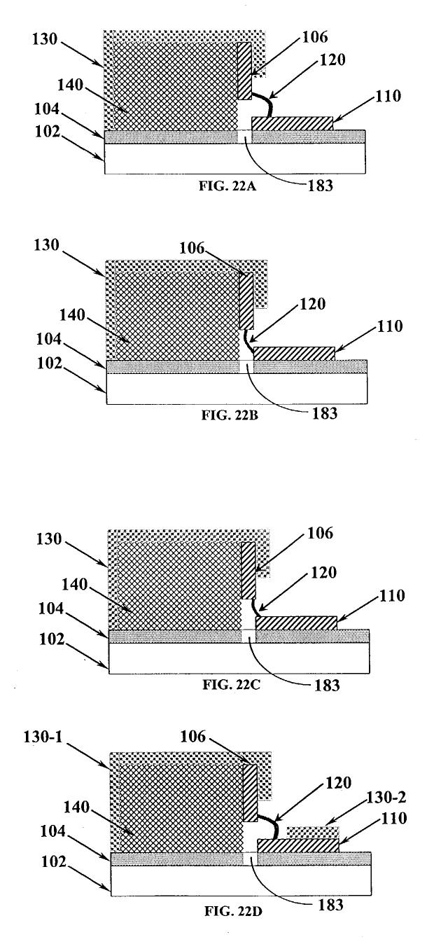

The biosensor illustrated in FIG. 21A includes a substrate 102 and an optional insulator 104 overlaid on substrate 102. In the case where optional insulator 104 is not used, material 110 is overlaid on a first portion of substrate 102 and spacer 140 is overlaid on a second portion of substrate 102. In the case where optional insulator 104 is used, material 110 is overlaid onto a first portion of insulator 104 and spacer 140 is overlaid on a second portion of insulator 104. Spacer 140 includes a sidewall 163 and material 106 is overlaid on a portion of sidewall 163. Passivation layer 130 is overlaid on spacer 140 and a portion of material 106. Macromolecule 120 spans between surface 173 of material 106 and the upper surface of material 110.

Referring again to the biosensor illustrated in FIG. 21A, one embodiment of the present invention provides a biosensor comprising a plurality of devices 144 on a substrate 102. Each device 144 in the plurality of devices 144 occupies a different region on an insulator layer 104. Furthermore, each device 144 in the plurality of devices 144 is capable of binding to a macromolecule 120. Insulator layer 104 is overlaid on substrate 102. Each device 144 in the plurality of devices 144 comprises (i) an electrically conducting material 110, (ii) a spacer overlaid on a second portion of the different region of insulator layer 104 occupied by device 144, (iii) an electrically conducting material 106 that abuts side-wall 163 of spacer 140; and (iv) a passivation layer 130 that overlays spacer 140 and a portion of electrically conducting material 106. In this embodiment, electrically conducting material 110 is overlaid on a first portion of the different region of the insulator layer 104 occupied by the device 144. Furthermore, the first portion of insulator layer 104 does not overlap with the second portion of insulator layer 104.

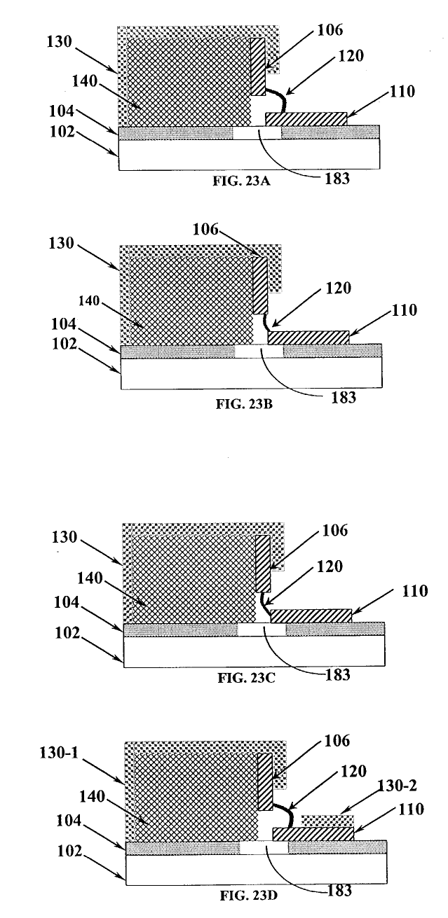

The configuration of the biosensor illustrated in FIG. 21B is identical to the configuration of the biosensor illustrated in FIG. 21A with the exception that macromolecule 120 spans between surface 175 of material 106 and the upper surface of material 110. The configuration of the biosensor illustrated in FIG. 21C is identical to the configuration of the biosensor illustrated in FIG. 21A with the exception that macromolecule 120 spans between surface 175 of material 106 and the a side-wall of material 110. The configuration of the biosensor illustrated in FIG. 21D is identical to the configuration of the biosensor illustrated in FIG. 21A with the exception that a passivation layer 130-2 overlays a portion of material 110. Passivation layer 130-2 prevents macromolecule 120 from binding to undesired regions of the biosensor illustrated in FIG. 21D. Thus, passivation layer 130-2 helps ensure that a first portion of macromolecule 120 binds to a region of material 110 that is proximate to side-wall 173 of material 106 so that a second portion of macromolecule can bind to side-wall 173. In the biosensors illustrated in FIGS. 21A through 22D, there is a gap between material 110 and spacer 140.

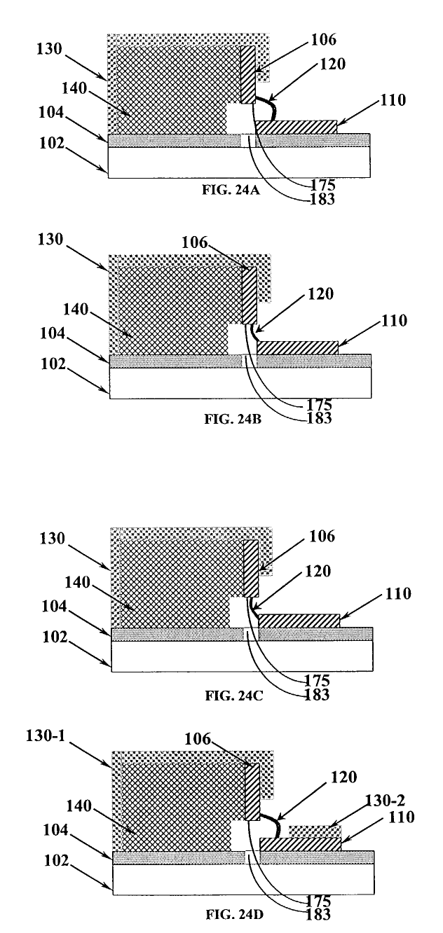

The configuration of the biosensors illustrated in FIGS. 22A through 22D are respectively identical to the configuration of the biosensors illustrated in FIGS. 21A through 21D with the exception that insulator layer 104 not optional in the biosensors illustrated in FIGS. 22A through 22D. Furthermore, there is a cavity 183 in the insulator layer 104 in the biosensors illustrated in FIGS. 22A through 22B that coincides with the separation between material 110 and spacer 140. Cavity 183 effectively increases the distance of the path from material 106 to material 110 (or vice versa) that current must travel if macromolecule 120 is not used as an electrical conduit. Thus, the biosensors illustrated in FIGS. 23A through 23D are advantageous because they have improved insulator properties that prevent the short-circuiting of materials 106 and 110.

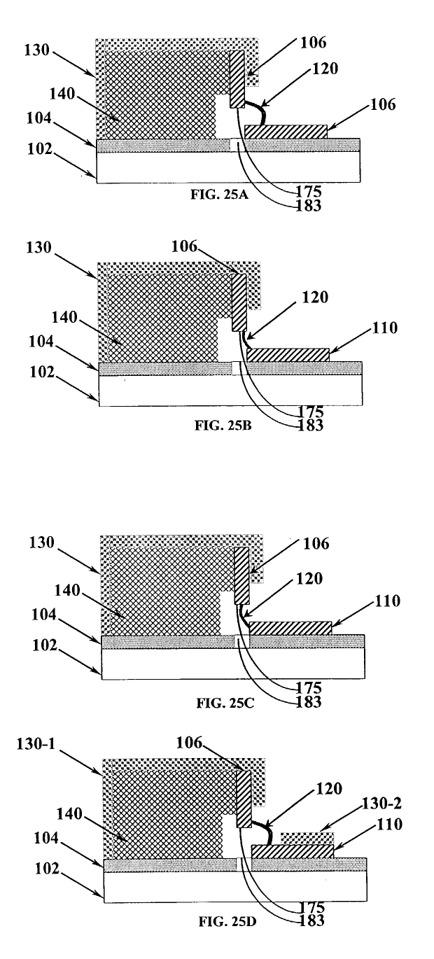

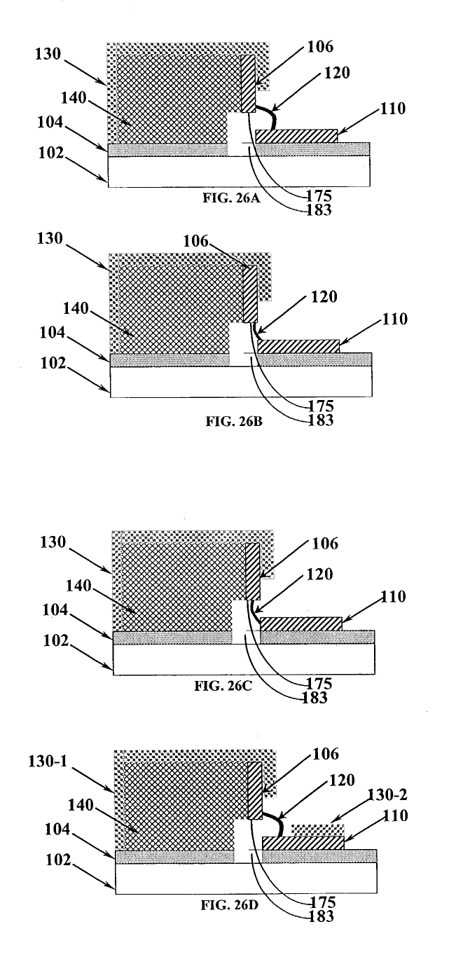

The configuration of the biosensors illustrated in FIGS. 23A through 23D are respectively identical to the configuration of the biosensors illustrated in FIGS. 22A through 22D with the exception that cavity 183 is extended in the biosensors illustrated in FIGS. 23A through 23D. Because cavity 183 is extended into layer 104, material 106 and material 110 overhang cavity 183 as illustrated in FIGS. 23A through 23D. The configuration of the biosensors illustrated in FIGS. 24A through 24D is respectively identical to the configuration of the biosensors illustrated in FIGS. 22A through 22D with the exception that cavity 183 is extended into spacer 140 in the biosensors illustrated in FIGS. 24A through 24D. In the biosensors illustrated in FIGS. 24A through 24D, the upper surface of cavity 183 is coextensive with surface 175 of material 106 as illustrated. The configuration of the biosensors illustrated in FIGS. 25A through 25D is respectively identical to the configuration of the biosensors illustrated in FIGS. 24A through 24D with the exception that cavity 183 is extended into spacer 140. Because of this extension in cavity 183, the top of cavity 183 is no longer coextensive with surface 175 of material 106. Rather, the top of cavity 183 extends past surface 175 as illustrated in FIGS. 25A through 25D.

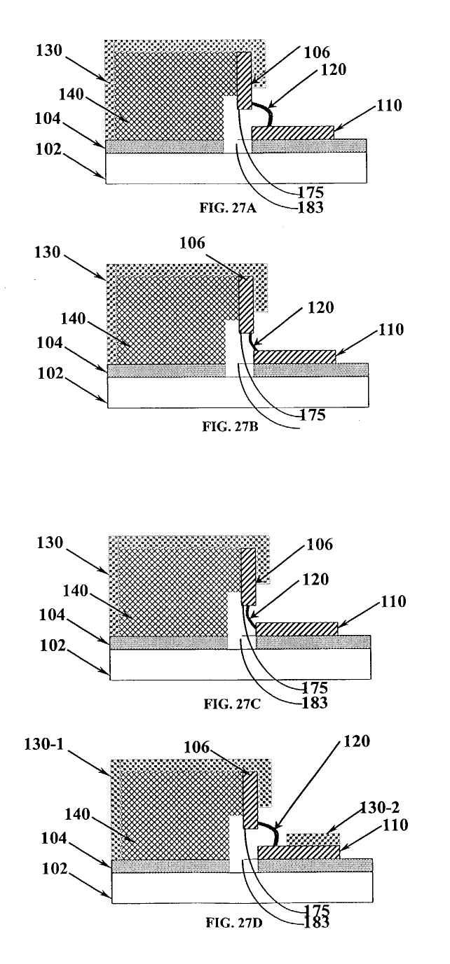

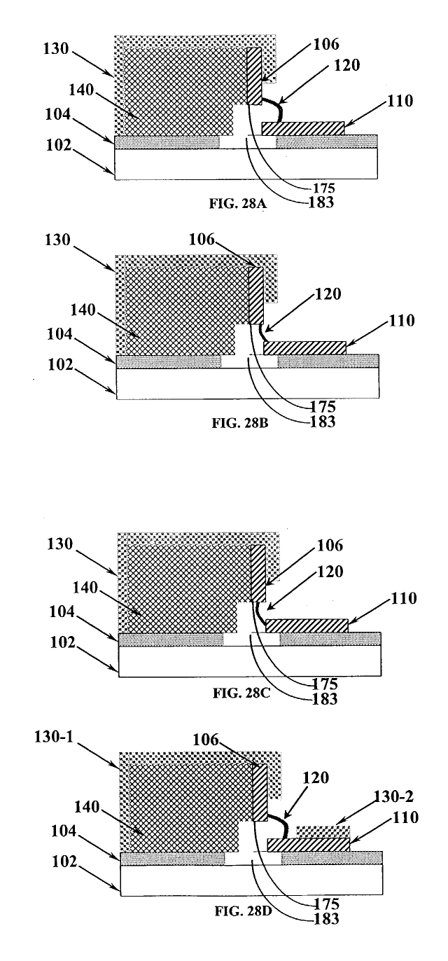

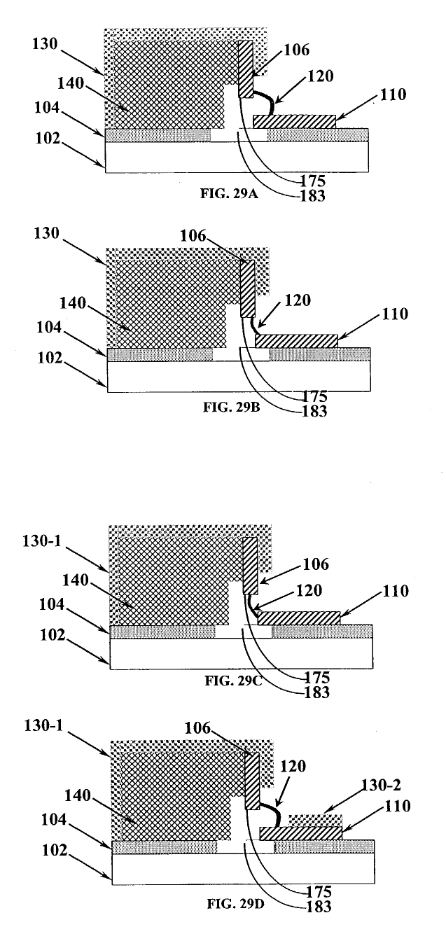

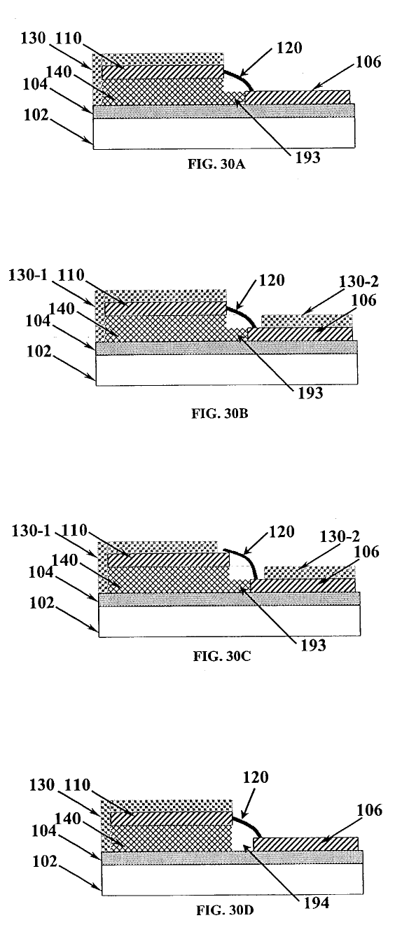

In the biosensors illustrated in FIGS. 25A through 25D, cavity 183 extends further into spacer 140 than into insulator 104. As a result, a portion of layer 104 is exposed by cavity 183. In other words, a portion of layer 104 protrudes into cavity 183 in the biosensors illustrated in FIGS. 25A through 25D. This is not the case with the biosensors illustrated in FIGS. 26A through 26B. In the biosensors illustrated in FIGS. 26A through 26B, cavity 183 extends into insulator 104 and spacer 140 to the same extent such that a portion of layer 104 does not extend into cavity 183. In the biosensors illustrated in FIGS. 26A through 26D, the upper surface of cavity 183 is coextensive with surface 175 of material 106 as illustrated. The configuration of the biosensors illustrated in FIGS. 27A through 27D is respectively identical to the configuration of the biosensors illustrated in FIGS. 26A through 26D with the exception that cavity 183 is extended into spacer 140 in the biosensors illustrated in FIGS. 27A through 27D. The configuration of the biosensors illustrated in FIGS. 28A through 28D is respectively identical to the configuration of the biosensors illustrated in FIGS. 26A through 26D with the exception that cavity 183 is further extended into layer 104. Because of the extension of cavity 183 into layer 104, a portion of spacer 140 and material 110 overhang into cavity 183. In the biosensors illustrated in FIGS. 28A through 28D, the upper surface of cavity 183 is coextensive with surface 175 of material 106 as illustrated. The configuration of the biosensors illustrated in FIGS. 29A through 29D is respectively identical to the configuration of the biosensors illustrated in FIGS. 28A through 28D with the exception that cavity 183 is extended into spacer 140 in the biosensors illustrated in FIGS. 29A through 29D. Because of this, the top of cavity 183 is not coextensive with surface 175 of material 106 in the biosensors illustrated in FIGS. 29A through 29D.

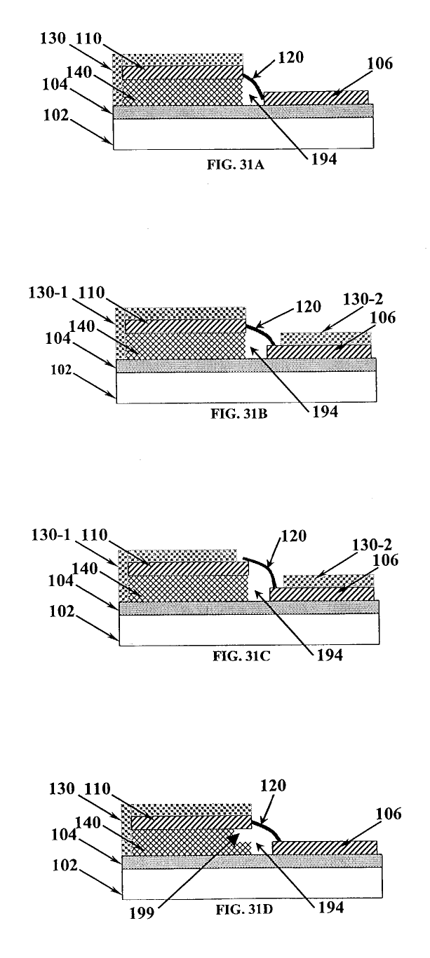

The configuration of the biosensor illustrated in FIG. 30A is identical to the configuration of the biosensor illustrated in FIG. 10D with the exception that material 106 does not abut the spacer 140/material 110 stack in the biosensor illustrated in FIG. 30A. Rather, extended portion 193 of spacer 140 separates the spacer 140/material 110 stack from material 106 in the biosensor illustrated in FIG. 30A. In contrast, in the biosensor illustrated in FIG. 10D, material 106 is juxtaposed against the spacer 140/material 110 stack and there is no extended portion 193. Extended portion 193 of spacer 140 provides the advantage of further separating material 106 and material 10 in order to prevent a short circuit. In some embodiments, extended portion 193 has a width that is more than 200 Angstroms, more than 500 Angstroms, or between 25 Angstroms and 700 Angstroms.

Referring to FIG. 30A, one embodiment of the present invention provides a biosensor including a plurality of devices 144 on a substrate 102. Each device 144 in the plurality of devices 144 occupies a different region on an insulator layer 104 and each device 144 in the plurality of devices 144 is capable of binding to a macromolecule 120. Insulator layer 104 is overlaid on substrate 102. Each device 144 in the plurality of devices 144 comprises (i) an electrically conducting material 106, (ii) a spacer 144 overlaid on a second portion of the different region of the insulator layer 104 that is occupied by the device 144, the spacer 144 including a main body and an extended portion 193, wherein extended portion 193 of spacer 144 abuts electrically conducting material 106, (iii) an electrically conducting material 110 that is overlaid on the main body of spacer 140, and (iv) a first passivation layer 130 that overlays the main body of spacer 140. In this embodiment, electrically conducting material 106 is overlaid on a first portion of the different region of the insulator layer that is occupied by the device.

The configuration of the biosensor illustrated in FIG. 30B is identical to the configuration of the biosensor illustrated in FIG. 11A with the exception that material 106 is not juxtaposed against the spacer 140/material 110 stack in the biosensor illustrated in FIG. 30B. Rather, extended portion 193 of spacer 140 separates the spacer 140/material 110 stack from material 106 in the biosensor illustrated in FIG. 30B. Referring to FIG. 30B, some embodiments of the present invention provide a biosensor in which a second passivation layer 130-2 overlays a portion of electrically conducting material 106. In such embodiments, a first portion of macromolecule 120 binds to a top portion of electrically conducting material 106 that is not covered by passivation layer 130-2 and a second portion of macromolecule 120 binds to a side portion of electrically conducting material 110.

The configuration of the biosensor illustrated in FIG. 30C is identical to the configuration of the biosensor illustrated in FIG. 11B with the exception that material 106 is not juxtaposed against the spacer 140/material 110 stack in the biosensor illustrated in FIG. 30C. Rather, extended portion 193 of spacer 140 separates the spacer 140/material 110 stack from material 106 in the biosensor illustrated in FIG. 30C. In some embodiments extended portion 193 has a width of more than 5 Angstroms, more than 20 Angstroms, more than 50 Angstroms, more than 100 Angstroms, or more than 150 Angstroms.

The configuration of the biosensor illustrated in FIG. 30D is identical to the configuration of the biosensor illustrated in FIG. 10D with the exception that material 106 is not juxtaposed against the spacer 140/material 110 stack in the biosensor illustrated in FIG. 30D. Rather, a space 194 separates the spacer 140/material 110 stack from material 106 in the biosensor illustrated in FIG. 30D. In contrast, in the biosensor illustrated in FIG. 10D, material 106 is juxtaposed against the spacer 104/material 110 stack and there is no space 194. Space 194 provides the advantage of further separating material 106 and material 110 in order to prevent a short circuit.

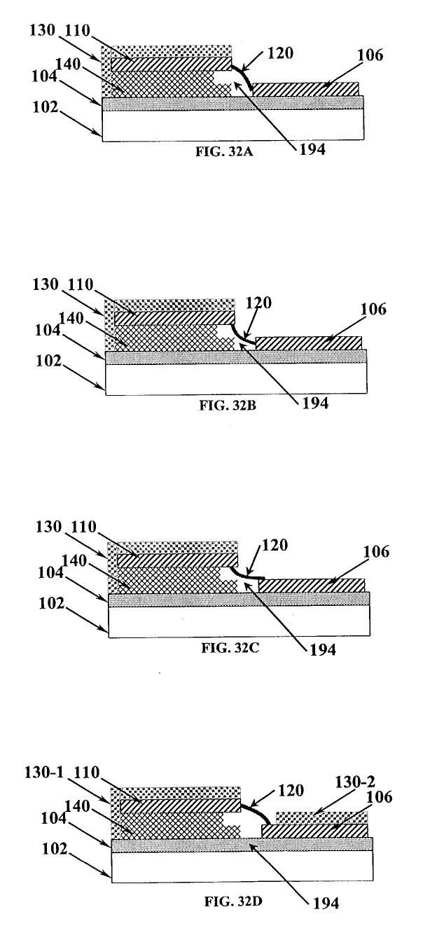

The configuration of the biosensor illustrated in FIG. 31A is identical to the configuration of the biosensor illustrated in FIG. 30D with the exception that a portion of macromolecule 120 binds to a side-wall of material 106 in the biosensor illustrated in FIG. 31A. In contrast, in the biosensor illustrated in FIG. 30D, a portion of macromolecule 120 binds to the top of material 106 as illustrated in FIG. 30D. The configuration of the biosensor illustrated in FIG. 31B is identical to the configuration of the biosensor illustrated in FIG. 11A with the exception that material 106 is not juxtaposed against the spacer 140/material 110 stack in the biosensor illustrated in FIG. 31B. Rather, a space 194 separates the spacer 140/material 110 stack from material 106 in the biosensor illustrated in FIG. 31B. In contrast, in the biosensor illustrated in FIG. 11A, material 106 is juxtaposed against the spacer 140/material 110 stack and there is no space 194. The configuration of the biosensor illustrated in FIG. 31C is identical to the configuration of the biosensor illustrated in FIG. 11B with the exception that material 106 is not juxtaposed against the spacer 140/material 110 stack in the biosensor illustrated in FIG. 31C. Rather, a space 194 separates the spacer 140/material 110 stack from material 106 in the biosensor illustrated in FIG. 31C. In contrast, in the biosensor illustrated in FIG. 11B, material 106 is juxtaposed against the spacer 140/material 110 stack and there is no space 194.

The configuration of the biosensor illustrated in FIG. 31D is identical to the configuration of the biosensor illustrated in FIG. 11C with the exception that material 106 is not juxtaposed against the spacer 140/material 110 stack in the biosensor illustrated in FIG. 31D. Rather, a space 194 separates the spacer 140/material 110 stack from material 106 in the biosensor illustrated in FIG. 3 ID. In contrast, in the biosensor illustrated in FIG. 11C, material 106 is juxtaposed against the spacer 140/material 110 stack and there is no space 194. Space 194 provides the advantage of further separating material 106 and material 110 in order to prevent a short circuit. Referring to FIG. 31D, some embodiments of the present invention provide a biosensor in which the extended portion 193 (FIG. 30A) of spacer 144 comprises gap 194. Furthermore, the main portion of spacer 144 includes a crevice 199 that exposes a bottom portion electrically conductive material 110. In such embodiments, a first portion of macromolecule 120 binds to an upper surface of electrically conducting material 106 and a second portion of macromolecule 120 binds to a side portion of electrically conducting material 110.

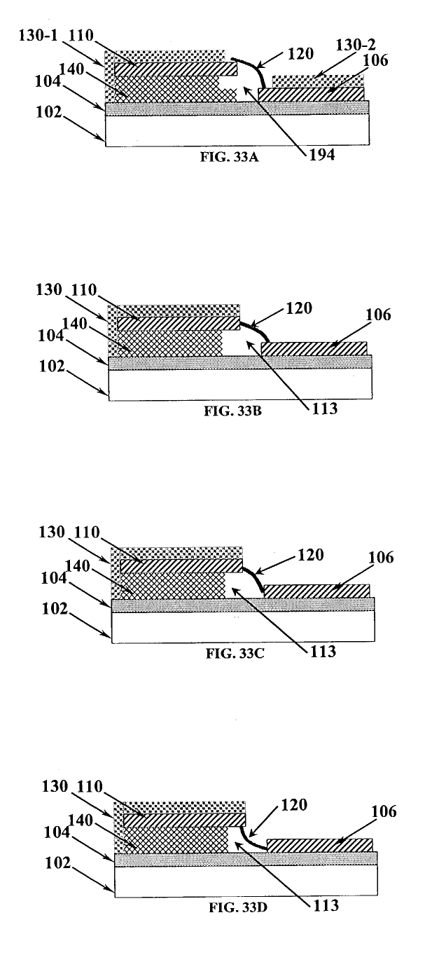

The configuration of the biosensor illustrated in FIG. 32A is identical to the configuration of the biosensor illustrated in FIG. 31D with the exception that a portion of macromolecule 120 binds to a side-wall of material 106 in the biosensor illustrated in FIG. 32A. In contrast, in the biosensor illustrated in FIG. 31D, a portion of macromolecule 120 binds to the top of material 106 as illustrated in FIG. 31D. The configuration of the biosensor illustrated in FIG. 32B is identical to the configuration of the biosensor illustrated in FIG. 32A with one exception. A portion of macromolecule 120 binds to the bottom of an exposed portion of material 106 that overhangs into space 194 in the biosensor illustrated in FIG. 32B. In contrast, in the biosensor illustrated in FIG. 32B, a portion of macromolecule 120 binds to the exposed sidewalk of material 110. The configuration of the biosensor illustrated in FIG. 32C is identical to the configuration of the biosensor illustrated in FIG. 31D with one exception. A portion of macromolecule 120 binds to the bottom of an exposed portion of material 106 that overhangs into space 194 in the biosensor illustrated in FIG. 32B. In contrast, in the biosensor illustrated in FIG. 31D, a portion of macromolecule 120 binds to the exposed sidewall of material 110. The configuration of the biosensor illustrated in FIG. 32D is identical to the configuration of the biosensor illustrated in FIG. 11C with the exception that material 106 is not juxtaposed against the spacer 140/material 110 stack in the biosensor illustrated in FIG. 30D. Rather, a space 194 separates the spacer 140/material 110 stack from material 106 in the biosensor illustrated in FIG. 32D.

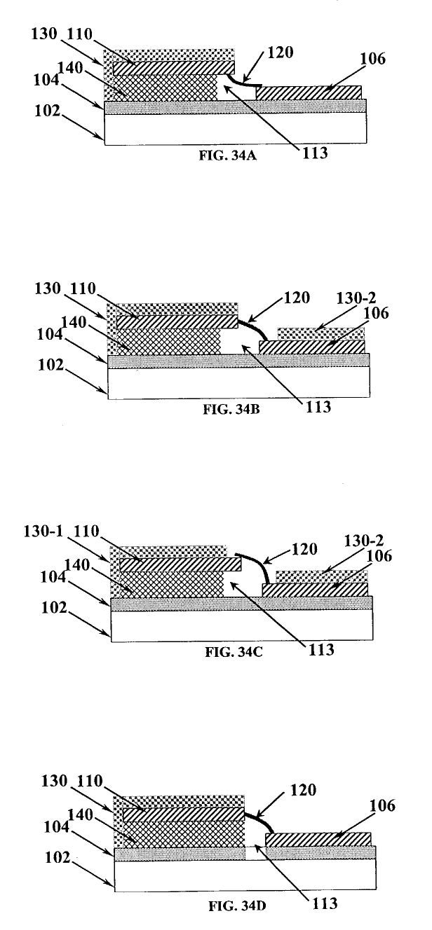

The configuration of the biosensor illustrated in FIG. 33A is identical to the configuration of the biosensor illustrated in FIG. 12B with the exception that material 106 is not juxtaposed against the spacer 140/material 110 stack in the biosensor illustrated in FIG. 33A. Rather, a space 194 separates the spacer 140/material 110 stack from material 106 in the biosensor illustrated in FIG. 33A. In some embodiments, space 194 in biosensors having a configuration illustrated in FIGS. 30D, 31A, 31B, 31C, 31D, 32A, 32B, 32C, 32D, or 33A, has a width that is between 5 Angstroms and 20 Angstroms, that is between 20 Angstroms and 50 Angstroms, or that is between 50 Angstroms and 150 Angstroms.

The configuration of the biosensor illustrated in FIG. 33B is identical to the configuration of the biosensor illustrated in FIG. 12C with the exception that space 113 is extended such that material 106 is further separated from the spacer 140/material 110 stack in the biosensor illustrated in FIG. 33B. Further separation of material 106 from the spacer 140/material 110 stack allows for biosensors having the configuration shown in FIG. 33C, in which a first portion of macromolecule 120 binds to the side-wall of material 106 and a second portion of macromolecule 120 binds to the side-wall of material 110. The configuration of the biosensor illustrated in FIG. 33D is identical to the configuration of the biosensor illustrated in FIG. 13A with the exception that space 113 is extended such that material 106 is further separated from the spacer 140/material 110 stack in the biosensor illustrated in FIG. 33D. In some embodiments in accordance with FIGS. 33B, 33C, or 33D, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms.

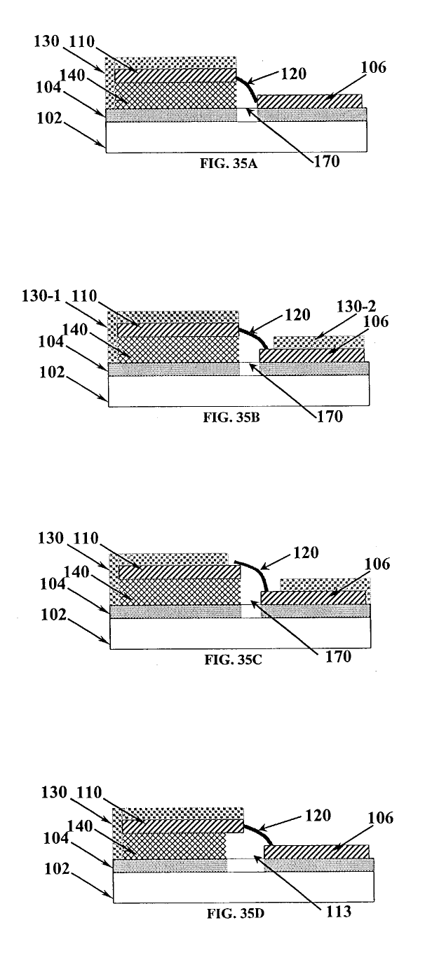

The configuration of the biosensor illustrated in FIG. 34A is identical to the configuration of the biosensor illustrated in FIG. 12D with the exception that space 113 is extended such that material 106 is further separated from the spacer 140/material 110 stack in the biosensor illustrated in FIG. 34A. The configuration of the biosensor illustrated in FIG. 34B is identical to the configuration of the biosensor illustrated in FIG. 13B with the exception that space 113 is extended such that material 106 is further separated from the spacer 140/material 110 stack in the biosensor illustrated in FIG. 34B. The configuration of the biosensor illustrated in FIG. 34C is identical to the configuration of the biosensor illustrated in FIG. 12B with the exception that space 113 is extended such that material 106 is further separated from the spacer 140/material 10 stack in the biosensor illustrated in FIG. 34C. The configuration of the biosensor illustrated in FIG. 34D is identical to the configuration of the biosensor illustrated in FIG. 12C with the exception that space 113 is extended such that material 106 is further separated from the spacer 140/material 110 stack in the biosensor illustrated in FIG. 34D. In some embodiments in accordance with FIGS. 34A, 34B, 34C, or 34D, material 106 and the spacer 140/material 110 stack are separated by a distance that is between 5 Angstroms and 20 Angstroms, between 20 Angstroms and 50 Angstroms, between 50 Angstroms and 100 Angstroms, or that is more 100 Angstroms.

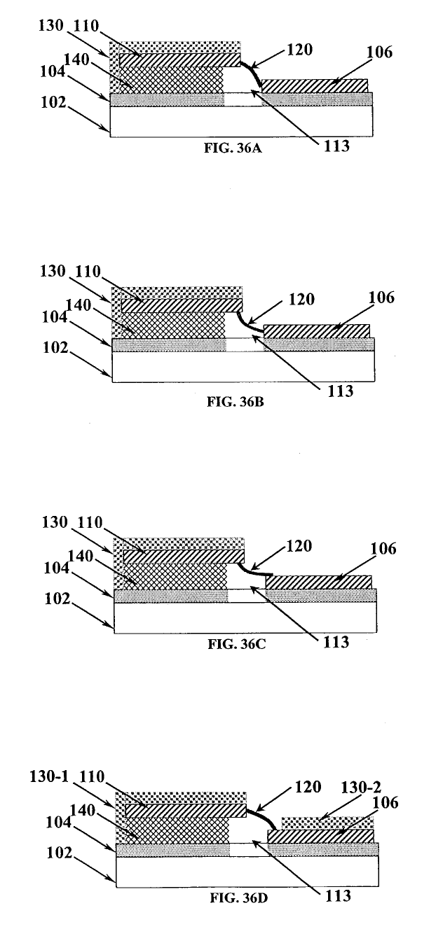

The configuration of the biosensor illustrated in FIG. 35A is identical to the configuration of the biosensor illustrated in FIG. 6D with the exception that material 106 is further separated from the spacer 140/material 110 stack in the biosensor illustrated in FIG. 35A than the biosensor illustrated in FIG. 6D. This separation allows for macromolecule 120 to bind to a sidewall of material 106 (FIG. 35A) rather than the top of material 106 (FIG. 6D). In some embodiments in accordance with FIG. 35A, material 106 and the spacer 140/material 110 stack are separated by a distance of that is between 5 Angstroms and 20 Angstroms, between 20 Angstroms and 50 Angstroms, between 50 Angstroms and 100 Angstroms, or that is more than 100 Angstroms.

The configuration of the biosensor illustrated in FIG. 35B is identical to the configuration of the biosensor illustrated in FIG. 7A with the exception that material 106 is further separated from the spacer 140/material 110 stack in the biosensor illustrated in FIG. 35B. In some embodiments in accordance with FIG. 35B, material 106 and the spacer 140/material 110 stack are separated by a distance of that is between five Angstroms and 20 Angstroms, between 20 Angstroms and 50 Angstroms, between 50 Angstroms and 100 Angstroms, or that is more than 100 Angstroms.

The configuration of the biosensor illustrated in FIG. 35C is identical to the configuration of the biosensor illustrated in FIG. 7B with the exception that material 106 is further separated from the spacer 140/material 110 stack in the biosensor illustrated in FIG. 35C. In some embodiments in accordance with FIG. 35C, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms.

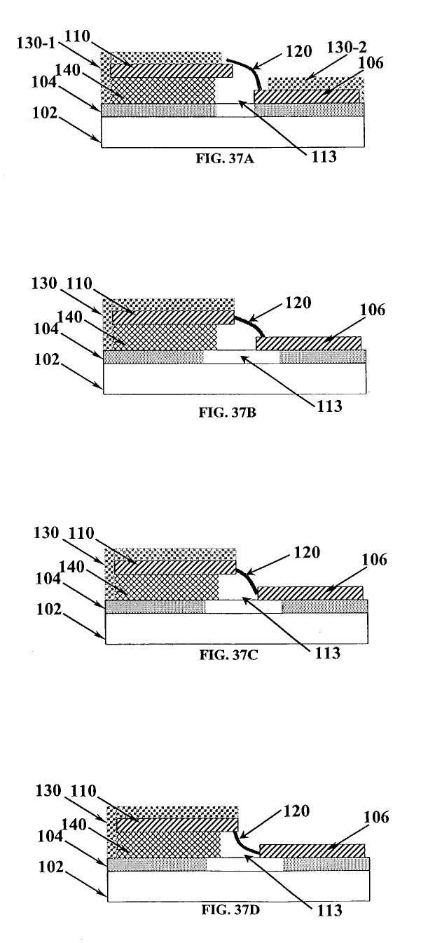

The configuration of the biosensor illustrated in FIG. 35D is identical to the configuration of the biosensor illustrated in FIG. 13D with the exception that material 106 is further separated from the spacer 140/material 110 stack in the biosensor illustrated in FIG. 35D. In some embodiments in accordance with FIG. 35D, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms. The additional separation found in the biosensor illustrated in FIG. 35D allows for the biosensor configuration illustrated in FIG. 36A in which a portion of macromolecule 120 binds to a side-wall of material 106 rather than the top of material 106 as shown in FIG. 35D.

The configuration of the biosensor illustrated in FIG. 36B is identical to the configuration of the biosensor illustrated in FIG. 14A with the exception that material 106 is further separated from the spacer 140/material 110 stack in the biosensor illustrated in FIG. 36B. In some embodiments in accordance with FIG. 36B, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms. The configuration of the biosensor illustrated in FIG. 36C is identical to the configuration of the biosensor illustrated in FIG. 36B with the exception that a portion of macromolecule 120 binds to a side-wall of material 106 rather than the top of material 106.

The configuration of the biosensor illustrated in FIG. 36D is identical to the configuration of the biosensor illustrated in FIG. 14B with the exception that material 106 is further separated from the spacer 140/material 110 stack in the biosensor illustrated in FIG. 36D. In some embodiments in accordance with FIG. 36D, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms.

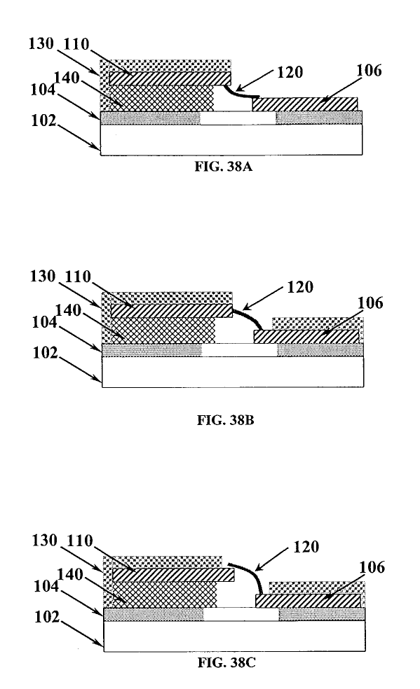

The configuration of the biosensor illustrated in FIG. 37A is identical to the configuration of the biosensor illustrated in FIG. 14C with the exception that material 106 is further separated from the spacer 140/material 110 stack in the biosensor illustrated in FIG. 37A. In some embodiments in accordance with FIG. 37A, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms.

The configuration of the biosensor illustrated in FIG. 37B is identical to the configuration of the biosensor illustrated in FIG. 14D with the exception that material 106 is further separated from the spacer 140/material 110 stack in the biosensor illustrated in FIG. 37B. The increased separation of the spacer 140/material 110 stack from material 106 in the biosensor illustrated in FIG. 37B allows for biosensor configurations such as that illustrated in FIG. 37C in which a portion of macromolecule 120 binds to the side-wall of material 106 rather than the top of material 106. In some embodiments in accordance with FIG. 37B or 37C, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms.

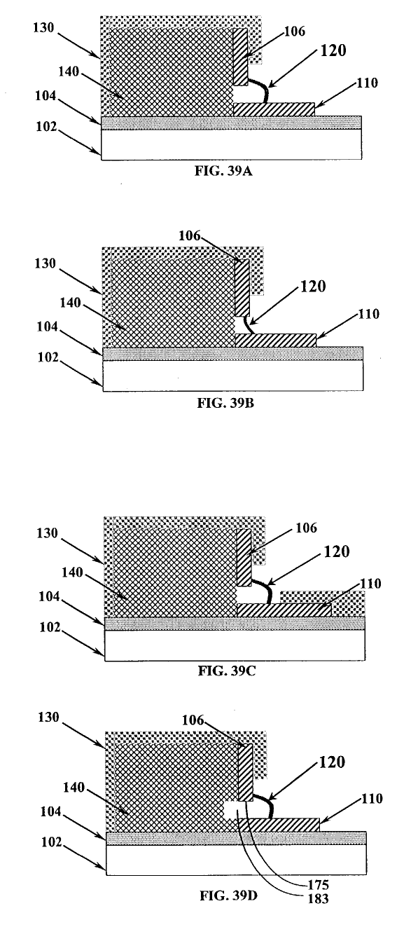

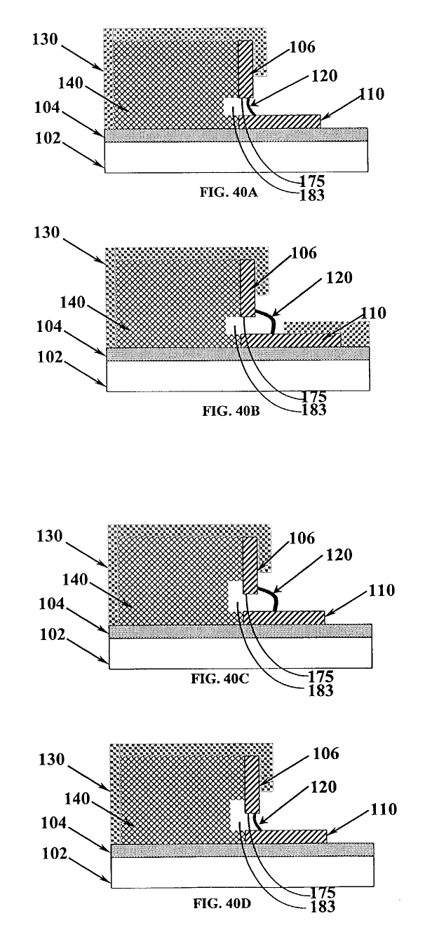

The configuration of the biosensor illustrated in FIG. 37D is identical to the configuration of the biosensor illustrated in FIG. 15B with the exception that material 106 is further separated from the spacer 140/material 110 stack in the biosensor illustrated in FIG. 37D. In some embodiments in accordance with FIG. 37D, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms. The configuration of the biosensor illustrated in FIG. 38A is identical to the configuration of the biosensor illustrated in FIG. 15A with the exception that material 106 is further separated from the spacer 140/material 110 stack in the biosensor illustrated in FIG. 38A. The configuration of the biosensor illustrated in FIG. 38B is identical to the configuration of the biosensor illustrated in FIG. 15C with the exception that material 106 is further separated from the spacer 140/material 110 stack in the biosensor illustrated in FIG. 38B. The configuration of the biosensor illustrated in FIG. 38C is identical to the configuration of the biosensor illustrated in FIG. 15D with the exception that material 106 is further separated from the spacer 140/material 110 stack in the biosensor illustrated in FIG. 38C. In some embodiments in accordance with FIGS. 38A, 38B, or 38C, material 106 and the spacer 140/material 110 stack are separated by a distance that is between 5 Angstroms and 20 Angstroms, between 20 Angstroms and 50 Angstroms, between 50 Angstroms and 100 Angstroms, or that is more than 100 Angstroms. The configuration of the biosensor illustrated in FIG. 39A is identical to the configuration of the biosensor illustrated in FIG. 21A with the exception that material 106 is juxtaposed against spacer 140 in the biosensor illustrated in FIG. 39A. The configuration of the biosensor illustrated in FIG. 39B is identical to the configuration of the biosensor illustrated in FIG. 21B with the exception that material 106 is juxtaposed against spacer 140 in the biosensor illustrated in FIG. 39B. The configuration of the biosensor illustrated in FIG. 39C is identical to the configuration of the biosensor illustrated in FIG. 21D with the exception that material 106 is juxtaposed against spacer 140 in the biosensor illustrated in FIG. 39C.

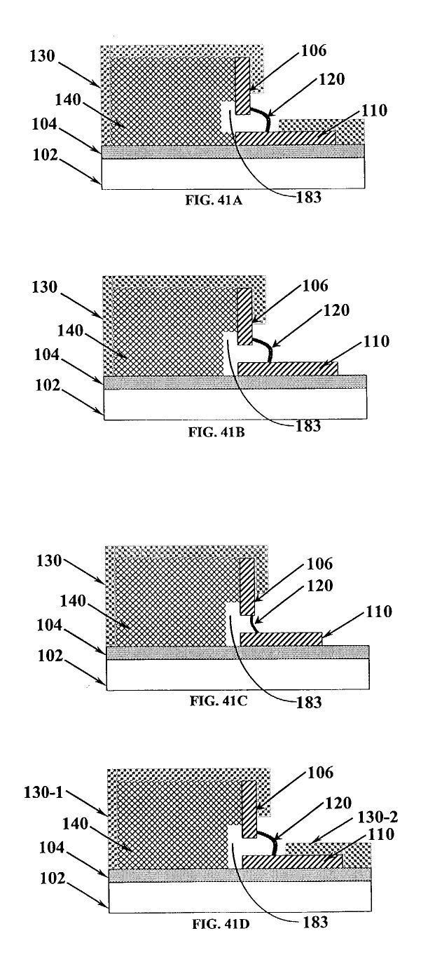

The configuration of the biosensors illustrated in FIGS. 39D, 40A, and 40B are respectively identical to the configuration of the biosensors illustrated in FIGS. 39A, 39B, and 39C with the exception of cavity 183 that is present in the biosensors illustrated in FIGS. 39D, 40A, and 40B. In the biosensors illustrated in FIGS. 39D, 40A, and 40B, cavity 183 extends into spacer 140 and is coextensive with lower edge 175 of material 106.

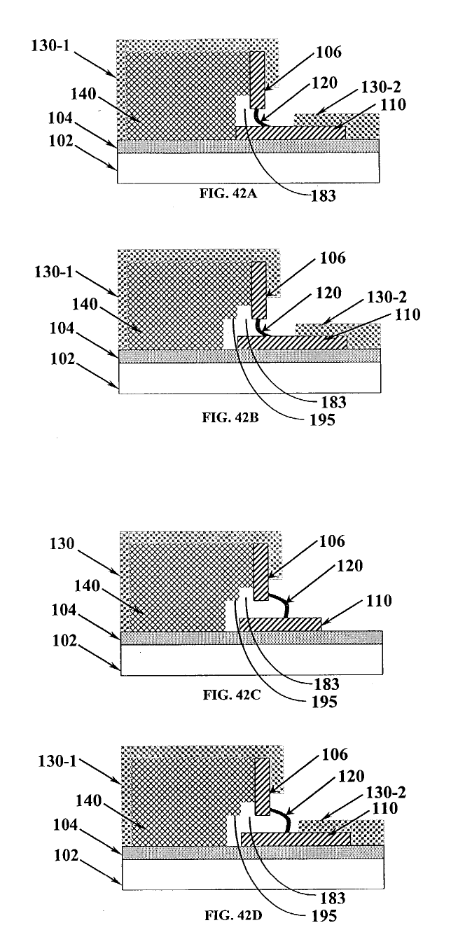

The configuration of the biosensors illustrated in FIGS. 40C, 40D, and 41A are respectively identical to the configuration of the biosensors illustrated in FIGS. 39A, 39B, and 39C with the exception of cavity 183 that is present in the biosensors illustrated in FIGS. 40C, 40D, and 41A. In the biosensors illustrated in FIGS. 40C, 40D, and 41A, cavity 183 extends into spacer 140 but is not coextensive with lower edge 175 of material 106. Rather, top of cavity 183 extends to a level in spacer 140 that is higher than lower edge 175 of material 176.

In the biosensors illustrated in FIGS. 40C, 40D and 41A, the bottom of cavity 183 is coextensive with the top of material 110. The configuration of the biosensors illustrated in FIGS. 41B, 41C, and 41D is respectively identical to the configuration of the biosensors illustrated in FIGS. 40C, 40D and 41A with the exception that the bottom of cavity 183 is not coextensive with the top of material 110. Rather, in the biosensors illustrated in FIGS. 41B, 41C, and 41D, the bottom of cavity 183 extends all the way to the upper surface of insulator 104.

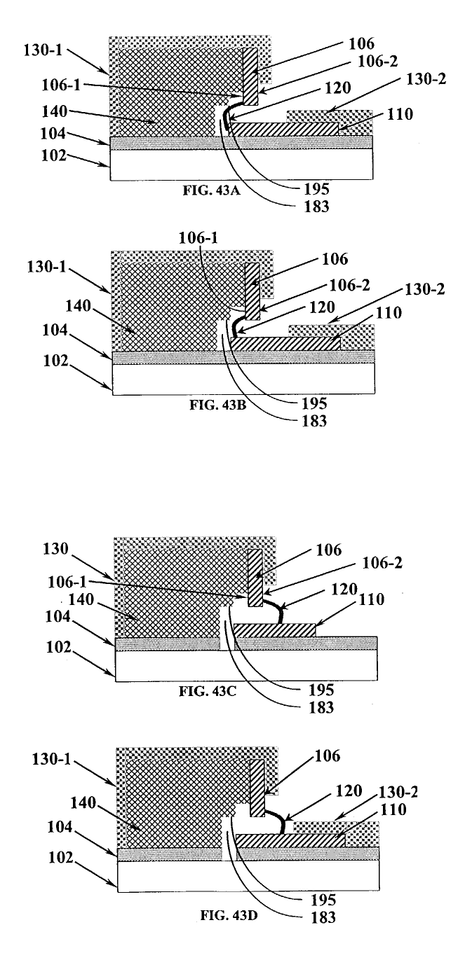

The configuration of the biosensor illustrated in FIG. 42A is identical to the configuration of the biosensor illustrated in FIG. 40D with the exception that a portion of material 110 is covered by a passivation layer 130-2 in the biosensor illustrated in FIG. 42A.

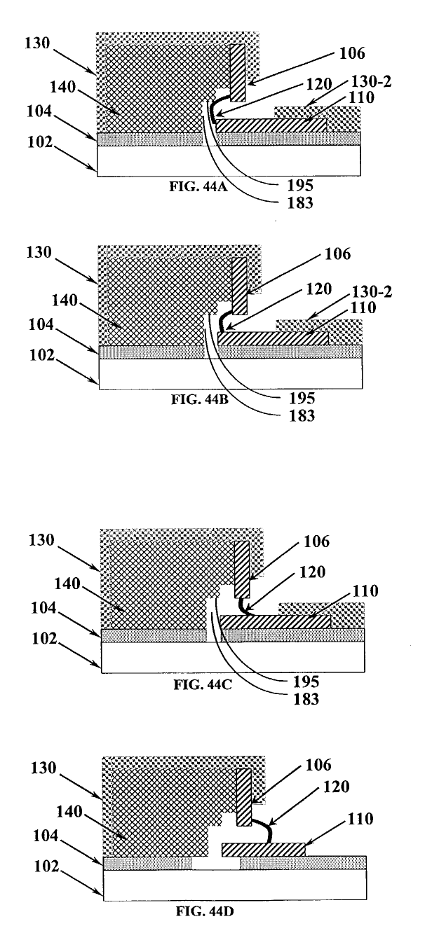

The configuration of the biosensors illustrated in FIGS. 42B, 42C, and 42D is respectively identical to the configuration of the biosensors illustrated in FIGS. 41B, 41C, and 41D with a few exceptions. A first exception is that cavity 183 is enlarged in the biosensors illustrated in FIGS. 42B, 42C, and 42D with respect to the biosensors illustrated in FIGS. 41B, 41C, and 41D. A second exception is that material 110 is situated such that a portion of material 106 lies to the left of material 106 in the biosensors illustrated in FIGS. 42B, 42C, and 42D. A third exception is that spacer 140 optionally includes protrusion 195 in the biosensors illustrated in FIGS. 42B, 42C, and 42D. Placement of material 110 such that a portion of material 106 lies to the left of material 106 in the biosensors illustrated in FIGS. 42B, 42C, and 42D allows for biosensors configurations illustrated in FIGS. 43A and 43B in which a portion of macromolecule 120 binds to face 106-1 of material 106 rather than face 106-2.

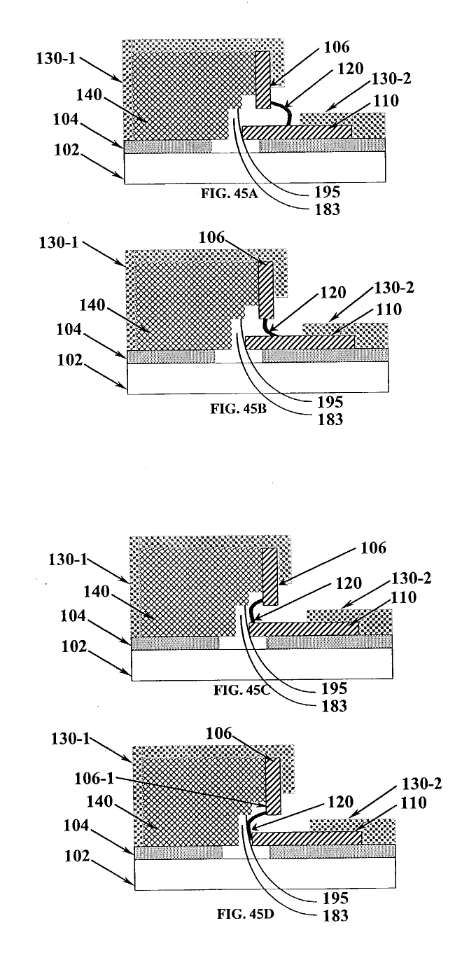

The configuration of the biosensors illustrated in FIGS. 43C, 43D, 44A, and 44B are respectively identical to the configuration of the biosensors illustrated in FIGS. 42C, 42D, 43A, and 43B with the exception that cavity 183 extends through insulator layer 104 to substrate 102 in the biosensors illustrated in FIGS. 43C, 43D, 44A, and 44B. The configuration of the biosensor illustrated in FIG. 44C is identical to the configuration of the biosensor illustrated in FIG. 44B with the exception that a portion of macromolecule 120 binds to bottom 106-3 rather than side 106-1 of material 106 in the biosensor illustrated in FIG. 44C.

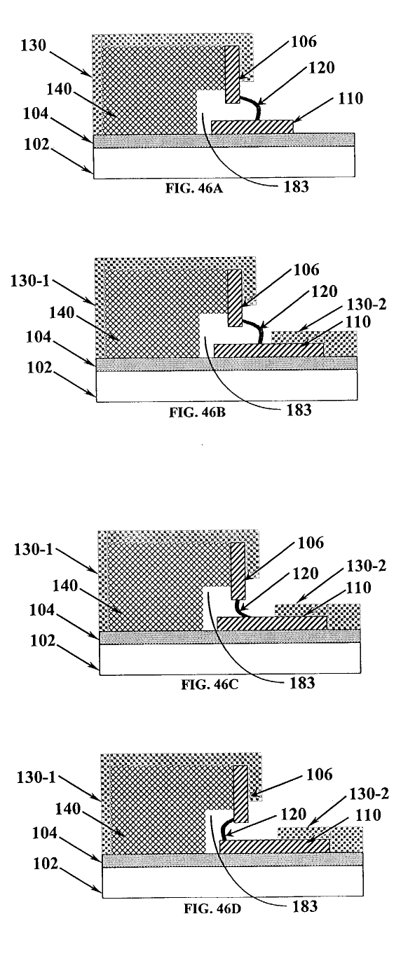

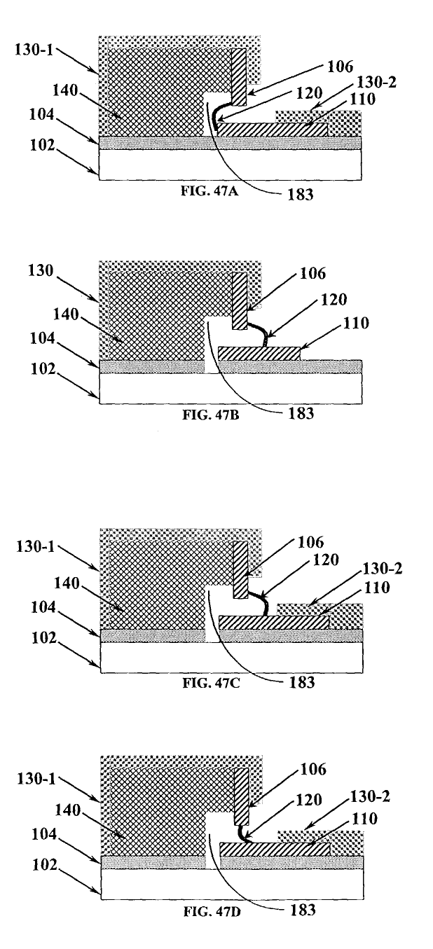

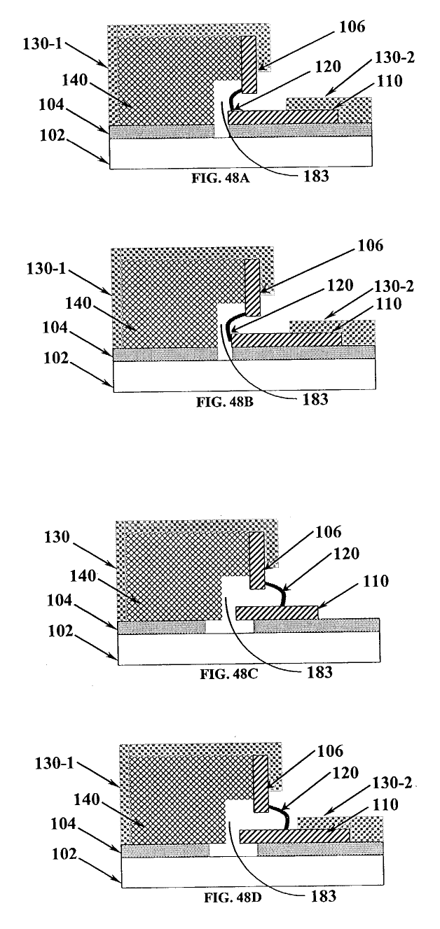

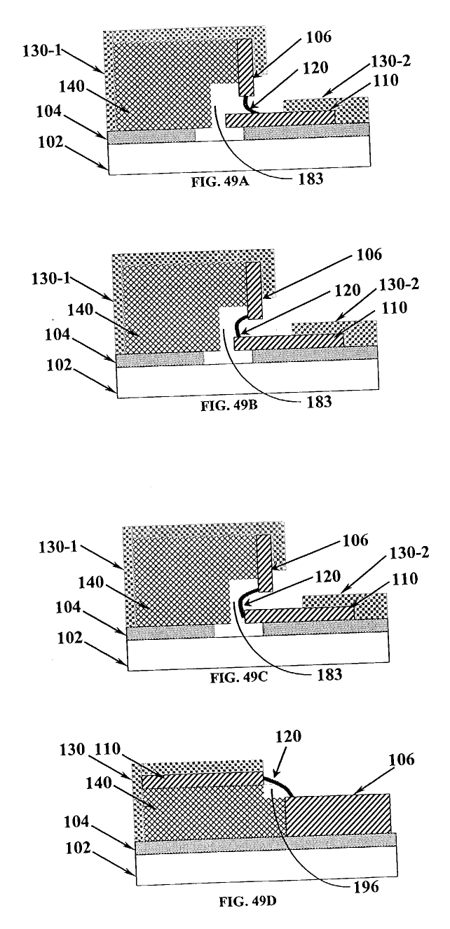

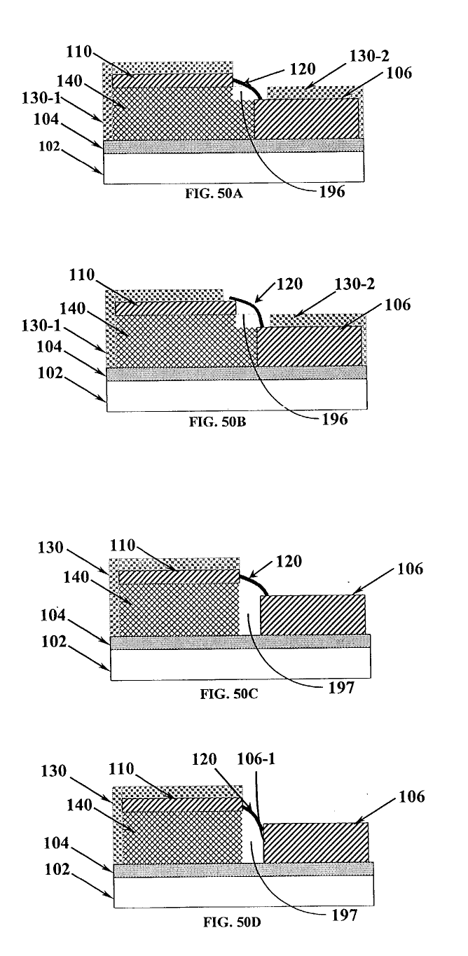

The configuration of the biosensors illustrated in FIGS. 44D, 45C, and 45D are respectively identical to the configuration of the biosensors illustrated in FIGS. 43C, 43B, and 44A with the exception that the portion of cavity 183 in insulator layer 104 is extended such that material 106 protrudes into cavity 183. The configuration of the biosensors illustrated in FIGS. 46A, 46B, 46C, 46D, and 47A are respectively identical to the configuration of the biosensors illustrated in FIGS. 42C, 42D, 42B, 43B, and 43A with the exception that the biosensors illustrated in FIGS. 46A, 46B, 46C, 46D, and 47A do not include protrusion 195. The configuration of the biosensors illustrated in FIGS. 47B, 47C, 47D, 48A, and 48B are respectively identical to the configuration of the biosensors illustrated in FIGS. 43C, 43D, 44C, 44B, and 44A with the exception that cavity 183 in the biosensors illustrated in FIGS. 47B, 47C, 47D, 48A, and 48B does not include protrusion 195. The configuration of the biosensors illustrated in FIGS. 48C, 48D, 49A, 49B, and 49C are respectively identical to the configuration of the biosensors illustrated in FIGS. 47B, 47C, 47D, 48A, and 48B with the exception that cavity 183 in the biosensors illustrated in FIGS. 48C, 48D, 49A, 49B, and 49C is extended in insulative layer 104 such that material 110 protrudes into cavity 183.

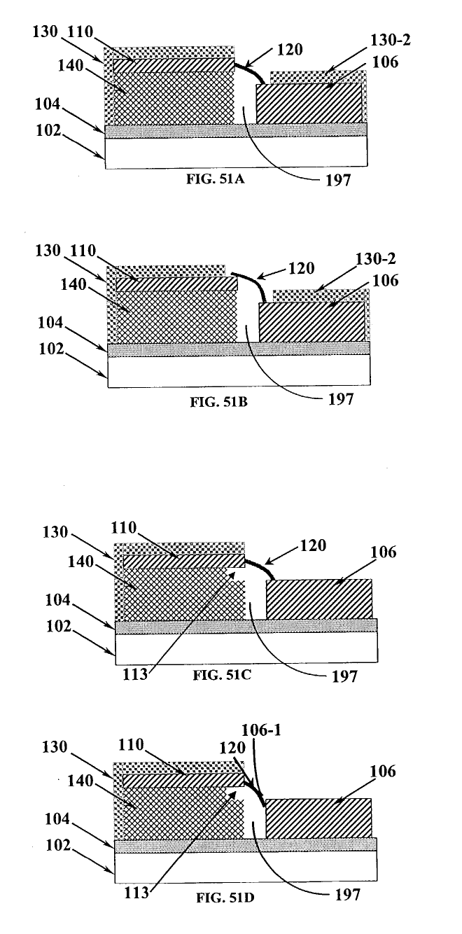

The configuration of the biosensors illustrated in FIGS. 49D, 50A, and 50B is identical to the configuration of the biosensors illustrated in FIGS. 10D, 11A, and 11B with the exception that the biosensors illustrated in FIGS. 49D, 50A, and 50B include a cleft 196 in spacer 140. In some embodiments, cleft 196 has a width of 5 Angstroms, 20 Angstroms, 50 Angstroms, 100 Angstroms, or more than 150 Angstroms. In some embodiments, cleft 196 has a height of 5 Angstroms, 20 Angstroms, 50 Angstroms, 100 Angstroms, or more than 150 Angstroms.

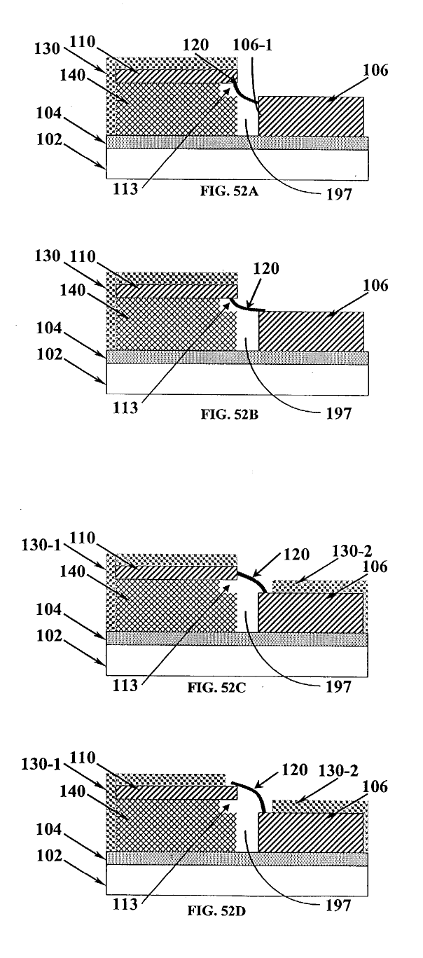

The configuration of the biosensor illustrated in FIG. 50C is identical to the configuration of the biosensor illustrated in FIG. 10D with the exception that material 106 is not juxtaposed against spacer 140 in the biosensor illustrated in FIG. 50C. Rather, a gap 197 separates material 106 and spacer 140 in the biosensor illustrated in FIG. 50C. In some embodiments of the present invention, gap 197 has a width of 5 Angstroms, a width of 20 Angstroms, a width of 50 Angstroms, a width of 100 Angstroms, or a width of more than 150 Angstroms. Gap 197 advantageously allows for biosensors having the configuration illustrated in FIG. 50D, in which a portion of macromolecule 120 binds side 106-1 of material 106 rather than the top of material 106. The configuration of the biosensors illustrated in FIGS. 51A and 51B are respectively identical to the configuration of the biosensors illustrated in FIGS. 11A and 11B with the exception that material 106 is not juxtaposed against spacer 140 in the biosensors illustrated in FIGS. 51A and 51B.

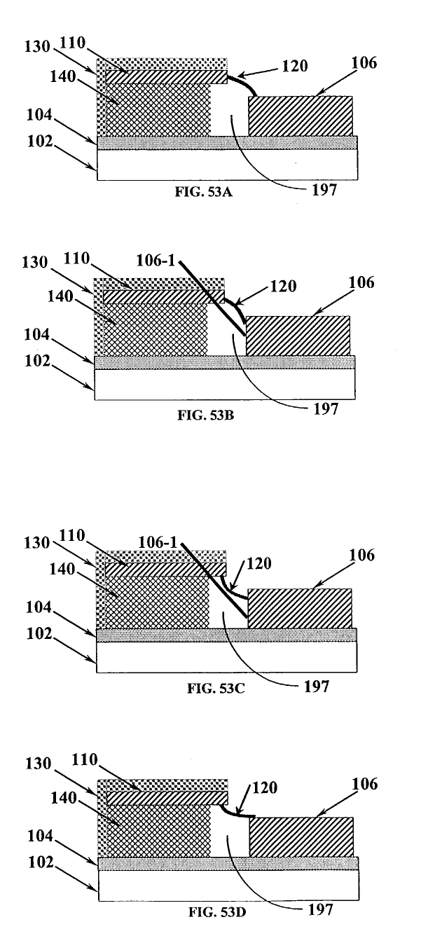

The configuration of the biosensor illustrated in FIG. 51C is identical to the configuration of the biosensor illustrated in FIG. 11C with the exception that material 106 is not juxtaposed against spacer 140 in the biosensor illustrated in FIG. 51C. Rather, a gap 197 separates material 106 and spacer 140 in the biosensor illustrated in FIG. 51C. Gap 197 advantageously allows for biosensors having the configurations illustrated in FIGS. 51D and 52A, in which a portion of macromolecule 120 binds side 106-1 of material 106 rather than the top of material 106. The configuration of the biosensors illustrated in FIGS. 42B, 52C, and 52D are respectfully identical to the configuration of the biosensors illustrated in FIGS. 11D, 12A, and 12B with the exception that material 106 is not juxtaposed against spacer 140 in the biosensor illustrated in FIG. 51C. Rather, a gap 197 separates material 106 and spacer 140 in the biosensors illustrated in FIGS. 42B, 52C, and 52D.

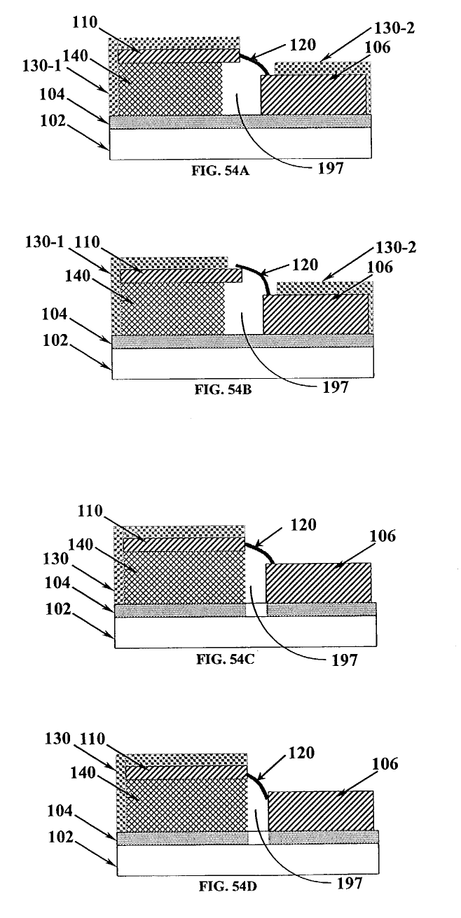

The configuration of the biosensor illustrated in FIG. 53A is identical to the configuration of the biosensor illustrated in FIG. 12C with the exception that the biosensor in FIG. 53A includes a gap 197 that separates material 106 and spacer 140 in the biosensor illustrated in FIG. 53A. In some embodiments of the present invention, gap 197 has a width of 5 Angstroms, a width of 20 Angstroms, a width of 50 Angstroms, a width of 100 Angstroms, or a width of more than 150 Angstroms. Gap 197 advantageously allows for biosensors having the configuration illustrated in FIGS. 53B and 53C, in which a portion of macromolecule 120 binds side 106-1 of material 106 rather than the top of material 106. The configuration of the biosensors illustrated in 53D, 54A, and 54B are respectively identical to the configuration of the biosensors illustrated in FIGS. 12D, 12A, and 12B with the exception that the biosensors in 53D, 54A, and 54B include a gap 197 that separates material 106 and spacer 140.

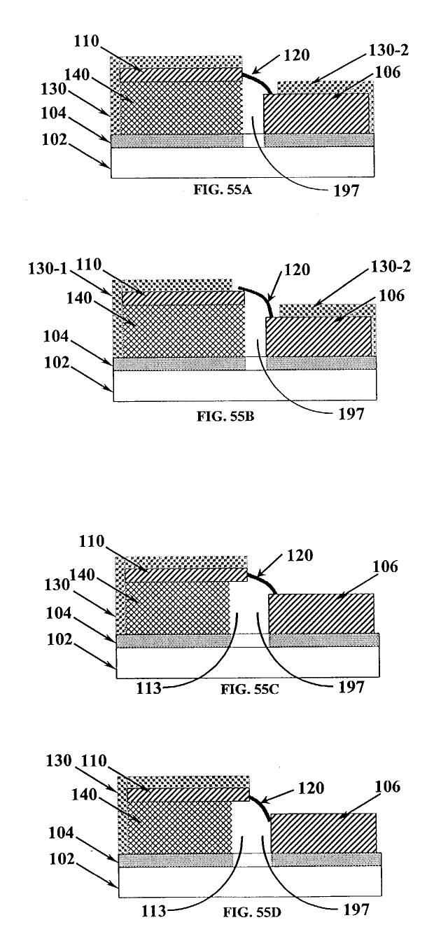

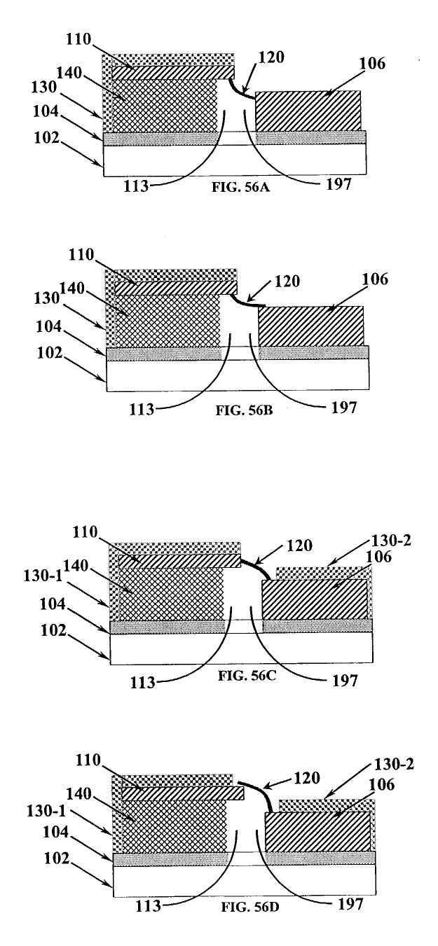

The configuration of the biosensors illustrated in FIGS. 54C, 54D, 55A, and 55B are respectively identical to the configuration of the biosensors illustrated in FIGS. 50C, 50D, 51A, and 51B with the exception that gap 197 extends through insulator layer 104 to substrate 102 in the biosensors illustrated in FIGS. 54C, 54D, 55A, and 55B.

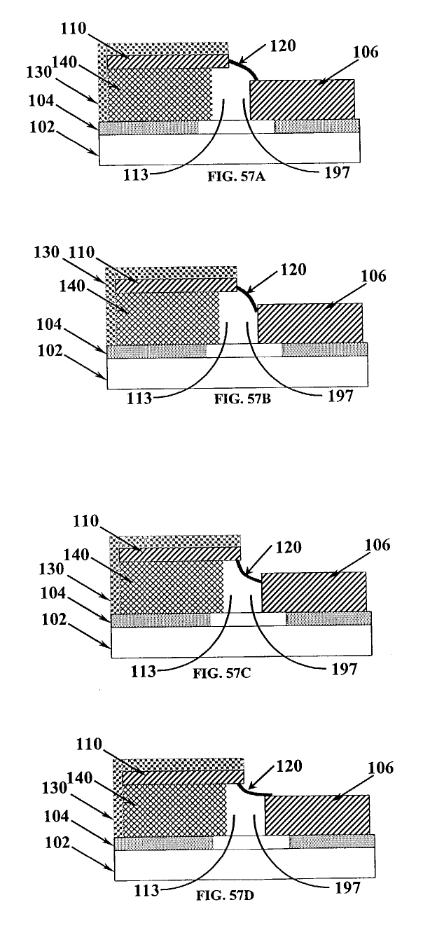

The configuration of the biosensors illustrated in FIGS. 55C and 55D are respectively identical to the configuration of the biosensors illustrated in FIGS. 54C and 54D with the exception that the biosensors illustrated in FIGS. 55C and 55D include a cavity 113 in spacer 140 such that a portion of material 110 overhangs cavity 113. Cavity 113 in the biosensors illustrated in FIGS. 55C and 55D allows for advantageous biosensor configurations such as those shown in FIGS. 56A and 56B in which a portion of macromolecule 120 binds to bottom surface 110-1 of material 110 rather than the side of material 110. The configuration of the biosensors illustrated in FIGS. 56C and 56D are identical to the configuration of the biosensors illustrated in FIGS. 55A and 545B with the exception that the biosensors illustrated in FIGS. 56C and 56D include a cavity 113 in spacer 140 such that a portion of material 110 overhangs cavity 113. In some embodiments, cavity 113 has a width of 5 Angstroms, a width of 20 Angstroms, a width of 50 Angstroms, a width of 100 Angstroms, or a width of more than 150 Angstroms.

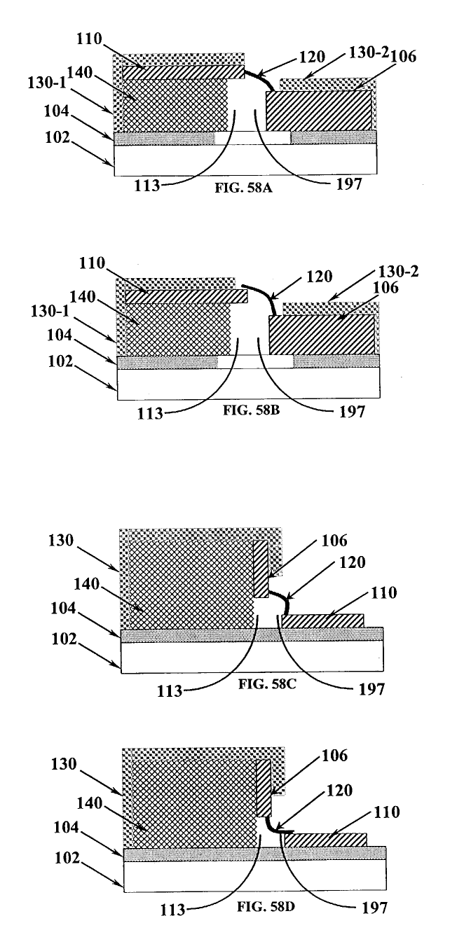

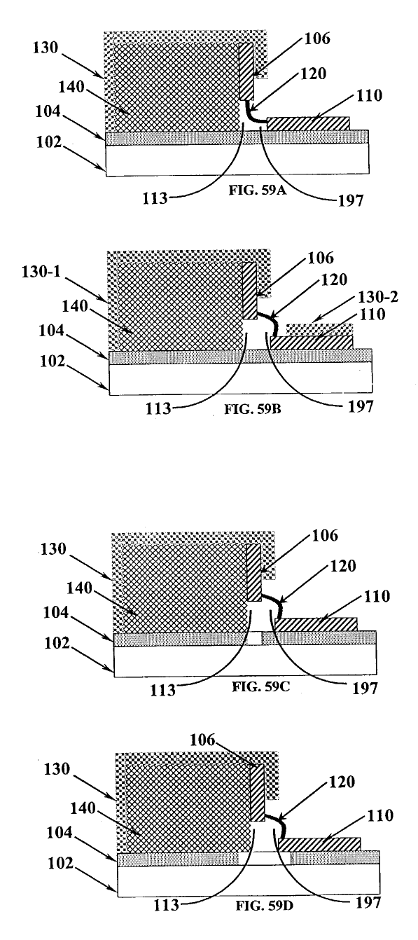

The configuration of the biosensor illustrated in FIG. 57A is identical to the configuration of the biosensor illustrated in FIG. 14D with the exception that the biosensor illustrated in FIG. 57A includes a gap 197 that separates material 110 from the spacer 140/material 110 stack. Gap 197 in the biosensor illustrated in FIG. 57A allows for advantageous biosensor configurations, such as those shown in FIGS. 57B and 57C, in which a portion of macromolecule 120 binds to side 106-1 of material 106 rather than the top of material 106. The configuration of the biosensors illustrated in FIGS. 57D, 58A, and 58B are respectively identical to the configurations of the biosensors illustrated in 15A, 15B, and 15C with the exception that the biosensors illustrated in FIGS. 57D, 58A, and 58B include a gap 197 that separates material 110 from the spacer 140/material 110 stack. In some embodiments, gap 197 has a width of 5 Angstroms, a width of 20 Angstroms, a width of 50 Angstroms, a width of 100 Angstroms, or a width of more than 150 Angstroms.

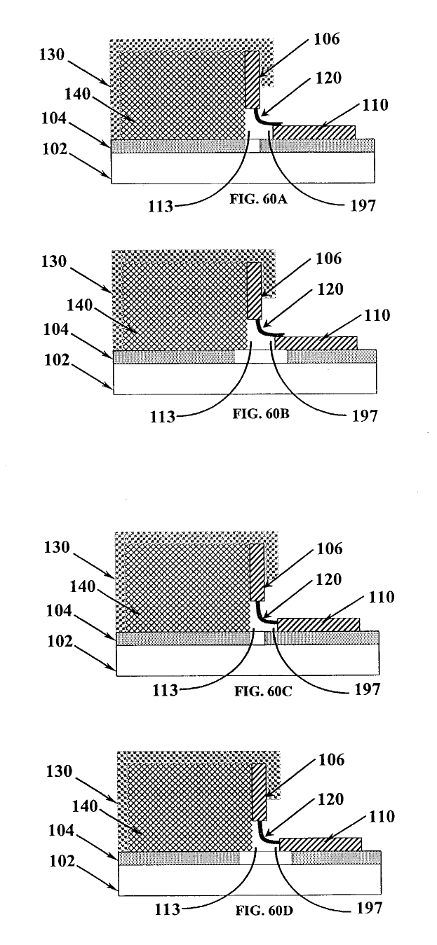

The configuration of the biosensors illustrated in FIGS. 58C, 58B, 59A, and 59B are respectively identical to the configuration of the biosensors illustrated in FIGS. 21A, 21B, 21C, and 21D with the exception that the biosensors illustrated in FIGS. 58C, 58B, 59A, and 59B include a gap 197 that separates material 110 from spacer 140. In some embodiments of FIGS. 58C, 58B, 59A, or 59B, gap 197 has a width of 5 Angstroms, a width of 20 Angstroms, a width of 50 Angstroms, a width of 100 Angstroms, or a width of more than 150 Angstroms.

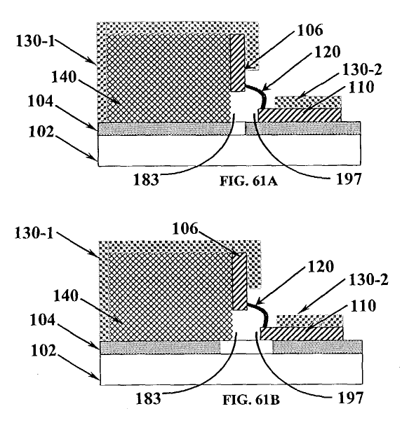

The configuration of the biosensors illustrated in FIGS. 58C, 58D, 59A, 59B, 59C, 59D, 60A, 60B, 60C, and 60D are respectively identical to the configuration of the biosensors illustrated in FIGS. 21A, 21B, 21C, 21D, 22A, 23A, 22C, 23B, 22B, and 23C with the exception that the biosensors illustrated in FIGS. 58C, 58D, 59A, 59B, 59C, 59D, 60A, 60B, 60C, and 60D include a gap 197 that separates material 110 from spacer 140. In some embodiments of FIGS. 58C, 58D, 59A, 59B, 59C, 59D, 60A, 60B, 60C, and 60D, gap 197 has a width of 5 Angstroms, a width of 20 Angstroms, a width of 50 Angstroms, a width of 100 Angstroms, or a width of more than 150 Angstroms.

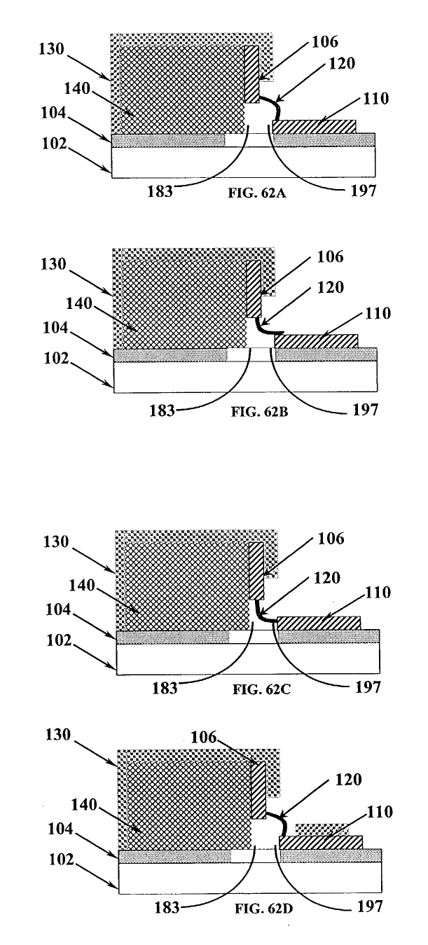

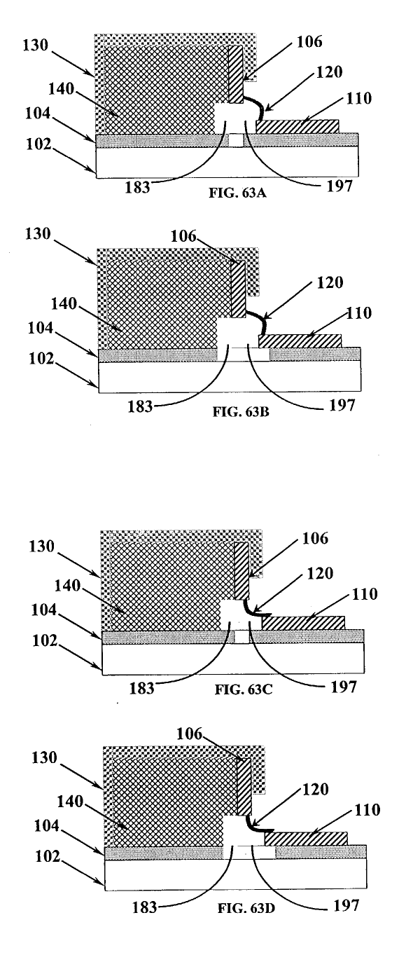

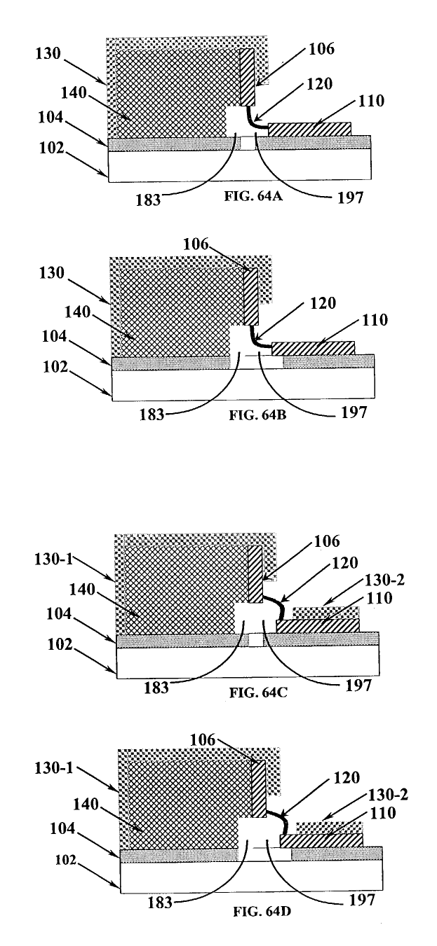

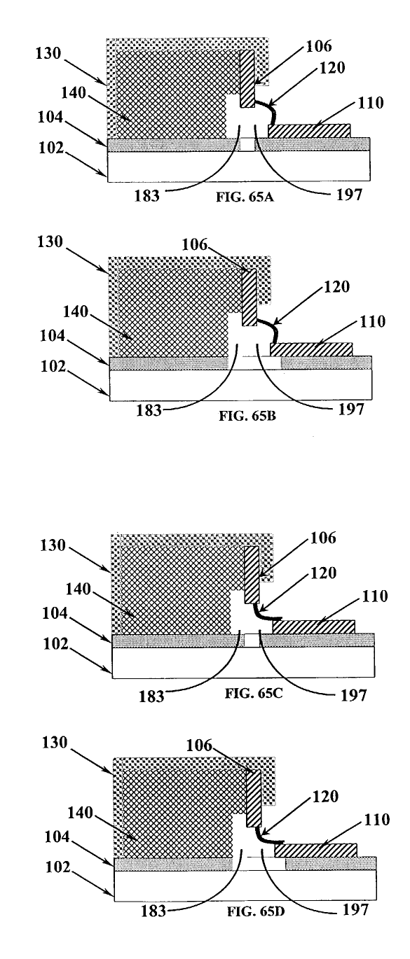

The configuration of the biosensors illustrated in FIGS. 61A, 61B, 62A, 62B, 62C, 62D, 63A, 63C, and 64C are respectively identical to the configuration of the biosensors illustrated in FIGS. 22D, 23D, 23A, 23B, 23C, 23D, 24A, 24B, and 24C with the exception that the biosensors illustrated in FIGS. 61A, 61B, 62A, 62B, 62C, 62D, 63A, 63C, and 64C include a gap 197 that further separates material 110 from spacer 140. In some embodiments of FIGS. 61A, 61B, 62A, 62B, 62C, 62D, 63A, 63C, and 64C, gap 197 has a width of 5 Angstroms, a width of 20 Angstroms, a width of 50 Angstroms, a width of 100 Angstroms, or a width of more than 150 Angstroms. The configuration of the biosensors illustrated in FIGS. 63B, 63D, and 64B are respectively identical to the configuration of the biosensors illustrated in FIGS. 63A, 63C, and 64A with the exception that spacer 197 is extended in the insulator 104 layer such that a portion of material 110 overhangs into cavity 183.

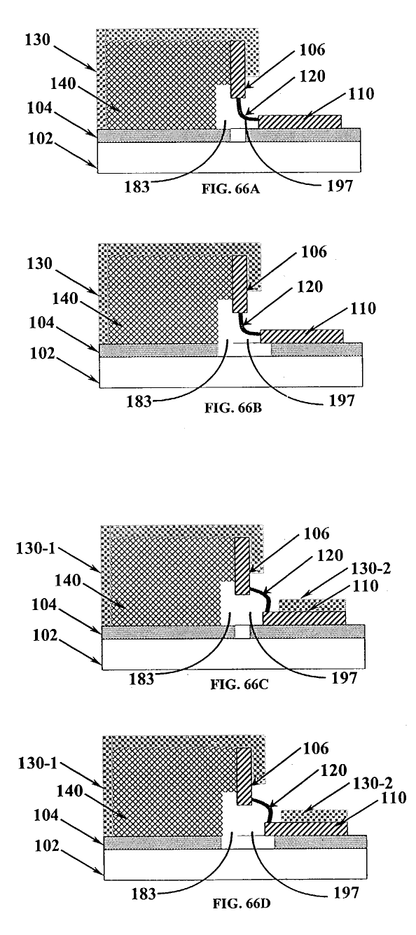

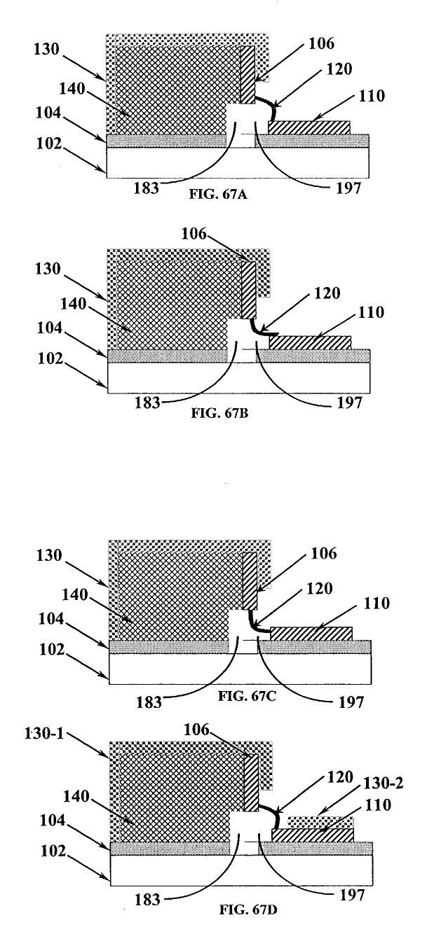

The configuration of the biosensors illustrated in FIGS. 64C, 65A, 65C, 65D, 66A, 66C, 67A, and 67B are respectively identical to the configuration of the biosensors illustrated in FIGS. 24D, 25A, 25B, 25C, 25D, 26A, and 26B with the exception that the biosensors illustrated in FIGS. 64C, 65A, 65C, 65D, 66A, 66C, 67A, and 67B include a gap 197 that further separates material 110 from spacer 140. In some embodiments of FIGS. 64C, 65A, 65C, 65D, 66A, 66C, 67A, and 67B, gap 197 has a width of 5 Angstroms, a width of 20 Angstroms, a width of 50 Angstroms, a width of 100 Angstroms, or a width of more than 150 Angstroms. The configuration of the biosensors illustrated in FIGS. 64D, 65B, 66B, and 66D are respectively identical to the configuration of the biosensors illustrated in 64C, 65A, 66A, and 66C with the exception that spacer 197 is extended in the insulator layer 104 such that a portion of material 110 overhangs into cavity 183. Furthermore, the left side of cavity 183 is uniform across the spacer 140/insulator layer 104 interface in the biosensors illustrated in FIGS. 64D, 65B, 66B, and 66D.

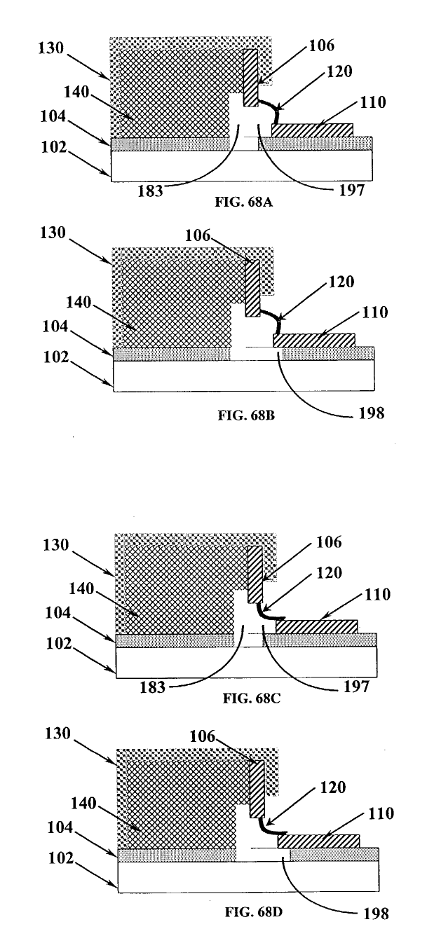

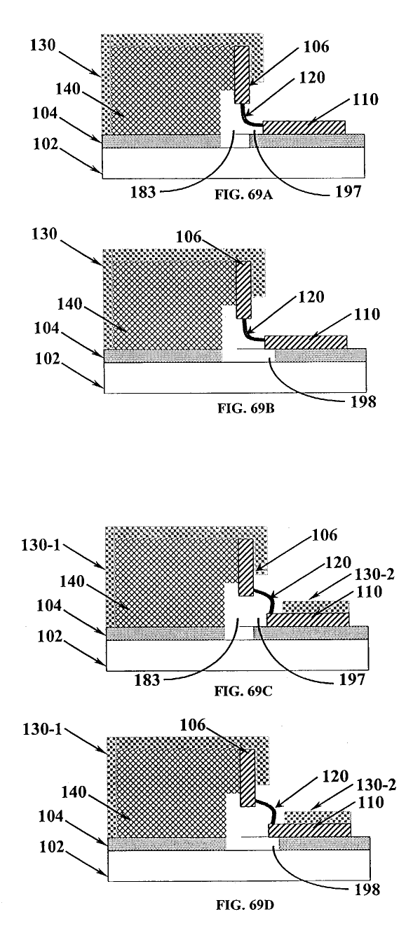

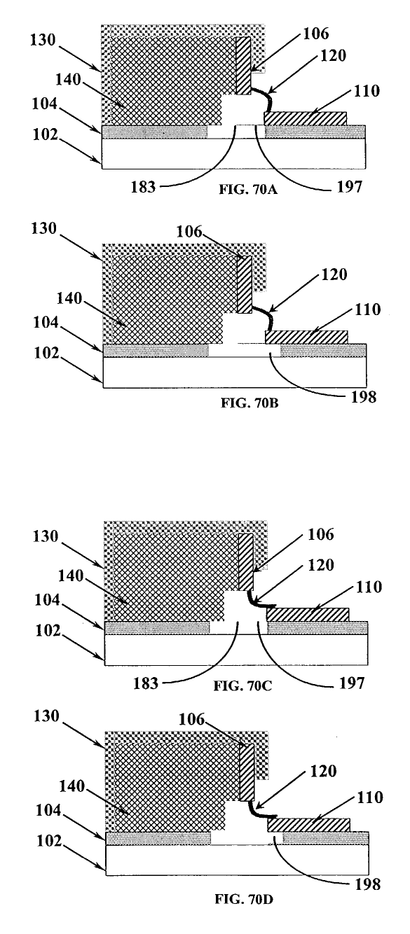

The configuration of the biosensors illustrated in FIGS. 67C, 67D, 68A, 68C, 69A, 69C, and 70A are respectively identical to the configuration of the biosensors illustrated in FIGS. 26C, 26D, 27A, 27B, 27C, 27D, and 28A with the exception that the biosensors illustrated in FIGS. 67C, 67D, 68A, 68C, 69A, 69C, and 70A include a gap 197 that further separates material 110 from spacer 140. In some embodiments of FIGS. 67C, 67D, 68A, 68C, 69A, 69C, and 70A, gap 197 has a width of 5 Angstroms, a width of 20 Angstroms, a width of 50 Angstroms, a width of 100 Angstroms, or a width of more than 150 Angstroms. The configuration of the biosensors illustrated in 68B, 68D, 69D, 69D, and 70B are respectively identical to the configuration of the biosensors illustrated in FIGS. 68A, 68C, 69A, 69C, and 70A with the exception that a cavity 198 in insulator layer 104 is present in the biosensors illustrated in FIGS. 68B, 68D, 69D, 69D, and 70B. Because of cavity 198, a portion of material 110 overhangs into cavity 198. Cavity 198 further isolates material 106 and 110, thereby preventing a short circuit.

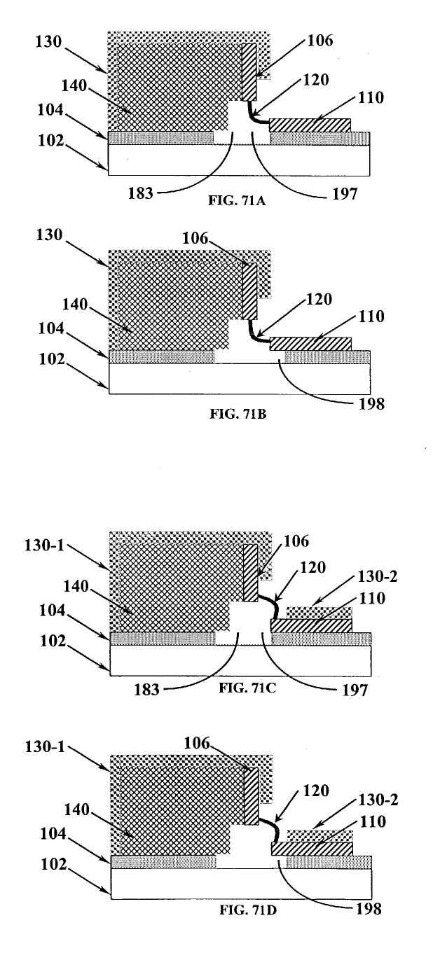

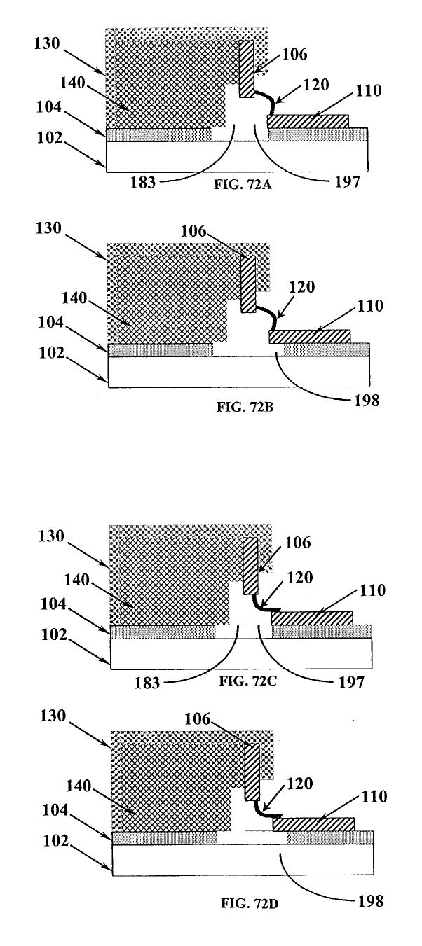

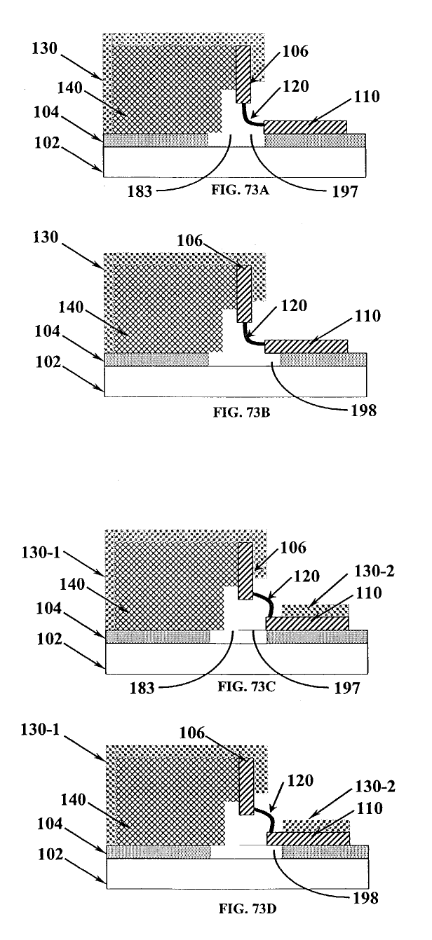

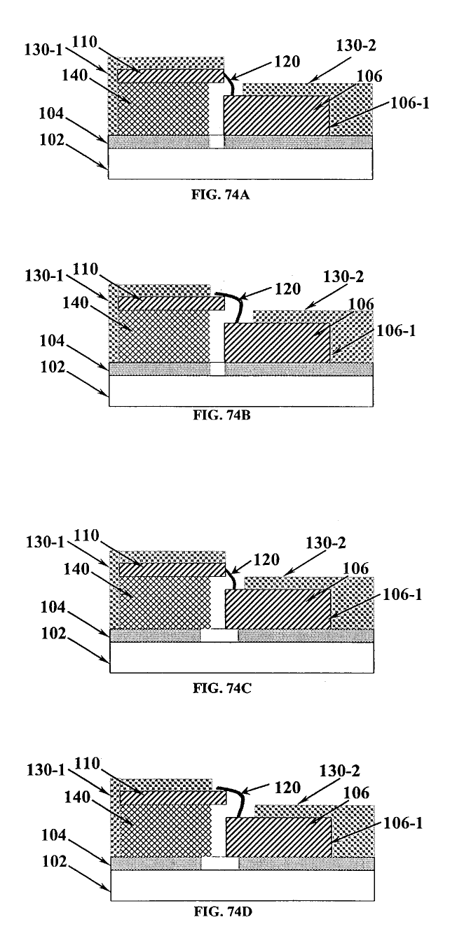

The configuration of the biosensors illustrated in FIGS. 70C, 71A, 71C, 72A, 72C, 73A, and 73C are respectively identical to the configuration of the biosensors illustrated in FIGS. 28B, 28C, 28D, 28A, 29B, 29C, and 29D with the exception that the biosensors illustrated in FIGS. 70C, 71A, 71C, 72A, 72C, 73A, and 73C include a gap 197 that further separates material 110 from spacer 140. In some embodiments of FIGS. 70C, 71A, 71C, 72A, 72C, 73A, and 73C, gap 197 has a width of 5 Angstroms, a width of 20 Angstroms, a width of 50 Angstroms, a width of 100 Angstroms, or a width of more than 150 Angstroms. The configuration of the biosensors illustrated in FIGS. 70D, 71B, 71D, 72B, 72D, 73B, and 73D are respectively identical to the configuration of the biosensors illustrated in FIGS. 70C, 71A, 71C, 72A, 72C, 73A, and 73C with the exception that a cavity 198 in insulator layer 104 is present in the biosensors illustrated in FIGS. 70D, 71B, 71D, 72B, 72D, 73B, and 73D.

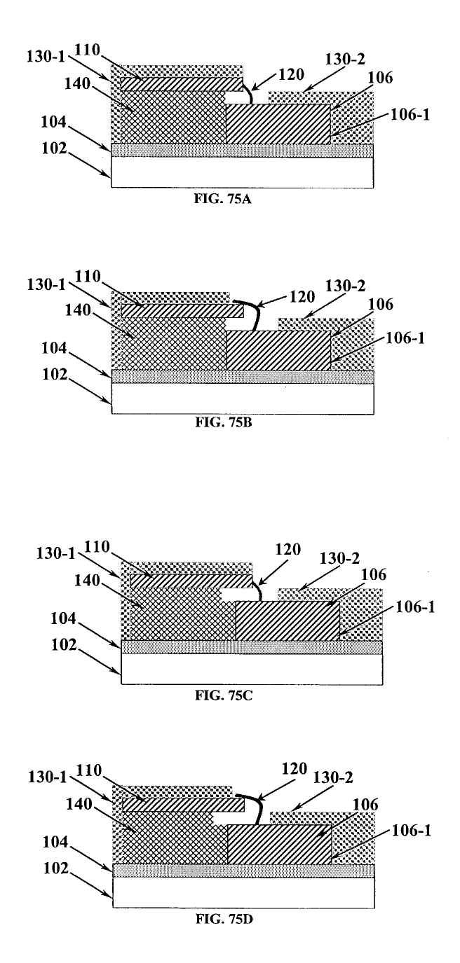

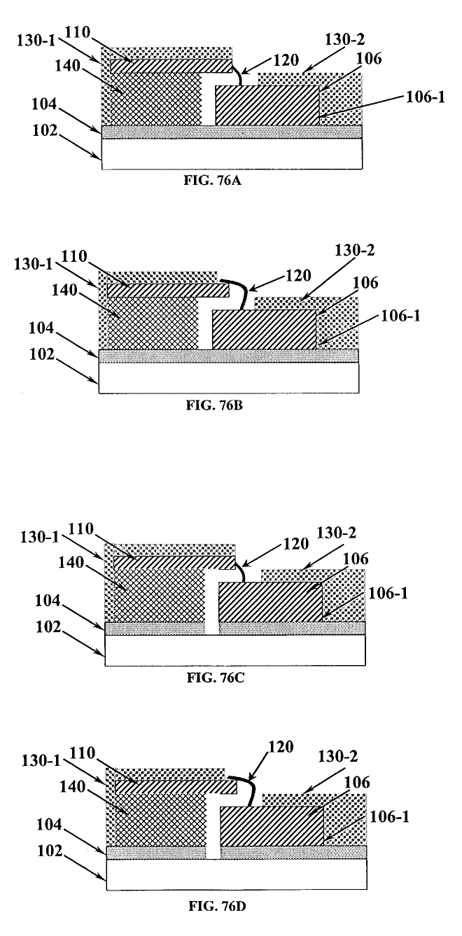

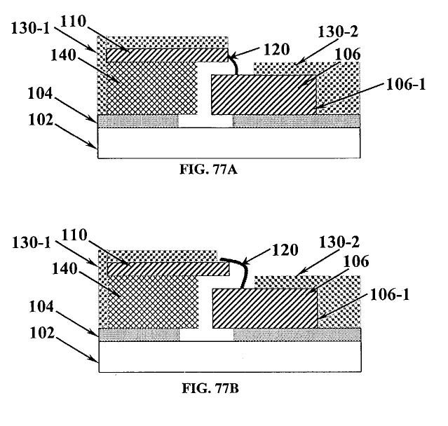

The configuration of the biosensors illustrated in FIGS. 74A, 74B, 74C, 74D, 75A, 75B, 75C, 75D, 76A, 76B, 76C, 76D, 77A, and 77B are respectively identical to the configuration of the biosensors illustrated in FIGS. 14B, 14C, 15C, 15D, 12A, 12B, 13B, 13C, 18C, 18D, 19C, 19D, 20C, and 20D with the exception that passivation layer 130-2 covers side 106-1 of material 106 in the biosensors illustrated in FIGS. 74A, 74B, 74C, 74D, 75A, 75B, 75C, 75D, 76A, 76B, 76C, 76D, 77A, and 77B.

6.1.5 Connecting Device Electrodes to an External Voltage Source

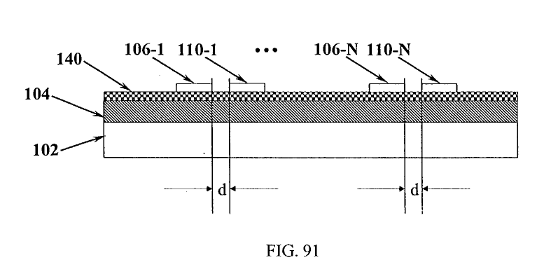

In some embodiments, macromolecules 120 are bound to electrically conducting materials 106 and 110 by applying a voltage to electrode 106 and/or 110. In such embodiments, electrically conducting material 106 and/or electrically conducting material 110 is connected to an external voltage source. For example, electrically conducting materials 106 and/or 110 may be packaged in a chip such as the one disclosed in Section 8.0, below. In some embodiments, electrically conducting materials 106 and 110 are connected to an external voltage source by electrically conducting vias that penetrate optional insulating layer 104 and/or spacer 140. In some embodiments, the conducting vias penetrate substrate 102. As used herein, the term “via” means a vertical opening filled with conducting material used to connect circuits on various layers of a device to one another and to the semiconducting substrate. See, for example, Van Zant, 2000, Microchip Fabrication, McGraw-Hill, New York. Although not shown, some embodiments of the present invention use vias that penetrate optional insulating layer 130, spacer 140, and/or substrate 102 in devices in accordance with FIGS. 1-3, FIGS. 4-77, FIG. 80L, FIGS. 81-91, FIG. 92F, and FIG. 93D.

6.1.6 Biosensors with One or More Attached Macromolecules

In some embodiments, a biosensor of the present invention further comprises a macromolecule 120 that is bound to a first electrically conducting material 106 and/or a second electrically conducting material 110 in a device 144 in the plurality of devices 144 of the biosensor. Such macromolecules 120 comprise a nucleic acid, a protein, a polypeptide, a peptide, an antibody, a carbohydrate, a polysaccharide, a lipid, a fatty acid or a sugar. In some embodiments, the macromolecule 120 is a nucleic acid sequence. In some embodiments, the macromolecule spans the first electrically conducting material 106 and the second electrically conducting material 110. That is, a first portion of the macromolecule 120 binds to the first electrically conducting material and a second portion of the macromolecule binds to the second electrically conducting material in a device 144 in the biosensor.

6.2 Biosensor Arrays

In various embodiments, there can exist multiple macromolecules 120 spanning a single pair of electrodes (e.g., a pair of materials 106 and 110) in a device 144. Furthermore, there can be a multiplicity of electrode pairs (e.g., a multiplicity of devices 144) where each electrode pair is spanned by one or more macromolecules 120. Because of the small size of devices 144, a large number of devices 144 can be placed in a relatively small area (e.g. on a chip) thereby increasing sensitivity and improving signal to noise (S/N) ratio. In addition, assays can be performed using small quantities of sample.

A single substrate/chip can incorporate a number of different devices 144 thereby facilitating detection/quantification of a number of different analytes. Accordingly, in some embodiments, the biosensors of the present invention are arranged into arrays. Each array includes N devices 144. In practice, N is any number. In some embodiments, the biosensors of the present invention each comprise at least one device 144, at least two devices 144, at least ten devices 144, at least 100 devices 144, 1000 to 250,000 devices 144, 10,000 to 60,000 devices 144, 60,000 devices to 10⁵ devices 144, 10⁵ devices to 10⁹ devices 144, 10⁹ devices to 10¹¹ devices 144, 10¹¹ devices to 10¹² devices 144, or more. In some embodiments, each device 144 on a biosensor of the present invention is the same. In some embodiments, materials 106 and 110 are electrodes and each device 144 has an electrode-insulator-electrode configuration. In some embodiments at least two devices 144 in the biosensors of the present invention is different. It will be appreciated that each device 144 in the biosensors of the present invention may serve as an independent sensor for a particular application. Thus, in certain embodiments, an array of devices 144 on a single substrate 102 (e.g., chip) can detect/quantify two or more different analytes, four or more different analytes, 10 or more different analytes, 100 or more different analytes, 1000 or more different analytes, 10,000 more different analytes, 100,000 or more different analytes, or 1,000,000 or more different analytes.

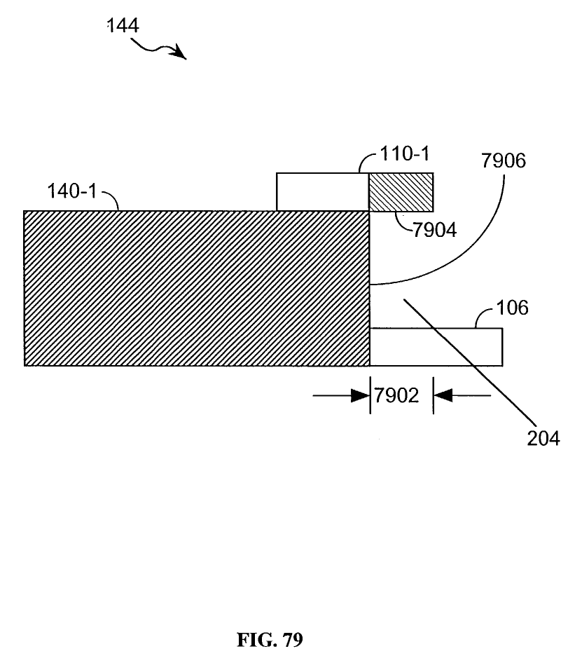

In some embodiments, the biosensors of the present invention comprise an array of discrete devices 144. An illustrative array of discrete devices in a biosensor of the present invention (e.g., biosensor 100, biosensor 200, biosensor 300, a biosensor illustrated in FIGS. 4A-77B) is illustrated in FIG. 78. In the illustrative array shown in FIG. 78, there are N columns and M rows of devices 144. In some embodiments, N and M may be the same or a different number. In some embodiments, N and/or M has a value that is at least two, at least ten, at least 100, 1000 to 10,000, 10,000 to 10⁵, 10⁵ to 10⁷, 10⁷ to 10⁹, 10⁹ to 10¹¹, 10¹¹ to 10¹² devices, or more.

In some embodiments, the biosensors of the present invention include a plurality of devices 144 that are organized into one or more arrays. Each such array may have the configuration shown in FIG. 78, with N columns and M rows, where N and M may be the same or a different number. In some embodiments, the biosensors of the present invention include at least two arrays of devices 144, at least 10 arrays of devices 144, at least 100 arrays of devices 144, or at least 10² to 10²⁰ arrays of devices 144.

In some embodiments, each device 144 in a biosensor of the present invention is overlaid on an optional insulator layer 104. Optional insulator layer 104 is overlaid on substrate 102 in biosensors of the present invention. In some embodiments, there is no optional insulator layer 104 present in all or a portion of biosensors of the present invention and devices 144 are overlaid directly onto substrate 102.

The devices 144 in the biosensors of the present invention can adopt a wide variety of configurations. Thus, for example, in some embodiments of the present invention a macromolecule 120 does not span to material 106 and 110 in an electrode pair. Rather, a first macromolecule 120 is attached to material 106 and a second macromolecule 120 is attached to material 110. Binding of the analyte to the two macromolecules 120 can form an electrically conductive moiety that spans the gap between the two electrodes thereby allowing current to flow between the electrodes. Detection and measurement of this current allow for the detection/quantification of the bound analyte. Thus, for example, in one embodiment; the first and second macromolecules 120 are each nucleic acids complementary to half of the target analyte. When the analyte contacts the first and second macromolecules 120 under conditions permitting hybridization, the two macromolecules hybridize to the analyte forming a double-stranded nucleic acid spanning materials 106 and 110. The use of a first and second macromolecules 120 in this way can be performed with any of the biosensors described herein.

In another embodiment of the present invention, macromolecule 120 is attached to a material 106 of a device 144. The target analyte is tagged with a binding agent that causes the analyte to interact with and/or bind material 110. In use, the analyte binds to, e.g. material 110 and is bound by macromolecule 120. Together, the target analyte, with its binding agent, and macromolecule 120 bridge the gap between the electrodes 106 and 110 resulting in a detectable change in conductance. The use of a macromolecule 120 and a target analyte in this way can be performed with any of the biosensors described herein.

While a single macromolecule can span an electrode pair in a device 144, typically, a plurality of macromolecules 120 span any given electrode-pair in devices 144. Thus, in some embodiments, between two and ten, between ten and fifty, between fifty and 100, between 100 and 1,000, between 1,000 and 10,000, between 10,000 and 100,0000, or at least 1,000,000 macromolecules 120 span an electrode or electrode pair in a device 144 in a biosensor of the present invention.

6.3 Substrates Used in the Biosensors of the Present Invention

In some embodiments, substrate 102 is nonconductive. In some embodiments, substrate 102 is an insulator. In some embodiments, substrate 102 is made of a material such as silicon, silicon oxide, silicon dioxide, silicon nitride, Teflon, or alumina. In some embodiments, substrate 102 is made of glass. For example, in some embodiments, substrate 102 is made from a 600 cm×800 cm motherglass, a 1 meter×1.2-meter motherglass, or larger. In some embodiments of the present invention substrate 102 is made of polyester. In some embodiments, substrate 102 is made out of sapphire, nitrides, arsenides, carbides, oxides, phosphides, or selinides. In some embodiments, substrate 102 is Alkali-free borosilicate glass (Shott AF45).

6.4 Composition of Biosensor Electrodes

In some embodiments, materials 106 and 110 serve as electrodes in biosensors of the present invention. Accordingly, in some embodiments of the present invention, materials 106 and 110 are formed from essentially any conductive material. For example, in some embodiments of the present invention, material 106 and/or material 110 has a resistivity of less than 10-3 ohm-meters, less than 10-4 ohm meters, less than 10⁻⁶ ohm meters, or less than 10⁻⁷ ohm meters.

In some embodiments of the present invention, materials 106 and 110 are made of the same composition. In other embodiments of the present invention, materials 106 and 110 are made of different compositions. In some embodiments, material 106 and/or material 110 comprises silicon, dense silicon carbide, boron carbide, Fe₃O₄, germanium, silicone germanium, silicon carbide, polysilicon, tungsten carbide, titanium carbide, indium phosphide, gallium nitride, gallium phosphide, aluminum phosphide, aluminum arsenide, mercury cadmium telluride, tellurium, selenium, ZnS, ZnO, ZnSe, CdS, ZnTe, GaSe, CdSe, CdTe, GaAs, InP, GaSb, InAs, Te, PbS, InSb, InSb, PbTe, PbSe, tungsten disulfide, or any combination thereof

In some embodiments, material 106 and/or material 110 comprises a metal. In some embodiments, material 106 and/or material 110 is made of a material selected from the group consisting of ruthenium, cobalt, rhodium, rubidium, lithium, sodium potassium, vanadium, cesium, magnesium, calcium, chromium, molybdenum, silicon, germanium, aluminum, iridium, nickel, palladium, platinum, iron, copper, titanium, tungsten, silver, gold, zinc, cadmium, indium tin oxide, carbon, carbon nanotube, or alloys or compounds of such materials.

In some embodiments, material 106 and/or material 110 comprises a metal carbide, metal nitride, or metal boride (e.g., tungsten, titanium, iron, niobium, vanadium, zirconium, hafnium, molybdenum, etc.). In some embodiments, material 106 and/or material 110 comprises a conductive oxide (e.g., transition element monoxide, dioxides and sequioxides, perovskite and perovskite related oxides such as strontinates and lanthanates). In some embodiments, material 106 and/or material 110 comprises a metal silicide or a metal sulfide. In some embodiments, material 106 and/or material 110 comprises a semiconductor or compound semiconductor material.

6.5 Composition of Biosensor Insulators

In some embodiments of the present invention spacer 140 and optional insulator 104 are made from the same materials. In other embodiments of the present invention, spacer 140 and optional insulator 104 are made from different materials. In some embodiments, materials used to make insulator 104 and/or spacer 140 include elements, compounds and substances that have a resistivity greater than 10⁻³ ohm-meters. In some embodiments, materials used to make insulator 104 and/or spacer 140 include elements, compounds and substances that have a resistivity greater than 10⁻² ohm-meters, greater than 10⁻¹ ohm-meters, or greater than 10 ohm-meters. In some embodiments of the present invention insulator 104 and/or spacer 140 is made of high resistivity plastic. A “high resistivity plastic” refers to a plastic with a resistivity greater than 10⁻³ ohm-meters, greater than 10⁻² ohm-meters, greater than 10⁻¹ ohm-meters, greater than 1 ohm-meter, or greater than 10 ohm-meters.

In some embodiments, spacer 140 and/or insulator 104 is made from a material such as TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene, or an insulating oxide or sulfide of a transition metal in the periodic table of elements. The transition metals comprise groups IIIB, IVB, VB, VIIB, VIIIB, IB, and IIB of the periodic table. This group of elements is defined herein as the d-block. In addition to the d-block, transition metals comprise lanthamides and main group elements having chemical properties similar to transition metals. As defined herein, lanthamides are the first row of the f-block of the periodic table and main group elements are those in groups IIIA, IVA, VA and VIIA of the periodic table, the first five groups of which is known to those of skill in the art as the p-block. (See, e.g., Huheey, Inorganic Chemistry, Harper & Row, New York, 1983).

In some embodiments, spacer 140 and/or insulator 104 comprises an air gap insulator, a stoichiometric oxide, a stoichiometric nitride, an off stoichiometric oxide, an off stoichiometric nitride, a polymeric film (e.g., polystyrene or Teflon), an insulting carbon, or an insulating sulfide.

In some embodiments, spacer 140 and/or insulator 104 comprises porcelain, Teflon, ceramics, polymers, or rubber. In some embodiments, where possible, spacer 140 and/or insulator 104 comprises dry air. In some specific embodiments, spacer 140 and/or insulator 104 comprises SiO₂, Al₂O₃, Fe₂O₃, MgO, SrTiO₃, MgAl₂O₄, YBa₂Cu₃O_{7-x,} Si₃N₄, TiN, AlN, GaN, BN, SiC, WC, or TiC. In still other embodiments, spacer 140 and/or insulator 104 comprises SiO₂, fluorinated silicate glass, polycrystalline diamond films, or diamond-like carbon (DLC),

6.6 Composition of Biosensor Passivation Layer

In one embodiment of the present invention, passivation layer 130 is made of any material that does not bind to sulfur. In another embodiment of the present invention, passivation layer 130 is a layer that does not bind to macromolecules 120. In some embodiments of the present invention, passivation layer 130 is a material such as silicon oxide, silicon dioxide, silicon nitride, or silicon oxy-nitride. In some embodiments of the present invention, passivation layer 130 is an organic film such as polyamide. In yet other embodiments of the present invention, passivation layer 130 comprises aluminum having a thin layer of oxidation (oxidized aluminum). The thin layer of oxidation prevents sulfur binding. In some embodiments of the present invention, macromolecule 120 includes sulfur-based groups (e.g., thiols, sulfides) that bind to materials 106 and 110 on the biosensors of the present invention thereby bridging materials 106 and 110.

6.7 Biosensor Electrode Overlap

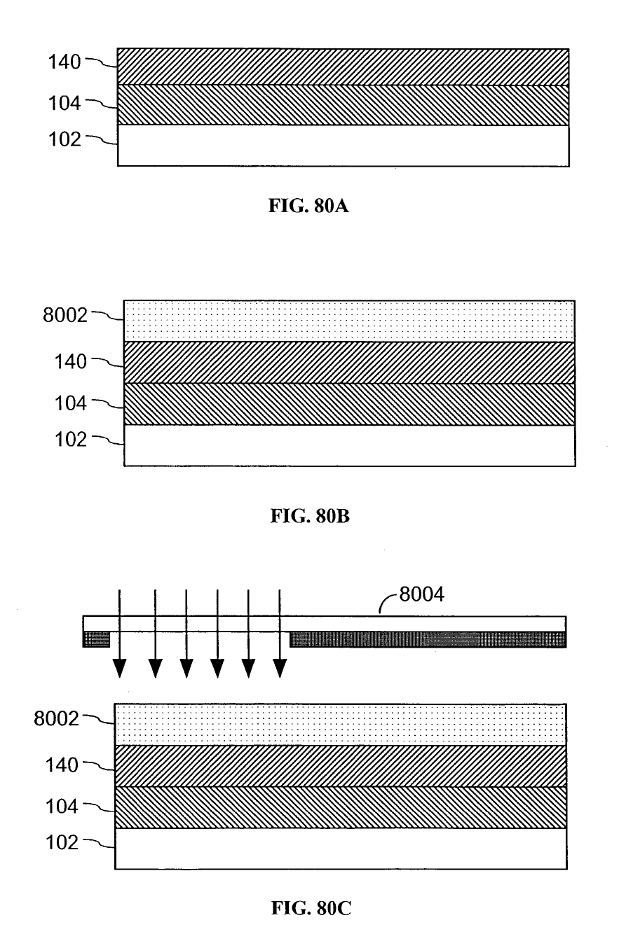

FIG. 79 illustrates an embodiment of device 144 in which material 110 and material 106 overlap each other by an amount 502. Thus, in such embodiments, material 110 includes an edge portion 7904 that overlies spacer 140. In some embodiments, edge portion 7904 (FIG. 5) is formed by removing a portion of spacer 140. In some embodiments, spacer 140 includes an edge 7906 that separates material 106 and material 110. In some embodiments, material 106 and material 110 respectively serve as first and second electrodes and edge 7906 of spacer 140 separates the first and second electrodes.

6.8 Composition of Target Biological Macromolecules

Macromolecule 120 is a biological molecule such as a polymer (e.g., nucleic acid, protein, polypeptide, peptide, antibody), carbohydrate, polysaccharide, lipid, fatty acid, sugar, and the like. Biological molecules 120 include, but are not limited to, receptors, ligands for receptors, antibodies or binding portions thereof (e.g., Fab, (Fab)′₂), proteins or fragments thereof, nucleic acids, oligonucleotides, glycoproteins, polysaccharides, antigens, epitopes, carbohydrate moieties, enzymes, enzyme substrates, lectins, protein A, protein G, organic compounds, organometallic compounds, lipids, fatty acids, lipopolysaccharides, peptides, cellular metabolites, hormones, pharmacological agents, tranquilizers, barbiturates, alkaloids, steroids, vitamins, amino acids, sugars, nonbiological polymers, biotin, avidin, streptavidin, organic linking compounds such as polymer resins, lipoproteins, cytokines, lymphokines, hormones, synthetic polymers, organic and inorganic molecules, etc.

The terms “polypeptide”, “peptide” and “protein” are used interchangeably herein to refer to a polymer of amino acid residues. Such polymers may include one or more amino acid residues that are an artificial chemical analogue of a corresponding naturally occurring amino. The term “nucleic acid” as used herein refers to a deoxyribonucleotide or ribonucleotide in either single- or double-stranded form. The term encompasses nucleic acids, e.g., oligonucleotides, containing known analogues of natural nucleotides that have similar or improved binding properties, for the purposes desired, as the reference nucleic acid. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156). The term nucleic acid is used interchangeably with gene, cDNA, mRNA, oligonucleotide primer, probe and amplification product.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental. Immunology, Third Edition, W. E. Paul, ed., Raven Press, N.Y. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies, those synthesized de novo using recombinant DNA methodologies (e.g., single chain Fv), and those found in display libraries (e.g. phage display libraries).

In one embodiment, macromolecule 120 is a single-stranded nucleic acid. In some embodiments in which macromolecule 120 is a single-stranded nucleic acid, the nucleic acid is derivatized at each terminus with a linker that physically and electrically couples the nucleic acid to respective materials 106 and 110 such that the nucleic acid spans the gap between the materials. Single-stranded nucleic acids are essentially non-conductive. However, when the nucleic acid binding agent is contacted with a complementary nucleic acid analyte under conditions that permit nucleic acid hybridization, the analyte nucleic acid binds to the sensor nucleic acid via complementary base pairing to form a double stranded hybrid duplex spanning the electrodes. This double stranded duplex is electrically conductive. The change in conductivity caused by such binding is readily detected using electrical/electrochemical means.

Macromolecule 120 is not limited to a nucleic acid. Any number of other macromolecules 120 can also be used in the biosensors of the present invention. Generally, macromolecules 120 are selected that are capable of specifically binding to a particular target analyte. Such macromolecules 120 include, but are not limited to, nucleic acids (including, but not limited to single stranded DNA or RNA, double stranded DNA or RNA, peptide nucleic acids, phosphorothioates, and the like), proteins, antibodies, lectins, sugars, lipids, polysaccharides, and the like.

In preferred embodiments, macromolecules 120 are utilized in the biosensors of the present invention that are not conductive in the absence of an analyte. These macromolecules 120 preferably form an electrically conductive complex when bound to an analyte. However, the biosensors of the present invention are not limited to macromolecules 120 that are not electrically conductive in the absence of an analyte. In certain embodiments it is sufficient that the analyte/macromolecule 120 complex exhibits a different electrical conductivity than the uncomplexed macromolecule 120.

Alternatively, where the analyte/macromolecule 120 complex shows the same conductivity as the uncomplexed macromolecule 120, it is possible to use various chemical agents that intercalate into the analyte/macromolecule 120 complex in order to change the effective conductivity of the complex. In some embodiments, an uncomplexed macromolecule 120 affords fewer intercalation sites relative to the analyte/macromolecule 120. Thus, the analyte/macromolecule 120 complex intercalates a greater number of intercalation agents relative to the uncomplexed macromolecule 120. Because of this, the analyte/macromolecule 120 exhibits a conductivity that is different than the conductivity exhibited by the uncomplexed macromolecule 120.

Intercalating reagents that change the conductivity of a macromolecule 120 or an analyte/macromolecule 120 complex are well known to those of skill in the art. Such intercalators include, but are not limited to, redox-active cations (e.g. Ru(NH₃)₆³⁺ and various transition metal/ligand complexes. Suitable transition metals for use in the invention include, but are not limited to, cadmium (Cd), magnesium (Mg), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metal, the platinum metals (Ru, Rh, Pd, Os, Ir and Pt), along with Re, W, Mo and Tc, are preferred. Particularly preferred are ruthenium, rhenium, osmium, platinum and iron.

In some embodiments, transition metals are complexed with a variety of ligands to form suitable transition metal complexes. Suitable ligands include, but are not limited to, amine, pyridine, pyrazine, isonicotinamide, imidazole, bipyridine, substituted derivatives of bipyridine, phenanthrolines (e.g., 1,10-phenanthroline), substituted derivatives of phenanthrolines (e.g., 4,7-dimethylphenanthroline), dipyridophenazine 1,4,5,8,9,12-hexaazatriphenylene, 9,10-phenanthrenequinone diimine, 1,4,5,8-tetraazaphenanthrene, 1,4,8,11-tetra-azacyclotetradecane, diaminopyridine; porphyrins and substituted derivatives of the porphyrin family.

Such intercalating reagents can also be used to detect mismatches between macromolecule 120 and the target analyte. Thus, for example, where macromolecule 120 and the analyte are nucleic acids, intercalating reagents comprising dimeric naphthyridines will specifically intercalate and localize where there is a G-G mismatch between the binding reagent and the target analyte (see, e.g., Nakatani et al., 2001, Nature/Biotechnology, 19: 51-55). Such mismatch specific reagents can be used to detect or screen for single nucleotide polymorphisms (SNPs).

In some embodiments of the present invention macromolecule 120 is modified to make the macromolecule non-insulative or more electrically conductive. For example, in some instances macromolecule 120 is a nucleic acid, the electrical conduction of the macromolecule 120 is controlled using hole doping. See, Lee et al., Abstract D11.006 of the March 2002 meeting of the American Physical Society. In this approach, the conductivity of the nucleic acid is increased by exposing the nucleic acid to oxygen gas or iodine. See also Lee et al., Applied Physics Letters 80, pp. 1670-1672 as well as Furukawa et al. “PES and NEXAFS study of DNA polynucleotides on silicon dioxide surfaces with and without iodine doping”, BL4B, 2001 UVSOR Activity Report. In some instances macromolelcule 120 made non-insulative or more electrically conducting by labeling it with gold, silver, platinum, copper, tin or other conductive metals. The labeling of such metals to macromolecules 120 can be accomplished using a variety of techniques including covalent attachment, photoreaction, intercalation. In the case where the macromolecule is a nucleic acid, the labeling can be accomplished by the attaction of positively charged metal (e.g., Nanogold) clusters to the negatively charged nucleic acid. See, for example, Hainfeld et al. “DNA Nanowires” in Microsc. Microanal. 7, (Suppl. 2: Proceedings) (Proceedings of the Fifty-ninth annual meeting, Microscopy Society of America); Bailey, Price, Voelkl and Mussleman eds., Springer-Verlag, New York, N.Y., 2001, pp. 1034-1035. In Hainfeld et al., double stranded DNA was labeled with 1.4 nm Nanogold clusters such that the spacing between the gold cluster was approximately 2 nm. Gu et al. has demonstrated enhanced activity of nucleic acids that have been intercalated with acridine organge in the presence of visible light. Accordingly, in some embodiments in which macromolecule 120 is a nucleic acid, the macromolecule is intercalated with acridine organge or similar intercalators and electrical measurements of the bound macromolecule 120 are performed in the presence of visible light.

In some embodiments in which macromolecule 120 is nucleic acid, the nucleic acid can be made more conductive or nonisolative by introducing conductive double strand specific intercalators. The use of conductive metal (e.g., nanoparticles) to alter the conductivity of macromolecules 120 is not limited to the case where the macromolecule 120 is a nucleic acid. Indeed, the macromolecule 120 can be any type of macromolecule 120 described herein, including but not limited to a protein.

6.9 Analytes Used to Bind to Target Biological Macromolecules in the Present Invention

Analytes used to bind to macromolecules 120 include, but are not limited to, whole cells, subcellular particles, viruses, prions, viroids, nucleic acids, proteins, antigens, lipoproteins, lipopolysaccharides, lipids, glycoproteins, carbohydrate moieties, cellulose derivatives, antibodies, fragments of antibodies, peptides, hormones, pharmacological agents, cellular components, organic compounds, non-biological polymers, synthetic organic molecules, organo-metallic compounds, and inorganic molecules.

In some embodiments of the present invention, the analyte is purified. In some embodiments, the analyte is found in a sample. The sample can be derived from, for example, a solid, emulsion, suspension, liquid or gas. Furthermore, the sample may be derived from, for example, body fluids or tissues, water, food, blood, serum, plasma, urine, feces, tissue, saliva, oils, organic solvents, earth, water, air, or food products. The sample may comprise a reducing agent or an oxidizing agent, solubilizer, diluent, preservative, or other suitable agents.

Macromolecule 120 and its target analyte can exist as a pair of “binding partners”, e.g. a ligand and its cognate receptor, an antibody and its epitope, etc. Thus, a biological “binding partner” or a member of a “binding pair” refers to a molecule or composition that specifically binds other molecules to form a binding complex such as antibody-antigen, lectin-carbohydrate, nucleic acid-nucleic acid, biotin-avidin, etc.

The analytes used in this invention are selected based upon the characteristics of the macromolecules 120 that are to be identified/quantified. Thus, for example, where macromolecule 120 is a nucleic acid the analyte is preferably a nucleic acid or a nucleic acid binding protein. Where macromolecule 120 is a protein, the analyte is preferably a receptor, a ligand, or an antibody that specifically binds macromolecule 120. Where the macromolecule 120 is a sugar or glycoprotein, the analyte is preferably a lectin, and so forth.

6.10 Preparation of Macromolecules and Analytes

Methods of synthesizing or isolating suitable macromolecules 120 are well known to those of skill in the art as explained below.

6.10.1 Preparation of Macromolecules or Analytes that are Nucleic Acids

Nucleic acids for use as macromolecules 120 or analytes that bind to macromolecules 120 are produced or isolated according to any of a number of known methods. In one embodiment, the nucleic acid is an isolated naturally occurring nucleic acid (e.g., genomic DNA, cDNA, mRNA, etc.). Methods of isolating naturally occurring nucleic acids are known. See, for example, Sambrook et al., 1989, Molecular Cloning—A Laboratory Manua, 2^{nd} Ed., volumes 1-3, Cold Spring Harbor Laboratory, Cold Spring Harbor, N.Y.

In one embodiment, the nucleic acid is created de novo. In one example, the nucleic acid is created through chemical synthesis, e.g., according to the solid phase phosphoramidite triester method described by Beaucage and Caruthers, 1981, Tetrahedron Letts., 22(20): 1859-1862, e.g., using an automated synthesizer, as described in Needham-VanDevanter et al., 1984, Nucleic Acids Res., 12: 6159-6168. Purification of oligonucleotides, where necessary, is typically performed by either native acrylamide gel electrophoresis or by anion-exchange HPLC as described in Pearson and Regnier, 1983, J. Chrom. 255: 137-149. The sequence of the synthetic oligonucleotides can be verified using the chemical degradation method of Maxam and Gilbert, 1980, in Grossman and Moldave (eds.) Academic Press, New York, Meth. Enzymol. 65: 499-560.

6.10.2 Preparation of Macromolecules or Analytes that are Antibodies or Antibody Fragments

Antibodies or antibody fragments for use as macromolecules 120 or as analytes that bind to macromolecules 120 can be produced by a number of methods well known to those of skill in the art. See, for example, Harlow & Lane, 1988, Antibodies: A Laboratory Manual, Cold Spring Harbor Laboratory, and Asai, 1993, Methods in Cell Biology Vol. 37: Antibodies in Cell Biology, Academic Press, Inc. N.Y. In one approach, antibodies are produced by immunizing an animal (e.g. a rabbit) with an immunogen containing a desired epitope. A number of immunogens may be used to produce specifically reactive antibodies. Recombinant protein is the preferred immunogen for the production of monoclonal or polyclonal antibodies. Naturally occurring proteins may also be used either in pure or impure form. Synthetic peptides can also be made using standard peptide synthesis chemistry (see, e.g., Barany and Merrifield, Solid-Phase Peptide Synthesis; pp. 3-284 in The Peptides: Analysis, Synthesis, Biology. Vol. 2: Special Methods in Peptide Synthesis, Part A., Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.)

Methods of production of polyclonal antibodies are known to those of skill in the art. In brief, an immunogen is mixed with an adjuvant and animals are immunized. The animal's immune response to the immunogen preparation is monitored by taking test bleeds and determining the titer of reactivity to the immunogen. When appropriately high titers of antibody to the immunogen are obtained, blood is collected from the animal and antisera are prepared. Further fractionation of the antisera to enrich for antibodies reactive to the immunogen can be done if desired. See, for example, Harlow & Lane, 1988, Antibodies: A Laboratory Manual, Cold Spring Harbor Laboratory.

Monoclonal antibodies may be obtained by various techniques familiar to those skilled in the art. Briefly, spleen cells from an animal immunized with a desired antigen are immortalized, commonly by fusion with a myeloma cell. See, for example, Kohler and Milstein, 1976, Eur. J. Immunol. 6: 511-519. Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., 1989, Science, 246:1275-1281.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment from a library of greater than 10¹⁰ nonbinding clones. To express antibody fragments on the surface of phage (phage display), an antibody fragment gene is inserted into the gene encoding a phage surface protein (pIII) and the antibody fragment-pill fusion protein is displayed on the phage surface (McCafferty et al., 1990, Nature, 348: 552-554; Hoogenboom et al., 1991, Nucleic Acids Res. 19: 4133-4137).

Since the antibody fragments on the surface of the phage are functional, phage bearing antigen binding antibody fragments can be separated from non-binding phage by antigen affinity chromatography (McCafferty et al., 1990, Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al., 1990, Nature, 348: 552-554). Thus even when enrichments are low (Marks et al., 1991, J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (e.g., several hundred) need to be analyzed for binding to antigen.

Human antibodies can be produced without prior immunization by displaying very large and diverse V-gene repertoires on phage (Marks et al., 1991, J. Mol. Biol. 222: 581-597). In one embodiment, natural V_{H} and V_{L} repertoires present in human peripheral blood lymphocytes are isolated from unimmunized donors by PCR. The V-gene repertoires are spliced together at random using PCR to create a scFv gene repertoire that is cloned into a phage vector to create a library of 30 million phage antibodies (Id.). From this single “naive” phage antibody library, binding antibody fragments are isolated against different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Furthermore, antibodies can be produced against self-proteins, including human thyroglobulin, immunoglobulin, tumor necrosis factor and CEA (Griffiths et al., 1993, EMBO J. 12: 725-734). It is also possible to isolate antibodies against cell surface antigens by selecting directly on intact cells. The antibody fragments are highly specific for the antigen used for selection and have affinities in the 1 μM to 100 nM range (Marks et al., 1991, J. Mol. Biol. 222: 581-597; Griffiths et al. (1993) EMBO J. 12: 725-734). Larger phage antibody libraries result in the isolation of more antibodies of higher binding affinity to a greater proportion of antigens.

6.10.3 Preparation of Macromolecules or Analytes that are Proteins

Suitable proteins for use as macromolecules 120 or analytes include, but are not limited to, receptors (e.g. cell surface receptors), receptor ligands, cytokines, transcription factors and other nucleic acid binding proteins, growth factors, etc.

The protein can be isolated from natural sources, mutagenized from isolated proteins, or synthesized de novo. Means of isolating naturally occurring proteins are well known to those of skill in the art. Such methods include, but are not limited to, well known protein purification methods including ammonium sulfate precipitation, affinity columns, column chromatography, gel electrophoresis and the like (see, generally, R. Scopes, 1982, Protein Purification, Springer-Verlag, N.Y.; Deutscher, 1990, Methods in Enzymology Vol. 182: Guide to Protein Purification, Academic Press, Inc. N.Y.).

Where the protein binds a target reversibly, affinity columns bearing the target can be used to affinity purify the protein. Alternatively, the protein can be recombinantly expressed with a HIS-Tag and purified using Ni²⁺/NTA chromatography. In another embodiment, the protein can be chemically synthesized using standard chemical peptide synthesis techniques. Where the desired subsequences are relatively short the molecule may be synthesized as a single contiguous polypeptide. Where larger molecules are desired, subsequences can be synthesized separately (in one or more units) and then fused by condensation of the amino terminus of one molecule with the carboxyl terminus of the other molecule thereby forming a peptide bond. This is typically accomplished using the same chemistry (e.g., Fmoc, Tboc) used to couple single amino acids in commercial peptide synthesizers.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany and Merrifield (1962) Solid-Phase Peptide Synthesis; pp. 3-284 in The Peptides: Analysis, Synthesis, Biology. Vol. 2: Special Methods in Peptide Synthesis, Part A., Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In a preferred embodiment, the protein can also be synthesized using recombinant DNA methodology. Generally, this involves creating a DNA sequence that encodes the binding protein, placing the DNA in an expression cassette under the control of a particular promoter, expressing the protein in a host, isolating the expressed protein and, if required, renaturing the protein.

DNA encoding binding proteins or subsequences of this invention can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

The nucleic acid sequences encoding the desired binding protein(s) may be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli, this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes. Once expressed, the recombinant binding proteins can be purified according to standard procedures of the art as described above.

6.10.4 Preparation of Macromolecules or Analytes that are Sugars or Carbohydrates

Sugars and carbohydrates can be isolated from natural sources, enzymatically synthesized or chemically synthesized. A route to production of specific oligosaccharide structures is through the use of the enzymes that make them in vivo; the glycosyltransferases. Such enzymes can be used as regio- and stereoselective catalysts for the in vitro synthesis of oligosaccharides (Ichikawa et al., 1992, Anal. Biochem. 202: 215-238). Sialyltransferase can be used in combination with additional glycosyltransferases. For example, a combination of sialyltransferase and galactosyltransferases can be used. A number of methods of using glycosyltransferases to synthesize desired oligosaccharide structures are known. Exemplary methods are described, for instance, in WO 96/32491; Ito et al., 1993, Pure Appl. Chem. 65:753, and U.S. Pat. Nos. 5,352,670, 5,374,541, and 5,545,553. The enzymes and substrates can be combined in an initial reaction mixture, or alternatively, the enzymes and reagents for a second glycosyltransferase cycle can be added to the reaction medium once the first glycosyltransferase cycle has neared completion. By conducting two glycosyltransferase cycles in sequence in a single vessel, overall yields are improved over procedures in which an intermediate species is isolated.

Methods of chemical synthesis are described by Zhang et al., 1999, J. Am. Chem. Soc., 121(4): 734-753. Briefly, in this approach, a set of sugar-based building blocks is created with each block preloaded with different protecting groups. The building blocks are ranked by reactivity of each protecting group. A computer program then determines exactly which building blocks must be added to the reaction so that the sequences of reactions from fastest to slowest produces the desired compound.

6.11 Spacing Between Biosensor Electrodes

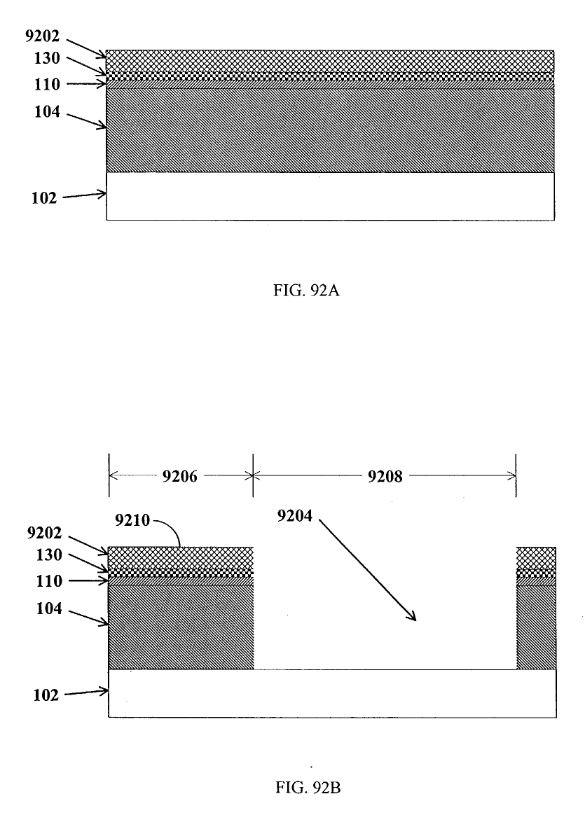

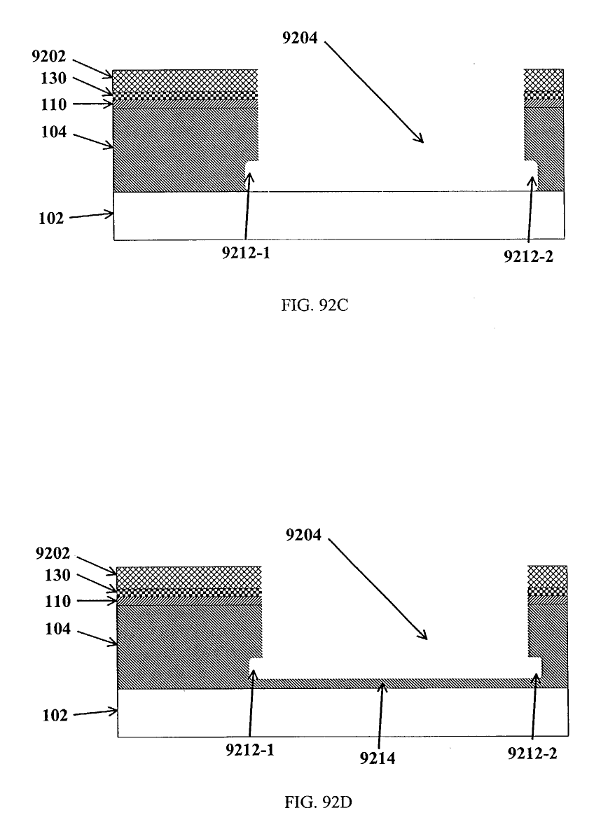

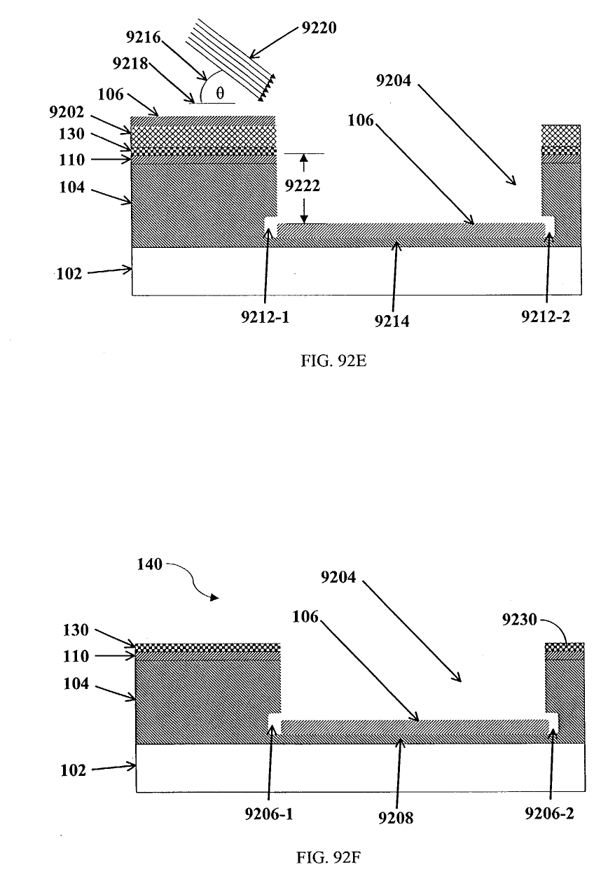

The spacing between materials 106 and 110 in the biosensors of the present invention is designed so that a macromolecule 120 can span materials 106 and 110 by binding to both materials 106 and 110. Accordingly, the spacing between materials 106 and 110 will depend upon the size of the macromolecules 120 analyzed. The spacing between the top of material 106 and the top of material 110 is illustrated in FIGS. 1, 2, and 3 as element 121. The spacing between materials 106 and 110 is further illustrated as element 8902 in FIGS. 89 and 90. The spacing between materials 106 and 110 is also illustrated as distance “d” in FIG. 91. In other instances, such as the biosensor illustrated in FIG. 25A, the spacing between materials 106 and 110 is defined as the shortest distance between (i) the portion (e.g., a point) of material 106 that is closest to material 110 and (ii) the portion (e.g., a point) of material 110 that is closest to material 106.

In certain embodiments, a plane including the top of material 106 and a plane including the top of material 110 are separated by a distance between 60 Angstroms and 500 Angstroms, between 10 Angstroms and 10⁵ Angstroms, between 25 Angstroms and 10⁴ Angstroms, or between 40 Angstroms and 10² Angstroms. In some embodiments of the present invention, a plane including the top of material 106 and a plane including the top of material 110 are separated by a distance that is less than 500 Angstroms, less than 400 Angstroms, less than 200 Angstroms, less than 100 Angstroms, less than 80 Angstroms, or less than 50 Angstroms. In some embodiments of the present invention, a plane including the top of material 106 and a plane including the top of material 110 are separated by a distance of between 60 Angstroms and 500 Angstroms, between 500 Angstroms and 1000 Angstroms, between 100 Angstroms and 200 Angstroms, between 60 Angstroms and 120 Angstroms, between 50 Angstroms and 70 Angstroms, between 90 Angstroms and 400 Angstroms, or between 40 Angstroms and 10,000 Angstroms. In some embodiments, distance 8902 (FIGS. 89 and 90) is between 60 Angstroms and 200 Angstroms, less than 500 Angstroms, or less than 1000 Angstroms.

In certain embodiments, a portion (e.g., a point) of material 106 and a portion (e.g., a point) of material 110 are separated by a distance between 60 Angstroms and 200 Angstroms, less than 500 Angstroms, less than 1000 Angstroms, between 300 Angstroms and 400 Angstroms, between 200 Angstroms and 300 Angstroms, less than 300 Angstroms, less than 200 Angstroms, less than 150 Angstroms, less than 100 Angstroms, or between 50 Angstroms and 80 Angstroms.

In some embodiments of the present invention, a portion of material 106 and a portion of material 110 are separated by a distance that is less than 200 Angstroms, less than 150 Angstroms, less than 100 Angstroms, less than 50 Angstroms, less than 40 Angstroms, or less than 30 Angstroms. In some embodiments of the present invention, a portion of material 106 and a portion of material 110 are separated by a distance of between 60 Angstroms and 500 Angstroms, between 40 Angstroms and 1000 Angstroms, between 100 Angstroms and 400 Angstroms, between 300 Angstroms and 700 Angstroms, between 100 Angstroms and 700 Angstroms, between 40 Angstroms and 90 Angstroms, or between 40 Angstroms and 10,000 Angstroms.

6.12 Attachment of Macromolecules to Biosensor Electrodes

In some embodiments of the present invention, macromolecules 120 are attached to materials 106 and 110 using methods well known to those of skill in the art. In some embodiments, macromolecule 120 includes reactive moieties (e.g. linkers) that facilitate attachment of macromolecule 120 to material 106 and/or material 110. Thus, in some embodiments, macromolecule 120 bears one or more reactive moieties (e.g. an aliphatic thiol linker) that react with the material 106 surface and/or the material 110 surface.

In some embodiments, material 106 and/or material 110 is coated with a reactive moiety, such as a functional group. In such embodiments, the reactivity moiety bound on material 106 and/or material 110 either binds to the macromolecule 120 directly or to a reactive moiety (e.g., a linker) that is bound to macromolecule 120, thereby attaching macromolecule 120 to material 106 and/or material 110.

In some embodiments, the reactive moiety attached to macromolecule 120 is electrically conductive and, when macromolecule 120 bridges materials 106 and 110, an electrical current passes directly or indirectly between materials 106 and 110 and macromolecule 120 through the reactive moiety.

The manner of linking a wide variety of compounds to surfaces, such as the surface of material 106 and/or material 110, in order to attach a reactive moiety to such surfaces is known in the art and is amply described in the literature. Furthermore, means for coupling a macromolecule 120 with a reactive moiety (e.g., a linker) are known to those of skill in the art. The coupling of macromolecule 120 with a reactive moiety can be covalent, or can be produced by ionic or other non-covalent interactions. Furthermore, in order to achieve binding between macromolecule 120 and materials 106 and/or 110, the surface of the material and/or macromolecule 120 may be specifically derivatized with a reactive moiety to provide convenient linking groups (e.g. sulfur, hydroxyl, amino, etc.).

In some embodiments, the reactive moieties that may be attached to macromolecule 120 and/or materials 106 and 110 are either hetero- or homo-bifunctional molecules that contain two or more reactive sites. Each such site is capable of forming a covalent bond with the respective binding partner (i.e., material 106 and/or material 110 surface or macromolecule 120). Reactive moieties used in the present invention include linkers such as any of a variety of a straight or branched aliphatic chains, heterocyclics, peptides, and the like. Exemplary linkers of the instant invention include, but are not limited to 4,4′-diphenylethyne, 4,4′-diphenylbutadiyne, 4,4′-biphenyl, 1,4-phenylene, 4,4′-stilbene, 1,4-bicyclooctane, 4,4′-azobenzene, 4,4′-benzylideneaniline, and 4,4″-terphenyl, oligophenylene vinylene, and the like (see, e.g., U.S. Pat. No. 6,208,553).

A wide variety of reactive moieties that are surface binding groups are known to those of skill in the art and are often used to produce self-assembling monolayers. All such linkers may be used in the present invention. Such linkers may be attached to macromolecule 120, material 106 and/or material 110. Such linkers include, but are not limited to, thiols, (e.g. alkanethiols) (which bind gold and other metals), alkyltrichlorosilane (e.g., which bind silicon/silicon dioxide), alkane carboxylic acids (e.g., which bind aluminum oxides), derivatives of ethylene glycol, as well as combinations thereof. See, for example, Ferguson et al., 1993, Macromolecules 26(22): 5870-5875; Prime et al., 1991, Science 252:1164-1167; Bain et al., 1989, Angew. Chem. 101: 522-528; Kumar et al., 1994, Langmuir 10: 1498-1511; Laibinis et al., 1989, Science 245: 845-847; Pale-Grosdemange et al., 1991, J. Am. Chem. Soc., 113: 12-20. In some embodiments, macromolecule 120 is attached to metal electrodes using thiol linkers (e.g., alkanethiol linkers).

In certain embodiments, macromolecules 120 are functionalized with a chemical group, or a linker bearing a chemical group, that can be activated by the application of an electrical potential. Such groups are well known to those of skill in the art and include, but are not limited to, S-benzyloxycarbonyl derivatives, S-benzyl thioethers, S-phenyl thioethers, S-4-picolyl thioethers, S-2,2,2-trichloroethoxycarbonyl derivatives, S-triphenylmethyl thioethers. In certain embodiments, macromolecules 120 are functionalized with a chemical group or a linker bearing a chemical group, that can be activated by light of wavelength ranging from 190 nm to 700 nm. Such chemical groups include, but are not limited to, an aryl azide, a flourinated aryl azide, a benzophenone, and (R,S)-1-(3,4-(methylene-dioxy)-6-nitrophenyl) ethyl cholorformate-(MeNPOC), N-((2-pyridyl, ethyl)-4-azido) salicylamide.

In some embodiments of the present invention, macromolecule 120 is derivatized with one of the groups described above and then placed in solution. While in solution, the derivatized macromolecule 120 contacts material 106 and/or material 110. Then, a charge is placed on material 106 to attract macromolecule 120 to material 106. Upon contact with material 106, the derivatized macromolecule 120 binds to material 106. The derivatized macromolecule 120 can bear two linkers, one for attachment to material 106 and one for attachment to material 110. In such embodiments, the second linker is blocked to prevent reaction with material 106. After derivatized macromolecule 120 has bound to material 106, the second linker is deprotected thereby permitting it to bind to material 110. Thus, for example, to span materials 106 and 110 in a given device 144 with a macromolecule 120 that is a nucleic acid, the nucleic acid is derivatized with a first linker and a second linker. In this example, the second linker is a protected (blocked) thiol and the first linker is a deprotected (unblocked) thiol. Material 106 is biased positive to attract the nucleic acid to material 106. The deprotected thiol linker binds to material 106. Material 106 is then biased negative and material 110 is biased positive to attract the free end of the nucleic acid to material 110. The blocked thiol linker is deprotected leaving it free to interact with material 110. Once the interaction between the thiol linker and material 110 occurs, the nucleic acid binds to both materials 106 and 110 and spans the gap between the first and the second electrode. It will be appreciated that there are many variations to the example described above. For example, the use of materials 106 and 110 are interchangeable. That is, the role of material 106 and 110 can be reversed in the example above.

In one specific embodiment, materials 106 and 110 in a biosensor of the present invention are made of gold. Materials 106 and 110 are dried under nitrogen or argon and connected to macro electrodes that are, in turn, connected to a voltage source. A voltage of less than ±3 volts, less than ±5 volts, or less than ±9 volts is applied. The materials 106 and 110 are then and tested for non-conductance, or a background conductance, using an EG&G high-speed potentiostat/galvanostat (e.g. Perkin-Elmer, Model 283). The biosensor is then contacted with a capture probe solution comprising derivatized oligonucleotides. The five prime end of the oligonucleotides is derivatized with a reactive thiol group that is masked with an S-2,2,2-trichloroethoxycarbonyl derivative. The thiocarbonate can be cleaved at −1.5 volts in the presence of LiClO₄/CH₃OH to reveal a reactive thiol group. The reactive thiol group can, in turn, form a covalent bond with an electrically conducting material such as gold. The three prime end of the oligonucleotides is derivatized with another electrolabile group, such as an s-benzyloxycarbonyl moiety, which can be removed at −2.6 volts in N,N-dimethylformamide and tetrabutyl ammonium chloride. Material 106 is biased with the activation voltage of the five prime electrolabile group (−1.5 volts) on the oligonucleotides thereby exposing the thiol group that then attaches to material 106. Then, material 110 is biased with the activation voltage of the 3 prime electrolabile group of the capture probe (−2.6 volts) thereby causing the nucleotide to bind to material 110. Materials 106 and 110 are then dried again under nitrogen or argon. A voltage of less than ±3 volts, less than ±5 volts, or less than ±9 volts is applied to the electrodes and the current is measured. The measured current of the hybridized nucleic acids is significantly greater then the current measured for the unhybridized electrodes.

The assembly approach described in the example above thus uses the biosensor itself, to direct the localization and ultimate attachment of macromolecule 120. Thus, the biosensors of this invention are able to electronically self-address each device 144 with a specific macromolecule 120. The assembly approach described in the example above is a self-assembly approach in the sense that no outside process or mechanism is needed to physically direct a specific macromolecule 120 on a specific material 106 or 110 in a device 144 in a biosensor of the present invention. This self-addressing process is both rapid and specific, and can be carried out in either a serial or parallel manner.

Each device 144 in the biosensors of the present invention can be serially addressed with a specific macromolecule 120. In one scheme, selected materials 106 and/or 110 in a targeted device 144 are set at the opposite charge (potential) to that of macromolecule 120 while materials 106 and/or 110 in nontargeted devices 144 are maintained at the same charge macromolecule 120.

One illustrative parallel process for addressing materials 106 and 110 in targeted devices 144 simply involves simultaneously activating a large number of electrodes (e.g., a particular group or line of devices 144). In this way, the same macromolecule 120 is transported, concentrated, and reacted with more than one specific material 106 and/or 110. Numerous other approaches can be used to attach macromolecule 120 to respective materials 106 and/or 110. Such approaches include, but are not limited to, attachment of chemical groups to the surface of material 106 and/or 110 through the use of photoactivatable chemistries. See, for example, Sundberg et al., 1995, J. Am. Chem. Soc. 117, pp. 12050-12057; Kumar et al., 1994, Langmuir 10, pp. 1498-1511; and Kumar et al., 1993, Appl. Phys. Lett. 63, pp. 2002-2004.

### 7.0 Methods for Making the Biosensors of the Present Invention

An overview of the biosensors of the present invention has been described. Reference will now be made to additional biosensor configurations as well as methods for manufacturing such biosensors.

7.1 Two Non-Overlapping Electrodes with two Insulator Layers

This section describes methods for making biosensors that have two non-overlapping conducting layers with two insulator layers. One biosensor in accordance with the present invention that has two non-overlapping conducting layers with two insulator layers is illustrated in FIG. 1, where the two non-overlapping conducting layers are materials 106 and 110 and the two insulator layers comprise spacer 140 and insulator layer 104. The methods described below are for instances where materials 106 and 110 are made from the same material. However, one of skill in the art can easily use the techniques described below to manufacture biosensors in which materials 106 and 110 are different. For example, the processes described in Sections 7.1.7 through 7.1.10 can be run twice, once for material 106 and once for material 110, in order to make a biosensor in which the composition of material 106 and material 110 is different. In addition to the techniques described below, many others techniques that are well known to those of skill in the art can be used. See, for example, Levinson, 2001, Principles of Lithography, SPIE Press, Bellingham. Wash.; and Rai-Choudhury, 1997, The Handbook of Microlithography, Micromachining, and Microfabrication, Soc. Photo-Optical Instru. Engineer Press, Bellingham, Wash.

7.1.1 INSULATOR FORMATION

In the first step of a process flow for making the biosensor, insulator layer 104 is overlaid onto substrate 102. There are a number of different ways in which insulator layer 104 can be overlaid (e.g., deposited) onto substrate 102. These methods are described in the following subsections. In addition to the methods described below, other methods may be used to deposit insulator layer 104 onto substrate 102. Such methods include, but are not limited to, sputter deposition (see Section 7.1.7.2, below), vacuum evaporation (see Section 7.1.7.1, below), laser ablated deposition (see Section 7.1.7.4, below), atomic layer deposition (see Section 7.1.7.8, below), molecular beam deposition (see Section 7.1.7.5, below), ion beam deposition (see Section 7.1.7.7, below), hot filament chemical vapor deposition (see Section 7.1.7.9, below), and screen printing (see Section 7.1.7.10, below). In some instance, insulator layer 104 is, in fact, made of a metal. In such instances, insulator layer 104 may be deposited using electrochemical means such as electroless metal deposition (see Section 7.1.7.11, below) or electroplating (see Section 7.1.7.12, below).

7.1.1.1 Thermal Oxidation of Silicon

In some embodiments, substrate 102 is made of silicon and insulator layer 104 comprises silicon dioxide. In such embodiments, insulator layer 104 may be formed by thermal oxidation of silicon substrate 102. Thermal oxide growth is a simple mechanical reaction in which solid silicon reacts with oxygen gas to form a layer of silicon oxide. The reaction of solid silicon and oxygen gas occurs at room temperature. However, the reaction is driven by heat. Consequently, in some embodiments, insulator layer 104 is formed by heating a silicon substrate 102 in the presence of oxygen at a temperature between 900° C. and 1200° C. Thermal oxidation can be used to make insulator layers 104 having a thickness that ranges from 60 Angstroms to 100 Angstroms, 100 Angstroms to 500 Angstroms or thicker. Thermal oxidation may be performed in a wide variety of apparatuses, including horizontal tube furnaces, vertical tube furnaces, fast ramp furnaces, a rapid thermal oxidation system, high pressure oxidation systems. For more information on thermal oxidation, devices used to perform thermal oxidation, and process conditions that may be used to perform thermal oxidation, see Van Zant, Microchip Fabrication Fourth Edition, McGraw-Hill, New York, pp. 157-187, which is hereby incorporated by reference in its entirety.

7.1.1.2 Chemical Vapor Deposition

In some embodiments, insulator layer 104 is deposited onto substrate 102 by chemical vapor deposition. In chemical vapor deposition (CVD), the constituents of a vapor phase, often diluted with an inert carrier gas, react at a hot surface (typically higher than 300° C.) to deposit a solid film. Generally, chemical vapor deposition reactions require the addition of energy to the system, such as heating the chamber or the wafer. For more information on chemical vapor deposition, devices used to perform chemical vapor deposition, and process conditions that may be used to perform chemical vapor deposition of silicon nitride, see Van Zant, Microchip Fabrication, Fourth Edition, McGraw-Hill, New York, 2000, pp. 363-393; and Madou, Fundamentals of Microfabrication, Second Edition, 2002, pp. 144-154, CRC Press, which are hereby incorporated by reference in their entireties.

7.1.1.3 Reduced Pressure Chemical Vapor Deposition

In some embodiments, insulator layer 104 is deposited onto substrate 102 by reduced pressure chemical vapor deposition (RPCVD). RPCVD is typically performed at below 10 Pa and at temperatures in the range of (550° C.-600° C.). The low pressure used in RPCVD results in a large diffusion coefficient, which leads to growth of layer 104 that is limited by the rate of surface reactions rather than the rate of mass transfer to the substrate. In RPCVD, reactants can typically be used without dilution. RPCVD may be performed, for example, in a horizontal tube hot wall reactor.

7.1.1.4 Low Pressure Chemical Vapor Deposition

In some embodiments, insulator layer 104 is deposited onto substrate 102 by low-pressure chemical vapor deposition (LPCVD) or very low pressure CVD. LPCVD is typically performed at below 1 Pa. Low-pressure operation is useful for single-crystalline silicon growth at relatively low temperatures. Low-pressure operation is also advantageous for the growth of Ill-V compound superlattices.

7.1.1.5 Atmospheric Chemical Vapor Deposition

In some embodiments, insulator layer 104 is deposited onto substrate 102 by atmospheric to slightly reduced pressure chemical vapor deposition. Atmospheric-pressure to slightly reduced pressure CVD (APCVD) is used, for example, to grow epitaxial (i.e., single-crystalline) films of silicon, GaAs, InP, and HgCdTe. APCVD is a relatively simplistic process that has the advantage of producing layer 104 at a high deposition rate and low temperatures (350° C.-400° C.).

7.1.1.6 Plasma Enhanced Chemical Vapor Deposition

In some embodiments, insulator layer 104 is deposited onto substrate 102 by plasma enhanced (plasma assisted) chemical vapor deposition (PECVD). PECVD systems feature a parallel plate chamber operated at a low pressure (e.g., 2-5 Torr) and low temperature (300° C.-400° C.). A radio-frequency-induced glow discharge, or other plasma source is used to induce a plasma field in the deposition gas. The combination of the low pressure and lower temperatures provides good insulator 104 uniformity. PECVD systems that may be used to deposit insulator layer 104 include, but are not limited to, horizontal vertical flow PECVD, barrel radiant-heated PECVD, and horizontal-tube PECVD. In some embodiments, insulator layer 104 is deposited onto substrate 102 by remote plasma CVD (RPCVD). Remote plasma CVD is described, for example, in U.S. Pat. No. 6,458,715 to Sano et al.

7.1.1.7 Anodization

In some embodiments, insulator layer 104 is deposited onto substrate 102 by anodization. Anodization is an oxidation process performed in an electrolytic cell. The material to be anodized (e.g. substrate 102) becomes the anode (+) while a noble metal is the cathode (−). Depending on the solubility of the anodic reaction products, an insoluble layer (e.g., an oxide) results. If the primary oxidizing agent is water, the resulting oxides generally are porous, whereas organic electrolytes lead to very dense oxides providing excellent passivation. See, for example, Madou et al., 1982, J. Electrochem. Soc. 129, pp. 2749-2752. In one example, substrate 102 is made of silicon and a SiO₂ insulator layer 104 is produced by anodization of the upper surface of substrate 102 using a highly concentrated hydrofluoric acid solution.

7.1.1.8 Sol-Gel Deposition Technique

In some embodiments, insulator layer 104 is deposited onto substrate 102 by a sol-gel process. In a sol-gel process solid particles, chemical precursors, in a colloidal suspension in a liquid (a sol) forms a gelatinous network (a gel). Upon removal of the solvent by heating a glass or ceramic insulator layer 104 results. Both sol and gel formation are low-temperature processes. For sol formation, an appropriate chemical precursor is dissolved in a liquid, for example, tetraethylsiloxane (TEOS) in water. The sol is then brought to its gel-point, that is, the point in the phase diagram where the sol abruptly changes from a viscous liquid to a gelatinous, polymerized network. In the gel state the material is shaped (e.g., a fiber or a lens) or applied onto a substrate by spinning, dipping, or spraying. In the case of TEOS, a silica gel is formed by hydrolysis and condensation using hydrochloric acid as the catalyst. Drying and sintering at temperatures between 200° C. to 600° C. transforms the gel into a glass and ultimately into silicon dioxide. More information on the sol-gel process is available at the Sol-Gel gateway at http://www.solgel.com/bookstore/bookstore.htm.

7.1.1.9 Plasma Spraying Technique

In some embodiments, insulator layer 104 is deposited onto substrate 102 by a plasma spraying process. With plasma spraying, almost any material can be coated on many types of substrates 102. Plasma spraying is a particle deposition method. Particles, a few microns to 100 microns in diameter, are transported from source to substrate. In plasma spraying, a high-intensity plasma arc is operated between a stick-type cathode and a nozzle-shaped water-cooled anode. Plasma gas, pneumatically fed along the cathode, is heated by the arc to plasma temperatures, leaving the anode nozzle as a plasma jet or plasma flame. Argon and mixtures of argon with other noble (He) or molecular gases (H₂, N₂, O₂, etc.) are frequently used for plasma spraying. Fine powder suspended in a carrier gas is injected into the plasma jet where the particles are accelerated and heated. The plasma jet may reach temperatures of 20,000 K and velocities up to 1000 ms-1. The temperature of the particle surface is lower than the plasma temperature, and the dwelling time in the plasma gas is very short. The lower surface temperature and short duration prevent the spray particles from being vaporized in the gas plasma. The particles in the plasma assume a negative charge, owing to the different thermal velocities of electrons and ions. As the molten particles splatter with high velocities onto a substrate, they spread, freeze, and form a more or less dense coating, typically forming a good bond with the substrate. Plasma spraying equipment is available from Sulzer Metco (Winterthur Switzerland). For more information on plasma spraying, see, for example, Madou, Fundamentals of Microfabrication, Second Edition, 2002, pp. 157-159, CRC Press.

7.1.1.10 Ink Jet Printing

In some embodiments, insulator layer 104 is deposited onto substrate 102 by ink jet printing. The ink-jet printing used to form insulator layer 104, in some embodiments of the present invention, is based on the same principles of commercial ink-jet printing. The ink-jet nozzle is connected to a reservoir filled with the chemical solution and placed above a computer-controlled x-y stage. Substrate 102 is placed on the x-y stage and, under computer control, liquid drops (e.g., 50 microns in diameter) are expelled through the nozzle onto a well-defined place on substrate 102. Different nozzles may print different spots in parallel. In one embodiment of the invention, a bubble jet, with drops as small as a few picoliters, is used to form insulator layer 104. In another embodiment, a thermal ink jet (Hewlett Packard, Palo Alto, Calif.) is used to form insulator layer 104. In a thermal ink jet, resistors are used to rapidly heat a thin layer of liquid ink. A superheated vapor explosion vaporizes a tiny fraction of the ink to form an expanding bubble that ejects a drop of ink from the ink cartridge onto the substrate. In still another embodiment of the present invention, a piezoelectric ink-jet head is used for ink-jet printing. A piezoelectric ink-jet head includes a reservoir with an inlet port and a nozzle at the other end. One wall of the reservoir consists of a thin diaphragm with an attached piezoelectric crystal. When voltage is applied to the crystal, it contracts laterally, thus deflecting the diaphragm and ejecting a small drop of fluid from the nozzle. The reservoir then refills via capillary action through the inlet. One, and only one, drop is ejected for each voltage pulse applied to the crystal, thus allowing complete control over the when a drop is ejected. In yet another embodiment of the present invention, an epoxy delivery system is used to deposit insulator 104 onto substrate 102. An example of an expoxy delivery system is the Ivek Digispense 2000 (Ivek Corporation, North Springfield, Vt.). For more information on jet spraying, see, for example, Madou, Fundamentals of Microfabrication, Second Edition, 2002, pp. 164-167, CRC Press.

7.1.2 Spacer Deposition and Resist Layer Deposition

In some embodiments of the present invention, spacer 140 is formed by first depositing a second insulator layer onto insulator layer 104 and then patterning the second insulator layer. In some embodiments, the second insulator layer that is overlaid onto insulator layer 104 is deposited by chemical vapor deposition of silicon oxide or silicon nitride. Then, the second insulator layer is patterned by semiconductor photolithographic photoresist coating and optical imaging through an optical mask, thereby forming spacer 140.

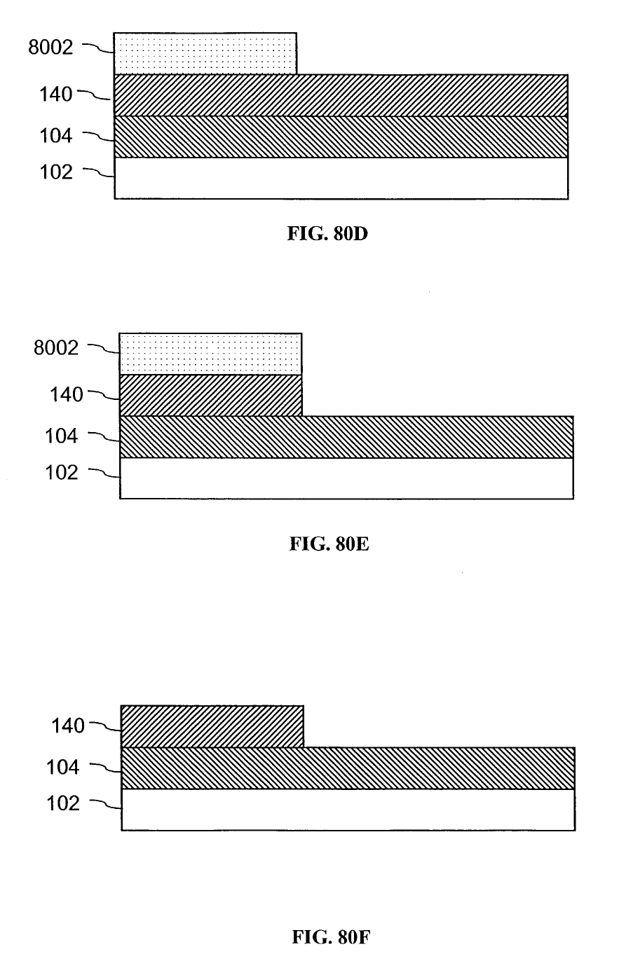

One form of photolithographic processing in accordance with the present invention is illustrated in FIG. 80. The process begins in FIG. 80A with a silicon wafer 102 on which is overlaid insulating layer 104 and spacer 140. Spacer 140 is coated with a resist layer 8002 (FIG. 80B). Resists used to form resist layer 8002 are typically comprised of organic polymers applied from a solution. Generally, to coat the wafers with resist, a small volume of the liquid is first dispensed on wafer 102. The wafer is then spun at a high rate of speed, flinging off excess resist and leaving behind, as the solvent evaporates, resist layer 8002. In some embodiments, resist layer 8002 has a thickness in the range of 0.1 μm to 2.0 μm. Furthermore, in some embodiments, resist layer 8002 has a uniformity of plus or minus 0.01 μm.

In some embodiments, resist layer 8002 is applied to spacer 140 using a spin technique such as a static spin process or a dynamic dispense process. In some embodiments, resist layer 8002 is applied using a manual spinner, a moving-arm resist dispenser, or an automatic spinner. See, for example, Van Zant, Microchip Fabrication, Fourth Edition, McGraw-Hill, New York, 2000, pp. 217-222.

In some embodiments, resist layer 8002 is an optical resist that is designed to react with ultraviolet or laser sources. In some embodiments, resist layer 8002 is a negative resist in which polymers in the resist form a cross-linked material that is etch resistant upon exposure to light. Examples of negative resists that can be used to make resist layer 8002 include, but are not limited to, azide/isoprene negative resists, polymethylmethacrylate (PMMA), polymethylisopropyl ketone (PMIPK), poly-butene-1-sulfone (PBS), poly-(trifluoroethyl chloroacrylate) TFECA, copolymer-α-cyano ethyl acrylate-α-amido ethyl acrylate) (COP), poly-(2-methyl pentene-1-sulfone) (PMPS). In other embodiments, resist layer 8002 is a positive resist. The positive resist is relatively unsoluble. After exposure to the proper light energy, the resist converts to a more soluble state. This reaction is called photosobulization. One positive photoresist in accordance with the present invention is the phenol-formaldehyde polymer, also called phenol-formaldehyde novolak resin. See, for example, DeForest, Photoresist: Materials and Processes, McGraw-Hill, New York, 1975, which is hereby incorporated by reference in its entirety. In some embodiments, resist layer 8002 is LOR 0.5A, LOR 0.7A, LOR 1A, LOR 3A, or LOR 5A (MICROCHEM, Newton, Mass.). LOR lift-off resists use polydimethylglutarimide.

After resist layer 8002 has been applied, the density is often insufficient to support later processing. Accordingly, in some embodiments of the present invention, a bake is used to densify resist layer 8002 and drive off residual solvent. This bake is referred to as a softbake, prebake, or post-apply bake. Several methods of baking resist layer 8002 are contemplated by the present invention including, but not limited to, convection ovens, infrared ovens, microwave ovens, or hot plates. See, for example, Levinson, Principles of Lithography, SPIE Press, Bellingham, Wash., 2001, pp. 68-70, which is hereby incorporated by reference in its entirety.

7.1.3 Mask Alignment and Resist Layer Exposure for Spacer Patterning

After spacer 140 has been coated with resist layer 8002 (FIG. 80B), the next step is alignment and exposure of resist layer 8002 (FIG. 80C). Alignment and exposure is, as the name implies, a two-purpose photomasking step. The first part of the alignment and exposure step is the positioning or alignment of the required image on the wafer surface. The image is found on a mask (e.g., mask 8004 of FIG. 80C). The second part is the encoding of the image in the resist layer 8002 from an exposing light or radiation source.

In the present invention, any conventional alignment system can be used to align mask 8004 with resist layer 8002, including but not limited to, contact aligners, proximity aligners, scanning projection aligners, steppers, step and scan aligners, x-ray aligners, and electron beam aligners. For a review of aligners that can be used in the present invention, see Solid State Technology, April 1993, p. 26; and Van Zant, Microchip Fabrication, Fourth Edition, McGraw-Hill, New York, 2000, pp. 232-241. FIG. 80C illustrates a negative mask 8004 that is used to develop a negative resist layer 8002. A positive mask (not shown) used to develop a positive resist would have the opposite pattern of mask 8004.

Both negative masks 8004 and positive masks (not shown) used in the methods of the present invention are fabricated with techniques similar to those used in wafer processing. A photomask blank, consisting of an opaque film (usually chromium) deposited on glass substrates, is covered with resist. The resist is exposed according to the desired pattern, is then developed, and the exposed opaque material etched. Mask patterning is accomplished primarily by means of beam writers, which are tools that expose mask blanks according to suitably formatted biosensor electrode patterns. In some embodiments electron or optical beam writers are used to pattern negative masks 8004 or positive masks (not shown). See for, example, Levison, Principles of Lithography, SPIE Press, Bellingham, Wash., 2001, pp. 229-256.

In one embodiment of the present invention, the tool used to project the pattern on mask 8004 onto wafer 102 is a wafer stepper. Wafer steppers exist in two configurations, step-and-repeat and step-and-scan. In a step-and-repeat system, the entire area of mask 8004 to be exposed is illuminated when a shutter is opened. In a step-and-scan system, only part mask 8004, and therefore only part of the exposure field on wafer 8004, is exposed when a shutter is opened. The entire field is exposed by scanning mask 8004 and wafer 102 synchronously. See for example, Levison, Principles of Lithography, SPIE Press, Bellingham, Wash., 2001, pp. 133-174.

7.1.4 Resist Layer Development for Spacer Patterning

After exposure through mask 8004 (FIG. 80C), the pattern for spacer 140, as illustrated in FIG. 1, is coded as a latent image in resist 8002 as regions of exposed and unexposed resist. The pattern is developed in the resist by chemical dissolution of the unpolymerized resist regions to form the structure illustrated in FIG. 80D. This section describes a number of development techniques that begin with the structure illustrated in FIG. 80C and end with the structure illustrated in FIG. 80D.

Development techniques are designed to leave in the resist layer an exact copy of the pattern that was on the mask or reticle. The successful development of the image coded in resist 8002 is dependent on the nature of the resist's exposure mechanisms. Negative resist, upon exposure to light, goes through a process of polymerization which renders the resist resistant to dissolution in the developer chemical. The dissolving rate between the two regions is high enough so that little of layer 8002 is lost from the polymerized regions. The chemical preferred for most negative-resist-developing situations is xylene or Stoddart solvent. The development step is done with a chemical developer followed by a rinse. For negative resists, the rinse chemical is usually n-butyl acetate. Positive resists 8002 present a different developing condition. The two regions, polymerized and unpolymerized, have a dissolving rate difference of 1:4. This means that during the developing step some resist is always lost from the polymerized region. Use of developers that are too aggressive or that have overly long developing times may result in an unacceptable thinning of resist 8002. Two types of chemical developers used with positive resists 8002 in accordance with the present invention are alkaline-water solutions and nonionic solutions. The alkaline-water solutions can be sodium hydroxide or potassium hydroxide. Typical nonionic solutions include, but are not limited to, tetramethylammonimum hydroxide (TMAH). The rinse chemical for positive-resist developers is water. A rinse is used for both positive and negative resists 8002. This rinse is used to rapidly dilute the developer chemical to stop the developing action.

There are several methods in which a developer may be applied to resist 8002 in order to develop the latent image. Such methods include, but are not limited to, immersion, spray development, and puddle development. In some embodiments of the present invention, wet development methods are not used. Rather, a dry (or plasma) development is used. In such dry processes, a plasma etcher uses energized ions to chemically dissolve away either exposed or unexposed portions of resist layer 8002.

In some embodiments of the present invention, resist is hard baked after is has been developed. The purpose of the hard bake is to achieve good adhesion of resist layer 8002 to spacer 140. A hard bake may be accomplished using a convection oven, in-line or manual hot plates, infrared tunneling ovens, moving-belt convection ovens, vacuum ovens and the like. General baking temperature and baking times are provided by the resist manufacture. Therefore, specific baking temperatures and times is application dependent. Nominal hard bake temperatures are from 130° C. to 200° C. for thirty minutes in a convection oven.

7.1.5 Spacer Etching

After development, an etching step is used to pattern spacer 140. This section describes a number of methods that start with the structure illustrated in FIG. 80D and end with the structure illustrated in 80E.

7.1.5.1 Wet Etching

In one embodiment of the present invention, the structure illustrated in FIG. 80D is immersed in a tank of an etchant for a specific time. Then the structure is transferred to a rinse station for acid removal, and transferred to a station for final rinse and a spin dry step, in order to achieve the structure illustrated in FIG. 80E. In the case where spacer 140 is made of silicon dioxide, the etchant could be hydroflouric acid, which has the advantage of dissolving silicon dioxide without attacking silicon. In some embodiments, the hydrofluoric acid is mixed with water or ammonium fluoride and water. Such solutions are known as buffered oxide etches or BOEs. In the case where spacer 140 is made of silicon nitride, hot (180° C.) phosphoric acid can be used. Since the acid evaporates rapidly at this temperature, the etch must be done in a closed reflux container equipped with a cooled lid to condense the vapors.

7.1.5.2 Wet Spray Etching or Vapor Etching

In some embodiments of the present invention, wet spray etching or vapor etching is used to pattern spacer 140. Wet spray etching offers several advantages over immersion etching. Primary is the added definition gained from the mechanical pressure of the spray. In vapor etching, the wafer is exposed to etchant vapors such as hydroflouric acid vapors.

7.1.5.3 Plasma Etching

In some embodiments of the present invention, plasma etching is used. Plasma etching is a chemical process that uses gases and plasma energy to cause the chemical reaction. Plasma etching is performed using a plasma etcher. Physically, a plasma etcher comprises a chamber, vacuum system, gas supply, and a power supply. The structure illustrated in FIG. 80D is loaded into the chamber and the pressure inside is reduced by the vacuum system. After the vacuum is established, the chamber is filled with the reactive gas. For the etching of silicon dioxide, the gas is usually CF₄ that is mixed with oxygen. A power supply creates a radio frequency (RF) field through electrodes in the chamber. The field energizes the gas mixture to a plasma state. In the energized state, the fluorine attacks the silicon dioxide, converting it into volatile components that are removed from the system by the vacuum system.

A wide variety of plasma etchers may be used to perform etching, in accordance with various embodiments of the present invention. Such etchers include, but are not limited to, barrel etchers, plasma planar systems, electron cyclotron resonance sources, high density reflected electron sources, helicon wave sources, inductively coupled plasma sources, and transformer coupled plasma sources.

7.1.5.4 Ion Beam Etching

Another type of etcher that may be used to perform the etching of spacer 140 in accordance with various aspects of the present invention is ion beam etching. Unlike chemical plasma systems, ion beam etching is a physical process. The wafer (e.g. the structure illustrated in FIG. 80D) is placed on a holder in a vacuum chamber and a stream of argon is introduced into the chamber. Upon entering the chamber, the argon is subjected to a stream of high-energy electrons from a set of cathode (−)-anode (+) electrodes. The electrons ionize the argon atoms to a high-energy state with a positive charge. The wafers are held on a negatively grounded holder that attracts the ionized argon atoms. As the argon atoms travel to the wafer holder they accelerate, picking up energy. At the wafer surface, they crash into the exposed wafer layer and blast small amounts from the wafer surface. No chemical reaction takes place between the argon atoms and the wafer material. The material removal (etching) is highly directional (anisotropic), resulting in good definition in small openings.

7.1.5.5 Reactive Ion Etching

Yet another type of etcher that may be used to perform the etching of spacer 140 is a reactive ion etcher. A reactive ion etcher system combines plasma etching and ion beam etching principles. The systems are similar in construction to the plasma systems but have a capability of ion milling. The combination brings the benefits of chemical plasma etching along with the benefits of directional ion milling. See, Van Zant, Microchip Fabrication, Fourth Edition, McGraw-Hill, New York, 2000, pp. 256-270, for more information on etching techniques and etching equipment that can be used in accordance with the present invention.

7.1.6 Residual Layer Removal

The result of the etching process described in Section 7.1.5 is the patterning of spacer 140 as illustrated in FIG. 80E. Next, residual layer 8002 is removed in a process known as resist stripping in order to yield the structure illustrated in FIG. 80F. In some embodiments, resist 8002 is stripped off with a strong acid such as H₂SO₄ or an acid-oxidant combination, such as H₂SO₄—Cr₂O₃, attacking resist 8002 but not spacer 140 to yield the fully patterned spacer 140 (FIG. 80F). Other liquid strippers include organic solvent strippers (e.g., phenolic organic strippers and solvent/amine strippers) and alkaline strippers (with or without oxidants).

In some embodiments of the present invention, a dry plasma process is applied to remove resist 8002. In such embodiments, the structure illustrated in FIG. 80E is placed in a chamber and oxygen is introduced. The plasma field energizes the oxygen to a high-energy state, which, in turn, oxidizes the resist components to gases that are removed from the chamber by the vacuum pump. In dry strippers, the plasma is generated by microwave, radio frequency, or ultraviolet-ozone sources.

More information on photolithographic processes that can be used to pattern spacer 140 is found in Madou, Fundamentals of Microfabrication Second Edition, CRC Press, Boca Raton, Fla., 2002, pp. 2-65; and Van Zant, Microchip Fabrication, Fourth Edition, McGraw-Hill, New York, 2000, which are hereby incorporated by reference in their entireties. Such methods include the use of a positive photoresist rather than a negative photoresist as well as extreme ultraviolet lithography, x-ray lithography, charged-particle-beam lithography, scanning probe lithography, soft lithography, and three-dimensional lithographic methods.

7.1.7 Deposition of Electrodes

In some embodiments of the present invention, materials 106 and 110 are formed in the biosensor illustrated in FIG. 1 in a two-step method. First, a layer of material 8010 is deposited on the structure illustrated in FIG. 80F to form the structure illustrated in FIG. 80G. Then, layer 8010 is patterned using applicable techniques described in the case of spacer 140, above.

In some instances, the deposition technique used to deposit material 8010 includes enough resolution and control to deposit the material into precisely defined regions on the structure illustrated in FIG. 80F in order to directly produce the structure illustrated in FIG. 80L. In such instances, subsequent patterning, as illustrated in FIGS. 80G through 80K and discussed in Sections 7.1.8 through 7.1.12 is not used.

Layer 8010 may be deposited by a variety of techniques. Some of the techniques that can be used to deposit layer 8010 are described in the following subsections. In addition to the techniques described in the following sections, layer 8010 may be deposited using chemical vapor deposition (see Section 7.1.1.2, above), low pressure chemical vapor deposition (see Section 7.1.1.3, above), reduced pressure chemical vapor deposition (see Section 7.1.1.4, above), atmospheric chemical vapor deposition (see Section 7.1.1.5, above), plasma assisted chemical vapor deposition (see Section 7.1.1.6, above), remote plasma chemical vapor deposition (see Section 7.1.1.6, above), anodic conversion (see Section 7.1.1.7, above), plasma spray deposition (see Section 7.1.1.9, above), jet printing (see Section 7.1.1.10, above), and sol-gel processes (see Section 7.1.1.8, above). In addition, those of skill in the art will recognize that there are number of other different methods by which layer 8010 may be deposited and all such methods are included within the scope of the present invention.

7.1.7.1 Vacuum Evaporation

In one embodiment of the present invention, vacuum evaporation is used to deposit layer 8010 onto the structure illustrated in FIG. 50F to form the structure illustrated in FIG. 80G. Vacuum evaporation takes place inside an evacuated chamber. The chamber can be, for example, a quartz bell jar or a stainless steal enclosure. Inside the chamber is a mechanism that evaporates the metal source, a wafer holder, a shutter, thickness and rate monitors, and heaters. The chamber is connected to a vacuum pump. There are any number of different ways in which the metal may be evaporated within the chamber, including filament evaporation, E-beam gun evaporation, and hot plate evaporation. See, for example, Van Zant, Microchip Fabrication, Fourth Edition, McGraw-Hill, New York, 2000, pp. 407-411, which is hereby incorporated by reference in its entirety.

7.1.7.2 Sputter Deposition/Physical Vapor Deposition

In another embodiment of the present invention, sputter deposition is used to deposit layer 8010 onto the structure illustrated in FIG. 80F to form the structure illustrated in FIG. 80G. Sputtering, like evaporation, takes place in a vacuum. However, it is a physical not a chemical process (evaporation is a chemical process), and is referred to as physical vapor deposition. Inside the vacuum chamber is a slab, called a target, of the desired film material. The target is electrically grounded. An inert gas such as argon is introduced into the chamber and is ionized to a positive charge. The positively charged argon atoms are attracted to the grounded target and accelerate toward it. During the acceleration they gain momentum, and strike the target, causing target atoms to scatter. That is, the argon atoms “knock off” atoms and molecules from the target into the chamber. The sputtered atoms or molecules scatter in the chamber with some coming to rest on the wafer.

A principal feature of a sputtering process is that the target material is deposited on the wafer with chemical or compositional change. In some embodiments of the present invention, direct current (DC) diode sputtering, radio frequency (RF) diode sputtering, triode sputtering, DC magnetron sputtering or RF magnetron sputtering is used. See, for example, Van Zant, Microchip Fabrication, Fourth Edition, McGraw-Hill, New York, 2000, pp. 411-415, U.S. Pat. No. 5,203,977, U.S. Pat. No. 5,486,277, and U.S. Pat. No. 5,742,471.

RF diode sputtering is a vacuum coating process where an electrically isolated cathode is mounted in a chamber that can be evacuated and partially filled with an inert gas. If the cathode material is an electrical conductor, a direct-current high-voltage power supply is used to apply the high voltage potential. If the cathode is an electrical insulator, the polarity of the electrodes is reversed at very high frequencies to prevent the formation of a positive charge on the cathode that would stop the ion bombardment process. Since the electrode polarity is reversed at a radio frequency, this process is referred to as RF sputtering. Magnetron sputtering is different form of sputtering. Magnetron sputtering uses a magnetic field to trap electrons in a region near the target surface thus creating a higher probability of ionizing a gas atom. The high density of ions created near the target surface causes material to be removed many times faster than in diode sputtering. The magnetron effect is created by an array of permanent magnets included within the cathode assembly that produce a magnetic field normal to the electric field.

7.1.7.3 Collimated Sputtering

In another embodiment of the present invention, collimated sputtering is used to deposit layer 8010 onto the structure illustrated in FIG. 80F in order to form the structure illustrated in FIG. 80G. Collimated sputtering is a sputtering process where the arrival of metal occurs at an angel normal to the wafer surface. The metal may be collimated by a thick honeycomb grid that effectively blocks off angle metal atoms. Alternatively, ionizing the metal atoms and attracting them towards the wafer may collimate the metal. Collimated sputtering improves filling of high aspect ratio contacts.

7.1.7.4 Laser Ablated Deposition

In another embodiment of the present invention, laser ablated deposition is used to deposit layer 8010 onto the structure illustrated in FIG. 80F in order to form the structure illustrated in FIG. 80G. In one form of laser ablated deposition, a rotating cylindrical target surface is provided for the laser ablation process. The target is mounted in a vacuum chamber so that it may be rotated about the longitudinal axis of the cylindrical surface target and simultaneously translated along the longitudinal axis. A laser beam is focused by a cylindrical lens onto the target surface along a line that is at an angle with respect to the longitudinal axis to spread a plume of ablated material over a radial arc. The plume is spread in the longitudinal direction by providing a concave or convex lateral target surface. The angle of incidence of the focused laser beam may be other than normal to the target surface to provide a glancing geometry. Simultaneous rotation about and translation along the longitudinal axis produce a smooth and even ablation of the entire cylindrical target surface and a steady evaporation plume. Maintaining a smooth target surface is useful in reducing undesirable splashing of particulates during the laser ablation process and thereby depositing high quality thin films. See, for example, U.S. Pat. No. 5,049,405, which is hereby incorporated by reference in its entirety.

7.1.7.5 Molecular Beam Deposition

In another embodiment of the present invention, molecular beam deposition is used to deposit layer 8010 onto the structure illustrated in FIG. 80F in order to form the structure illustrated in FIG. 80G. Molecular beam deposition is a method of growing films, under vacuum conditions, by directing one or more molecular beams at a substrate. In some instances, molecular beam deposition involves epitaxial film growth on single crystal substrates by a process that typically involves either the reaction of one or more molecular beams with the substrate or the deposition on the substrate of the beam particles. The term “molecular beam” refers to beams of monoatomic species as well as polyatomic species. The term molecular beam deposition includes both epitaxial growth and nonepitaxial growth processes. Molecular beam deposition is a variation of simple vacuum evaporation. However, molecular beam deposition offers better control over the species incident on the substrate than does vacuum evaporation. Good control over the incident species, coupled with the slow growth rates that are possible, permits the growth of thin layers having compositions (including dopant concentrations) that are precisely defined. Compositional control is aided by the fact that growth is generally at relatively low substrate temperatures, as compared to other growth techniques such as liquid phase epitaxy or chemical vapor deposition, and diffusion processes are very slow. Essentially arbitrary layer compositions and doping profiles may be obtained with precisely controlled layer thickness. In fact, layers as thin as a monolayer are grown by MBE. Furthermore, the relatively low growth temperature permits growth of materials and use of substrate materials that could not be used with higher temperature growth techniques. See for example, U.S. Pat. No. 4,681,773, which is hereby incorporated by reference in its entirety.

7.1.7.6 Ionized Physical Vapor Deposition

In another embodiment of the present invention, ionized physical vapor deposition (I-PVD), also known as ionized metal plasma (IMP) is used to deposit layer 8010 onto the structure illustrated in FIG. 80F in order to form the structure illustrated in FIG. 80G. In I-PVD, metal atoms are ionized in an intense plasma. Once ionized, the metal is directed by electric fields perpendicular to the wafer surface. Metal atoms are introduced into the plasma by sputtering from the target. A high density plasma is generated in the central volume of the reactor by an inductively coupled plasma (ICP) source. This electron density is sufficient to ionize approximately 80% of the metal atoms incident at the wafer surface. The ions from the plasma are accelerated and collimated at the surface of the wafer by a plasma sheath. The sheath is a region of intense electric field that is directed toward the wafer surface. The field strength is controlled by applying a radio frequency bias.

7.1.7.7 Ion Beam Deposition

In another embodiment of the present invention, ion beam deposition (IBD) is used to deposit layer 8010 onto the structure illustrated in FIG. 80F in order to form the structure illustrated in FIG. 80G. IBD uses an energetic, broad beam ion source carefully focused on a grounded metallic or dielectric sputtering target. Material sputtered from the target deposits on a nearby substrate to create a film. Most applications also use a second ion source, termed an ion assist source (IAD), that is directed at the substrate to deliver energetic noble or reactive ions at the surface of the growing film. The ion sources are “gridded” ion sources and are typically neutralized with an independent electron source. IBD processing yields excellent control and repeatability of film thickness and properties. Process pressures in IBD systems are ˜10-4 Torr. Hence, there is very little scattering of either ions delivered by the ion sources or material sputtered from the target of the surface. Compared to sputter deposition using magnetron or diode systems, sputter deposition by IBD is highly directional and more energetic. In combination with a substrate fixture that rotates and changes angle, IBD systems deliver a broad range of control over sidewall coatings, trench filling and liftoff profiles.

7.1.7.8 Atomic Layer Deposition

In another embodiment of the present invention, atomic layer deposition is used to deposit layer 8010 onto the structure illustrated in FIG. 80F in order to form the structure illustrated in FIG. 80G. Atomic layer deposition is also known as atomic layer epitaxy, sequential layer deposition, or pulsed-gas chemical vapor deposition. Atomic layer deposition involves use of a precursor based on self-limiting surface reactions. Generally, the structure illustrated in FIG. 80F is exposed to a first species that deposits as a monolayer. Then, the monolayer is exposed to a second species to form a fully reacted layer plus gaseous byproducts. The process is typically repeated until a desired thickness is achieved. Atomic layer deposition and various methods to carry out the same are described in U.S. Pat. No. 4,058,430 to Suntola et al., entitled “Method for Producing Compound Thin Films,” U.S. Pat. No. 4,413,022 to Suntola et al., entitled “Method for Performing Growth of Compound Thin Films,” to Ylilammi, and George et al., 1996, J. Phys. Chem. 100, pp. 13121-13131. Atomic layer deposition has also been described as a chemical vapor deposition operation performed under controlled conditions that cause the deposition to be self-limiting to yield deposition of, at most, a monolayer. The deposition of a monolayer provides precise control of film thickness and improved compound material layer uniformity. Atomic layer deposition may be performed using equipment such as the Endura Integrated Cu Barrier/Seed system (Applied Materials, Santa Clara, Calif.).

7.1.7.9 Hot Filament Chemical Vapor Deposition

In another embodiment of the present invention, hot filament chemical vapor deposition (HFCVD) is used to deposit layer 8010 onto the structure illustrated in FIG. 80F in order to form the structure illustrated in FIG. 80G. In HFCVD, reactant gases are flowed over a heated filament to form precursor species that subsequently impinge on the substrate surface, resulting in the deposition of high quality films. HFCVD has been used to grow a wide variety of films, including diamond, boron nitride, aluminum nitride, titanium nitride, boron carbide, as well as amorphous silicon nitride. See for example, Deshpande et al., 1995, J. Appl. Phys. 77, pp. 6534-6541, which is hereby incorporated by reference in its entirety.

7.1.7.10 Screen Printing

In another embodiment of the present invention, a screen printing (also known as silk-screening) process is used to deposit layer 8010 onto the structure illustrated in FIG. 80F in order to form the structure illustrated in FIG. 80G. A paste or ink is pressed onto portions of the structure illustrated in FIG. 80F through openings in the emulsion on a stainless steel screen. See, for example, Lambrechts and Sansen, Biosensors: Microelectrochemical devices, The Institute of Physics Publishing, Philadelphia, 1992. The paste consists of a mixture of the material of interest, an organic binder, and a solvent. The organic binder determines the flow properties of the paste. The bonding agent provides adhesion of particles to one another and to the substrate. The active particles make the ink a conductor, a resistor, or an insulator. The lithographic pattern in the screen emulsion is transferred onto portions of the structure illustrated in FIG. 80F by forcing the paste through the mask openings with a squeegee. In a first step, paste is put down on the screen. Then the squeegee lowers and pushes the screen onto the substrate, forcing the paste through openings in the screen during its horizontal motion. During the last step, the screen snaps back, the thick film paste that adheres between the screening frame and the substrate shears, and the printed pattern is formed on the substrate. The resolution of the process depends on the openings in the screen and the nature of the paste. With a 325-mesh screen (i.e., 325 wires per inch or 40 μM holes) and a typical paste, a lateral resolution of 100 μM can be obtained. For difficult-to-print pastes, a shadow mask may complement the process, such as a thin metal foil with openings. However, the resolution of this method is inferior (>500 μM). After printing, the wet films are allowed to settle for a period of time (e.g., fifteen minutes) to flatten the surface while drying. This removes the solvents from the paste. Subsequent firing bums off the organic binder, metallic particles are reduced or oxidized, and glass particles are sintered. Typical temperatures range from 500° C. to 1000° C. After firing, the thickness of resulting layer 8010 ranges from 10 μM to 50 μM. One silk-screening setup is the DEK 4265 (Universal Instrument Corporation, Binghamton, N.Y.).

Commercially available inks (pastes) that can be used in the screen printing include conductive (e.g., Au, Pt, Ag/Pd, etc.), resistive (e.g., RuO₂, IrO₂), overglaze, and dielectric (e.g., Al₂O₃, ZrO₂). The conductive pastes are based on metal particles, such as Ag, Pd, Au, or Pt, or a mixture of these combined with glass. Resistive pastes are based on RuO2 or Bi2Ru2O7 mixed with glass (e.g., 65% PBO, 25% SiO2, 10% Bi2O3). The resistivity is determined by the mixing ratio. Overglaze and dielectric pastes are based on glass mixtures. Different melting temperatures can be achieved by adjusting the paste composition. See, for example, Madou, Fundamentals of Microfabrication Second Edition, CRC Press, Boca Raton, Fla., 2002, pp. 154-156.

7.1.7.11 Electroless Metal Deposition

In another embodiment of the present invention, electroless metal deposition is used to deposit layer 8010 onto the structure illustrated in FIG. 80F in order to form the structure illustrated in FIG. 80G. In electroless plating, layer 8010 is built a metal deposit by chemical means without applying a voltage and with consuming the substrate. Electroless plating baths can be used to form Au, Co—P, Cu, Ni—Co, Ni—P, Pd, or Pt layers 8010. See, for example, Madou, Fundamentals of Microfabrication Second Edition, CRC Press, Boca Raton, Fla., 2002, pp. 344-345.

7.1.7.12 Electroplating

In another embodiment of the present invention, electroplating is used to deposit layer 8010 onto the structure illustrated in FIG. 80F in order to form the structure illustrated in FIG. 80G. Electroplating takes place in an electrolytic cell. The reactions that take place in electroplating involve current flow under an imposed bias. In some embodiments, layer 8010 is deposited as part of a damascene process. See, for example, Madou, Fundamentals of Microfabrication Second Edition, CRC Press, Boca Raton, Fla., 2002, pp. 346-357.

7.1.8 Resist Layer Deposition for the Biosensor Electrodes

Layer 8010 (FIG. 80G) is patterned by a process that begins with covering the layer with resist layer 8012 to form the structure illustrated in FIG. 80H. The particular resist used to form resist layer 8012 is application dependent. A variety of resists that may be used to form resist layer 8012 are described in Section 7.1.2, above.

7.1.9 Mask Alignment and Resist Layer Exposure for the Biosensor Electrodes

After resist layer 8012 has been overlaid onto layer 8010, the next step is alignment and exposure of resist layer 8012. Techniques described in Section 7.1.3, above, may be used to align a mask 8020 to resist layer 8012 as well as to expose the resist layer (FIG. 80I).

7.1.10 Resist Layer Development For The Biosensor Electrodes

After exposure through mask 8020 (FIG. 801), the pattern for material 106 and 110, as illustrated in FIG. 1, is coded as a latent image in resist 8012 as regions of exposed and unexposed resist. The pattern is developed in the resist by chemical dissolution of unpolymerized resist regions to form the structure illustrated in FIG. 80J. Section 7.1.4, above, describes a number of development techniques that process the structure illustrated in FIG. 801 into the structure illustrated in FIG. 80J.

7.1.11 Biosensor Electrode Etching

After the development described in Section 7.1.9, an etching step is used to pattern materials 106 and 110 into the shape they have in FIG. 1. The type of etching depends on the composition used to form materials 106 and 110. For example, if the material is aluminum or aluminum alloy, phosphoric acid can be used for etching. In one example, a 16:1:1:2 solution of phosphoric acid, nitric acid, acetic acid, and water can be used. In other embodiments, plasma etching (Section 7.1.5.3), ion beam etching (7.1.5.4) or reactive ion etching (7.1.5.5) is used. The result of etching step is the selective removal of unprotected regions of layer 8010 from the structure illustrated in FIG. 80J in order to achieve the structure illustrated in FIG. 80K.

7.1.12 Electrode Residual Layer Removal

The result of the etching process described in Section 7.1.10 is the structure illustrated in FIG. 80K. Next, residual layer 8012 is removed in a process such as any one of those described in Section 7.1.6, above. One of skill in the art will appreciate that there are mask removal processes in addition to those described in Section 7.1.6 and any such process may be used to remove mask 8012. The result of mask 8012 removal is the structure illustrated in FIG. 80L, which is equivalent to the structure illustrate in FIG. 1.

7.1.13 Alternative Lithographic Techniques

Methods for manufacturing the biosensors of the present invention, using optical lithography, have been described. However, the present invention contemplates a broad range of alternative lithographic techniques that can be used to manufacture the biosensors of the present invention. One such technique is proximity x-ray lithography. X-ray lithography involves the use of proximity printing, where the mask is brought to within a few microns of wafer 102 and the x rays are passed directly through the mask and onto the wafer. This is in contrast to optical lithography, which can project the image using a lens. X-ray masks are comprised of very thin membranes (thickness less than two microns) of low-atomic numbered materials, on which the electrode pattern (material 106 and 110 pattern) is placed in the form of high-numbered material. See, for example, Levinson, Principles of Lithography, SPIE Press, Bellingham, Wash., 2001, pp. 335-341.

Another method for manufacturing the biosensors of the present invention is extreme ultraviolet lithography. Extreme ultraviolet lithography involves the use of wavelengths in the range of 11-14 μm, and offers the possibility of improved resolution. See, for example, Levinson, Principles of Lithography, SPIE Press, Bellingham, Wash., 2001, pp. 341-347.

Yet another method for manufacturing the biosensors of the present invention is electron-beam direct-write lithography. In electron-beam direct-write lithography, electron beams having energies in the range of 50 keV to 100 keV are used to pattern wafers (substrates) 102. Electron beams have produced features as small as 10 nm. See, for example, Craighead, 1984, J. Appl. Phys. 44, pp. 4430-4435. Electron-beam direct-write lithography has the advantage over optical lithography in that a mask 8004 is not required in electron-beam direct-write lithography. See, for example, Levinson, Principles of Lithography, SPIE Press, Bellingham, Wash., 2001, pp. 347-349.

Additional techniques and apparatus that may be used to make the biosensors of the present invention include, electron-projection lithography, small-field EPL systems, large-field EPL systems as well as ion-projection lithography. See, for example, Levinson, Principles of Lithography, SPIE Press, Bellingham, Wash., 2001, pp. 349-355; Nakayama et al., 1993, Proceedings of SPIE 1924, pp. 183-192; Berger and Gibson, 1990, Appl. Phys. Lett. 47, 153-155; and Stengel et al., 1985, Proceedings of SPIE 537, 138-145.

7.2 Formation of Non-Overlapping Electrodes by Deposition at an Angle

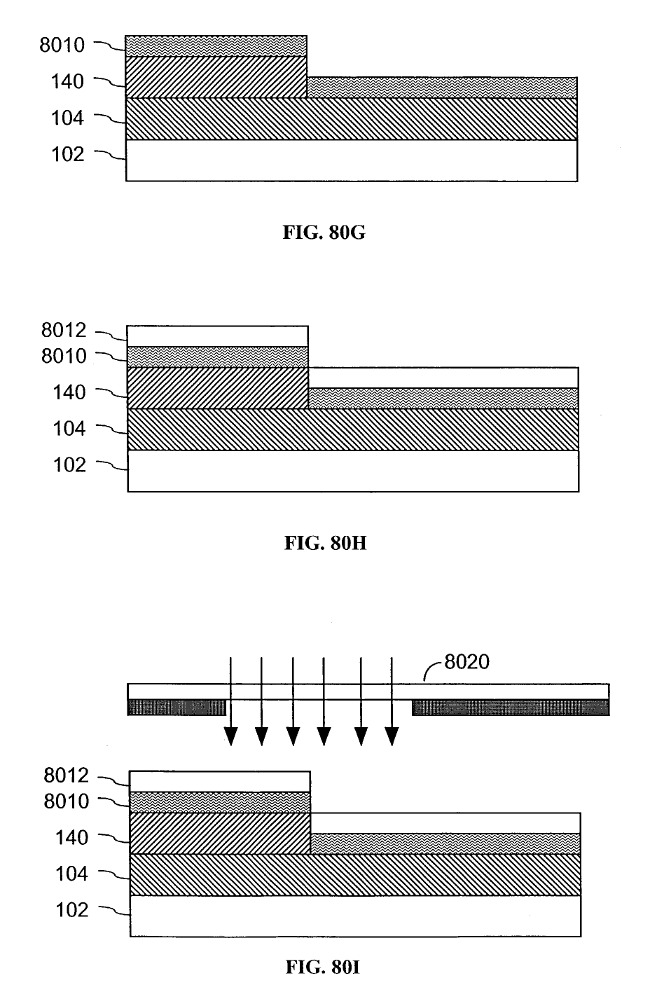

In one aspect of the present invention, materials 106 and 110 are deposited at an angle. Some embodiments in accordance with this aspect of the invention begin with the structure illustrated in FIG. 80F. Then, rather than depositing a layer 8010 (as described in Section 7.1.7) and using a resist layer to pattern layer 8010 (as described in Sections 7.1.7 through 7.1.11), the composition used to form materials 106 and 110 is deposited at an angle 8106 as illustrated in FIG. 81. In one embodiment, the angle 8106 at which the composition used to form materials 106 and 110 is deposited is defined as the angle between the plane 8102 formed by the upper surface of substrate 102 and vector 8104 (FIG. 81). Vector 8104 is the path that the composition takes when it is deposited onto the structure illustrated in FIG. 80F. Angle 8106 is defined herein as the angle with respect to substrate 104.

In some embodiments in accordance with this aspect of the invention, physical vapor deposition, direct current diode sputtering, radio frequency diode sputtering, direct current magnetron sputtering, or radio frequency magnetron sputtering is used to deposit the composition used to form materials 106 and 110 at angle 8106. Such deposition techniques are described in Section 7.1.7.2, above. In some embodiments, chemical vapor deposition is used to deposit the composition used to form materials 106 and 110 at angle 8106. Chemical vapor deposition described in Section 7.1.1.2, above. In some embodiments, reduced pressure chemical vapor deposition (Section 7.1.1.3), low pressure chemical vapor deposition (Section 7.1.1.4), atmospheric pressure chemical vapor deposition (Section 7.1.1.5), or plasma enhanced chemical vapor deposition (Section 7.1.1.6) is used to deposit the composition used to form materials 106 and 110 at an angle 8106. In some embodiments, vacuum evaporation (Section 7.1.7.1, e.g., E-beam evaporation) is used to deposit the composition used to form materials 106 and 110 at an angle 8106.

In some embodiments in accordance with this aspect of the invention, angle 8106 (FIG. 81) is any angle between 0 and 2π radians. In one embodiment, angle 8106 is zero radians. In another embodiment, angle 8106 is 2π radians. In still another embodiment, angle 8106 is π/2 radians or π/4 radians. In some embodiments in accordance with this aspect of the invention, the biosensor illustrated in FIG. 81 is manufactured using the techniques described in Section 7.1, above.

In this aspect of the invention, insulator layer 104 is optional. Thus, there are embodiments that are identical to the biosensor illustrated in FIG. 81 with the exception that insulator layer 104 is absent (not shown). Such embodiments can be manufactured using the techniques described in Section 7.1, above, beginning with the step described in Section 7.1.2 (i.e., Section 7.1.1 is skipped). In some embodiments of the present invention, materials 106 and 110 are different. In such embodiments, the methods described in Section 7.1 above are used to pattern materials 106 and 110 so that they have the configuration illustrated in FIG. 81.

7.3 Formation of Two Non-Overlapping Electrodes with Two Insulator Layers and Introduction of a Cavity

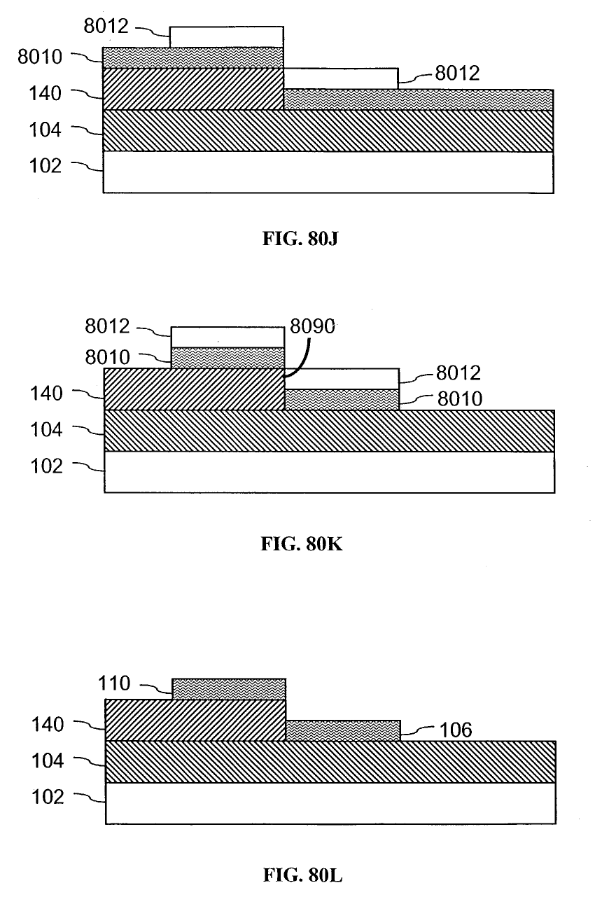

In another aspect of the present invention, a portion of spacer 140 (FIG. 82, element 8202) is removed to provide cavity 8202. The presence of cavity 8202 in the biosensor illustrated in FIG. 82 increases the path between material 106 and 110 that current must travel in order to short circuit the biosensor.

In one embodiment in accordance with this aspect of the present invention, the manufacture of the biosensor illustrated in FIG. 82 begins with the structure illustrated in FIG. 80F. Then, rather than depositing a layer 8010 (as described in Section 7.1.7) and using a resist layer to pattern layer 8010 (as described in Sections 7.1.7 through 7.1.11), the composition used to form materials 106 and 110 is deposited at an angle 8106 as illustrated in FIG. 82. Then, the structure is overlaid with a resist layer and cavity 8202 is formed using a semiconductor wet chemical etch described in Section 7.1.5.1, above. Once the etching is finished, the resist layer is removed to yield the structure illustrated in FIG. 82.

In another embodiment of the present invention, the manufacture of the biosensor illustrated in FIG. 82 is performed using the techniques used to build the biosensor illustrated in FIG. 80, (e.g., the techniques described in Section 7.1) with the addition of a wet chemical etch in order to create cavity 8202. There are two different points at which the wet chemical etch can be performed. The first is after the formation of FIG. 80K (i.e., after Section 7.1.11, above). If a wet chemical etch is performed at this stage, it is important that resist 8012 have a thickness less than that illustrated in FIG. 80K so that a portion of the side-wall 8090 of spacer 140 is exposed to the etchant. The second point at which a wet chemical etch can be performed in order to create cavity 8202 (FIG. 82) is after the completion of Section 7.1.12 (i.e., after mask 8012 removal).

In this aspect of the invention, insulator layer 104 is optional. Thus, there are embodiments that are identical to the biosensor illustrated in FIG. 82 with the exception that insulator layer 104 is absent (not shown). Such embodiments can be manufactured using the techniques described above, with the exception that the deposition described in Section 7.1.1 is skipped. In some embodiments of the present invention, materials 106 and 110 are different. In such embodiments, the methods described in Section 7.1 above are used to pattern materials 106 and 110 so that they have the configuration illustrated in FIG. 82.

7.4 Formation of Two Non-Overlapping Electrodes Using a π/2 Delivery Mechanism

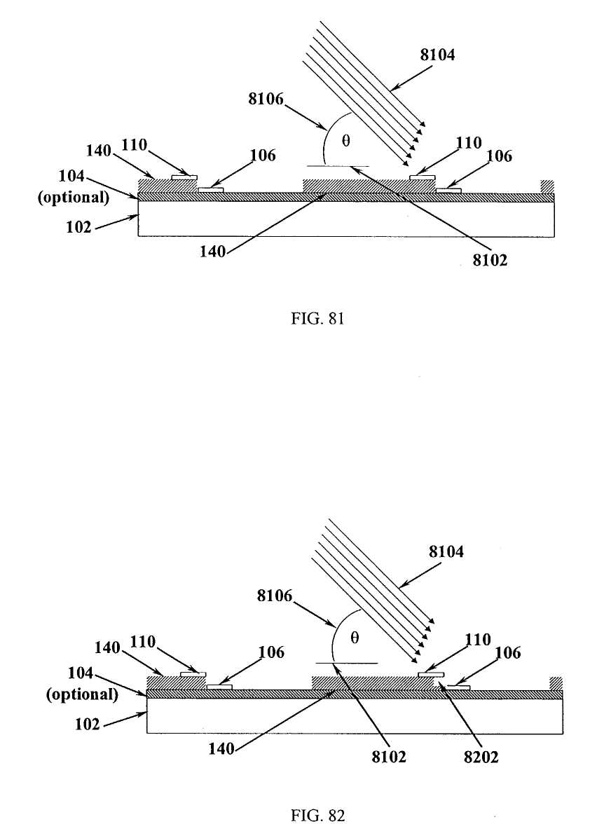

In yet another aspect of the present invention, materials 106 and 110 are deposited at an angle 8106 (FIG. 83) that is about ninety degrees (i.e., about π/2 radians). In one example, materials 106 and 110 are deposited at an angle in the range 85 to 95 degrees. In another example, materials 106 and 110 are deposited at an angle in the range of 80 to 100 degrees. Such embodiments begin with the structure illustrated in FIG. 80F. Then, rather than depositing a layer 8010 (as described in Section 7.1.7) and using a resist layer to pattern layer 8010 (as described in Sections 7.1.7 through 7.1.11), the composition used to form materials 106 and 110 is deposited at an angle 8106 as illustrated in FIG. 83. This deposition is performed using one of the techniques described in Section 7.2, above.

In embodiments in accordance with this aspect of the invention, two materials 106 (i.e. 106-1 and 106-2) are formed. Thus, a first population of macromolecules 120 may span material 106-1 and material 110 and a second population of macromolecules 120 may span material 106-2 and material 110 (not shown). In some embodiments in accordance with this aspect of the invention, the techniques described in Section 7.1 are used to make the biosensor illustrated in FIG. 83.

In this aspect of the invention, insulator layer 104 is optional. Thus, there are embodiments that are identical to the biosensor illustrated in FIG. 83 with the exception that insulator layer 104 is absent (not shown). Such embodiments can be manufactured using the techniques described above, with the exception that the deposition described in Section 7.1.1 is skipped. In some embodiments of the present invention, materials 106 and 110 are different. In such embodiments, the methods described in Section 7.1 above are used to pattern materials 106 and 110 so that they have the configuration illustrated in FIG. 83.

7.5 Formation of Two Non-Overlapping Electrodes with Cavities Using a π/2 Delivery Mechanism

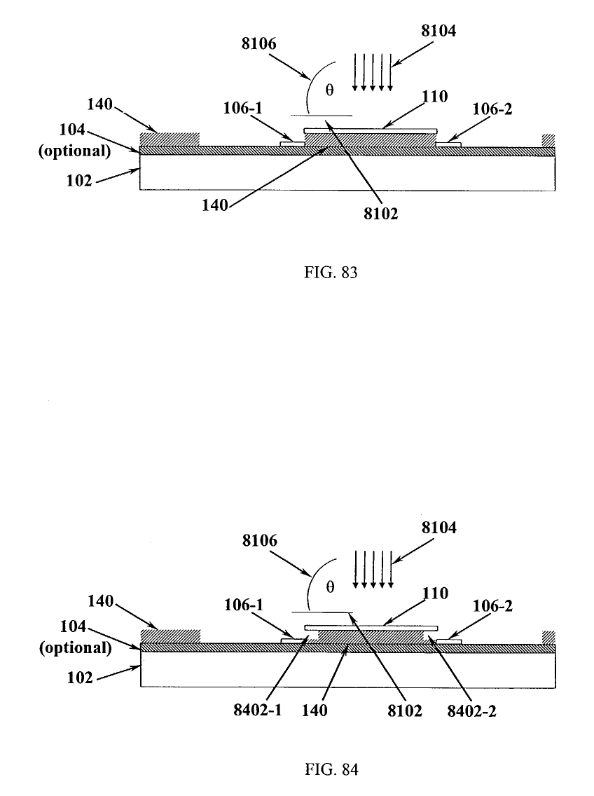

In still another aspect of the invention, a portion of spacer 140 is removed to provide cavities 8402-1 and 8402-2 in the biosensor illustrated in FIG. 84. The presence of cavities 8402-1 and 8402-2 in the biosensor illustrated in FIG. 84 increases the path between materials 106 and 110 that current must travel in order to short circuit the biosensor.

In one embodiment in accordance with this aspect of the present invention, the manufacture of the biosensor illustrated in FIG. 84 begins with the structure illustrated in FIG. 80F. Then, rather than depositing a layer 8010 (as described in Section 7.1.7) and using a resist layer to pattern layer 8010 (as described in Sections 7.1.7 through 7.1.11), the composition used to form materials 106 and 110 is deposited at an angle 8106 as illustrated in FIG. 84. This deposition is performed using one of the techniques described in Section 7.2, above.

In one aspect of the present invention, materials 106 and 110 are deposited at an angle 8106 (FIG. 84) that is about ninety degrees (e.g., π2 radians). In one example, materials 106 and 110 are deposited at an angle in the range 85 to 95 degrees. In another example, materials 106 and 110 are deposited at an angle in the range of 80 to 100 degrees. Then, the structure is overlaid with a resist layer and cavities 8402-1 and 8402-2 are formed using a semiconductor wet chemical etch described in Section 7.1.5.1, above. Once the etching is finished, the resist layer is removed to yield the structure illustrated in FIG. 84.

In another embodiment, in accordance with this aspect of the invention, the manufacture of the biosensor illustrated in FIG. 84 is performed using the techniques used to build the biosensor illustrated in FIG. 80, (e.g., the techniques described in Section 7.1) with the addition of a wet chemical etch. The additional wet chemical etch step is creates cavities 8402-1 and 8402-2. There are two different points at which the additional wet chemical etch step can be performed. The first is after the formation of FIG. 80K (i.e., after Section 7.1.11, above). The second point at which a wet chemical etch can be performed in order to create cavities 8402-1 and 8402-2 (FIG. 84) is after the completion of Section 7.1.12 (i.e., after mask 8012 removal).

In embodiments in accordance with this aspect of the invention, two materials 106 (i.e. 106-1 and 106-2) are formed (FIG. 84). Thus, a first population of macromolecules 120 may span material 106-1 and material 110 and a second population of macromolecules 120 may span material 106-2 and material 110 (not shown).

In this aspect of the invention, insulator layer 104 is optional. Thus, there are embodiments that are identical to the biosensor illustrated in FIG. 84 with the exception that insulator layer 104 is absent (not shown). Such embodiments can be manufactured using the techniques described above, with the exception that the deposition described in Section 7.1.1 is skipped. In some embodiments of the present invention, materials 106 and 110 are different. In such embodiments, the methods described in Section 7.1 above are used to pattern materials 106 and 110 so that they have the configuration illustrated in FIG. 84.

7.6 Formation of Two Non-Overlapping Electrodes with a Portion of the Insulator And Electrode Removed

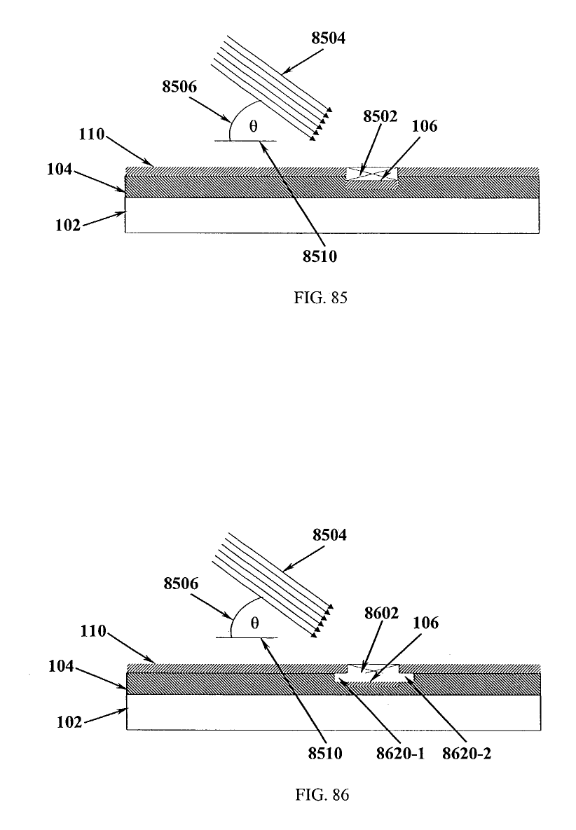

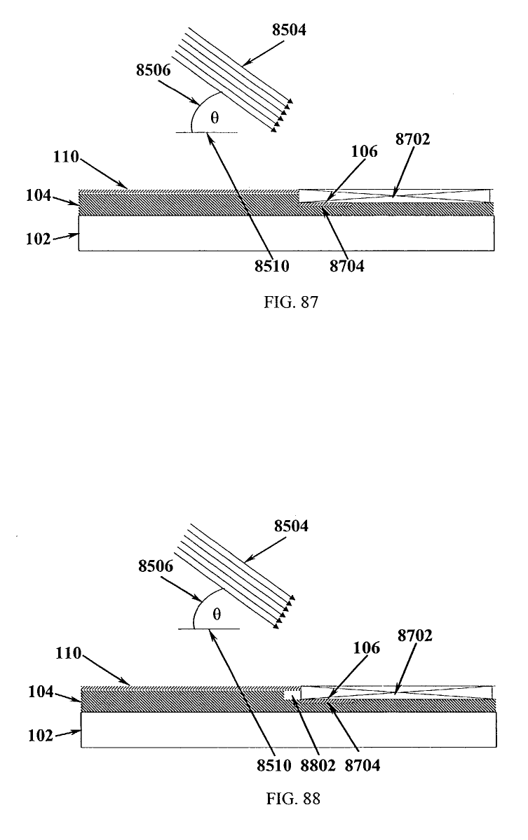

Reference will now be made to FIG. 85, which illustrates another biosensor in accordance with an embodiment of the present invention. The biosensor illustrated in FIG. 85 includes a substrate 102. Insulator layer 104 is overlaid on substrate 102. A portion of insulator 104 is removed to form cavity 8502. A composition is deposited on the structure to form material 106 (at the bottom of cavity 8502) and material 110 (on insulator 104). In some biosensors in accordance with FIG. 86, material 106 and material 110 are electrodes.

Biosensors having the configuration illustrated in FIG. 85 can be manufactured using a modified form of the process flow described in Section 7.1, above. In one embodiment, deposition of insulator 104 is accomplished using any of the techniques described or referenced in Section 7.1. Next, insulator 104 is patterned using a resist layer deposition, mask alignment, resist layer exposure, resist layer development, and etching using the techniques described, for example, in Sections 7.1.2, 7.1.3, 7.1.4, and 7.1.5. The only difference between the techniques described in Sections 7.1.2 through 7.1.5 and the instant process is that insulator layer 104 is patterned rather than spacer 140. Spacer 140 is not used in the instant process. The etching step yields cavity 8502 (FIG. 85). Upon mask removal, as described in Section 7.1.6 for example, materials 106 and 110 are deposited. In some embodiments, materials 106 and 110 are deposited at the same time using a technique described or referenced in Section 7.1.7, above. Matter that is deposited at the bottom of cavity 8502 forms material 106 and matter that is deposited on the upper surface of insulator layer 104 forms material 110, as illustrated in FIG. 85.

In one embodiment, the angle 8506 at which the composition used to form materials 106 and 110 is deposited is defined as the angle between the plane 8510 formed by the upper surface of substrate 102 and vector 8504 (FIG. 85). Vector 8504 is the path that the composition used to form materials 106 and 110 takes when it is deposited onto the structure.

In some embodiments in accordance with this aspect of the invention, physical vapor deposition, direct current diode sputtering, radio frequency diode sputtering, direct current magnetron sputtering, or radio frequency magnetron sputtering is used to deposit the composition used to form materials 106 and 110 at angle 8506. Such deposition techniques are described in Section 7.1.7.2, above. In some embodiments, chemical vapor deposition is used to deposit the composition used to form materials 106 and 110 at angle 8506. Chemical vapor deposition described in Section 7.1.1.2, above. In some embodiments, reduced pressure chemical vapor deposition (Section 7.1.1.3), low pressure chemical vapor deposition (Section 7.1.1.4), atmospheric pressure chemical vapor deposition (Section 7.1.1.5), or plasma enhanced chemical vapor deposition (Section 7.1.1.6) is used to deposit the composition used to form materials 106 and 110 at an angle 8506. In some embodiments, vacuum evaporation (Section 7.1.7.1, e.g., E-beam evaporation) is used to deposit the composition used to form materials 106 and 110 at an angle 8506.

In some embodiments in accordance with this aspect of the invention, angle 8506 (FIG. 85) is any angle between 0 and 2π radians. In one embodiment, angle 8506 is zero radians. In another embodiment, angle 8506 is 2π radians. In still another embodiment, angle 8506 is π/2 radians or π/4 radians. In some embodiments in accordance with this aspect of the invention, the biosensor illustrated in FIG. 85 is manufactured using the techniques described in Section 7.1, above. In some embodiments of the present invention, materials 106 and 110 are different. In such embodiments, the methods described in Section 7.1 above are used to pattern materials 106 and 110 so that they have the configuration illustrated in FIG. 85.

7.7 Formation of Two Non-Overlapping Electrodes with Additional Insulator Removal

Reference will now be made to FIG. 86, which illustrates another biosensor in accordance with an embodiment of the present invention. The biosensor illustrated in FIG. 86 includes a substrate 102. Insulator layer 104 is overlaid on substrate 102. A portion of insulator 104 is removed to form cavity 8602. A composition is deposited on the structure to form material 106 (at the bottom of cavity 8602) and material 110 (on insulator 104). In some biosensors in accordance with FIG. 86, material 106 and material 110 are electrodes.

Biosensors having the configuration illustrated in FIG. 86 can be manufactured using the structure illustrated in FIG. 85 as a starting point. In such embodiments, a resist layer is optionally deposited on top of materials 106 and 110 using techniques described, for example, in Section 7.1.2. Then, the structure is subjected to a wet chemical etch using the techniques described in, for example, Section 7.1.5.1 in order to yield cavities 8620-1 and 8620-2. Finally, the resist layer is developed in order to remove the optional resist layer. In some embodiments of the present invention, materials 106 and 110 are different. In such embodiments, the methods described in Section 7.1 above are used to pattern materials 106 and 110 so that they have the configuration illustrated in FIG. 86.

7.8 Formation of Two Non-Overlapping Electrodes with Insulator Removal

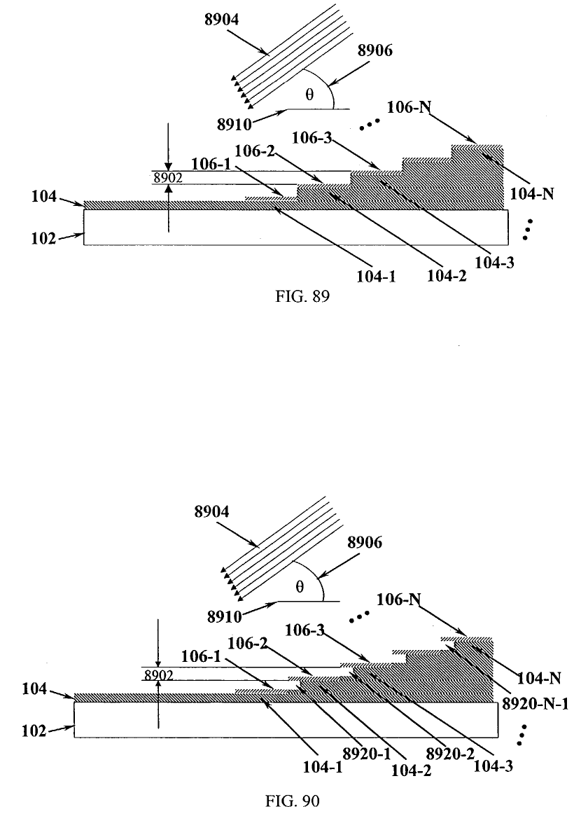

Reference will now be made to FIG. 87, which illustrates another biosensor in accordance with an embodiment of the present invention. The biosensor illustrated in FIG. 87 includes a substrate 102. Insulator layer 104 is overlaid on substrate 102. A portion of insulator 104 is removed to form shelf 8702. A portion of shelf 8702 is removed to form cavity 8704. A composition is deposited on the structure to form material 106 (in cavity 8704) and material 110 (on the upper surface of insulator 104). In some biosensors in accordance with FIG. 87, material 106 and material 110 are electrodes.

Biosensors having the configuration illustrated in FIG. 87 can be manufactured using a modified form of the process flow described in Section 7.1, above. In one embodiment, deposition of insulator 104 is accomplished using any of the techniques described or referenced in Section 7.1. Next, insulator 104 is patterned using a resist layer deposition, mask alignment, resist layer exposure, resist layer development, and etching using the techniques described, for example, in Sections 7.1.2, 7.1.3, 7.1.4, and 7.1.5. The only difference between the techniques described in Sections 7.1.2 through 7.1.5 and the instant process is that insulator layer 104 is patterned rather than spacer 140. Spacer 140 is not used in the instant process. The etching step yields shelf 8702 and cavity 8704 (FIG. 87). Upon mask removal, as described in Section 7.1.6 for example, materials 106 and 110 are deposited. In some embodiments, materials 106 and 110 are deposited at the same time using a technique described or referenced in Section 7.1.7, above. Matter that is deposited at the bottom of shelf 8702 forms material 106 and matter that is deposited in cavity 8704 forms material 110, as illustrated in FIG. 87. Materials 106 and 110 are patterned using the techniques described in, for example, Sections 7.1.8, 7.1.9, 7.1.10, 7.1.11, and 7.1.12, above. In some embodiments of the present invention, materials 106 and 110 are different. In such embodiments, the methods described in Section 7.1 above are used to pattern materials 106 and 110 so that they have the configuration illustrated in FIG. 85.

7.9 Stacked Non-Overlapping Electrodes

Reference will now be made to FIG. 89, which illustrates another biosensor in accordance with an embodiment of the present invention. The biosensor illustrated in FIG. 89 includes a substrate 102. Insulator layer 104 is overlaid on substrate 102. Insulator layer 104 is patterned to include steps 104-1 through 104-N. Steps 104-1 through 104-N are illustrated in FIG. 89.

In one embodiment in accordance with FIG. 89, a composition is deposited on each step 104-X of insulator 104 to form materials 106 through material 106-N. In this embodiment, a first pool of macromolecules 120 bridge material 106-1 and material 106-2, a second pool of macromolecules 120 bridge material 106-3 and material 106-4, and so forth, where each pool of macromolecules 120 is the same or different.

In other embodiments in accordance with FIG. 89, steps in the set of steps 104-1 through 104-N are alternatively overlaid with materials 106 and 110 (not shown). For example, in one nonlimiting embodiment of the present invention, step 104-1 (FIG. 89) is overlaid with material 106-1, step 104-2 is overlaid with material 110-1, step 104-3 is overlaid with material 106-2, step 104-4 is overlaid with material 110-2, and so forth. In this embodiment, a first pool of macromolecules 120 bridge material 106-1 and material 110-1, a second pool of macromolecules 120 bridge material 106-2 and material 110-2, and so forth, where each pool of macromolecules 120 is the same or different.

Referring to FIG. 89, one embodiment of the present invention provides a biosensor. The biosensor comprises a substrate 102 and an insulator layer 104 overlaid on substrate 102. In the biosensor, the insulator layer 104 comprises a plurality of steps 104-X and a first step in the plurality of steps is at a different height, with respect to substrate 102, than a second step in the plurality of steps. Furthermore, each step in the plurality of steps is associated with a different electrically conducting layer 106 that is overlaid on the step. Each electrically conducting layer 106 on each step in the plurality of steps is electrically insulated from all other electrically conducting layers in the biosensor by insulator layer 104. In some embodiments, each electrically conducting layer 106 in the biosensor is addressable by an electrical source. For example, a voltage or electrical current may be applied to any desired electrically conducting layer 106 in the biosensor.

In some embodiments of biosensors in accordance with FIG. 89, the difference in height, with respect to substrate 102, between a first step in the plurality of steps and a second step in the plurality of steps is between 60 Angstroms and 200 Angstroms. In some embodiments of biosensors in accordance with FIG. 89, the difference in height, with respect to substrate 102, between a first step in the plurality of steps and a second step in the plurality of steps is less than 500 Angstroms, or less than 1000 Angstroms.

In some embodiments of the present invention, each step 104-N has a height 8902 (FIG. 89) of between 60 Angstroms and 200 Angstroms, between 300 Angstroms and 400 Angstroms, between 200 Angstroms and 300 Angstroms, less than 300 Angstroms, less than 200 Angstroms, less than 150 Angstroms, less than 100 Angstroms, or between 50 Angstroms and 80 Angstroms.

Referring to FIG. 89, some embodiments of the present invention provide a biosensor having a plurality of steps in which a first step and a second step are adjacent to each other. Furthermore, a first portion of a macromolecule binds to the first step in the plurality of steps and a second portion of the macromolecule binds to the second step.

Biosensors having the configuration illustrated in FIG. 89 can be manufactured using a modified form of the process flow described in Section 7.1, above. In one embodiment, deposition of insulator 104 is accomplished using any of the techniques described or referenced in Section 7.1. Next, insulator 104 is patterned using a resist layer deposition, mask alignment, resist layer exposure, resist layer development, and etching using the techniques described, for example, in Sections 7.1.2, 7.1.3, 7.1.4, and 7.1.5. One difference between the techniques described in Sections 7.1.2 through 7.1.6 and the instant process is that insulator layer 104 is patterned rather than spacer 140. Spacer 140 is not used in the instant process. The etching step yields step 104-1 (FIG. 89). To form steps 104-2 through steps 104-N, the process of depositing (or growing) insulator layer 104 using any of the techniques described or referenced in Section 7.1 and patterning the freshly formed insulator 104 layer using the techniques described in Section 7.1.2 through 7.1.6 is repeated N−1 times.

In one embodiment of the present invention, once the step configuration has been formed, material 106 is deposited onto each step using a technique described or referenced in Section 7.1.7, above. In some embodiments of the present invention, the composition used to form material 106 is deposited at an angle 8906. Angle 8906 is defined as the angle between the plane 8910 formed by the upper surface of substrate 102 and vector 8904 (FIG. 89). Vector 8904 is the path that the composition used to form material 106 takes when it is deposited onto the structure.

In some embodiments in accordance with this aspect of the invention, physical vapor deposition, direct current diode sputtering, radio frequency diode sputtering, direct current magnetron sputtering, or radio frequency magnetron sputtering is used to deposit the composition used to form material 106 at angle 8906. Such deposition techniques are described in Section 7.1.7.2, above. In some embodiments, chemical vapor deposition is used to deposit the composition used to form material 106 at angle 8906. Chemical vapor deposition is described in Section 7.1.1.2, above. In some embodiments, reduced pressure chemical vapor deposition (Section 7.1.1.3), low pressure chemical vapor deposition (Section 7.1.1.4), atmospheric pressure chemical vapor deposition (Section 7.1.1.5), or plasma enhanced chemical vapor deposition (Section 7.1.1.6) is used to deposit the composition used to form material 106 at an angle 8906. In some embodiments, vacuum evaporation (Section 7.1.7.1, e.g., E-beam evaporation) is used to deposit the composition used to form material 106 at an angle 8906.

In some embodiments in accordance with this aspect of the invention, angle 8906 (FIG. 89) is any angle between 0 and 2π radians. In one embodiment, angle 8906 is zero radians. In another embodiment, angle 8906 is 2π radians. In still another embodiment, angle 8906 is π/2 radians or π/4 radians. In some embodiments of the present invention, materials 106 and 110 are different. In such embodiments, the methods described in Section 7.1 above are used to pattern materials 106 and 110 so that they have the configuration illustrated in FIG. 89.

7.10 Stacked Non-Overlapping Electrodes with Insulator Removal

In still another aspect of the invention, a portion of insulator 104 is removed from the biosensor illustrated in FIG. 89 to provide cavities 8920-1 through 8920-N−1 that are found in the biosensor illustrated in FIG. 90. The presence of cavities 8920-1 through 8920-N−1 in the biosensor illustrated in FIG. 90 increases the path that a short circuiting current must travel between electrodes in the biosensor illustrated in FIG. 90.

Referring to FIG. 90, some embodiments of the present invention provide a biosensor comprising a plurality of steps. A different electrically conducting layer is associated with each step in the plurality of steps. Furthermore, the electrically conducting layer associated with a step in the plurality of steps is electrically insulated from other electrically conducting layers in the biosensor by a cavity in the step associated with the electrically conducting layer.

Biosensors having the configuration illustrated in FIG. 90 can be manufactured using the structure illustrated in FIG. 89 as a starting point. In such embodiments, a resist layer is optionally deposited on top of materials 106 (and 110) using techniques described, for example, in Section 7.1.2. Then, the structure is subjected to a wet chemical etch using the techniques described in, for example, Section 7.1.5.1 in order to yield cavities 8920-1 through 8920-N−1. Finally, the resist layer is developed in order to remove it. In some embodiments of the present invention, materials 106 and 110 are different. In such embodiments, the methods described in Section 7.1 above are used to pattern materials 106 and 110 so that they have the configuration illustrated in FIG. 90.

7.11 Planar Arrays of Biosensors

The present invention further provides planar arrays of biosensors such at the array illustrated in FIG. 91. In such arrays, paired materials 106 and 110 are positioned adjacent to each other such that a macromolecule 120 can span the paired materials (not shown). In some embodiments of the present invention, paired materials 106 and 110 (e.g. material 106-1 and 110-1, materials 106-2 and 110-2, and so forth) are separated by a distance “d” (FIG. 91) that is between 10 Angstroms and 15 Angstroms, between 15 Angstroms and 20 Angstroms, between 20 Angstroms and 25 Angstroms, between 25 Angstroms and 30 Angstroms, between 30 Angstroms and 35 Angstroms, between 35 Angstroms and 40 Angstroms, between 40 Angstroms and 45 Angstroms, between 45 Angstroms and 50 Angstroms, between 50 Angstroms and 55 Angstroms, between 55 Angstroms and 60 Angstroms, between 60 Angstroms and 70 Angstroms, between 70 Angstroms and 85 Angstroms, between 85 Angstroms and 100 Angstroms, or more than 100 Angstroms. The biosensor array illustrated in FIG. 91 can be manufactured using the techniques described in Section 7.1 with the exception that spacer 140 (e.g., the second insulator layer) is not patterned. In some embodiments in accordance with FIG. 91, materials 106 and 110 are electrodes. In some embodiments in accordance with FIG. 91, materials 106 and 110 are different. In such embodiments, the methods outlined in Section 7.1 above are used to pattern materials 106 and 110 so that they have the configuration illustrated in FIG. 91. In some embodiments of the present invention, insulator 104 and spacer 140 are made of the same material. In other embodiments of the present invention, insulator 104 and 140 are made of different materials.

7.12 Analyte Detection

This section describes a number of different novel methods that can be used to detect an analyte using the biosensors of the present invention. Section 7.12.1 describes the type of analyte samples that can be used. Section 7.12.2 describes sample delivery mechanisms that can be used to deliver analytes to the biosensors of the present invention. Section 7.12.3 describes various methods that can be used to attach macromolecules 120 to the biosensors of the present invention. Section 7.12.4 describes methods for sample detection and quantification once analytes have been introduced into devices 144 of the present invention. Section 7.12.5 describes additional methods for analyte detection in accordance with various embodiments of the present invention.

7.12.1 Sample Preparation

Virtually any sample containing an analyte can be analyzed using biosensors of this invention. Such samples include, but are not limited to, body fluids or tissues, water, food, blood, serum, plasma, urine, feces, tissue, saliva, oils, organic solvents, earth, water, air, or food products. In one embodiment, the sample is a biological sample. The term “biological sample”, as used herein, refers to a sample obtained from an organism or from components (e.g., cells) of an organism. In some embodiments, the sample is any biological tissue or fluid. Frequently, the sample is a “clinical sample” which is a sample derived from a patient. Such samples include, but are not limited to, sputum, cerebrospinal fluid, blood, blood fractions (e.g. serum, plasma), blood cells (e.g., white cells), tissue or fine needle biopsy samples, urine, peritoneal fluid, and pleural fluid, or cells therefrom. Biological samples may also include sections of tissues such as frozen sections taken for histological purposes.

Biological samples, (e.g. serum) may be analyzed directly or they may be subject to some preparation prior to use in the assays of this invention. Such preparation can include, but is not limited to, suspension/dilution of the sample in water or an appropriate buffer or removal of cellular debris, e.g. by centrifugation, selection of particular fractions of the sample before analysis.

7.12.2 Sample Delivery System

The sample that includes an analyte can be introduced into the biosensors of the present invention according to standard methods well known to those of skill in the art. Thus, for example, the sample can be introduced into the channel through an injection port, such as those used in high pressure liquid chromatography systems.

7.12.3 Sample Reaction with a Macromolecule

In one embodiment, the sample that potentially contains an analyte is provided to one or more devices 144 of a biosensor of the present invention under conditions that facilitate binding of the analyte to one or more macromolecules 120 bound to electrically conducting materials 106 and 110 of respective devices 144. Thus, for example, when macromolecules 120 bound to electrically conducting materials 106 and 110 in devices 144 of a biosensor are antibodies or proteins, reaction conditions are provided that facilitate antibody binding. Such reaction conditions are well known to those of skill in the art. See, for example, Coligan, 1991, Current Protocols in Immunology, Wiley/Greene, NY; Harlow and Lane, 1989, Antibodies: A Laboratory Manual Cold Spring Harbor Press, NY; Stites et al. (eds.) Basic and Clinical Immunology (4th ed.), Lange Medical Publications, Los Altos, Calif., and references cited therein; Goding, 1986, Monoclonal Antibodies: Principles and Practice (2nd edition) Academic Press, New York, N.Y.; and Kohler and Milstein, 1975, Nature 256: 495-497.

In some embodiments, macromolecule 120 is a nucleic acid and the biosensor is maintained under conditions that facilitate binding of the target nucleic acid (analyte) to macromolecules 120 bound to respective electrically conducting materials 106 and 110 in target devices 144 of the biosensor. Stringency of the reaction can be adjusted until the sensor shows adequate/desired specificity and selectivity. Conditions suitable for nucleic acid hybridization are well known to those of skill in the art. See, for example, Berger and Kimmel, Guide to Molecular Cloning Techniques, Methods in Enzymology, 152 Academic Press, Inc., San Diego, Calif.; Sambrook et al., 1989, Molecular Cloning—A Laboratory Manual (2nd ed.) Vol. 1-3, Cold Spring Harbor Laboratory, Cold Spring Harbor Press, NY; Ausubel et al., 1994, Current Protocols in Molecular Biology, Greene Publishing Associates, Inc. and John Wiley & Sons, Inc., New York; U.S. Pat. No. 5,017,478; and European Patent Number 0,246,864. Once the analyte is bound to the macromolecule 120 in one or more devices 144 of the biosensor, the sensor is optionally dehydrated and then read.

In one embodiment of the present invention, macromolecule 120 is a single stranded nucleic acid (e.g., DNA or RNA). Methods disclosed in Kunwar et al. are used to localize the single nucleic acid to a predetermined electrically conducting material 106 or electrically conducting material 110. See related application U.S. patent application Ser. No. ______ to be assigned, filed Dec. 26, 2002, titled “ASSOClATION OF MOLECULES WITH ELECTRODES OF AN ARRAY OF ELECTRODES,” invented by Kunwar et al. and having attorney docket number 11210-019-999 and incorporated herein by reference in its entirety. For example, electrically conducting materials 106 and 110 can be coated with a protective compound such as alkylsiloxane, an alkanetholate, and/or a fatty acid. Then, a voltage can be applied to one of material 106 and material 110 in a device 144 in a plurality of devices 144 in a biosensor of the present invention, thereby stripping the protecting groups from the material 106 or material 110 to which voltage is applied.

In the next step, macromolecule 120 is exposed to the biosensor. In one embodiment, a first portion of macromolecule 120 includes a reactive sulfur group that binds to the unprotected electrode (i.e., the unprotected material 106 or 110) and a second portion of macromolecule 120 includes a reactive group that is masked by an electrolabile masking group, a photosensitive masking group, or a chemically sensitive masking group.

The biosensor is optionally washed to remove macromolecules 120 that did not bind to the unprotected electrode. In optional embodiments, the steps of (i) unmasking a different predetermined electrode, (ii) exposing the biosensor to a different macromolecule 120, and (iii) optionally washing the biosensor are repeated until a different macromolecule 120 is bound to respective first electrodes in a portion or all of the devices 144 in a biosensor.

After one or more macromolecules 120 have been bound to respective first electrodes in a portion or all of the devices 144 in a biosensor, the biosensor is exposed to a solution that potentially comprises an analyte. In some embodiments the one or more macromolecules 120 bound to respective first electrodes in a portion or all of the devices 144 in a biosensor are each single stranded nucleic acids. In some embodiments, the analyte binds to a bound macromolecule 120 when the analyte is a single stranded nucleic acid that is complementary to one or more macromolecules 120 bound to the biosensor. In some embodiments, the analyte binds to a bound macromolecule 120 when the analyte is a single stranded nucleic acid that is capable of binding to the one or more macromolecules 120 bound to the biosensor under conditions of high stringency as defined in Section 7.12.3.1, below. In some embodiments, the analyte binds to a bound macromolecule 120 when the analyte is a single stranded nucleic acid that is capable of binding to the one or more macromolecules 120 bound to the biosensor under conditions of intermediate stringency as defined in Section 7.12.3.2, below. In still other embodiments, the analyte binds to a bound macromolecule 120 when the analyte is a single stranded nucleic acid that is capable of binding to the one or more macromolecules 120 bound to the biosensor under conditions of low stringency as defined in Section 7.12.3.3, below.

The solution potentially comprising one or more analytes is allowed to incubate with the biosensor for a period of time. In some embodiments, this incubation period has a duration of less than one minute, less than five minutes, less than 15 minutes, less than 30 minutes, less than an hour, less than four hours, or less than one day. In some embodiments, the incubation period is between one second and one minute, between one minute and five minutes, between five minutes and fifteen minutes, between fifteen minutes and 30 minutes, between 30 minutes and one hour, or more than one hour.

After the incubation period, the biosensor is washed to remove unbound analyte. Then, a voltage is applied to the electrode in each electrode pair in devices 144 of the biosensor that are still protected, thereby causing the protective groups that coat the electrode to strip away from the electrode. In embodiments where a second portion of macromolecule includes a reactive group that is masked by a electrolabile masking group, a voltage is applied at the newly unprotected electrode in order to strip away the electrolabile masking group from the second portion of macromolecule 120 thereby revealing a reactive group that binds to the second electrode. In embodiments where a second portion of macromolecule includes a reactive group that is masked by a photosensitive masking group, a light source is applied to the biosensor in order to strip away the photosensitive masking group from the second portion of macromolecule 120 thereby revealing a reactive group that binds to the second electrode. In embodiments where a second portion of macromolecule includes a reactive group that is masked by a chemically sensitive masking group, an appropriate chemical is applied to the biosensor in order to strip away the chemcially sensitive masking group from the second portion of macromolecule 120 thereby revealing a reactive group that binds to the second electrode. Suitable electrolabile masking groups, photosensitive masking groups and chemically sensitive masking groups as well as the respective voltages, light sources, and chemicals needed to strip such protecting groups from macromolecules 120 are disclosed in U.S. patent application Ser. No. ______ to be determined, titled “METHODS FOR ATTACHING MOLECULES,” inventors Freeman and Pisharody, attorney docket number 11210-017-99, filed Dec. 26, 2002, which is incorporated by reference in its entirety.

The method continues with a drying step in which the electrodes are dried, and a measuring step in which a voltage differential is applied across electrode pairs in devices 144 in the biosensor in order to measure a current across such electrode pairs.

The methods described above provide highly advantageous tools for detecting analyte binding events. It is well known that single stranded nucleic acids are poor electrical conductors. Thus, only those devices 144 in which an analyte successfully hybridized to an analyte will conduct electricity.

Another embodiment of the present invention provides a method of detecting an analyte with a biosensor. The biosensor comprises a plurality of devices 144. In some embodiments, each device 144 in the plurality of devices 144 occupies a different region on an insulator layer 104 that, in turn, overlays substrate 102. In some embodiments, each device 144 in the plurality of devices 144 occupies a different region on a substrate 102. The method in accordance with this embodiment of the invention can be used with any of the biosensors of the present invention. In the method, a first portion of a macromolecule 120 is attached to a first electrically conducting material (e.g., material 106) and a second portion of the macromolecule 120 is attached to a second electrically conducting material 110 in a device 144 in the plurality of devices. Methods by which this attachment can be accomplished are disclosed in copending U.S. patent application Ser. No. ______ to be assigned, filed Dec. 26, 2002 titled “METHODS FOR ATTACHING MOLECULES” invented by Freeman and Pisharody and having attorney docket number 11210-017-999 and U.S. patent application Ser. No. ______ to be assigned, filed Dec. 26, 2002, titled “ASSOClATION OF MOLECULES WITH ELECTRODES OF AN ARRAY OF ELECTRODES,” invented by Kunwar et al., and having attorney docket number 11210-019-999, each of which is hereby incorporated by reference in their entireties. In some embodiments, macromolecule 120 is a single stranded nucleic acid. Then, a connection between the first electrically conducting material and the second electrically conducting material is detected in order to establish a baseline value. In the next step of the method, the macromolecule 120 is contacted with the analyte under conditions that allow the analyte to bind to the macromolecule, thereby forming a macromolecule/analyte complex that comprises bound macromolecule 120 and the analyte. A difference in the connection between the first electrically conducting material and the second electrically conducting material is then detected. In some embodiments, the conditions that allow the analyte to bind to the macromolecule are conditions of high stringency (e.g., conditions disclosed in Section 7.12.4.1), intermediate stringency (e.g., conditions disclosed in Section 7.12.4.2), or low stringency (e.g., conditions disclosed in Section 7.12.4.3).

In some embodiments of the present invention, macromolecule 120 is a double stranded nucleic acid and a first portion of macromolecule 120 is bound to a first electrode (e.g., electrically conducting material 106 or 110) in an electrode pair and a second portion of macromolecule 120 is bound to a second electrode in the electrode pair. The electrode pair is optionally dried and a voltage is optionally applied across the electrode pair in order to measure a current. Next, a solution that potentially comprises a DNA binding protein is exposed to the biosensor for a period of time in order to allow for the DNA binding protein to bind to the macromolecule 120. After a suitable incubation time, the analyte solution is washed away, the electrode pair is dried (e.g., using a gas), and a voltage is applied across the electrode pair in order to measure a current. In this way, the biosensors of the present invention can be used to advantageously detect interactions between DNA binding proteins and nucleic acids.

7.12.3.1 High Stringency

High stringency conditions are known in the art; see for example Maniatis et al., Molecular Cloning: A Laboratory Manual, 2d Edition, 1989, and Short Protocols in Molecular Biology, ed. Ausubel et al., both of which are hereby incorporated by reference in their entireties. High stringency conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30 C for short probes (e.g. 10 to 50 nucleotides) and at least about 60° C. for long probes (e.g. greater than 50 nucleotides). Stringent conditions may also be achieved with the addition of destabilizing agents, such as formamide.

By way of example and not limitation, procedures using conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency that may be used depend on the nature of the nucleic acid (e.g. length, GC content, etc.) and the purpose of the hybridization (detection, amplification, etc.) and are well known in the art. For example, stringent hybridization of an oligonucleotide of approximately 15 to 40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C. The skilled artisan will recognize that the temperature, salt concentration, and chaotrope composition of hybridization and wash solutions may be adjusted as necessary according to factors such as the length and nucleotide base composition of the probe. Another embodiment of the present invention provides a nucleic acid that hybridizes under conditions of moderate stringency to about nucleotide 760 through about nucleotide 1215 of SEQ ID NO: 2. Still another embodiment of the present invention provides a nucleic acid that hybridizes under conditions of moderate stringency to a polynucleotide that is complementary to nucleotides 760 through 1215 of SEQ ID NO: 2. As used herein, conditions of moderate stringency, as known to those having ordinary skill in the art, and as defined by Sambrook et al., Molecular Cloning: A Laboratory Manual, 2nd Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, or Stark's solution, in 50% formamide at 42° C.), and washing conditions of about 60° C., 0.5×SSC, 0.1% SDS. See also, Ausubel et al., eds., in the Current Protocols in Molecular Biology series of laboratory technique manuals, © 1987-1997, Current Protocols, © 1994-1997, John Wiley and Sons, Inc.). The skilled artisan will recognize that the temperature, salt concentration, and chaotrope composition of hybridization and wash solutions may be adjusted as necessary according to factors such as the length and nucleotide base composition of the probe.

7.12.3.2 Intermediate Stringency

As used herein, conditions of moderate stringency, as known to those having ordinary skill in the art, and as defined by Sambrook et al., Molecular Cloning: A Laboratory Manual, 2nd Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, or Stark's solution, in 50% formamide at 42° C.), and washing conditions of about 60° C., 0.5×SSC, 0.1% SDS. See also, Ausubel et al., eds., in the Current Protocols in Molecular Biology series of laboratory technique manuals, © 1987-1997, Current Protocols, © 1994-1997, John Wiley and Sons, Inc.). The skilled artisan will recognize that the temperature, salt concentration, and chaotrope composition of hybridization and wash solutions may be adjusted as necessary according to factors such as the length and nucleotide base composition of the probe.

7.12.3.3 Low Stringency

By way of example and not limitation, procedures using conditions of low stringency for regions of hybridization of over 90 nucleotides are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10 cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

7.12.4 Analyte Detection and Quantification

Once analytes are introduced into the devices 144 of the present invention, the analytes are detected/quantified using standard methods such as amperometry, voltammetry, coulometry. In some embodiments, the measurement results are compared to a standard curve, e.g. a series or measurement results are plotted as a function of analyte concentration. This permits determination of analyte concentration.

In some embodiments of the present invention, an electromagnetic property of a macromolecule 120 bound to an electrode in a device 144 within a biosensor of the present invention is measured using a conductive atomic force microscope (AFM) tip. In such embodiments, the tip of the AFM serves as a second electrode. A measurement is taken across the bound macromolecule 120 using the AFM by measuring an electromagnetic property between the AFM tip and the electrode in the device 144 to which the macromolecule 120 is bound. For more information on the measurement of macromolecules using an AFM see, for example, Gu et al., 2002, Applied Physics Letters 80, p. 688, which is hereby incorporated by reference in its entirety.

7.12.5 Additional Methods for Detecting an Analyte with a Biosensor

One embodiment of the present invention provides a method of detecting an analyte with a biosensor. The biosensor comprises a plurality of devices 144. Any of the devices 144 described herein may be used in this method. For the sake of illustration purposes only, and not by way of limitation, a particular device 144 will be described. In one example, each device 144 in the plurality of devices 144 occupies a different region on an insulator layer 104 and insulator layer 104 is overlaid on a substrate 102. Futhermore, in this example, each device 144 in the plurality of devices 144 comprises:

(i) a first electrically conducting material 106, wherein the first electrically conducting material 106 is overlaid on a first portion of the different region of the insulator layer 104 occupied by the device 144;

(ii) a spacer 140 overlaid on a second portion of the different region of the insulator layer 104 that is occupied by the device 144, wherein the first portion of the different region on the insulator 104 does not overlap the second portion of the different region on the insulator 104; and

(iii) a second electrically conducting material 110, wherein the second electrically conducting material 110 is overlaid on at least a portion of the spacer 144.

In this embodiment, the method comprises (a) attaching a first portion of a macromolecule 120 to the first electrically conducting material 106 and a second portion of the macromolecule to the second electrically conducting material 110 in a device 144 in the plurality of devices. Then, an electromagnetic property is detected between the first electrically conducting material and the second electrically conducting material. As used in the present invention, the term electromagnetic property means a direct electric current, an alternating electric current, a permitivity, a resistivity, an electron transfer, electron tunneling, electron hopping, electron transport, electrical (electron) conductance, a voltage, an electrical impedance, a signal loss, a dissipation factor, a resistance, a capacitance, an inductance, a magnetic field, an electrical potential, a charge, a magnetic potential, and the like. Next, the macromolecule is contacted with an analyte such that the analyte binds to the macromolecule thereby forming a macromolecule/analyte complex that comprises the macromolecule and the analyte. Then, any difference in the electromagnetic property between the first electrically conducting material and the second electrically conducting material is detected.

Another embodiment of the present invention provides a method of detecting an analyte with a biosensor. The biosensor comprises a plurality of devices. Each device in the plurality of devices occupies a different region on an insulator layer. The insulator layer is overlaid on the substrate. Any of the device structures or biosensors may be used in this embodiment of the present invnetion. One such example is provided for the purposes of illustration and not by way of limitation. In this example one or more devices 144 in the plurality of devices 144 comprises a first electrically conducting material 106. The first electrically conducting material 106 is overlaid on a first portion of the different region of the insulator layer 104 occupied by the device 144. A spacer 140 is overlaid on a second portion of the different region of the insulator layer 104 that is occupied by the device 144. The first portion of the different region on the insulator 104 does not overlap the second portion of the different region on the insulator 104. The device 144 further includes a second electrically conducting material 110. The second electrically conducting material 110 is overlaid on at least a portion of the spacer 140.

The method in accordance with this embodiment of the invention comprises attaching a first portion of a macromolecule 120 to the first electrically conducting material 106 in a device 144 in the plurality of devices 144. Then an electromagnetic property is detected between the first electrically conducting material and the second electrically conducting material in the device. The macromolecule 120 is contacted with a sample (e.g., a solution) potentially comprising (e.g., suspected of comprising) the analyte under conditions such that any analyte in the sample can bind to the macromolecule 120 thereby forming a macromolecule/analyte complex that comprises the macromolecule and the analyte. A second portion of any macromolecule/analyte complex so formed is attached to the second electrically conducting material in the device 144. Then any difference in the electromagnetic property is detected between the first electrically conducting material and the second electrically conducting material.

7.13 Cassettes

In certain embodiments, this invention provides a cassette. In some embodiments, a cassette comprises one or more devices 144 or arrays of devices 144. In some embodiments, a cassette comprises a plurality of macromolecules 120, where each macromolecule 120 is attached to a material 106/material 110 pair in a device 144. In such embodiments, material 106 and material 110 serve are electrodes. In some embodiments, counter electrodes are provided.

In one embodiment of the present invention, a cassette or apparatus of the present invention comprises a sample port and/or reservoir and one or more channels for sample delivery into the devices 144 present in the cassette. The means for sample delivery can be stationary or movable and can be any known in the art, including, but not limited to, one or more inlets, holes, pores, channels, pipes, microfluidic guides (e.g., capillaries), tubes. The one or more channels in the cassette can take the form of a channel network. This channel network might include microchannels. Reservoirs in which the desired analysis takes place are typically included within a given channel network. Additionally, the channel network optionally includes channels for delivering reagents, buffers, diluents, sample material and the like to the analysis channels.

The cassettes of the present invention optionally include separation channels or matrices separating/fractionating materials transported down the length of these channels, for analysis. For example, such separation channels or matrices may separate particles within a fluid by size or charge. Suitable separation matrices for use in such channels or matrices include, for example, GeneScan™ polymers (Perkin Elmer-Applied Biosystems Division, Foster City, Calif.). In other embodiments, analysis channels are devoid of any separation matrix, and instead, merely provide a channel within which an interaction, reaction etc., takes place. Examples of microfluidic devices incorporating such analysis channels are described in, for example, PCT Application No. WO 98/00231, and U.S. Pat. No. 5,976,336.

Fluids can be moved through the cassette channel system by a variety of well known methods. Some examples include pumps, pipettes, syringes, gravity flow, capillary action, wicking, electrophoresis, electroosmosis, pressure, and vacuum. The means used for fluid movement may be located on the cassette or on a separate unit.

The test sample can be placed on all of the devices 144 in a cassette. Alternatively, a sample may be placed on particular devices 144 in a cassette. One method for placing a sample on select devices 144 in a cassette is the use of capillary fluid transport means. Alternatively, samples may be placed on the devices 144 by an automatic pipetter for delivery of fluid samples directly to sensor array, or into a reservoir in a cassette or cassette holder for later delivery directly to devices 144 in a cassette.

The cassettes of the present invention can be fabricated from a wide variety of materials including, but not limited to glass, plastic, ceramic, polymeric materials, elastomeric materials, metals, carbon or carbon containing materials, alloys, composite foils, silicon and/or layered materials. Supports may have a wide variety of structural, chemical and/or optical properties. They may be rigid or flexible, flat or deformed, transparent, translucent, partially or fully reflective or opaque and may have composite properties, regions with different properties, and may be a composite of more than one material.

Reagents for conducting assays may be stored on the cassette and/or in a separate container. Reagents can be stored in a dry and/or wet state. In one embodiment, dry reagents in the cassette are rehydrated by the addition of a test sample. In a different embodiment, the reagents are stored in solution in “blister packs” that are burst open due to pressure from a movable roller or piston. The cassettes may contain a waste compartment or sponge for the storage of liquid waste after completion of the assay. In one embodiment, the cassette includes a device for preparation of the biological sample to be tested. Thus, for example, a filter may be included for removing cells from blood. In another example, the cassette may include a device such as a precision capillary for the metering of sample.

A cassette or apparatus of the present invention can, optionally, comprise reference electrodes, e.g., Ag/AgCl or a saturated calomel electrode (SCE) and/or various biasing/counter-electrodes. The cassette can also comprise more one layer of electrodes. Thus, for example, different electrode sets (e.g. arrays of sensor elements) can exist in different lamina of the cassette and thus form a three dimensional array of sensor elements.

7.14 Integrated Assay Device/Apparatus

State-of-the-art chemical analysis systems for use in chemical production, environmental analysis, medical diagnostics and basic laboratory analysis are often capable of complete automation. Such total analysis systems (TAS) automatically perform functions ranging from introduction of sample into the system, transport of the sample through the system, sample preparation, separation, purification and detection, including data acquisition and evaluation. See, for example, Fillipini et al., 1991, J. Biotechnol. 18: 153; Gam et al, 1989, Biotechnol. Bioeng. 34: 423; Tshulena, 1988, Phys. Scr. T23: 293; Edmonds, 1985, Trends Anal. Chem. 4: 220; Stinshoff et al., 1985, Anal. Chem. 57:114R; Guibault, 1983, Anal. Chem Symp. Ser. 17: 637; Widmer, 1983, Trends Anal. Chem. 2: 8.

Recently, sample preparation technologies have been successfully reduced to miniaturized formats. Thus, for example, gas chromatography (Widmer et al., 1984, Int. J. Environ. Anal. Chem. 18: 1), high pressure liquid chromatography (Muller et al., 1991, J. High Resolut. Chromatogr. 14: 174) and capillary electrophoresis (Manz et al., 1992, J. Chromatogr. 593: 253) have been reduced to miniaturized formats. Similarly, in certain embodiments, the present invention provides an integrated assay device (e.g., a TAS) for detecting and/or quantifying one or more analytes using the devices 144, device 144 arrays, or cassettes described above.

Thus, in certain embodiments, the cassettes of this invention are designed so that they insert into an apparatus that contains means for reading one or more devices 144 in the cassette. The apparatus optionally includes means for applying one or more test samples onto the devices 144 of the cassette or into a receiving port or reservoir associated with the cassette. Such an apparatus may be derived from conventional apparatus suitably modified according to the invention to conduct a plurality of assays based on a support or cassette. Such modifications may include the provision for sample and/or cassette handling, multiple sample delivery, multiple electrode reading by a suitable detector, and signal acquisition and processing means.

Some apparatus in accordance with the present invention include instrumentation suitable for performing electrochemical measurements and associated data acquisition and subsequent data analysis. One such apparatus also provides means to hold cassettes, optionally provide reagents and/or buffers and to provide conditions compatible with binding agent/target analyte binding reactions. In addition to such features, one such apparatus also includes an electrode contact means that is able to electrically connect the array of separately addressable electrode connections of the cassette to an electronic-voltage/waveform generator, e.g., a potentiostat. The waveform generator delivers signals sequentially or simultaneously to independently read a plurality of sensor elements in the cassette. In some embodiments, the apparatus optionally comprises a digital computer or microprocessor to control the functions of the various components of the apparatus. In some embodiments, the apparatus also comprises signal-processing means. In one exemplary embodiment, the signal processing means comprises a digital computer for transferring, recording, analyzing and/or displaying the results of each assay.

The sensor element arrays of this invention are particularly well suited for use as detectors in “low sample volume” instrumentation. Such applications include, but are not limited to, genomic applications, such as monitoring gene expression in plants or animals, parallel gene expression studies, high throughput screening, clinical diagnosis, single nucleotide polymorphism (SNP) screening, and genotyping. Some embodiments include miniaturized molecular assay systems (e.g., “labs-on-a-chip”), that are capable of performing thousands of analyses simultaneously.

7.15 Kits

In some embodiments, this invention provides kits for practice of the methods and/or assembly of the inventive devices. Preferred kits comprise a container containing a biosensor of the present invention. In certain embodiments, the kits optionally include one or more reagents and/or buffers for use with the inventive biosensors. In some embodiments, the kits include materials for sample acquisition and data processing.

In some embodiments, the kits include instructional materials containing directions (e.g., protocols) for the practice of the assay methods of this invention. While the instructional materials typically comprise written or printed materials, they are not so limited. Any medium capable of storing such instructions and communicating them to a user is contemplated by this invention. Such media includes, but is not limited to, electronic storage media (e.g., magnetic discs, tapes, cartridges, chips) and optical media (e.g., CD ROM). Such media may include addresses to Internet sites that provide such instructional materials.

In one embodiment, kits of the present invention comprise a biosensor of the present invention. Each such biosensor comprises a plurality of devices. Each device in the plurality of devices has a first and second electrode. Further, a different macromolecule 120 spans the first and second electrode in one or more devices in the plurality of devices. That is, a first portion of a first macromolecule 120 binds to a first electrically conducting material in a first device in the plurality of devices and a second portion of the first macromolecule 120 binds to a second electrically conducting material in the first device. A first portion of a second macromolecule 120 binds to a first electrically conducting material in a second device in the plurality of devices and a second portion of the second macromolecule 120 binds to a second electrically conducting material in the second device, and so forth. In some kits in accordance with the present invention, each cDNA (or other nucleic acid form such as mRNA) molecule in a library of cDNA molecules spans an electrode pair (i.e., first and second electrically conducting materials) in a different device in a plurality of devices in a biosensor of the present invention. In some embodiments, the library of cDNA molecules (or other nucleic acid form such as an mRNA library) comprises more than fifty percent, more than sixty five percent, more than eighty percent, more than ninety percent, or more than ninety-five percent of the genome of a mammal (e.g., mouse, rat, pig, human, cow) or a plant (e.g., corn, wheat).

7.16 Monitoring Electron Transfer Through Bound Macromolecule/Analyte Complexes

In a some embodiments of the present invention, electron transfer through bound macromolecule 120/target analyte complexes is performed using amperometric detection. In some embodiments, the amperometric detector used for such detection resembles the numerous enzyme-based biosensors currently used to monitor blood glucose, for example. This method of detection involves applying a potential (as compared to a separate reference electrode) between materials 106 and 110 in a given device 144 in an inventive biosensor. Electron transfer of differing efficiencies is induced in samples in the presence or absence of analyte. For example, in the case where macromolecule 120 is a single stranded nucleic acid and the analyte is the complement to the single stranded nucleic acid, bound macromolecule 120 exhibits a different current than the corresponding bound macromolecule/analyte complex. The differing efficiencies of electron transfer result in differing currents being generated.

In some embodiments, the amperometric devices used herein use sensitive (nanoamp to picoamp) current detection and include a means of controlling the voltage potential, such as a potentiostat. In other embodiments, alternative electron detection methods are utilized. For example, potentiometric (or voltammetric) measurements involving non-faradaic (no net current flow) processes that are traditionally utilized in pH detectors can be used to monitor electron transfer through bound macromolecule 120/target analyte complexes. In addition, other properties of insulators, such as resistance, and of conductors, such as conductivity, impedance and capacitance, can be used to monitor electron transfer through bound macromolecule 120/target analyte complexes. Finally, any system that generates a current, such as electron transfer, also generates a magnetic field. Therefore, magnetic fields can be monitored in some embodiments of the present invention.

In some embodiments, the relatively fast rates of electron transfer through the binding agent/target analyte complex facilitates analysis of the frequency (time) domain and thereby dramatically improves signal to noise (S/N) ratios. Thus, in certain embodiments, electron transfer is initiated and detected using alternating current (AC) methods. In general, the use of AC techniques can result in good signals and low background noise. Without being bound by any particular theory, it is believed that there are a number of possible contributors to background noise, or “parasitic” signals, i.e. detectable signals that are inherent to the system but are not the result of the presence of the target sequence. However, all of the contributors to parasitic noise generally give relatively fast signals. That is, the rate of electron transfer through the bound macromolecule 120/target analyte complex is generally significantly slower than the rate of electron transfer of the parasitic components, such as the contribution of charge carriers in solution, and other “short circuiting” mechanisms. As a result, the parasitic components are generally all phase related. That is, they exhibit a constant phase delay or phase shift that will scale directly with frequency. The bound macromolecule 120/target analyte complex, in contrast, exhibits a time delay between the input and output signals that is independent of frequency. Thus, signal produced by analyte binding will remain relatively constant and relatively large as compared to parasitic background. As a consequence, at different frequencies, the phase of the system will change. This is very similar to the time domain detection used in fluorescent systems. This difference can be exploited in various methods of the present invention to decrease the signal to noise ratio. Accordingly, the preferred detection methods comprise applying an AC input signal to a bound macromolecule 120/target analyte complex. The presence of the bound macromolecule 120/target analyte complex is detected via an output signal characteristic of electron transfer through the bound macromolecule 120/target analyte complex. That is, the output signal is characteristic of the bound macromolecule 120/target analyte complex rather than the parasitic components or unbound binding agent. Thus, for example, the output signal will exhibit a time delay dependent on the rate of electron transfer through the bound macromolecule 120/target analyte complex.

In some embodiments of the present invention, the input signals are applied at a plurality of frequencies, since this again allows the distinction between true signal and noise. “Plurality” in this context means at least two, and preferably more, frequencies. In general, the AC frequencies will range from 0.1 Hz to 10 mHz or from 1 Hz to 100 KHz. In certain preferred embodiments, data analysis is preformed in the time domain (frequency domain). Thus, for example, cyclic voltammetry is performed where the signal is analyzed at a harmonic of the fundamental frequency. Such measurements can significantly improve the signal to noise (S/N) ratio.

In some embodiments of the present invention, a cyclic (e.g., sinusoidal sweeping voltage) is applied to materials 106 and 110. The response of the bound macromolecule 120/target analyte complex to the sinusoidal voltage is selectively detected at a harmonic of the fundamental frequency of the cyclic voltage rather than at the fundamental frequency. As a result, a complete frequency spectrum can be obtained within one cycle.

### 8.0 Packaged Biosensors

In some embodiments of the present invention, devices 144 are processed in order to form packaged biosensors. Packaging is advantageous because it protects the integrity of the biosensors. Furthermore, packaging is advantageous because it provides a format that is suitable for electronically addressing large numbers of devices 144. Such electronic addressing can be used, for example, to apply a voltage to specific electrodes within predetermined devices 144 in the package and/or to measure current or charge transfer through predetermined devices 144 in the package.

In Section 8.1, techniques for manufacturing a device 144 in accordance with one embodiment of the present invention are described. The techniques disclosed in subsection 8.1 optionally use an etch stop (not shown). Next, in Section 8.2, arrays of devices 144 are disclosed. In Section 8.3, methods for packaging device arrays are disclosed. The techniques described in Section 8.3 can be used to package any of the devices 144 disclosed in the present invention. In Section 8.4, methods and equipment for interfacing a packaged biosensor with data acquisition and signal generation equipment are disclosed. In Section 8.5, exemplary binding event detection methods are disclosed.

8.1 Processing Steps Used to Manufacture an Illustrative Device

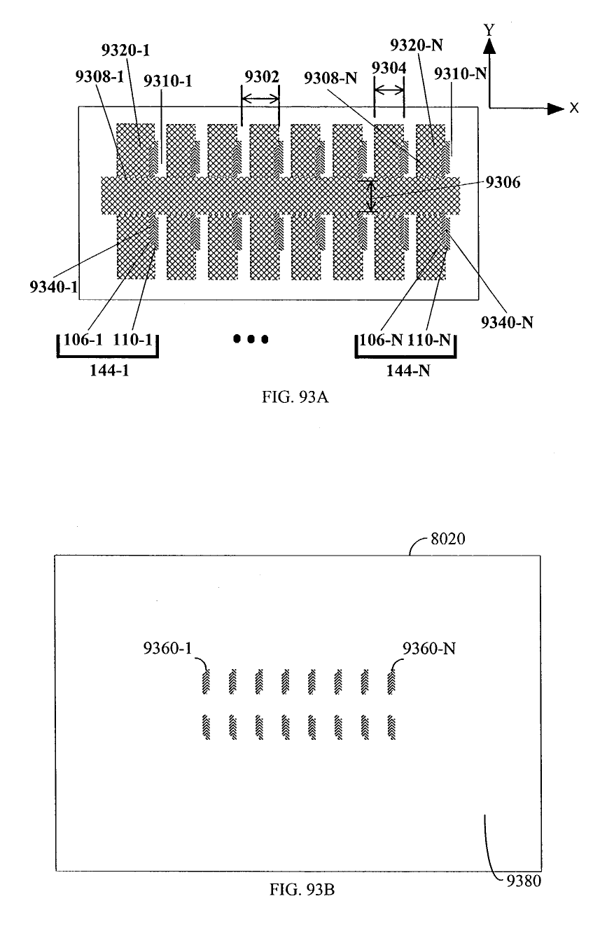

Processing steps in accordance with one embodiment of the present invention will now be described in conjunction with FIGS. 92A through 92F. These figures illustrate the process flow for creating a packaged biosensor. The process begins with the structure illustrated in FIG. 92A. FIG. 92A illustrates a substrate 102. The substrate is made out of, for example, any of the materials described in Section 6.3. Insulator 104 is overlaid on substrate 102. Next, material 110 is overlaid on insulator 104 and passivation layer 130, in turn, is overlaid on material 110. Finally, a sacrificial insulator 9202 is overlaid on passivation layer 130. There are a number of different ways in which the structure illustrated in FIG. 92A can be manufactured. In one embodiment, insulator 104, material 110, passivation layer 130 and sacrificial insulator 9202 are deposited using, for example, any of the deposition techniques described in Sections 7.1.1.1 through 7.1.1.10 or Sections 7.1.7.1 through 7.1.7.12.

In some embodiments of the present invention, insulator layer 104 has a thickness between 10 Angstroms and 10,000 Angstroms. In some embodiments, insulator layer 104 has a thickness between 20 Angstroms and 5,000 Angstroms. In some embodiments, insulator layer 104 has a thickness between 100 Angstroms and 2000 Angstroms. In still other embodiments, insulator layer 104 has a thickness between 400 Angstroms and 800 Angstroms. In one particular embodiment, insulator layer 104 has a thickness between 300 Angstroms and 500 Angstroms. In one embodiment, insulator layer 104 has a thickness between 275 Angstroms and 325 Angstroms. In one particular embodiment, insulator layer 104 has a thickness between 200 Angstroms and 500 Angstroms and is made of SiO₂. In still another particular embodiment, insulator layer 104 has a thickness between 700 Angstroms and 1300 Angstroms. In some embodiments, insulator layer 104 is made out of any of the materials described in Section 6.5, above, such as silicon oxide.

In some embodiments of the present invention, material 110 has a thickness between 50 Angstroms and 1000 Angstroms. In some embodiments, material 110 has a thickness between 80 Angstroms and 350 Angstroms. In some embodiments, material 110 has a thickness between 100 Angstroms and 600 Angstroms. In still other embodiments, material 110 has a thickness between 40 Angstroms and 2000 Angstroms. In one particular embodiment, material 110 has a thickness between 50 Angstroms and 150 Angstroms. In one embodiment, material 110 has a thickness between 95 Angstroms and 105 Angstroms. In one particular embodiment, material 110 has a thickness of 100 Angstroms and is made of platinum or gold. In some embodiments of the present invention, material 110 is made out of any of the materials described in Section 6.4, above.

In some embodiments of the present invention, passivation layer 130 has a thickness that is less than 10 Angstroms. In some embodiments, passivation layer 130 has a thickness between 10 Angstroms and 100 Angstroms. In some embodiments, passivation layer 130 has a thickness between 2 Angstroms and 30 Angstroms. In still other embodiments, passivation layer 130 has a thickness between 4 Angstroms and 15 Angstroms. In one particular embodiment, passivation layer 130 has a thickness between 3 Angstroms and 400 Angstroms. In one particular embodiment, passivation layer 130 has a thickness of between 80 Angstroms and 120 Angstroms. In some embodiments of the present invention, passivation layer 130 is made out of any of the materials described in Section 6.6.

In some embodiments of the present invention, sacrificial insulator 9202 has a thickness between 100 Angstroms and 1500 Angstroms. In some embodiments, sacrificial insulator 9202 has a thickness between 200 Angstroms and 1200 Angstroms. In some embodiments, sacrificial insulator 9202 has a thickness between 300 Angstroms and 1000 Angstroms. In still other embodiments, sacrificial insulator 9202 has a thickness between 400 Angstroms and 800 Angstroms. In one particular embodiment, sacrificial insulator 9202 has a thickness between 400 Angstroms and 600 Angstroms. In one embodiment, sacrificial insulator 9202 has a thickness between 480 Angstroms and 520 Angstroms.

The process continues with the structure illustrated in FIG. 92B, where a cavity 9204 is etched into sacrificial insulator 9202, passivation layer 130, material 110, and insulator 104 until substrate 102 is reached. In some embodiments of the present invention, an etch stop layer overlays substrate 102 (not shown). In such embodiments, the etch stop is used to protect substrate 102 from the etching process used to form cavity 9204. In some embodiments of the present invention, the etch stop is made of silicon nitride and silicon carbide. In some embodiments, the etch stop layer has a thickness that is between 40 Angstroms and 500 Angstroms. In some embodiments, the etch stop layer has a thickness between 50 Angstroms and 400 Angstroms. In one particular embodiment, the etch stop layer has a thickness between 80 Angstroms and 120 Angstroms.

In some embodiments of the present invention, cavity 9204 is formed by a wet etching process disclosed in Section 7.1.5.1. In some embodiments of the present invention, a wet spray etching technique or a vapor etching process described in Section 7.1.5.2, above, forms cavity 9204. In some embodiments of the present invention, cavity 9204 is formed by plasma etching described in Section 7.1.5.3, above. In still other embodiments, cavity 9204 is formed by ion beam etching as described in Section 7.1.5.4. In one embodiment, cavity 9204 is formed by reactive ion etching as described in Section 7.1.5.5.

Referring to FIG. 92B, in some embodiments of the present invention, cavity 9204 has a width 9208 of between 0.09 microns and 2 microns. In some embodiments of the present invention, cavity 9204 has a width 9208 of between 0.13 microns and 0.35 microns. In still other embodiments of the present invention, cavity 9204 has a width 9208 of between 0.35 microns and 0.5 microns. In some embodiments of the present invention, stack 9210 has a width 9206 of between 0.09 microns and 2.0 microns. In some embodiments of the invention, stack 9210 has a width 9206 of between 0.13 microns and 0.35 microns. In still other embodiments of the present invention, stack 9210 has a width 9206 of between 0.35 microns and 0.5 microns.

Referring to FIG. 92C, the process continues with an optional undercut etch step in which crevices 9212-1 and 9212-2 are formed in insulator layer 104. One of skill in the art will appreciate that there are a number of different etching techniques that may be used to form crevices 9212-1 and 9212-2 and all such techniques are included within the scope of the present invention. In some embodiments of the present invention, any of the etching techniques described in Section 7.1.5 are used.

Referring to FIG. 92D, in one embodiment of the present invention, the process continues with an oxide growth step in which a layer 9214 is grown from the underlying substrate 102 using the techniques described in Section 7.1.1.1. In such embodiments, layer 9214 is made of SiO₂ and substrate 102 is made out of silicon. In other embodiments of the present invention, layer 9214 is formed using the semiconductor manufacturing techniques described in Section 7.1. Such embodiments typically include deposition, resist layer deposition, mask alignment and resist layer exposure, followed by resist layer development.

In embodiments that use an etch stop (not shown), layer 9214 is not grown or deposited. Rather, the etch stop layer is used instead of layer 9214.

Referring to FIG. 92E, in one embodiment of the present invention, the process continues with a metal deposition step in which material 106 is deposited. The metal deposition step can be accomplished in any of a variety of ways. For example, in one technique, material 106 is deposited at an angle. That is, the composition used to form material 106 is deposited at the angle 9216 illustrated in FIG. 92E. In one embodiment, angle 9216 is defined as the angle between (i) the plane 9218 formed by the upper surface of substrate 102 and (ii) vector 9220 (FIG. 92E). Vector 9220 is the path that the composition used to form material 106 takes as it is deposited onto the structure illustrated in FIG. 92E. In some embodiments of the present invention, angle 9216 is between zero and 180 degrees. In other embodiments of the present invention, angle 9216 is about ninety degrees. In still other embodiments of the present invention, angle 9216 is between forty-five and ninety degrees.

In some embodiments of the present invention, the distance 9222 from the top of material 106 to the top of material 110 is between 60 Angstroms and 200 Angstroms, less than 500 Angstroms, less than 1000 Angstroms, between 300 Angstroms and 400 Angstroms, between 50 Angstroms and 300 Angstroms, between 100 Angstroms and 250 Angstroms, between 200 Angstroms and 300 Angstroms, less than 300 Angstroms, less than 200 Angstroms, less than 150 Angstroms, less than 100 Angstroms, or between 50 Angstroms and 80 Angstroms. In one embodiment, distance 9222 is in the range of 180 Angstroms and 220 Angstroms.

Referring to FIG. 92F, in one embodiment of the present invention, the process continues with the removal of sacrificial insulator 9202 and the material 106 that is overlaid on the sacrificial insulator 9202. In some embodiments of the present invention, this removal is performed using the resist layer development techniques described in Section 7.1.4. Furthermore, in some embodiments of the present invention, an additional etch step is performed at this stage in order to enlarge cavities 9206 using, for example, any of the etching techniques described in Section 7.1.5, above. In addition, in some embodiments of the present invention, stack 9230 is removed using, for example, any of the etching techniques described in Section 7.1.5, above. In some embodiments, cavity 9204 (FIG. 92B) is dimensioned such that stack 9230 is removed during the formation of cavity 9204, rather than relying on a terminal etching step to remove stack 9230.

Thus, some embodiments of the present invention provide a method of processing a biosensor for binding a macromolecule 120. The method comprises etching a stack. This stack comprises a substrate 102, a first insulator layer 104 overlaid on substrate 102, a first electrically conducting material 110 overlaid on the first insulator layer 104; a passivation layer 130 overlaid on the first electrically conducting material 110 and a sacrificial insulator layer 9202 overlaid on the passivation layer; 130 (FIG. 92A). The etching forms a cavity 9204 that extends through the sacrificial insulator layer 9202, the passivation layer 130, the first electrically conducting material 110, and the first insulator layer 104 (FIG. 92C). Next a second insulator layer 9214 is formed at a bottom of cavity 9204 (FIG. 92D) and a second electrically conducting material 106 is deposited on the second insulator layer (FIG. 92E). Finally, the sacrificial insulator layer 9202 overlaid on passivation layer 130 is removed (FIG. 92F).

Referring again to FIG. 92F, one aspect of the present invention provides a biosensor comprising a plurality of devices 144, one of which is illustrated in FIG. 92F. Thus, it will be appreciated that, while FIG. 92F illustrates a single device 144, in practice, the techniques disclosed in the present invention are typically used to generate multiple devices 144 on a substrate 102. Each of these devices 144 is for binding a macromolecule 120. One biosensor in accordance with this aspect of the invention comprises a substrate 102, a first insulator layer 104 overlaid on substrate 102, a first electrically conducting material 110 overlaid on insulator 104, and a passivation layer 130 overlaid on the first electrically conducting material 110. Furthermore, each device in the plurality of devices in the biosensor comprises a crevice 9204 in extending through the first insulator layer 104, a second insulator layer 9208 in the crevice 9204, and a second electrically conducting material 106 on the second insulator layer 9208. In some embodiments, first insulator layer 104 has a thickness of between 10 Angstroms and 1500 Angstroms. In some embodiments, first insulator layer 104 has a thickness of between 250 Angstroms and 350 Angstroms. In yet other embodiments, first insulator layer 104 has a thickness of about 300 Angstroms and comprise silicon oxide. In still other embodiments, the first insulator layer 104 has a thickness between 700 Angstroms and 1300 Angstroms.

In one aspect of the present invention, a plurality of crevices 9204 are formed using the techniques disclosed in U.S. Pat. No. 5,252,294 to Kroy et al, which is hereby incorporated by reference in its entirety. In such embodiments, crevices 9204 are formed directly in substrate 102 rather than in insulator 104. In one embodiment, substrate 102 is (100) silicon, which has laterally limiting (111) planes that make an angle of 54.7 degrees with respect to the wafer (substrate 102) surface. In such embodiments, insulator layer 9208 is deposited or grown at the bottom of crevices 9204 that are formed directly in substrate 102 using the techniques described above. Next, insulator 106 is deposited on insulator layer 9208 using the techniques described above. This yields a structure similar to that disclosed in 92F. The only exception is that such devices do not require an insulator layer 104. It will be appreciated, however, that an insulator layer 104 can be used in the biosensor made in accordance with this aspect of invention. For example, insulator layer 104 can be deposited after anisotropic etching.

In still another embodiment of the present invention, crevices 9204 are formed in an substrate 102 using techniques including, but not limited to, stamping techniques, molding techniques, and microetching techniques. In some embodiments, crevices 9204 are formed in substrate 102 using the techniques disclosed in U.S. Pat. No. 6,429,029 to Chee et al., which is hereby incorporated by reference in its entirety.

8.2 Device Arrays

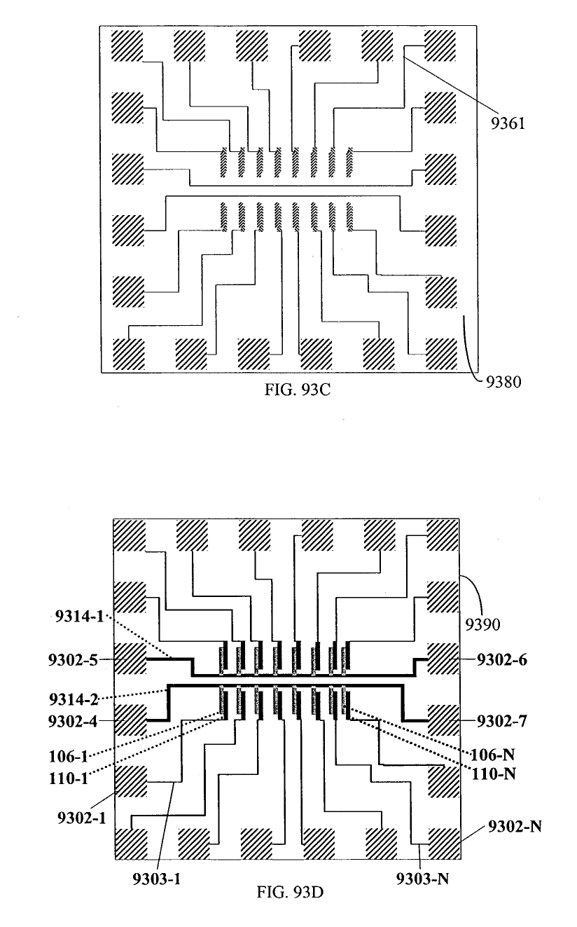

In some embodiments of the present invention, N devices 144 are arrayed on a substrate 102 that includes a plurality of upper steps 9310 and a plurality of lower steps 9308 (FIG. 93A). Each upper step 9310 in the plurality of upper steps is associated with a lower step 9308 in the plurality of lower steps. In various embodiments of the present invention, each upper step 9310 and associated lower step 9308 is separated in the Z-dimension (vertical dimension, i.e., perpendicular to the X-Y plane drawn in FIG. 93A) by 5 Angstroms to 100 Angstroms, 20 Angstroms to 80 Angstroms, 30 Angstroms to 60 Angstroms, more than 40 Angstroms, more than 50 Angstroms, more than 75 Angstroms, more than 80 Angstroms, more than 100 Angstroms, more than 125 Angstroms, more than 150 Angstroms, more than 200 Angstroms, or less than 100 Angstroms. In practice, the number N of devices 144 arrayed on the substrate illustrated in FIG. 93A is any number. In some embodiments, N is 1, 2, 10, at least 100, 1000 to 10,000, 10,000 to 10⁵, 10⁵ to 10⁷, 10⁷ to 10⁹, 10⁹ to 10¹⁰, 10¹¹ to 10¹², or more.

In some embodiments of the present invention, material 110 of each device 144 is overlaid or integrated into upper step 9310 of substrate 102 and material 106 of each device 144 is overlaid or integrated into lower step 9308 of substrate 102 as illustrated in FIG. 93A. Stepped substrate 102 can be manufactured using standard semiconductor processing techniques such as those disclosed Section 7.1. For example, substrate 102 of FIG. 93. A can be manufactured using a patterned resist layer and etching.

In some embodiments, substrate 102 illustrated (FIG. 93A) is patterned so that lower step 9308 includes elements 9320. Each element 9320 is bordered by portions of upper step 9310 as illustrated in FIG. 93A. In some embodiments of the present invention, each element 9320 has a width 9304 of between two and forty microns. In still other embodiments of the present invention, each element 9320 has a width 9304 of between four and thirty microns. In yet other embodiments of the present invention, each element 9320 has a width 9304 of between eight and twenty microns. In one embodiment of the present invention, each element 9320 has a width of about fifteen microns.

In some embodiments, the spacing 9302 between materials 110 of neighboring devices 144 overlaid on the substrate 102, as illustrated in FIG. 93A, is between 3 and 100 microns, between 5 and 80 microns, between 8 and 70 microns, between 10 and 50 microns, or between 15 and 40 microns. In some embodiments, the spacing 9302 between the material 110 of each neighboring device 144 overlaid on the substrate 102, as illustrated in FIG. 93A, is between 15 and 25 microns. In one embodiment, the spacing 9302 between the material 110 of each neighboring device 144 overlaid on the substrate 102 as illustrated in FIG. 93A is about twenty microns.

In some embodiments, the spacing 9306 between the material 106 of each neighboring device 144 overlaid on the substrate 102 as illustrated in FIG. 93A is between 3 and 80 microns, between 5 and 70 microns, between 7 and 60 microns, between 9 and 45 microns, or between 10 and 20 microns. In some embodiments, the spacing 9306 between materials 106 of each neighboring device 144 overlaid on the substrate 102 as illustrated in FIG. 93A is about fifteen microns.

Referring again to FIG. 93A, one aspect of the present invention provides a biosensor comprising a plurality of devices 144 on a substrate 102. Each device 144 in the plurality of devices 144 is for binding a macromolecule 120. In this aspect of the invention, substrate 102 comprises a plurality of upper steps 9310 and a plurality of lower steps 9308. Each upper step 9310 in the plurality of upper steps 9310 is associated with a lower step 9308 in the plurality of lower steps. An upper step 9310 is associated with a lower step 9308 when the two steps are adjacent to each other and a first electrically conducting material 110 overlays upper step 9310 and a corresponding second electrically conducting material 106 overlays the associated lower step 9308. A first electrically conducting material 106 and a second electrically conducting material 110 correspond to each other when they are within the same device 144. Thus, for each device 144 in the plurality of devices in the biosensor, a first electrically conducting material 110 in device 144 overlays an upper step 9310 in the plurality of upper steps and a second electrically conducting material 106 in device 144 overlays the lower step 9308, in the plurality of lower steps, that is associated with the upper step 9310.

8.3 Processing Steps Used to Package Devices

Referring to FIG. 93A, each device 144 in an array of devices has at least two electrodes, including an upper electrode 106 and a lower electrode 110. In some embodiments of the present invention, upper electrode 106 and lower electrode 110 of each device 144 in the array of devices is connected to external circuitry. The external circuitry functions to, among other things, complete a closed path through which current can flow when an electrically conductive object (e.g. macromolecule 120) spans a gap 9340 of a given device 144 by binding to both electrode 106 and electrode 110 of the given device 144.

In one embodiment of the present invention, metallization is used to electrically connect electrodes 106 and 110 with external circuitry. Metallization is a well-known process in the art of integrated circuit (IC) manufacturing. Metallization has been described previously in Sections 7.1.7 through 7.1.12 in conjunction with FIG. 80. Specifically, FIG. 801 illustrates one step in the manufacture of a device or array of devices such as that illustrated in FIG. 93A. In FIG. 80I, mask 8020 is used to selectively illuminate photoresist layer 8012. In Section 7.1.9, the use of a single mask 8020 was described.

The process described in this section expands upon the techniques disclosed or referenced in Section 7.1.9 in order to pattern devices 140 in such a way that they are amendable to packaging. In one embodiment, multiple masks are used to pattern resist layer 8012, beginning with a mask 8020. FIG. 93B depicts one possible configuration of such a mask 8020 in accordance with this embodiment of the invention. In FIG. 93B, region 9380 of mask 8020 is opaque, and therefore blocks all light incident upon it. The plurality of hole regions 9360, on the other hand, allow light to pass. Hole regions 9360 of mask 8020 (FIG. 93B) define the portions of metal layer 8010 (FIG. 801) that will form electrodes (materials) 106 and 110. After illumination of the wafer with light through mask 8020, mask 8022, depicted in FIG. 93C, is similarly interposed between the light source and photoresist layer 8012. A second illumination of photoresist layer 8012 is then performed. As shown in FIG. 93C, mask 9360 has a plurality of narrow gaps 9361 that allow light to pass. After the illumination of the wafer through mask 9360 and subsequent etching step and resist removal step as described in Sections 7.1.11, and 7.1.12, the pattern of metallization depicted in FIG. 93D results. In FIG. 93D, bonding pads 9302, interconnects 9303, and electrodes 110 and 106 have all been fabricated from the original metal layer 8010 (FIG. 801) using masks. As shown in FIG. 93D, lower electrode 110-1, for example, is electrically coupled by interconnect 9303-1 to bonding pad 9302-1. Furthermore, upper electrodes 106 are electrically coupled by interconnect 9314 to one another and to bonding pads 9302-4, 9302-5, 9302-6, and 9302-8 in the manner illustrated in FIG. 93D.

Region 9390 (FIG. 93D) is referred to as a die. The steps in the metallization process disclosed above are one of a number of methods of achieving the metallization pattern depicted in FIG. 93D. It will be appreciated that a large number of die (regions 9390) may be arranged on the same substrate 102. Thus, in alternative methods of manufacture, in accordance with the present invention, many identical copies of die 9390 are manufactured simultaneously on a common substrate 102 (e.g. a wafer), as illustrated in FIG. 93E. In some embodiments of the present invention, there is ten or more die on a substrate (wafer) 102. In some embodiments of the present invention, there are 100 or more die on a substrate 102. In still other embodiment of the present is 1000 or more die on a substrate 102, 10000 or more die on a substrate 102, 100,000 or more die on a substrate 102, or 1,000,000 or more die on a substrate 102. Furthermore, each die (region 9390) may have any number of devices 144 connected to bonding pads 9302 using interconnect 9303. In some embodiments, there are 10 or more devices 144, 100 or more devices 144, 1000 or more devices 144, 10000 or more devices 144, 100,000 or more devices 144, or 1,000,000 or more device 144 in a given die. In some embodiments, there are between 100 and 1000 devices 144 on a substrate 102, between 1000 and 10,000 devices 144 on a substrate 102, or between 10,000 devices 144 and 100,000 devices 144 on a substrate.

In some embodiments of the present invention, each device 144 may not be directly connected to a bonding pad 9302 through an interconnect 9303. Rather, conventional circuit elements may be employed to selectively connect one device 144 to a given bonding pad 9302 via an address signal supplied via another one or more of bonding pads 9302. One circuit element that can be used for to accomplish this wiring scheme is a demultiplexer. A demultiplexer, having a complementary metal-oxide semiconductor (CMOS) architecture, can easily be incorporated into the biosensors of the present invention using techniques such as the fabrication steps described above in, for example, Sections 7.1.7 through 7.1.12. See, also, Horowitz and Hill, The Art of Electronics, 2nd edition, Cambridge University Press, 1989 at pp. 143-144. As will described in more detail below, each bonding pad 9302 is wired to a corresponding pin in a chip package. However, the number of pins in a package is limited. Therefore, the use of circuit elements, such as demultiplexers, is advantageous because it allows for chip configurations in which the number of devices 144 in the chip is much larger than the number of pins in the chip.

After patterning and metallization, it is desired to place a particular die 9390 into a package (a process referred to as packaging) to protect interconnects 9303 on the die from liquids used to expose devices 144 to macromolecules 120 and/or analytes used to bind to macromolecules 120. Furthermore, the packaging facilitates connection of the die with a printed circuit (PC) board (not shown). A printed circuit (PC) board is a rigid, insulating sheet of material with thin plated cooper lines forming circuit paths used to facilitate connection of devices 144 on die 9390 to external circuitry. As such, a printed circuit board contains the logic used to electronically address each device 144 in die 9390. In alternative embodiments in accordance with the present invention, die 9390 is part of a multi-chip module (MCM) or is integrated directly with the external circuitry.

The first step in a packaging process in accordance with one embodiment of the present invention is to separate a selected die 9390 (FIG. 93E) from wafer (substrate) 102. One possible method of achieving this is to use sawing. In sawing, a diamond-impregnated saw is first passed over first-oriented scribe lines (e.g., FIG. 93E, 9391-1) on the wafer and then second-oriented scribe lines (e.g., FIG. 93, 9392-1) on the wafer. In one version of the sawing method, the saw cuts completely through the thickness of the wafer 102, separating a die 9390. Alternatively, the saw is used to make trenches of some depth in the wafer 102 (substrate), collocated with the scribe lines. Subsequently, the wafer is mechanically stressed by moving a roller over the surface, separating die 9390 from wafer 102.

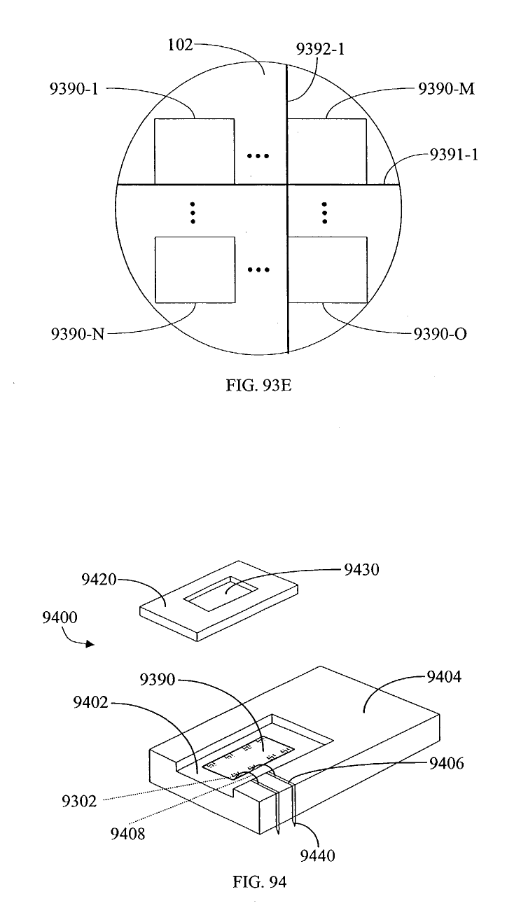

After a die 9390 has been separated from wafer 102, it is placed in a package. As depicted in FIG. 94, in one embodiment die 9390 is placed on die-attach surface 9402 of package body 9404. To create a strong mechanical coupling between the die 9390 and package body 9404, a die-attachment bond is formed between the underside of the die 9402 and the die-attach surface 9402. In one possible embodiment, a thick liquid epoxy adhesive such as silver filled epoxy is used to achieve the desired bond. In this embodiment, before die 9390 is placed on die-attach surface 9402, the epoxy is deposited on the die-attach surface 9402. Then, die 9390 is placed onto the die-attach surface 9402, forcing the epoxy to form a thin layer of uniform thickness. Finally, entire assembly 9400 is placed into a curing oven and heated. The elevated temperature induced by the curing oven causes the epoxy to create a permanent bond between die 9390 and die-attach surface 9402.

In another embodiment, die 9390 is attached to die-attach surface 9402 by eutectic die attachment. The eutetic method is named for the phenomenon that takes place when two materials melt together (alloy) at a much lower temperature than either of them separately. For die attach, two eutectic materials are gold and silicon. On one eutetic method, gold is plated on die attach surface 9402. Then, when die 9390 is pressed onto surface 9402, the gold alloys with the silicon substrate upon heating. See, for example, Van Zant, Microchip Fabrication: A Practical Guide to Semiconductor Processing, 4th edition, McGraw-Hill, 2000 at p. 570.

After die attachment, the next step in the packaging process is to attach each die bonding pad 9302 (see also FIG. 93D) to each package lead via 9406 with bond wires 9408. In one embodiment of the present invention, this step is performed using wire bonding. Alternatively, flip-chip or beam-lead techniques can be used. See, for example, Streetman, Solid State Electronic Devices, 4th Edition, Prentice Hall (NJ), 1995 at p. 371. In wire bonding, bond wire 9408 is a thin (0.7-1.0 mil) wire, sometimes composed of gold (Au) or aluminum (Al). The process of wire bonding begins by placing bond wire 9408 into capillary device. The capillary device is then positioned such that the end of bond wire 9408 is positioned directly over die bonding pad 9302. A combination of downward mechanical pressure applied by the capillary device and heat (as in thermocompression bonding) or ultrasonic energy (as in thermosonic bonding) then causes the end of bonding wire 9408 to form a strong metallic bond between bond wire 9408 and bonding pad 9302. Next, the capillary device is positioned such that a portion of bond wire 9408 is positioned directly over package lead 9406. Again, a combination of pressure and heat or ultrasonication serves to form a metallic bond between bond wire 9408 and package lead 9406. See, for example, Streetman, Solid State Electronic Devices, 4th Edition, Prentice Hall (NJ), 1995 at pp. 368-370. Subsequently, the process is repeated with a new bonding wire 9408, which is bonded to a different contact pad 9302 and package lead 9406. This process is repeated until all of the contact pads 9302 of the die 9390 that are desired to be connected to external circuitry are wire bonded by a separate bond wire 9408 to a separate package lead 9406.

FIG. 94 illustrates a dual in-line package (DIP), so-named for the two linear series of pins 9440 (only one linear series is shown). In other embodiments of the present invention, other package types are used. Such package types include, but are not limited to, single in-line package (SIP) or ball grid array (BGA) packages. See also Van Zant, Microchip Fabrication: A Practical Guide to Semiconductor Processing, 4th edition, McGraw-Hill, 2000, at p. 570.

In a DIP package, die 9390 is enclosed in a ceramic or plastic case for mechanical, thermal, and electrical protection of the die. In one embodiment of the present invention, the last step in the packaging process is to fully enclose die 9390 by placing upper piece 9420 onto the die attach surface of package body 9404. In one embodiment, package body 9404 and upper piece 9420 are composed of a ceramic. Epoxy is deposited on the die-attach surface 9402. Then, upper piece 9420 is placed onto die-attach surface 9402, forcing the epoxy to form a thin layer of uniform thickness. Finally, the entire assembly 9400 is placed into a curing oven and heated. The elevated temperature induced by the oven causes the epoxy to create a permanent bond between upper piece 9420 and package body 9404. In some embodiments of the present invention, upper piece 9420 has access hole 9430 so that, after sealing to package body 9404, it is still possible to access the active area of die 9390. The active area of die 9390 is that portion of die 9390 that has one or more devices 144. Access hole 9430 is used, for example, to expose devices 144 to macromolecules 120, analytes, or rinse solutions.

Referring to FIG. 93, one aspect of the present invention provides a method of manufacturing a biosensor. The method comprises depositing an electrically conducting layer onto a substrate 102. The substrate 102 comprising a plurality of upper steps 9310 and a plurality of lower steps 9308. Each upper step 9310 in the plurality of upper steps is associated with a lower step in the plurality of lower steps. As define herein, an upper step 9310 is associated with a lower step 9308 when the two steps support the same device 144. That is, an upper step 9310 is associated with a lower step 9308 when a device 144 overlays the associated upper step 9310 and the lower step 9308. Therefore, associated upper steps 9310 and lower steps 9308 are adjacent to each other. For example, in FIG. 93A, lower step 9308-1 is associated with upper step 9310-1 because a device 144 overlays the two steps. Furthermore, lower step 9308-1 and upper step 9310-1 are adjacent to each other.

The method continues with the patterning of the electrically conducting layer to form a plurality of electrode pairs (e.g., electrodes 106-1 and 110-1 of FIG. 93D) a plurality of bonding pads 9302 and a plurality of interconnects 9303. In this patterning, an interconnect 9303 in the plurality of interconnects joins an electrode (e.g. 106 or 110) in the plurality of electrode pairs to a bonding pad 9302 in the plurality of bonding pads. Each electrode pair comprises a first electrode (e.g. electrode 110) and a second electrode (e.g., electrode 106) wherein first electrode is on an upper step 9310 in the plurality of upper steps and the second electrode 106 is on the lower step 9320 in the plurality of lower steps that is associated with the upper step 9310.

The method continues with sealing the substrate 102 to a die attach surface 9402 (FIG. 94) 9402 of a package body 9404. This package body 9404 includes a plurality of leads 9406. Then, a bonding pad 9302 in the plurality of bonding pads is attached to a lead 9406 in the plurality of leads. In some embodiments, the step of attaching a bonding pad 9302 to a lead 9406 is repeated a number of times. Once this process is completed, package body 9404 is enclosed with an upper piece 9420 thereby forming the packaged biosensor.

8.4 Processing Steps Used to Manufacture an Illustrative Packaged Device

One aspect of the present invention provides methods for interfacing a biosensor with data acquisition and signal generation equipment. Such an interface allows for automated measurement of a current through a device 144 in the biosensor when a voltage is applied across device 140. Other electrical properties, including but not limited to capacitance, inductance, and resistance, of a device 144 may also be determined. The voltage applied to a device 144 may be a direct current (DC) voltage, an alternating current (AC) voltage of a given frequency, or an arbitrary waveform, such as sawtooth. This aspect of the present invention allows for the automated measurement of the current response of each individual device 144 to application of such a voltage.

In one embodiment in accordance with this aspect of the invention, the automated system incorporates a computer having a microprocessor. This computer can have any of a wide range of architectures such as, for example the personal computer (PC) architecture. Software stored on computer is used to automatically measure the properties of devices 144 that are of interest and to store the results for either subsequent or immediate interpretation.

To facilitate connection of the devices 144 in a packaged biosensor with a computer, package 9404 is attached to a printed circuit board. A printed circuit board is a piece of rigid insulating material with holes for the insertion of package pins 9440 and thin plated copper lines for forming the circuit paths between the pins 9440 and devices external to the printed circuit board. In one embodiment, each of the package pins 9440 is connected by a copper line on the printed circuit board to a corresponding lead of an edge connector. The edge connector itself can mate directly with other connectors in a variety of industry standard ways. Other methods may also be used to connect the packaged biosensor to devices external to the board. See, for example, Horowitz and Hill, The Art of Electronics, 2nd edition, Cambridge University Press, 1989 at pp. 837-838.

The printed circuit boards may then be electrically interfaced to the computer. Any one of a number of commercially available data acquisition cards (DAC) can be used for this purpose. For example, part ADAC/5503HR (IOtech, Inc. Cleveland, Ohio) provides a number of user-programmable analog output channels. These output channels could be used to apply a voltage across the electrodes in a device 144. To measure the current that results, a digital multimeter such as the 34401A Digital Multimeter (Agilent Technologies, Palo Alto, Calif.) can be connected in series in the circuit loop formed by the output channel of the DAC card and the device 144. The 34401 A digital multimeter can be interfaced to the computer using, for example, a standard RS232 serial interface. The DAC ADAC/5503HR can be interfaced to the PC using, for example, a PCI (Peripheral Component Interconnect) bus card slot in the computer In addition to the hardware described above that is used for coupling the computer to the device 144, it is advantageous to provide software instruction to the computer as to how to automatically apply voltages across specific device pair 144 and measure the result. In one embodiment, LabView© (National Instruments Corporation, Austin, Tex.) is used to provide a high-level computer programming language that facilitates the development of computer software for this purpose. The software can be adapted to instruct the DAC to apply a voltage to a specific device 144 in the packaged biosensor by applying a voltage to the corresponding channel of the output of the DAC. Then, the software can request that the current measured by the digital multimeter be acquired and stored in the memory or hard drive of the computer. Furthermore, IntuiLink© software (Agilent Technologies, Palo Alto, Calif.) can be used to facilitate communication between the multimeter and the computer. One of skill in the art will recognize that any one of a number of high-level computer programming languages can be used for this purpose (C, C++, Perl, Fortran, Visual Basic, etc.). The process of applying a voltage to specific device 144 in the package biosensor and measuring the resulting current can then be repeated for each desired device 144 in package 9404 in any manner desired.

8.5 Measuring Analyte Binding Events

The biosensors of the present invention may be used to detect macromolecule 120/analyte binding events. Such binding events may arise through, for example, ligand/receptor, enzyme/substrate, DNA/DNA, DNA/RNA, RNA/RNA, nucleic acid/protein interactions.

In one embodiment of the present invention, macromolecule 120 is a single stranded DNA bound two an electrode pair (materials 106 and 110) in a given device 144 in the biosensor and the analyte is a single stranded DNA. In this embodiment, an alternating current (AC) conductance test is used to determine whether a binding event has occurred in the device 144. This is done by measuring the AC conductance G_{AC}=ε″A/d at the device 144, where A is the effective area of one electrode and d is the effective distance between electrodes. At the relaxation frequency of a given double stranded DNA molecule (e.g., macromolecule 120 bound to an analyte to form a double stranded nucleic acid) should be different (e.g., larger) than the conductance when no analyte bound to the macromolecule 120 bound in the device 144. A pulsed or frequency-scanned waveform is applied across the electrode pair in the device 144. The presence of hybridized DNA is detected at a resonant frequency of DNA. An LCR meter may be used to measure G or R=1/G at a discrete frequency. Alternatively, G can be measured as a function of frequency.

In another embodiment, a frequency scanned or chirped voltage waveform Vᵢ is applied across the electrodes at each site and the resultant response waveform Vₒ, depending upon whether frequency is increasing or decreasing, is analyzed to determine the presence of hybridized DNA as indicated by a maxima at a hybridized DNA frequency. The measurement of the relaxation frequency of the hybridized DNA using a frequency-scanned waveform gives additional information about the properties of the hybridized DNA, e.g., crosslinked versus non-crosslinked.

### 9.0 Device Density

The present invention is advantageous because it provides for device 144 density that is more than sufficient to allow for the representation of each gene in the genome on a single die (chip). The maximum density for devices 144 in the biosensors of the present invention may be calculated using FIG. 5B and FIG. 78 as a guide. In FIG. 5B, the width 40 and length (not shown) of electrically conducting material 106 and the width 20 and length (not shown) of electrically conducting material 110 is presently limited by the current technology node of photolithography, which is 0.09 microns. Accordingly, in some embodiments of the present invention, materials 106 and 110 have a width and/or length of 0.09 microns, 0.11 microns, 0.13, microns, 0.15 microns, between 0.09 microns and 0.5 microns, or more than 0.5 microns. It will be appreciated that, as the technology limit (technology node) for photolithography improves over time, embodiments of the present invention in which the widths and lengths of materials 106 and 110 are respectively less than 0.09 microns will be possible.

Referring to FIG. 5B, another component that determines the total width 60 of one device in accordance with the present invention is the separation distance 30 between material 106 and material 110. In some embodiments of the present invention, the separation distance 30 between material 106 and material 110 is determined by the limits of photolithography. In such embodiments, distance 30 has a width of 0.09 microns, 0.11 microns, 0.13, microns, 0.15 microns, between 0.09 microns and 0.5 microns, or more than 0.5 microns. In some embodiments of the present invention, the separation distance 30 between material 110 and 110 is not determined by the limits of photolithography. For example, a thin sacrificial etch layer may be used to form the gap between materials 106 and 110. In such embodiments, therefore, it is possible for distance 30 to be between 60 Angstroms and 200 Angstroms, between 200 Angstroms and 700 Angstroms, or greater than 700 Angstroms.

Based on the ranges for lengths 20, 30, and 40 (FIG. 5B) provided above, in some embodiments of the present invention, length 60 is 90 nm plus 6 nm plus 90 nm, or 186 nm. In some embodiments of the present invention, length 60 is between, between 186 nm and 266 nm, between 266 nm and 300 nm, between 300 nm and 500 nm, between 500 nm and 1000 nm, between 1000 nm and 10000 nm, or more than 10000 nm.

Referring to FIG. 78, the area of a given device 144 in a biosensor of the present invention is the product of distance 60 and distance 80. Distance 80 is simply the length of material 106 when materials 106 and material 110 have the same length. When materials 106 and 110 do not have the same length, then distance 80 is the longer of the length of material 106 and material 110. As described in conjunction with FIG. 5B above, the length of material 106 and material 110 is determined by the limitations of photolithography. Accordingly, in some embodiments of the present invention distance 80 is 0.09 microns (90 nm).

Given a minimum length 60 (186 nm) and a minimum width 80 (90 nm) (FIG. 78) based on present day limits of photolithography, the minimum square area of a device 144 in accordance with the embodiment illustrated in FIG. 5B is 186 nm×90 nm, or 16.74 microns.

To compute device 144 density, two other dimensions beside dimensions 80 and 60 need to be considered. They are the separation of devices 144 in the X dimension 90 and the Y dimension 95 as illustrated in FIG. 78. In some embodiments of the present invention, dimensions 90 and 95 are determined by the limits of photolithography. Accordingly, in some embodiments of the present invention, dimension 90 and dimension 95 each have a width that is 0.09 microns, between 0.09 microns and 0.11 microns, between 0.11 microns and 0.13, microns, between 0.13 microns and 0.15 microns, between 0.09 microns and 0.5 microns, or more than 0.5 microns.

In some embodiments of the present invention, dimensions 90 and 95 are not determined by the limits of photolithography. For example, a thin sacrificial resist layer can be used to achieve dimensions 90 and 95 (FIG. 78) that are in the range of 60 Angstroms and 200 Angstroms.

In embodiments where dimensions 90 and 95 are determined by the limits of photolithography, the pitch of each device 144 along the x-axis is dimension 60 plus dimension 90 and the picth of each device 144 along the y-axis is dimension 80 plus dimension 95. Given the present photolithography technology node of 0.09 microns, the pitch along the x-axis is (186 nm plus 90 nm)=276 nm and the pitch along the y-axis is (90 nm plus 90 nm)=180 nm in such embodiments. The maximum number of columns of devices 144 per ten microns on the x-dimension would be 10 microns/0.276 microns or about 36. The maximum number of columns of device 144 per ten microns on the y-dimension would be 10 microns/0.180 microns or about 55. Thus, 1980 (i.e., 36×55) devices could be packed into a 100 micron square of substrate in such embodiments.

In embodiments where dimensions 90 and 95 are not determined by the limits of photolithography, the pitch along the x-axis is (186 nm+6 nm) and the pitch along the y-axis is (90 nm+6 nm). In such embodiments, the maximum number of columns of devices 144 per ten microns on the x-dimension would be 10 microns/0.192 microns or about 52. The maximum number of columns of device 144 per ten microns on the y-dimension would be 10 microns/0.096 microns or about 104. Thus, 5408 (i.e., 52×10⁴) devices could be packed into a 100 micron square of substrate in such embodiments.

Advantageously, in some embodiments of the present invention, even higher device 144 densities are achieved. For example, consider the device 144 illustrated in FIG. 1. In the device 144 illustrated in FIG. 1, there is no separation distance 30 (FIG. 5B) between material 106 and material 110 because the two materials are separated in the Z dimension. Accordingly, arrays of devices 144 that have no gap between materials 106 and 110 can have dimensions 60 and 80 (FIG. 78) that are respectively 180 nm and 90 nm, given the present day photolithography limit of 0.09 microns (90 nm). Given the present photolithography technology node of 0.09 microns, the pitch along the x-axis is (180 μm plus 90 nm)=270 nm and the pitch along the y-axis is (90 nm plus 90 nm)=180 μm in such embodiments. The maximum number of columns of devices 144 per ten microns on the x-dimension would be 10 microns/0.270 microns or about 37. The maximum number of columns of device 144 per ten microns on the y-dimension would be 10 microns/0.180 microns or about 55. Thus, 2035 (i.e., 37×55) devices could be packed into a 100 micron square of substrate in such embodiments. In embodiments where dimensions 90 and 95 are not determined by the limits of photolithography, the pitch along the x-axis is (180 nm+6 nm) and the pitch along the y-axis is (90 nm+6 nm). In such embodiments, the maximum number of columns of devices 144 per ten microns on the x-dimension would be 10 microns/0.186 microns or about 53. The maximum number of columns of device 144 per ten microns on the y-dimension would be 10 microns/0.096 microns or about 104. Thus, 5512 (i.e., 53×10⁴) devices could be packed into a 100 micron square of substrate in such embodiments.

In some biosensors of the present invention, there are between 1 and 100 devices 144, between 100 and 500 devices 144, between 500 and 1000 devices 144, between 1000 and 2000 devices 144, between 2000 and 3000 devices 144, between 3000 and 4000 devices 144, between 4000 and 5000 devices 144, or between 5000 and 6000 devices 144 on a 100 micron square of substrate 102 and/or insulator 104 surface. One embodiment of the present invention provides a biosensor comprising a substrate and a plurality of devices 144 overlaid on substrate 102. Each device in the plurality of devices 144 comprises an electrode pair. Each electrode pair comprises a first electrically conducting material and a second electrically conducting material (e.g., materials 106 and 110). Each respective first electrically conducting material and second electrically conducting material in each electrode pair is separated by a distance that is between 60 Angstroms and 500 Angstroms. Further, at least one device 144 in the plurality of devices occupies 1/100 or less of a 100 micron square of surface area on the substrate. In some embodiments, at least one device 144 in the plurality of devices occupies 1/1000 or less of a 100 micron square of surface area on the substrate. In some embodiments, an insulator layer overlays the substrate and each device 144 in the plurality of devices 144 overlays the insulator layer. In some embodiments of the present invention, a first portion of a macromolecule 120 is bound to a first electrically conducting material in a device 144 in the plurality of devices and a second portion of a macromolecule 120 is bound to a second electrically material in the device 144.

In some embodiments of the present invention, the size of the substrate 102 used in a biosensor in accordance with the present invention is between 1 mm² and 10 mm², between 10 mm² and 20 mm², between 20 mm² and 50 mm², between 50 mm² and 100 mm², or between 1 mm² and 100 mm². In some embodiments, the size of the substrate 102 used in a biosensor of the present invention is greater than 100 mm². In some embodiments the length and width of substrate 102 is the same while in other embodiments, the length and width of substrate 102 is different.

### 10.0 Array Of Arrays

In some embodiments of the present invention, an array of arrays is provided. For example, some embodiments of the present invention provide a plurality of the arrays illustrated in FIG. 78. In some embodiments, the array of arrays is dimensioned and configured for use in a conventional microwell plate, such as a 96 well, 384 well, or 1584 microwell plate.

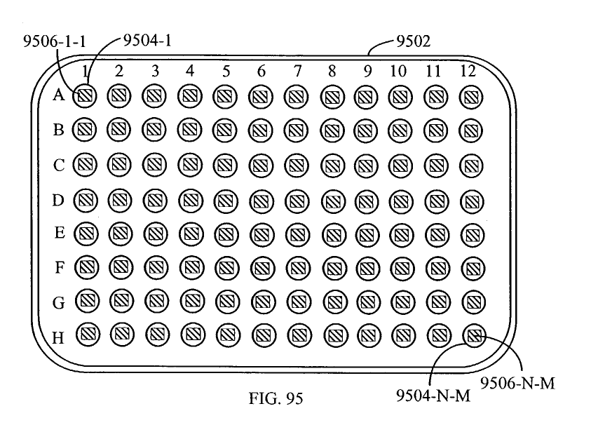

FIG. 95 illustrates an array of arrays 9502 in accordance with one embodiment of the present invention. The array of arrays 9502 is in the form of a microtiter (microwell) plate. The array of arrays 9502 includes a plurality of wells 9504. In some embodiments, there are 96, 384, or 1584 wells 9504. Each well is capable of holding at least one microliter of liquid. Within each well 9504 (e.g., at the well bottom) there is an array 9506. Each array 9506 illustrated in FIG. 95 is an array of devices 144, such as the array of devices 144 illustrated in FIG. 78. Each respective array 9506 in the array of arrays 9502 is connected to external circuitry (e.g., an electrical source) that is capable of driving a voltage through the electrode pair (e.g., materials 106 and 110) in each device 144 in the array (not shown).

In some embodiments, each well 9504 has a well diameter of 7.1 mm at the top, a well diameter of 6.5 mm at the bottom, and a depth of 11.2 mm. In some embodiments, the distance between well centers is 9 mm. In some embodiments, each array 9506 has dimensions of 3 mm by 3 mm. In some embodiments, there are at least 10,000 devices 144, at least 40,000 devices 144, at least 60,000 devices 144, at least 120,000 devices 144, or at least 250,000 devices 144 in one or more arrays 9506 in the array of arrays 9502.

Array of arrays 9502 provides a highly advantageous tool for detecting analytes. In some embodiments, all or a portion of an entire genome is populated on each array 9506 in array of arrays 9502. Then, the same or a different solution is placed in each well 9504 in array of arrays 9502 for an incubation period in accordance with the various methods disclosed above. After the incubation period, array of arrays 9502 is washed and any connection between respective electrode pairs in array of arrays 9502 is detected. Because of the microtiter (microwell) plate format, this sampling method can be automated using standard programmable robots that include an x-y plate. Therefore, a large number of analytes can be tested in parallel for their ability to bind to macromolecules 120 that are bound to the devices 144 within the array of arrays.

### 11.0 REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those of skill in the art. The specific embodiments described herein are offered by way of example only, and the invention is to be limited only by the terms of the appended claims, along with the fill scope of equivalents to which such claims are entitled.

DEVICE STRUCTURE FOR CLOSELY SPACED ELECTRODES
TABLE OF CONTENTS

| | |
|---|---|
| 1. CROSS-REFERENCE TO RELATED APPLICATIONS | 1 |
| 2. FIELD OF THE INVENTION | 1 |
| 3. BACKGROUND OF THE INVENTION | 1 |
| 4. SUMMARY OF THE INVENTION | 3 |
| 5. BRIEF DESCRIPTION OF THE DRAWINGS | 17 |
| 6. DETAILED DESCRIPTION | 26 |
| 6.1 BIOSENSOR CONFIGURATIONS IN ACCORDANCE WITH VARIOUS | 26 |
| EMBODIMENTS OF THE PRESENT INVENTION | |
| 6.1.1 ILLUSTRATIVE BIOSENSOR WITH NON-OVERLAPPING ELECTRODES | 27 |
| 6.1.2 ILLUSTRATIVE BIOSENSOR WITH OVERLAPPING ELECTRODES | 29 |
| 6.1.3 ILLUSTRATIVE BIOSENSOR WITH CAVITY IN THE INSULATOR LAYER | 29 |
| 6.1.4 ADDITIONAL BIOSENSOR CONFIGURATIONS | 30 |
| 6.1.5 CONNECTING DEVICE ELECTRODES TO AN EXTERNAL VOLTAGE | 59 |
| SOURCE | |
| 6.1.6 BIOSENSORS WITH ONE OR MORE ATTACHED MACROMOLECULES | 59 |
| 6.2 BIOSENSOR ARRAYS | 60 |
| 6.3 SUBSTRATES USED IN THE BIOSENSORS OF THE PRESENT INVENTION | 62 |
| 6.4 COMPOSITION OF BIOSENSOR ELECTRODES | 62 |
| 6.5 COMPOSITION OF BIOSENSOR INSULATORS | 63 |
| 6.6 COMPOSITION OF BIOSENSOR PASSIVATION LAYER | 64 |
| 6.7 BIOSENSOR ELECTRODE OVERLAP | 65 |
| 6.8 COMPOSITION OF TARGET BIOLOGICAL MACROMOLECULES | 65 |
| 6.9 ANALYTES USED TO BIND TO TARGET BIOLOGICAL MACROMOLECULES IN | 70 |
| THE PRESENT INVENTION | |
| 6.10 PREPARATION OF MACROMOLECULES AND ANALYTES | 71 |
| 6.10.1 PREPARATION OF MACROMOLECULES OR ANALYTES THAT ARE | 71 |
| NUCLEIC ACIDS | |
| 6.10.2 PREPARATION OF MACROMOLECULES OR ANALYTES THAT ARE | 71 |
| ANTIBODIES OR ANTIBODY FRAGMENTS | |
| 6.10.3 PREPARATION OF MACROMOLECULES OR ANALYTES THAT ARE | 74 |
| PROTEINS | |
| 6.10.4 PREPARATION OF MACROMOLECULES OR ANALYTES THAT ARE | 75 |
| SUGARS OR CARBOHYDRATES | |
| 6.11 SPACING BETWEEN BIOSENSOR ELECTRODES | 76 |
| 6.12 ATTACHMENT OF MACROMOLECULES TO BIOSENSOR ELECTRODES | 78 |
| 7.0 METHODS FOR MAKING THE BIOSENSORS OF THE PRESENT INVENTION | 81 |
| 7.1 TWO NON-OVERLAPPING ELECTRODES WITH TWO INSULATOR LAYERS | 82 |
| 7.1.1 INSULATOR FORMATION | 82 |
| 7.1.1.1 THERMAL OXIDATION OF SILICON | 83 |
| 7.1.1.2 CHEMICAL VAPOR DEPOSITION | 83 |
| 7.1.1.3 REDUCED PRESSURE CHEMICAL VAPOR DEPOSITION | 84 |
| 7.1.1.4 LOW PRESSURE CHEMICAL VAPOR DEPOSITION | 84 |
| 7.1.1.5 ATMOSPHERIC CHEMICAL VAPOR DEPOSITION | 84 |
| 7.1.1.6 PLASMA ENHANCED CHEMICAL VAPOR DEPOSITION | 84 |
| 7.1.1.7 ANODIZATION | 85 |
| 7.1.1.8 SOL-GEL DEPOSITION TECHNIQUE | 85 |
| 7.1.1.9 PLASMA SPRAYING TECHNIQUE | 86 |
| 7.1.1.10 INK JET PRINTING | 86 |
| 7.1.2 SPACER DEPOSITION AND RESIST LAYER DEPOSITION | 87 |
| 7.1.3 MASK ALIGNMENT AND RESIST LAYER EXPOSURE FOR SPACER | 89 |
| PATTERNING | |
| 7.1.4 RESIST LAYER DEVELOPMENT FOR SPACER PATTERNING | 90 |
| 7.1.5 SPACER ETCHING | 91 |
| 7.1.5.1 WET ETCHING | 91 |
| 7.1.5.2 WET SPRAY ETCHING OR VAPOR ETCHING | 91 |
| 7.1.5.3 PLASMA ETCHING | 92 |
| 7.1.5.4 ION BEAM ETCHING | 92 |
| 7.1.5.5 REACTIVE ION ETCHING | 93 |
| 7.1.6 RESIDUAL LAYER REMOVAL | 93 |
| 7.1.7 DEPOSITION OF ELECTRODES | 94 |
| 7.1.7.1 VACUUM EVAPORATION | 94 |
| 7.1.7.2 SPUTTER DEPOSITION/PHYSICAL VAPOR DEPOSITION | 95 |
| 7.1.7.3 COLLIMATED SPUTTERING | 96 |
| 7.1.7.4 LASER ABLATED DEPOSITION | 96 |
| 7.1.7.5 MOLECULAR BEAM DEPOSITION | 97 |
| 7.1.7.6 IONIZED PHYSICAL VAPOR DEPOSITION | 98 |
| 7.1.7.7 ION BEAM DEPOSITION | 98 |
| 7.1.7.8 ATOMIC LAYER DEPOSITION | 99 |
| 7.1.7.9 HOT FILAMENT CHEMICAL VAPOR DEPOSITION | 99 |
| 7.1.7.10 SCREEN PRINTING | 100 |
| 7.1.7.11 ELECTROLESS METAL DEPOSITION | 101 |
| 7.1.7.12 ELECTROPLATING | 101 |
| 7.1.8 RESIST LAYER DEPOSITION FOR THE BIOSENSOR ELECTRODES | 101 |
| 7.1.9 MASK ALIGNMENT AND RESIST LAYER EXPOSURE FOR THE | 102 |
| BIOSENSOR ELECTRODES | |
| 7.1.10 RESIST LAYER DEVELOPMENT FOR THE BIOSENSOR ELECTRODES | 102 |
| 7.1.11 BIOSENSOR ELECTRODE ETCHING | 102 |
| 7.1.12 ELECTRODE RESIDUAL LAYER REMOVAL | 102 |
| 7.1.13 ALTERNATIVE LITHOGRAPHIC TECHNIQUES | 103 |
| 7.2 FORMATION OF NON-OVERLAPPING ELECTRODES BY DEPOSITION AT AN | 104 |
| ANGLE | |
| 7.3 FORMATION OF TWO NON-OVERLAPPING ELECRODES WITH TWO INSULATOR | 105 |
| LAYERS AND INTRODUCTION OF A CAVITY | |
| 7.4 FORMATION OF TWO NON-OVERLAPPING ELECTRODES USING A π/2 DELIVERY | 106 |
| MECHANISM | |
| 7.5 FORMATION OF TWO NON-OVERLAPPING ELECTRODES WITH CAVITIES USING | 107 |
| A π/2 DELIVERY MECHANISM | |
| 7.6 FORMATION OF TWO NON-OVERLAPPING ELECTRODES WITH A PORTION OF | 108 |
| THE INSULATOR AND ELECTRODE REMOVED | |
| 7.7 FORMATION OF TWO NON-OVERLAPPING ELECTRODES WITH ADDITIONAL | 110 |
| INSULATOR REMOVAL | |
| 7.8 FORMATION OF TWO NON-OVERLAPPING ELECTRODES WITH INSULATOR | 110 |
| REMOVAL | |
| 7.9 STACKED NON-OVERLAPPING ELECTRODES | 111 |
| 7.10 STACKED NON-OVERLAPPING ELECTRODES WITH INSULATOR REMOVAL | 114 |
| 7.11 PLANAR ARRAYS OF BIOSENSORS | 115 |
| 7.12 ANALYTE DETECTION | 115 |
| 7.12.1 SAMPLE PREPARATION | 116 |
| 7.12.2 SAMPLE DELIVERY SYSTEM | 116 |
| 7.12.3 SAMPLE REACTION WITH A MACROMOLECULE | 116 |
| 7.12.3.1 HIGH STRINGENCY | 121 |
| 7.12.3.2 INTERMEDIATE STRINGENCY | 123 |
| 7.12.3.3 LOW STRINGENCY | 123 |
| 7.12.4 ANALYTE DETECTION AND QUANTIFICATION | 124 |
| 7.12.5 ADDITIONAL METHODS FOR DETECTING AN ANALYTE WITH A | 124 |
| BIOSENSOR | |
| 7.13 CASSETTES | 126 |
| 7.14 INTEGRATED ASSAY DEVICE/APPARATUS | 128 |
| 7.15 KITS | 129 |
| 7.16 MONITORING ELECTRON TRANSFER THROUGH BOUND MACROMOLECULE/ | 130 |
| ANALYTE COMPLEXES | |
| 8.0 PACKAGED BIOSENSORS | 133 |
| 8.1 PROCESSING STEPS USED TO MANUFACTURE AN ILLUSTRATIVE DEVICE | 133 |
| 8.2 DEVICE ARRAYS | 139 |
| 8.3 PROCESSING STEPS USED TO PACKAGE DEVICES | 140 |
| 8.4 PROCESSING STEPS USED TO MANUFACTURE AN ILLUSTRATIVE PACKAGED | 146 |
| DEVICE | |
| 8.5 MEASURING ANALYTE BINDING EVENTS | 147 |
| 9.0 DEVICE DENSITY | 148 |
| 10.0 ARRAY OF ARRAYS | 152 |
| 11.0 REFERENCES CITED | 153 |

What is claimed:

1. A biosensor comprising a plurality of devices, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer occupied by said device;
a spacer overlaid on a second portion of the different region of said insulator layer that is occupied by said device, wherein said first portion of the different region on said insulator does not overlap said second portion of the different region on said insulator; and
a second electrically conducting material, wherein the second electrically conducting material is overlaid on at least a portion of said spacer.

2. A biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region on said substrate occupied by said device;
a spacer overlaid on a second portion of the different region on said substrate that is occupied by said device, wherein said first portion of the different region on said substrate does not overlap said second portion of the different region on said substrate; and
a second electrically conducting material, wherein the second electrically conducting material is overlaid on at least a portion of said spacer.

3. The biosensor of claim 1 or 2 wherein said second electrically conducting material overlaps said first electrically conducting material of a device in said plurality of devices by a distance, thereby forming a cavity.

4. The biosensor of claim 3 wherein said distance is 150 Angstroms or less.

5. The biosensor of claim 3 wherein said distance is 100 Angstroms or less.

6. The biosensor of claim 3 wherein said distance is 50 Angstroms or less.

7. The biosensor of claim 1 or 2 wherein a passivation layer overlays said second electrically conducting material.

8. The biosensor of claim 7 wherein said passivation layer comprises silicon oxide, silicon dioxide, silicon nitride, silicon oxy-nitride, polyamide, oxidized aluminum, or photoresist.

9. The biosensor of claim 1 or 2, wherein a first portion of a macromolecule binds to a top portion of said first electrically conducting material and a second portion of said macromolecule binds to a side-wall of said second electrically conducting material in a device in said plurality of devices.

10. The biosensor of claim 1 or 2 wherein
a first passivation layer overlays a portion of said first electrically conducting material and a second passivation layer overlays said second electrically conducting material, and wherein
a first portion of a macromolecule binds to a top portion of said first electrically conducting material that is not covered by said first passivation layer and a second portion of said macromolecule binds to a side portion of said second electrically conducting material.

11. The biosensor of claim 1 or 2 wherein
a first passivation layer overlays a portion of said first electrically conducting material and a second passivation layer overlays a portion said second electrically conducting material, and wherein
a first portion of a macromolecule binds to a top portion of said first electrically conducting material that is not covered by said first passivation layer and a second portion of said macromolecule binds to a top portion of said second electrically conducting material that is not covered by said second passivation layer.

12. The biosensor of claim 11 wherein said first passivation layer and said second passivation layer each independently comprise silicon oxide, silicon dioxide, silicon nitride, silicon oxy-nitride, polyamide, oxidized aluminum, or photoresist.

13. The biosensor of claims 1 or 2 wherein
a passivation layer overlays said second electrically conducting material and said spacer comprises a gap exposing a portion of the bottom of said second electrically conducting material, and
a first portion of a macromolecule binds to a top portion of said first electrically conducting material and a second portion of said macromolecule binds to a side portion of said second electrically conducting material.

14. The biosensor of claim 13 wherein said passivation layer comprises silicon oxide, silicon dioxide, silicon nitride, silicon oxy-nitride, polyamide, oxidized aluminum, or photoresist.

15. The biosensor of claim 1 or 2 wherein a passivation layer overlays said second electrically conducting material and said spacer comprises a gap exposing a portion of the bottom of said second electrically conducting material.

16. The biosensor of claim 15 wherein said passivation layer comprises silicon oxide, silicon dioxide, silicon nitride, silicon oxy-nitride, polyamide, oxidized aluminum, or photoresist.

17. The biosensor of claim 16 wherein a first portion of a macromolecule binds to a top surface of said first electrically conducting material and a second portion of said macromolecule binds to said portion of the bottom of said second electrically conducting material that is exposed by said gap.

18. The biosensor of claim 16 wherein a first portion of a macromolecule binds to a top surface of said first electrically conducting material and a second portion of said macromolecule binds to a side-wall of said second electrically conducting material.

19. The biosensor of claim 16 wherein a first portion of a macromolecule binds to a side-wall of said first electrically conducting material and a second portion of said macromolecule binds to said portion of the bottom of said second electrically conducting material that is exposed by said gap.

20. The biosensor of claim 1 wherein a passivation layer overlays said second electrically conducting material and said spacer comprises a gap exposing a portion of the bottom of said second electrically conducting material and wherein said gap extends to said insulation layer.

21. The biosensor of claim 20 wherein said passivation layer comprises silicon oxide, silicon dioxide, silicon nitride, silicon oxy-nitride, polyamide, oxidized aluminum, or photoresist.

22. The biosensor of claim 1 wherein
a passivation layer overlays said second electrically conducting material and said spacer comprises a gap exposing a portion of the bottom of said second electrically conducting material, and wherein
said gap extends to said substrate through said insulation layer.

23. A biosensor comprising a plurality of devices on a substrate, each device in said plurality of devices occupying a different region on an insulator layer, wherein the insulator layer is overlaid on said substrate, each device in said plurality of devices comprising a different cavity in said insulator layer, each device in said plurality of devices comprising:
a first electrically conducting material, wherein the first electrically conducting material is in said different cavity of said device;
a second electrically conducting material, wherein the second electrically conducting material is overlaid on said insulator layer outside of said different cavity associated with said device; and
a passivation layer overlaid on said second electrically conducting material.

24. The biosensor of claim 23 wherein said passivation layer comprises silicon oxide, silicon dioxide, silicon nitride, silicon oxy-nitride, polyamide, oxidized aluminum, or photoresist.

25. The biosensor of claim 23 wherein said different cavity associated with a device in said plurality of devices has a width between 900 Angstroms and 20,000 Angstroms.

26. The biosensor of claim 23 wherein said different cavity associated with each device in said plurality of devices has a width between 500 Angstroms and 900 Angstroms.

27. A biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by the device; and
a second electrically conducting material, wherein the second electrically conducting material is overlaid on a second portion of the different region of said insulator layer that is occupied by said device, wherein said first portion of the different region on said insulator does not overlap said second portion of the different region on said insulator.

28. A biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by the device; and
a second electrically conducting material, wherein the second electrically conducting material is overlaid on a second portion of the different region of said substrate that is occupied by said device, wherein said first portion of the different region on said substrate does not overlap said second portion of the different region on said substrate.

29. The biosensor of claim 27 or 28 wherein said first electrically conducting material and said second electrically conducting material of a device in said plurality of devices are separated by a distance between 60 Angstroms and 500 Angstroms.

30. The biosensor of claim 27 or 28 wherein a passivation layer overlays a portion of said second electrically conducting material in a device in said plurality of devices.

31. The biosensor of claim 30 wherein said passivation layer comprises silicon oxide, silicon dioxide, silicon nitride, silicon oxy-nitride, polyamide, oxidized aluminum, or photoresist.

32. The biosensor of claim 27 or 28 wherein a first passivation layer overlays a portion of said first electrically conducting material and a second passivation layer overlays a portion of said second electrically conducting material in a device in said plurality of devices.

33. The biosensor of claim 32 wherein said first passivation layer and said second passivation layer each independently comprises silicon oxide, silicon dioxide, silicon nitride, silicon oxy-nitride, polyamide, oxidized aluminum, or photoresist.

34. The biosensor of claim 27 or 28 wherein a first portion of a macromolecule binds to a top portion of said first electrically conducting material and a second portion of said macromolecule binds to a side portion of said second electrically conducting material in a device in said plurality of devices.

35. The biosensor of claim 27 or 28 wherein a first portion of a macromolecule binds to a side portion of said first electrically conducting material and a second portion of said macromolecule binds to a side portion of said second electrically conducting material in a device in said plurality of devices.

36. The biosensor of claim 27 or 28 wherein a first portion of a macromolecule binds to a top portion of said first electrically conducting material and a second portion of said macromolecule binds to a top portion of said second electrically conducting material in a device in said plurality of devices.

37. The biosensor of claim 27 or 28 wherein said second electrically conducting material is thicker than said first electrically conducting material in a device in said plurality of devices.

38. The biosensor of claim 27 or 28 wherein said second electrically conducting material and said first electrically conducting material in a device in said plurality of devices have the same thickness.

39. The biosensor of claim 27 wherein
said first electrically conducting material and said second electrically conducting material in a device in said plurality of devices are separated by a distance, and
there is a gap in said insulator layer between said first electrically conducting material and said second electrically conducting material in said device.

40. The biosensor of claim 39 wherein said gap has a width of between 60 Angstroms and 500 Angstroms.

41. The biosensor of claim 38 wherein said gap has a width that exceeds a distance that separates said first electrically conducting material and said second electrically conducting material of said device.

42. The biosensor of claim 38 wherein said gap has a width that is between 60 Angstroms and 10,000 Angstroms.

43. The biosensor of claim 38 wherein said gap has a width that is between 60 Angstroms and 30,000 Angstroms.

44. The biosensor of claim 38, wherein said gap has a width that is between 60 Angstroms and 100,000 Angstroms.

45. A biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material.

46. A biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material.

47. The biosensor of claim 45 or 46 wherein said passivation layer comprises silicon oxide, silicon dioxide, silicon nitride, silicon oxy-nitride, polyamide, oxidized aluminum, or photoresist.

48. The biosensor of claim 45 or 46 wherein a distance between said first electrically conducting material and said second electrically conducting material in a device in said plurality of devices is between 60 Angstroms and 103 Angstroms.

49. The biosensor of claim 45 or 46 wherein a distance between said first electrically conducting material and said second electrically conducting material in a device in said plurality of devices is between 80 Angstroms and 300 Angstroms.

50. The biosensor of claim 45 or 46 wherein a distance between said first electrically conducting material and said second electrically conducting material in a device in said plurality of devices is between 100 Angstroms and 200 Angstroms.

51. The biosensor of claim 45 or 46 wherein a first portion of a macromolecule binds to a side portion of said first electrically conducting material and a second portion of said macromolecule binds to a side portion of said second electrically conducting material in a device in said plurality of devices.

52. The biosensor of claim 45 or 46 wherein
said thin segment of said spacer in a device in said plurality of devices comprises a cavity, and wherein
a first portion of a macromolecule binds to a top portion of said first electrically conducting material and a second portion of said macromolecule binds to a bottom portion of said second electrically conducting material in said cavity.

53. The biosensor of claim 45 or 46 wherein
a portion of the upper surface of said second electrically conducting material is not covered by said passivation layer; and
a first portion of a macromolecule binds to a side portion of said first electrically conducting material and a second portion of said macromolecule binds to said portion of the upper surface of said second electrically conducting material that is not covered by said passivation layer.

54. A biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material.

55. A biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material.

56. The biosensor of claim 54 or 55 wherein said passivation layer comprises silicon oxide, silicon dioxide, silicon nitride, silicon oxy-nitride, polyamide, oxidized aluminum, or photoresist.

57. The biosensor of claim 54 or 55 wherein a first portion of a macromolecule binds to a side portion of said first electrically conducting material and a second portion of said macromolecule binds to a side portion of said second electrically conducting material in a device in said plurality of devices.

58. The biosensor of claim 54 or 55 wherein a first portion of a macromolecule binds to a top portion of said first electrically conducting material and a second portion of said macromolecule binds to a bottom portion of said second electrically conducting material in said cavity in a device in said plurality of devices.

59. The biosensor of claim 54 or 55 wherein
a portion of the upper surface of said second electrically conducting material is not covered by said passivation layer; and
a first portion of a macromolecule binds to a side portion of said first electrically conducting material and a second portion of said macromolecule binds to said portion of the upper surface of said second electrically conducting material that is not covered by said passivation layer.

60. A biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material.

61. A biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate occupied by said device, wherein said first portion of said substrate does not overlap with said second portion of said substrate;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material.

62. The biosensor of claim 60 or 61 wherein said first passivation layer comprises silicon oxide, silicon dioxide, silicon nitride, silicon oxy-nitride, polyamide, oxidized aluminum, or photoresist.

63. The biosensor of claim 60 or 61 wherein a second passivation layer overlays said first electrically conducting material.

64. The biosensor of claim 63 wherein said second passivation layer comprises silicon oxide, silicon dioxide, silicon nitride, silicon oxy-nitride, polyamide, oxidized aluminum, or photoresist.

65. The biosensor of claim 60 or 61 wherein a first portion of a macromolecule binds to a top portion of said first electrically conducting material and a second portion of said macromolecule binds to a side-wall of said second electrically conducting material in a device in said plurality of devices.

66. The biosensor of claim 60 or 61 wherein
a second passivation layer overlays a portion of said first electrically conducting material; and
a first portion of a macromolecule binds to a top portion of said first electrically conducting material that is not covered by said second passivation layer and a second portion of said macromolecule binds to a side-wall of said second electrically conducting material.

67. The biosensor of claim 66 wherein said second passivation layer comprises silicon oxide, silicon dioxide, silicon nitride, silicon oxy-nitride, polyamide, oxidized aluminum, or photoresist.

68. The biosensor of claim 60 wherein said insulator comprises a gap that is between said first electrically conducting material and said spacer.

69. The biosensor of claim 60 or 61 wherein said spacer comprises a crevice that exposes a portion of said second electrically conducting material.

70. A biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device, the spacer including a main body and an extended portion, wherein said extended portion of said spacer abuts said first electrically conducting material and wherein said first portion of said insulator layer does not overlap with said second portion of said insulator layer;
a second electrically conducting material, wherein the second electrically conducting material is overlaid on said main body of said spacer; and
a first passivation layer overlays said second electrically conducting material.

71. A biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate that is occupied by said device, the spacer including a main body and an extended portion, wherein said extended portion of said spacer abuts said first electrically conducting material and wherein said first portion of said substrate does not overlap with said second portion of said substrate;
a second electrically conducting material, wherein the second electrically conducting material is overlaid on said main body of said spacer; and
a first passivation layer overlays said second electrically conducting material.

72. The biosensor of claim 70 or 71 wherein said first passivation layer comprises silicon oxide, silicon dioxide, silicon nitride, silicon oxy-nitride, polyamide, oxidized aluminum, or photoresist.

73. The biosensor of claim 70 or 71 wherein a second passivation layer overlays said first electrically conducting material.

74. The biosensor of claim 73 wherein said second passivation layer comprises silicon oxide, silicon dioxide, silicon nitride, silicon oxy-nitride, polyamide, oxidized aluminum, or photoresist.

75. The biosensor of claim 70 or 71 wherein a first portion of a macromolecule binds to a top portion of said first electrically conducting material and a second portion of said macromolecule binds to a side-wall of said second electrically conducting material in a device in said plurality of devices.

76. The biosensor of claim 70 or 71 wherein a second passivation layer overlays a portion of said first electrically conducting material and wherein a first portion of a macromolecule binds to a top portion of said first electrically conducting material that is not covered by said first passivation layer and a second portion of said macromolecule binds to a side portion of said second electrically conducting material in a device in said plurality of devices.

77. The biosensor of claim 70 or 71 wherein said extended portion of said spacer has a width of more than 200 Angstroms in a device in said plurality of devices.

78. The biosensor of claim 70 or 71 wherein said extended portion of said spacer has a width of more than 500 Angstroms in a device in said plurality of devices.

79. The biosensor of claim 70 or 71 wherein said extended portion of said spacer has a width between 25 Angstroms and 700 Angstroms in a device in said plurality of devices.

80. The biosensor of claim 70 or 71 wherein said extended portion of said spacer comprises a gap in a device in said plurality of devices.

81. The biosensor of claim 80 wherein said main portion of said spacer comprises a crevice that exposes a bottom portion of said second electrically conductive material.

82. The biosensor of claim 81 wherein a first portion of a macromolecule binds to an upper surface of said first electrically conducting material and a second portion of said macromolecule binds to a side portion of said second electrically conducting material.

83. The biosensor of claim 80 wherein a first portion of a macromolecule binds to side-wall of said first electrically conducting material and a second portion of said macromolecule binds to a portion of said second electrically conducting material that is exposed by said crevice.

84. A biosensor comprising:
a substrate;
an insulator layer overlaid on said substrate, wherein
said insulator layer comprises a plurality of steps, and a first step in said plurality of steps is at a different height, with respect to said substrate, than a second step in said plurality of steps;
a different electrically conducting layer is overlaid on each step in said plurality of steps; and
each said different electrically conducting layer overlaid on a step in said plurality of steps is electrically insulated from all other electrically conducting layers in said biosensor.

85. The biosensor of claim 84 wherein each electrically conducting layer in said biosensor is addressable by an electrical source.

86. The biosensor of claim 84 wherein an electrically conducting layer associated with a step in said plurality of steps is electrically insulated from all other electrically conducting layers in said biosensor by a cavity in the step.

87. The biosensor of claim 84 wherein the difference in height, with respect to said substrate, between a first step in said plurality of steps and a second step in said plurality of steps is between 60 Angstroms and 200 Angstroms.

88. The biosensor of claim 84 wherein the difference in height, with respect to said substrate, between a first step in said plurality of steps and a second step in said plurality of steps is less than 500 Angstroms.

89. The biosensor of claim 84 wherein the difference in height, with respect to said substrate, between a first step in said plurality of steps and a second step in said plurality of steps is less than 1000 Angstroms.

90. The biosensor of claim 84 wherein said first step and said second step are adjacent to each other and a first portion of a macromolecule binds to said first step in said plurality of steps and a second portion of said macromolecule binds to said second step.

91. The biosensor of claim 1, 2, 23, 27, or 28 wherein a plane including a top surface of said first electrically conducting material and a plane comprise a top surface of said second electrically conducting material are separated by a distance between 60 Angstroms and 200 Angstroms in a device in said plurality of devices.

92. The biosensor of claim 1, 2, 23, 27, or 28 wherein a plane that comprises a top surface of said first electrically conducting material and a plane that comprises a top surface of said second electrically conducting material are separated by a distance that is less than 500 Angstroms in a device in said plurality of devices.

93. The biosensor of claim 1, 2, 23, 27, or 28 wherein a plane that comprises a top surface of said first electrically conducting material and a plane that comprises a top surface of said second electrically conducting material are separated by a distance that is less than 1000 Angstroms in a device in said plurality of devices.

94. The biosensor of claim 1, 2, 23, 27, or 28 wherein a plane that comprises a top surface of said first electrically conducting material and a plane that comprises a top surface of said second electrically conducting material are separated by a distance that is between 300 Angstroms and 400 Angstroms in a device in said plurality of devices.

95. The biosensor of claim 1, 2, 23, 27, or 28 wherein a plane that comprises a top surface of said first electrically conducting material and a plane that comprises a top surface of said second electrically conducting material are separated by a distance that is between 200 Angstroms and 300 Angstroms in a device in said plurality of devices.

96. The biosensor of claim 1, 2, 23, 27, or 28 wherein a plane that comprises a top surface of said first electrically conducting material and a plane that comprises a top surface of said second electrically conducting material are separated by a distance that is less than 300 Angstroms in a device in said plurality of devices.

97. The biosensor of claim 1, 2, 23, 27, or 28 wherein a plane that comprises a top surface of said first electrically conducting material and a plane that comprises a top surface of said second electrically conducting material are separated by a distance that is less than 200 Angstroms in a device in said plurality of devices.

98. The biosensor of claim 45, 46, 54, 55, 60, 61, 70 or 71 wherein a portion of said first electrically conducting material and a portion of said second electrically conducting material are separated by a distance that is less than 150 Angstroms in a device in said plurality of devices.

99. The biosensor of claim 45, 46, 54, 55, 60, 61, 70 or 71 wherein a portion of said first electrically conducting material and a portion of said second electrically conducting material are separated by a distance that is less than 100 Angstroms in a device in said plurality of devices.

100. The biosensor of claim 45, 46, 54, 55, 60, 61, 70 or 71 wherein a portion of said first electrically conducting material and a portion of said second electrically conducting material are separated by a distance that is between 50 Angstroms and 80 Angstroms in a device in said plurality of devices.

101. The biosensor of claim 1, 2, 23, 27, 28, 45, 46, 54, 55, 60, 61, 70, or 71 wherein said plurality of devices comprises 10 to 250,000 devices.

102. The biosensor of claim 1, 2, 23, 27, 28, 45, 46, 54, 55, 60, 61, 70, or 71 wherein said plurality of devices comprises 10,000 to 60,000 devices.

103. The biosensor of claim 1, 2, 23, 27, 28, 45, 46, 54, 55, 60, 61, 70, or 71 wherein said plurality of devices are arranged in an array having at least 200 rows and at least 200 columns on said substrate.

104. The biosensor of claim 1, 2, 23, 27, 28, 45, 46, 54, 55, 60, 61, 70, 71, or 84 wherein said substrate is an insulator.

105. The biosensor of claim 1, 2, 23, 27, 28, 45, 46, 54, 55, 60, 61, 70, 71 or 84 wherein said substrate comprises silicon, silicon oxide, silicon dioxide, silicon nitride, Teflon, alumina, glass, sapphire, a selinide, or polyester.

106. The biosensor of claim 1, 2, 23, 27, 28, 45, 46, 54, 55, 60, 61, 70, or 71 wherein said first electrically conducting material and said second electrically conducting material each has a resistivity less than 10-6 ohm-meters in a device in said plurality of devices.

107. The biosensor of claim 1, 2, 23, 27, 28, 45, 46, 54, 55, 60, 61, 70, or 71 wherein said first electrically conducting material and said second electrically conducting material are comprised of the same composition in a device in said plurality of devices.

108. The biosensor of claim 1, 2, 23, 27, 28, 45, 46, 54, 55, 60, 61, 70, or 71 wherein said first electrically conducting material and said second electrically conducting material are comprised of different compositions in a device in said plurality of devices.

109. The biosensor of claim 1, 2, 23, 27, 28, 45, 46, 54, 55, 60, 61, 70, or 71 wherein said first electrically conducting material comprises aluminum, nickel, platinum, iron, copper, silver, gold, indium tin oxide, chromium, titanium, zinc, tin, an alloy of aluminum, an alloy of nickel, an alloy of platinum, an alloy of iron, an alloy of copper, an alloy of silver, an alloy of gold, an alloy of chromium, an alloy of titanium, an alloy of zinc or an alloy of tin in a device in said plurality of devices.

110. The biosensor of claim 1, 2, 23, 27, 28, 45, 46, 54, 55, 60, 61, 70, or 71 wherein said first electrically conducting material comprises a metal carbide, a metal nitride, a metal boride, a conductive oxide, a metal silicide or a metal sulfide in a device in said plurality of devices.

111. The biosensor of claim 1, 23, 27, 45, 54, 60, 70 or 84 wherein said insulator comprises a material having a resistivity greater than 10-1 ohm-meters in a device in said plurality of devices.

112. The biosensor of claim 1, 23, 27, 45, 54, 60, 70 or 84 wherein said insulator comprises TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene or a high resistivity plastic in a device in said plurality of devices.

113. The biosensor of claim 1, 2, 45, 46, 54, 55, 60, 61, 70, or 71 wherein said spacer comprises a metal carbide, a metal nitride, a metal boride, a conductive oxide, a metal silicide or a metal sulfide in a device in said plurality of devices.

114. The biosensor of claim 1, 2, 45, 46, 54, 55, 60, 61, 70, or 71 wherein said spacer comprises a material having a resistivity greater than 10⁻¹ ohm-meters in a device in said plurality of devices.

115. The biosensor of claim 1, 2, 38, 39, 46, 47, 51, 52, 58, or 59 wherein said spacer comprises TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene or a high resistivity plastic in a device in said plurality of devices.

116. The biosensor of claim 1, 2, 23, 27, 28, 45, 46, 54, 55, 60, 61, 70, 71, or 84 wherein a macromolecule is bound to a first electrically conducting material and/or a second electrically conducting material in a device in said plurality of devices and said macromolecule comprises a nucleic acid, a protein, a polypeptide, a peptide, an antibody, a carbohydrate, a polysaccharide, a lipid, a fatty acid or a sugar.

117. A method of manufacturing a biosensor, the method comprising:
(a) depositing a first insulator layer onto a substrate;
(b) depositing a second insulator layer on said first insulator layer;
(c) patterning said second insulator layer, thereby forming a spacer and exposing a portion of said first insulator layer;
(d) depositing electrically conducting material on said spacer and said portion of said first insulator layer that is exposed;
(e) patterning said electrically conducting material deposited on said portion of said first insulator layer to form a first electrically conducting material; and
(f) patterning said electrically conducting material deposited on said spacer to form a second electrically conducting material.

118. The method of claim 117 wherein said depositing step (a) is performed by thermal oxidation of silicon, chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, sputter deposition, vacuum evaporation, laser ablated deposition, atomic layer deposition, molecular beam deposition, ion beam deposition, hot filament chemical vapor deposition or screen printing.

119. The method of claim 117 wherein said depositing step (b) is performed by chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, sputter deposition, vacuum evaporation, laser ablated deposition, atomic layer deposition, molecular beam deposition, ion beam deposition, hot filament chemical vapor deposition or screen printing.

120. The method of claim 117 wherein said depositing step (b) comprises chemical vapor deposition of silicon oxide or silicon nitride.

121. The method of claim 117 wherein said patterning step (c) comprises:
application of a photolithographic photoresist coating to said second insulator layer;
optical imaging of said photolithographic photoresist coating through an optical mask;
developing said photolithographic photoresist coating;
etching said spacer; and
removing said photolithographic photoresist coating.

122. The method of claim 121 wherein said photolithographic photoresist coating is a negative resist or a positive resist.

123. The method of claim 121 wherein said photolithographic photoresist coating is an azide/isoprene negative resist, polymethylmethacrylate (PMMA), polymethylisopropyl ketone (PMIPK), poly-butene-1-sulfone (PBS), poly-(trifluoroethyl chloroacrylate) TFECA, copolymer-(α-cyano ethyl acrylate-α-amido ethyl acrylate) (COP), poly-(2-methyl pentene-1-sulfone) (PMPS), phenol-formaldehyde novolak resin, or polydimethylglutarimide.

124. The method of claim 121 wherein said photolithographic photoresist coating is developed by exposing said photolithographic photoresist coating to xylene, Stoddart solvent, n-butlyl acetate, sodium hydroxide, potassium hydroxide, or tetramethylammonium hydroxide.

125. The method of claim 121 wherein said etching said spacer comprises wet etching, wet spray etching, vapor etching, plasma etching, ion beam etching or reactive ion etching.

126. The method of claim 121 wherein said removing said photolithographic photoresist coating comprises exposing said photolithographic photoresist coating to a strong acid, an acid-oxidant combination, an organic solvent stripper, or an alkaline stripper.

127. The method of claim 117 wherein said depositing step (d) is performed by chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, direct current diode sputtering, radio frequency diode sputtering, direct current magnetron sputtering, radio frequency magnetron sputtering, vacuum evaporation, collimated sputtering, laser ablated deposition, atomic layer deposition, molecular beam deposition, ionized physical vapor deposition, ion beam deposition, atomic layer deposition, hot filament chemical vapor deposition, screen printing, electroless metal deposition, electroplating, or electroless/immersion gold.

128. The method of claim 117 wherein said patterning step (e) and said patterning step (f) each comprises:
(i) applying a photolithographic photoresist coating to said electrically conducting material;
(ii) optically imaging said photolithographic photoresist coating through an optical mask;
(iii) developing said photolithographic photoresist coating;
(iv) etching said electrically conducting material; and
(v) removing said photolithographic photoresist coating.

129. The method of claim 128 wherein said photolithographic photoresist coating is a negative resist or a positive resist.

130. The method of claim 128 wherein said photolithographic photoresist coating is an azide/isoprene negative resist, polymethylmethacrylate (PMMA), polymethylisopropyl ketone (PMIPK), poly-butene-1-sulfone (PBS), poly-(trifluoroethyl chloroacrylate) TFECA, copolymer-(α-cyano ethyl acrylate-α-amido ethyl acrylate) (COP), poly-(2-methyl pentene-1-sulfone) (PMPS), phenol-formaldehyde novolak resin, or polydimethylglutarimide.

131. The method of claim 128 wherein said photolithographic photoresist coating is developed by exposure to xylene, Stoddart solvent, n-butlyl acetate, sodium hydroxide, potassium hydroxide, or tetramethylammonium hydroxide.

132. The method of claim 128 wherein said etching step (iv) comprises wet etching, wet spray etching, vapor etching, plasma etching, ion beam etching or reactive ion etching.

133. The method of claim 128 wherein said removing step (v) comprises exposing said photolithographic photoresist coating to a strong acid, an acid-oxidant combination, an organic solvent stripper, or an alkaline stripper.

134. The method of claim 117 wherein said depositing step (d) is performed by chemical vapor deposition,

135. The method of claim 117, wherein said depositing step (d) is performed by depositing material at an angle with respect to the substrate.

136. The method of claim 135, wherein said angle is between 0 radians and 2π radians.

137. The method of claim 135, wherein said angle is π/2 radians.

138. A method of processing a biosensor, the method comprising:
(a) etching a stack, the stack comprising
a substrate;
a first insulator layer overlaid on said substrate;
a first electrically conducting material overlaid on said first insulator layer;
a passivation layer overlaid on said first electrically conducting material; and
a sacrificial insulator layer overlaid on said passivation layer;
wherein said etching forms a cavity that extends through said sacrificial insulator layer, said passivation layer, said first electrically conducting material, and said first insulator layer;
(b) forming a second insulator layer at a bottom of said cavity;
(c) depositing a second electrically conducting material on said second insulator layer; and
(d) removing said sacrificial insulator layer overlaid on said passivation layer.

139. The method of claim 138 wherein said etching step (a) comprises a wet etching process, a wet spray etching technique, a vapor etching process, plasma etching, ion beam etching, or reactive ion etching.

140. The method of claim 138 wherein said substrate is made out of silicon and said forming a second insulator layer comprises growing silicon oxide on said substrate.

141. The method of claim 138 wherein said depositing step (c) comprises depositing at an angle with respect to said substrate.

142. The method of claim 141 wherein said angle is between 0 degrees and 180 degrees.

143. The method of claim 141 wherein said angle is ninety degrees.

144. A biosensor comprising:
a substrate;
a first insulator layer overlaid on said substrate;
a first electrically conducting material overlaid on said insulator;
a passivation layer overlaid on said first electrically conducting material;
a plurality of devices; wherein each device in said plurality of devices comprises:
a cavity that extends through said passivation layer, said first electrically conducting material, and said first insulator layer;
a second insulator layer in said cavity; and
a second electrically conducting material on said second insulator layer.

145. The biosensor of claim 144 wherein said first insulator layer has a thickness that is between 10 Angstroms and 10,000 Angstroms.

146. The biosensor of claim 144 wherein said first insulator layer has a thickness that is between 100 Angstroms and 2000 Angstroms.

147. The biosensor of claim 144 wherein said first insulator layer has a thickness that is between 400 Angstroms and 800 Angstroms and wherein said first insulator layer comprises silicon oxide.

148. The biosensor of claim 144 wherein said first insulator layer has a thickness that is between 400 Angstroms and 800 Angstroms.

149. The biosensor of claim 144 wherein said substrate comprises silicon, silicon oxide, silicon dioxide, silicon nitride, Teflon, alumina, glass, sapphire, a selinide, or polyester.

150. The biosensor of claim 144 wherein said first electrically conducting material and said second electrically conducting material each has a resistivity less than 10-6 ohm-meters.

151. The biosensor of claim 144 wherein said first electrically conducting material and said second electrically conducting material are comprised of the same composition.

152. The biosensor of claim 144 wherein said first electrically conducting material and said second electrically conducting material are comprised of different compositions.

153. The biosensor of claim 144 wherein said first electrically conducting material comprises aluminum, nickel, platinum, iron, copper, silver, gold, indium tin oxide, chromium, titanium, zinc, tin, an alloy of aluminum, an alloy of nickel, an alloy of platinum, an alloy of iron, an alloy of copper, an alloy of silver, an alloy of gold, an alloy of chromium, an alloy of titanium, an alloy of zinc, or an alloy of tin.

154. The biosensor of claim 144 wherein said first electrically conducting material comprises a metal carbide, a metal nitride, a metal boride, a conductive oxide, a metal silicide or a metal sulfide.

155. The biosensor of claim 144 wherein said first insulator layer comprises a material having a resistivity greater than 10⁻¹ ohm-meters.

156. The biosensor of claim 144 wherein said first insulator layer comprises TiO, ZrO₂, Al₂O₃, CaF₂, Cr₂O₃, Er₂O₃, HfO₂, MgF₂, MgO, Si₃N₄, SnO₂, SiO₂, quartz, porcelain, tantalum pentoxide, silicon oxide, silicon nitride, ceramic, polystyrene, Teflon, insulating carbon derivatives, glass, clay, polystyrene or a high resistivity plastic.

157. The biosensor of claim 144 wherein said first electrically conducting material has a thickness between 50 Angstroms and 1000 Angstroms.

158. The biosensor of claim 144 wherein said first electrically conducting material has a thickness between 100 Angstroms and 600 Angstroms.

159. The biosensor of claim 144 wherein said first electrically conducting material has a thickness between 100 Angstroms and 600 Angstroms and wherein said first electrically conducting material is made of platinum or gold.

160. The biosensor of claim 144 wherein said passivation layer has a thickness that is less than 10 Angstroms.

161. The biosensor of claim 144 wherein said passivation layer has a thickness between 10 Angstroms and 100 Angstroms.

162. The biosensor of claim 144 wherein said passivation layer comprises silicon oxide, silicon dioxide, silicon nitride, silicon oxy-nitride, polyamide, oxidized aluminum, or photoresist.

163. The biosensor of claim 144 wherein an etch stop overlays said substrate and said first insulator layer overlays said etch stop.

164. The biosensor of claim 163 wherein said etch stop has a thickness that is between 40 Angstroms and 500 Angstroms.

165. The biosensor of claim 163 wherein a cavity in a device in said plurality of devices has a width of between 0.09 microns and 2.0 microns.

166. The biosensor of claim 163 wherein said cavity in a device in said plurality of devices has a width between 0.13 microns and 0.35 microns.

167. The biosensor of claim 163 wherein a distance from the top of said first electrically conducting material and the top of said second electrically conducting material is between 60 Angstroms and 200 Angstroms.

168. The biosensor of claim 163 wherein a distance from the top of said first electrically conducting material and the top of said second electrically conducting material is between 50 Angstroms and 300 Angstroms.

169. The biosensor of claim 163 wherein a distance from the top of said first electrically conducting material and the top of said second electrically conducting material is between 100 Angstroms and 250 Angstroms.

170. A biosensor comprising a plurality of devices on a substrate, wherein
said substrate comprises a plurality of upper steps and a plurality of lower steps;
each upper step in the plurality of upper steps is associated with a lower step in the plurality of lower steps; and
for each device in said plurality of devices, a first electrically conducting material in the device overlays an upper step in said plurality of upper steps and a second electrically conducting material in the device overlays the lower step in said plurality of lower steps that is associated with the upper step.

171. The biosensor claim 170 wherein said substrate is sealed onto a die attach surface of a package body and said package body comprises a plurality of leads.

172. The biosensor of claim 171 wherein said package body is enclosed with an upper piece in a package.

173. The biosensor of claim 172 wherein said upper piece is ceramic.

174. The biosensor of claim 172 wherein said upper piece has an access hole.

175. The biosensor of claim 172 wherein said package is a dual in-line package, a single in-line package, or a ball grid array package.

176. The biosensor of claim 172 wherein said package is attached to a printed circuit board.

177. The biosensor of claim 176 wherein said printed circuit board is interfaced with a data acquisition card.

178. The biosensor of claim 176 wherein said printed circuit board is interfaced with a digital multimeter.

179. The biosensor of claim 171 the biosensor further comprising a plurality of bonding pads and a plurality of interconnects on said substrate, wherein
an interconnect in said plurality of interconnects joins a bonding pad in said plurality of bonding pads to a first electrically conducting material or a second electrically conducting material in a device in said plurality of devices.

180. The biosensor of claim 179 wherein a bonding pad in said plurality of bonding pads is connected to a lead in said plurality of leads.

181. The biosensor of claim 179 the biosensor further comprising a demultiplexer wherein said demultiplexer selectively connects a first electrically conducting material or a second electrically conducting material in a device in said plurality of devices to a bonding pad in said plurality of bonding pads.

182. The biosensor of claim 181 wherein said demultiplexer has a complementary metal-oxide semiconductor architecture.

183. The biosensor of claim 170 wherein an insulator layer is overlaid on said substrate and each device in said plurality of devices is overlaid on said insulator layer.

184. The biosensor of claim 170 wherein an upper step in said plurality of upper steps and the lower step associated with said upper step are separated by a vertical distance that is between 10 Angstroms and 10,000 Angstroms.

185. The biosensor of claim 170 wherein an upper step in said plurality of upper steps and the lower step associated with said upper step are separated by a vertical distance that is between 100 Angstroms and 1000 Angstroms.

186. The biosensor of claim 170 wherein an upper step in said plurality of upper steps and the lower step associated with said upper step are separated by a vertical distance that is between 200 Angstroms and 500 Angstroms.

187. The biosensor of claim 170 wherein an upper step in said plurality of upper steps and the lower step associated with said upper step are separated by a vertical distance that is between 300 Angstroms and 400 Angstroms.

188. The biosensor of claim 170 wherein said plurality of devices comprises at least 100 devices.

189. The biosensor of claim 170 wherein said plurality of devices comprises at least 10,000 devices.

190. The biosensor of claim 170 wherein said plurality of devices comprises 10,000 to 105 devices.

191. The biosensor of claim 170 wherein said plurality of devices comprises 10⁷ to 10⁹ devices.

192. The biosensor of claim 170 wherein a macromolecule binds to both a first electrically conducting material and a second electrically conducting material in a device in said plurality of devices.

193. The biosensor of claim 170 wherein said substrate comprises silicon, silicon oxide, silicon dioxide, silicon nitride, Teflon, alumina, glass, sapphire, a selinide, or polyester.

194. The biosensor of claim 170 wherein said first electrically conducting material and said second electrically conducting material each has a resistivity less than 10⁻⁶ ohm-meters in a device in said plurality of devices.

195. The biosensor of claim 170 wherein said first electrically conducting material and said second electrically conducting material are comprised of the same composition in a device in said plurality of devices.

196. The biosensor of claim 170 wherein said first electrically conducting material and said second electrically conducting material are comprised of different compositions in a device in said plurality of devices.

197. The biosensor of claim 170 wherein said first electrically conducting material or said second electrically conducting material comprises aluminum, nickel, platinum, iron, copper, silver, gold, indium tin oxide, chromium, titanium, zinc, tin, an alloy of aluminum, an alloy of nickel, an alloy of platinum, an alloy of iron, an alloy of copper, an alloy of silver, an alloy of gold, an alloy of chromium, an alloy of titanium, an alloy of zinc or an alloy of tin in a device in said plurality of devices.

198. The biosensor of claim 170 wherein said first electrically conducting material or said second electrically conducting material comprises a metal carbide, a metal nitride, a metal boride, a conductive oxide, a metal silicide or a metal sulfide in a device in said plurality of devices.

199. The biosensor of claim 170 wherein said first electrically conducting material and said second electrically conducting material in a device in said plurality of devices is connected to external circuitry.

200. A method of manufacturing a packaged biosensor, the method comprising:
(a) depositing an electrically conducting layer onto a substrate, said substrate comprising a plurality of upper steps and a plurality of lower steps, wherein each upper step in said plurality of upper steps is associated with a lower step in the plurality of lower steps;
(b) patterning said electrically conducting layer to form a plurality of electrode pairs, a plurality of bonding pads, and a plurality of interconnects, wherein
an interconnect in said plurality of interconnects joins an electrode in said plurality of electrode pairs to a bonding pad in said plurality of bonding pads, and
each electrode pair comprises a first electrode and a second electrode, wherein said first electrode is on an upper step in said plurality of upper steps and said second electrode is on the lower step in said plurality of lower steps that is associated with said upper step;
(c) sealing said substrate to a die attach surface of a package body wherein said package body comprises a plurality of leads;
(d) attaching a bonding pad in said plurality of bonding pads to a lead in said plurality of leads; and
(e) enclosing said package body with an upper piece, thereby manufacturing said packaged biosensor.

201. The method of claim 200 wherein said upper piece is ceramic.

202. The method of claim 200 wherein said upper piece has an access hole.

203. The method of claim 200 wherein said enclosing step (e) comprises applying epoxy to said die attach surface and then placing said upper piece on said epoxy.

204. The method of claim 200, the method further comprising curing said biosensor.

205. The method of claim 204 wherein said curing comprises heating said biosensor in a curing oven.

206. The method of claim 200, the method further comprising depositing an insulation layer on said substrate prior to said depositing an electrically conducting layer onto said substrate.

207. The method of claim 200 wherein said patterning step (b) creates a plurality of die, each die comprising a plurality of electrode pairs, a plurality of bonding pads and a plurality of interconnects on said substrate, and wherein said method further comprises separating a die from said plurality of die.

208. The method of claim 207 wherein said separating comprises sawing.

209. The method of claim 200 wherein said sealing step (c) uses an epoxy die attachment technique.

210. The method of claim 200 wherein said sealing step (c) uses a eutectic die attachment technique.

211. The method of claim 200 wherein said attaching step (d) is repeated.

212. The method of claim 200 wherein said attaching step (d) uses a wire bonding technique, a flip-chip technique, or a beam-lead technique.

213. The method of claim 200 wherein said package body is a dual in-line package, single-in-line package, or a ball grid array package.

214. The method of claim 200 wherein said patterning step (b) also forms a demultiplexer and said demultiplexer selectively connects an electrode in said plurality of electrode pairs to a bonding pad in said plurality of bonding pads.

215. The method of claim 214 wherein said demultiplexer has a complementary metal-oxide semiconductor architecture.

216. The method of claim 200, the method further comprising attaching said biosensor to a printed circuit board.

217. The method of claim 216, the method further comprising interfacing said printed circuit board with a data acquisition card.

218. The method of claim 200, the method further comprising interfacing said biosensor with a data acquisition card.

219. The method of claim 200, the method further comprising interfacing said biosensor with a digital multimeter.

220. The method of claim 200, wherein an upper step in said plurality of upper steps and the lower step associated with said upper step are separated by a vertical distance that is between 60 Angstroms and 200 Angstroms.

221. The method of claim 200, wherein an upper step in said plurality of upper steps and the lower step associated with said upper step are separated by a vertical distance that is less than 500 Angstroms.

222. The method of claim 200, wherein an upper step in said plurality of upper steps and the lower step associated with said upper step are separated by a vertical distance that is less than 1000 Angstroms.

223. The method of claim 200, wherein an upper step in said plurality of upper steps and the lower step associated with said upper step are separated by a vertical distance that is between 300 Angstroms and 400 Angstroms.

224. The method of claim 200, wherein said plurality of electrode pairs comprises at least 100 electrode pairs.

225. The method of claim 200, wherein said plurality of electrode pairs comprises at least 10,000 electrode pairs.

226. The method of claim 200, wherein said plurality of electrode pairs comprises 10,000 to 10⁵ electrode pairs.

227. The method of claim 200, wherein said plurality of electrode pairs comprises 10⁷ to 10⁹ electrode pairs.

228. The method of claim 200, wherein a macromolecule binds to both a first electrode and a second electrode in an electrode pair in said plurality of electrode pairs.

229. The method of claim 200, wherein said substrate comprises silicon, silicon oxide, silicon dioxide, silicon nitride, Teflon, alumina, glass, sapphire, a selinide, or polyester.

230. The method of claim 200 wherein said first electrode and said second electrode each has a resistivity less than 10⁻⁶ ohm-meters in an electrode pair in said plurality of electrode pairs.

231. The method of claim 200 wherein a first electrode and a second electrode in an electrode pair in said plurality of electrode pairs are comprised of the same composition.

232. The method of claim 200 wherein a first electrode and a second electrode in an electrode pair in said plurality of electrode pairs are each comprised of a different composition.

233. The method of claim 200 wherein said first electrode or said second electrode in an electrode pair in said plurality of electrodes comprises aluminum, nickel, platinum, iron, copper, silver, gold, indium tin oxide, chromium, titanium, zinc, tin, an alloy of aluminum, an alloy of nickel, an alloy of platinum, an alloy of iron, an alloy of copper, an alloy of silver, an alloy of gold, an alloy of chromium, an alloy of titanium, an alloy of zinc or an alloy of tin.

234. The method of claim 200 wherein the first electrode or the second electrode comprises a metal carbide, a metal nitride, a metal boride, a conductive oxide, a metal silicide or a metal sulfide in an electrode pair in said plurality of electrode pairs.

235. The method of claim 200 wherein said patterning step (b) comprises:
(i) applying a photolithographic photoresist coating to said electrically conducting layer;
(ii) optically imaging said photolithographic photoresist coating through an optical mask;
(iii) developing said photolithographic photoresist coating;
(iv) etching said spacer; and
(v) removing said photolithographic photoresist coating.

236. The method of claim 200 wherein said depositing step (a) is performed by chemical vapor deposition, reduced pressure chemical vapor deposition, low pressure chemical vapor deposition, atmospheric chemical vapor deposition, plasma enhanced chemical vapor deposition, anodization, sol-gel deposition, plasma spraying, ink jet printing, direct current diode sputtering, radio frequency diode sputtering, direct current magnetron sputtering, radio frequency magnetron sputtering, vacuum evaporation, collimated sputtering, laser ablated deposition, atomic layer deposition, molecular beam deposition, ionized physical vapor deposition, ion beam deposition, atomic layer deposition, hot filament chemical vapor deposition, screen printing, electroless metal deposition, electroplating, or electroless/immersion gold.

237. A method of detecting an analyte with a biosensor; wherein said biosensor comprises a plurality of devices, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer occupied by said device;
a spacer overlaid on a second portion of the different region of said insulator layer that is occupied by said device, wherein said first portion of the different region on said insulator does not overlap said second portion of the different region on said insulator; and
a second electrically conducting material, wherein the second electrically conducting material is overlaid on at least a portion of said spacer, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

238. A method of detecting an analyte with a biosensor; wherein said biosensor comprises a plurality of devices on a substrate, each device in said plurality of devices occupying a different region on said substrate, each device in said plurality of devices comprising:
a first electrically conducting material, wherein the first electrically conducting material is overlaid on a first portion of the different region on said substrate occupied by said device;
a spacer overlaid on a second portion of the different region on said substrate that is occupied by said device, wherein said first portion of the different region on said substrate does not overlap said second portion of the different region on said substrate; and
a second electrically conducting material, wherein the second electrically conducting material is overlaid on at least a portion of said spacer, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

239. A method of detecting an analyte with a biosensor; the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by the device; and
a second electrically conducting material, wherein the second electrically conducting material is overlaid on a second portion of the different region of said insulator layer that is occupied by said device, wherein said first portion of the different region on said insulator does not overlap said second portion of the different region on said insulator, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

240. A method of detecting an analyte with a biosensor; the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by the device; and
a second electrically conducting material, wherein the second electrically conducting material is overlaid on a second portion of the different region of said substrate that is occupied by said device, wherein said first portion of the different region on said substrate does not overlap said second portion of the different region on said substrate, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

241. A method of detecting an analyte with a biosensor; the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

242. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

243. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

244. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic proprty between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

245. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

246. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate occupied by said device, wherein said first portion of said substrate does not overlap with said second portion of said substrate;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

247. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device, the spacer including a main body and an extended portion, wherein said extended portion of said spacer abuts said first electrically conducting material and wherein said first portion of said insulator layer does not overlap with said second portion of said insulator layer;
a second electrically conducting material, wherein the second electrically conducting material is overlaid on said main body of said spacer; and
a first passivation layer overlays said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

248. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate that is occupied by said device, the spacer including a main body and an extended portion, wherein said extended portion of said spacer abuts said first electrically conducting material and wherein said first portion of said substrate does not overlap with said second portion of said substrate;
a second electrically conducting material, wherein the second electrically conducting material is overlaid on said main body of said spacer; and
a first passivation layer overlays said second electrically conducting material, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

249. A method of detecting an analyte with a biosensor, the biosensor comprising:
a substrate;
a first insulator layer overlaid on said substrate;
a first electrically conducting material overlaid on said insulator;
a passivation layer overlaid on said first electrically conducting material;
a plurality of devices; wherein each device in said plurality of devices comprises:
a cavity that extends through said passivation layer, said first electrically conducting material, and said first insulator layer;
a second insulator layer in said cavity; and
a second electrically conducting material on said second insulator layer, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

250. A method of detecting an analyte with a biosensor, the biosensor comprising a plurality of devices on a substrate, wherein
said substrate comprises a plurality of upper steps and a plurality of lower steps;
each upper step in the plurality of upper steps is associated with a lower step in the plurality of lower steps; and
for each device in said plurality of devices, a first electrically conducting material in the device overlays an upper step in said plurality of upper steps and a second electrically conducting material in the device overlays the lower step in said plurality of lower steps that is associated with the upper step, wherein
a first portion of a macromolecule is attached to said first electrically conducting material and a second portion of said macromolecule is attached to said second electrically conducting material in a device in said plurality of devices; the method comprising:
(a) detecting an electromagnetic property between said first electrically conducting material and said second electrically conducting material;
(b) contacting the macromolecule with said analyte such that said analyte binds to said macromolecule thereby forming a macromolecule/analyte complex that comprises said macromolecule and said analyte; and
(c) detecting a difference in said electromagnetic property between said first electrically conducting material and said second electrically conducting material.

251. The biosensor of claim 1, 23, 27, 45, 54, 60, 70 or 84 wherein a via penetrates said insulator layer and wherein said via is in electrical communication with (i) said second electrically conducting material and (ii) an external electromagenetic source.

252. The biosensor of claim 251 wherein said external electromagnetic source is a voltage source.

253. The biosensor of claim 1, 2, 23, 27, 28, 45, 46, 54, 55, 60, 61, 70, or 71 wherein a via penetrates said substrate and wherein said via is in electrical communication with (i) said first electrically conducting material or said second electrically conducting material and (ii) an external voltage source.

254. A method of detecting an analyte with a biosensor using a single stranded nucleic acid, wherein
said first portion of said single stranded nucleic is derivatized with a first reactive group that is not masked and wherein said second portion of said single stranded nucleic acid is derivatized with a second reactive group that is masked by a masking group;
the biosensor comprising a plurality of devices on a substrate, wherein each device in said plurality of devices comprises an electrode pair, the method comprising:
(a) exposing said unmasked reactive group to a first electrode in an electrode pair in a device in said plurality of devices in said biosensor under a first set of conditions that allow said single stranded nucleic acid to bind to said first electrode;
(b) incubating said single stranded nucleic acid that is bound to said first electrode with a solution that potentially includes an analyte under a second set of conditions for a period of time;
(c) removing said masking group from said second reactive group, thereby causing said second reactive group to bind to said second electrode in said electrode pair in said device in said plurality of devices; and
(d) detecting any connection between said first electrically conducting material and said second electrically conducting material.

255. The method of claim 254 wherein said first reactive group or said second reactive group is sulfur.

256. The method of claim 254 wherein said masking group is a photosensitive masking group and said removing comprises exposing said biosensor to light.

257. The method of claim 254 wherein said masking group is an electrolabile group and said removing comprises exposing said masking group to a voltage.

258. The method of claim 254 wherein said masking group is a chemically sensitive group and said removing comprises exposing said masking group to a chemical.

259. The method of claim 254 wherein said analyte comprises a nucleic acid sequence and said second set of conditions comprises conditions of low stringency.

260. The method of claim 254 wherein said period of time comprises less than one minute.

261. The method of claim 254 wherein said period of time comprises less than 15 minutes.

262. The method of claim 254 wherein said period of time comprises less than one day.

263. The method of claim 254 wherein said period of time comprises more than one hour.

264. The method of claim 254 wherein said method further comprises:
washing said biosensor; and
drying said first electrode and said second electrode prior to said detecting.

265. A biosensor comprising:
a substrate; and
a plurality of devices overlaid on said substrate, wherein (i) each device in said plurality of devices comprises an electrode pair, each said electrode pair comprising a first electrically conducting material and a second electrically conducting material and (ii) each respective said first electrically conducting material and said second electrically conducting material in each said electrode pair is separated by a distance that is between 60 Angstroms and 500 Angstroms;
wherein at least one device in said plurality of devices occupies 1/100 or less of a 100 micron square of surface area on said substrate.

266. The biosensor of claim 265 wherein there are between 100 devices and 500 devices on a 100 micron square of substrate surface.

267. The biosensor of claim 265 wherein there are between 500 devices and 1000 devices on a 100 micron square of substrate surface.

268. The biosensor of claim 265 wherein there are between 1000 devices and 2000 devices on a 100 micron square of substrate surface.

269. The biosensor of claim 265 wherein there are between 2000 devices and 3000 devices on a 100 micron square of substrate surface.

270. The biosensor of claim 265 wherein there are between 3000 devices and 4000 devices on a 100 micron square of substrate surface.

271. The biosensor of claim 265 wherein there are between 4000 devices and 5000 devices on a 100 micron square of substrate surface.

272. The biosensor of claim 265 wherein there are between 5000 devices and 6000 devices on a 100 micron square of substrate surface.

273. The biosensor of claim 265 wherein said substrate has a surface area size that is between 1 mm² and 10 mm².

274. The biosensor of claim 265 wherein said substrate has a surface area size that is between 10 mm² and 100 mm².

275. A biosensor comprising:
a substrate;
an insulator layer overlaid on said substrate; and
a plurality of devices overlaid on said substrate, wherein (i) each device in said plurality of devices comprises an electrode pair, each said electrode pair comprising a first electrically conducting material and a second electrically conducting material and (ii) each respective said first electrically conducting material and said second electrically conducting material in each said electrode pair is separated by a distance between 60 Angstroms and 500 Angstroms;
wherein at least one device in said plurality of devices occupies 1/100 or less of a 100 micron square of surface area on said substrate.

266. The biosensor of claim 275 wherein there are between 2000 devices and 3000 devices on a 100 micron square of substrate surface.

277. The biosensor of claim 275 wherein there are between 3000 devices and 4000 devices on a 100 micron square of substrate surface.

278. The biosensor of claim 275 wherein there are between 4000 devices and 5000 devices on a 100 micron square of substrate surface.

279. The biosensor of claim 275 wherein there are between 5000 devices and 6000 devices on a 100 micron square of substrate surface.

280. The biosensor of claim 275 wherein said substrate has a surface area size that is between 10 mm² and 100 mm².

281. An apparatus comprising:
a plurality of wells; and
a plurality of arrays, wherein
(a) each array in said plurality of arrays is in a well in said plurality of wells; and
(b) each array comprises a plurality of devices overlaid on a substrate, wherein (i) each device in said plurality of devices comprises an electrode pair, each said electrode pair comprising a first electrically conducting material and a second electrically conducting material and (ii) each respective said first electrically conducting material and said second electrically conducting material in each said electrode pair is separated by a distance between 60 Angstroms and 500 Angstroms.

282. The apparatus of claim 281 wherein said plurality of wells comprises 96 wells.

283. The apparatus of claim 281 wherein said plurality of wells comprises 384 wells.

284. The apparatus of claim 281 wherein said plurality of wells comprises 1584 wells.

285. The apparatus of claim 281 wherein there are at least 10,000 devices in an array in said plurality of arrays.

286. The apparatus of claim 281 wherein there are at least 40,000 devices in an array in said plurality of arrays.

287. The apparatus of claim 281 wherein there are at least 60,000 devices in an array in said plurality of arrays.

288. The apparatus of claim 281 wherein there are at least 120,000 devices in an array in said plurality of arrays.

289. The apparatus of claim 281 wherein there are at least 250,000 devices in an array in said plurality of arrays.

290. The method of claim 237, 238, 239, 240, 241, 242, 243, 244, 245, 246, 247, 248, 249 or 250 wherein said electromagnetic property is direct electric current, alternating electric current, permitivity, resistivity, electron transfer, electron tunneling, electron hopping, electron transport, electron conductance, voltage, electrical impedance, signal loss, dissipation factor, resistance, capacitance, inductance, magnetic field, electrical potential, charge or magnetic potential.

291. The method of claim 116 wherein said macromolecule is modified so that it is more electrically conductive then the corresponding unmodified macromolecule.

292. The method of claim 116 wherein said macromolecule is modified so that it is noninsulative.

293. The method of claim 291 or 292 wherein said macromolecule is modified by oxygen doping or iodine doping.

294. The method of claim 291 or 292 wherein said macromolecule is modified by labeling the macromolecule with a conductive metal.

295. The method of claim 294 wherein said conductive metal is gold, silver, platinum, coppper or tin.

296. The method of claim 294 wherein said labeling is performed using covalent attachment, photoreaction, or intercalation.

297. A method of detecting an analyte with a biosensor; wherein said biosensor comprises a plurality of devices, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer occupied by said device;
a spacer overlaid on a second portion of the different region of said insulator layer that is occupied by said device, wherein said first portion of the different region on said insulator does not overlap said second portion of the different region on said insulator; and
a second electrically conducting material, wherein the second electrically conducting material is overlaid on at least a portion of said spacer, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

298. A method of detecting an analyte with a biosensor; wherein said biosensor comprises a plurality of devices on a substrate, each device in said plurality of devices occupying a different region on said substrate, each device in said plurality of devices comprising:
a first electrically conducting material, wherein the first electrically conducting material is overlaid on a first portion of the different region on said substrate occupied by said device;
a spacer overlaid on a second portion of the different region on said substrate that is occupied by said device, wherein said first portion of the different region on said substrate does not overlap said second portion of the different region on said substrate; and
a second electrically conducting material, wherein the second electrically conducting material is overlaid on at least a portion of said spacer, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

299. A method of detecting an analyte with a biosensor; the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by the device; and
a second electrically conducting material, wherein the second electrically conducting material is overlaid on a second portion of the different region of said insulator layer that is occupied by said device, wherein said first portion of the different region on said insulator does not overlap said second portion of the different region on said insulator, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

300. A method of detecting an analyte with a biosensor; the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by the device; and
a second electrically conducting material, wherein the second electrically conducting material is overlaid on a second portion of the different region of said substrate that is occupied by said device, wherein said first portion of the different region on said substrate does not overlap said second portion of the different region on said substrate, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

301. A method of detecting an analyte with a biosensor; the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

302. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaid on said first electrically conducting material, wherein said spacer comprises a thin segment and a thick segment and wherein said thin segment of said spacer is not as thick as said thick segment of said spacer;
a second electrically conducting material overlaid on said spacer; and
a passivation layer overlaid on said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

303. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on said different region of said insulator layer occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

304. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on said different region of said substrate occupied by the device;
a spacer overlaying a portion of said first electrically conducting material;
a second electrically conducting material overlaid on said spacer and protruding past an end of said spacer, over said first electrically conducting material, so that a gap is formed from an end of the first electrically conducting material and the portion of said second electrically conducting material that protrudes past said end of said spacer; and
a passivation layer overlaid on said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

305. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

306. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate occupied by said device, wherein said first portion of said substrate does not overlap with said second portion of said substrate;
a second electrically conducting material that abuts a side-wall of said spacer facing said first electrically conducting material; and
a first passivation layer that covers (i) the top of said spacer, (ii) a first side of said second electrically conducting material, and (iii) a portion of a second side of said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

307. A method of detecting an analyte with a biosensor, the biosensor comprising a plurality of devices on a substrate, each device in said plurality of devices occupying a different region on an insulator layer, wherein the insulator layer is overlaid on said substrate, each device in said plurality of devices comprising:
a first electrically conducting material, wherein the first electrically conducting material is overlaid on a first portion of the different region of said insulator layer that is occupied by said device;
a spacer overlaid on a second portion of the different region of said insulator layer that is occupied by said device, the spacer including a main body and an extended portion, wherein said extended portion of said spacer abuts said first electrically conducting material and wherein said first portion of said insulator layer does not overlap with said second portion of said insulator layer;
a second electrically conducting material, wherein the second electrically conducting material is overlaid on said main body of said spacer; and
a first passivation layer overlays said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

308. A method of detecting an analyte with a biosensor, the biosensor comprising a plurality of devices on a substrate, each device in said plurality of devices occupying a different region on said substrate, each device in said plurality of devices comprising:
a first electrically conducting material, wherein the first electrically conducting material is overlaid on a first portion of the different region of said substrate that is occupied by said device;
a spacer overlaid on a second portion of the different region of said substrate that is occupied by said device, the spacer including a main body and an extended portion, wherein said extended portion of said spacer abuts said first electrically conducting material and wherein said first portion of said substrate does not overlap with said second portion of said substrate;
a second electrically conducting material, wherein the second electrically conducting material is overlaid on said main body of said spacer; and
a first passivation layer overlays said second electrically conducting material, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

309. A method of detecting an analyte with a biosensor, the biosensor comprising:
a substrate;
a first insulator layer overlaid on said substrate;
a first electrically conducting material overlaid on said insulator;
a passivation layer overlaid on said first electrically conducting material;
a plurality of devices; wherein each device in said plurality of devices comprises:
a cavity that extends through said passivation layer, said first electrically conducting material, and said first insulator layer;
a second insulator layer in said cavity; and
a second electrically conducting material on said second insulator layer, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

310. A method of detecting an analyte with a biosensor, the biosensor comprising a plurality of devices on a substrate, wherein
said substrate comprises a plurality of upper steps and a plurality of lower steps;
each upper step in the plurality of upper steps is associated with a lower step in the plurality of lower steps; and
for each device in said plurality of devices, a first electrically conducting material in the device overlays an upper step in said plurality of upper steps and a second electrically conducting material in the device overlays the lower step in said plurality of lower steps that is associated with the upper step, the method comprising:
(a) attaching a first portion of a macromolecule to said first electrically conducting material in a device in said plurality of devices;
(b) detecting an electromagnetic property between said first electrically conducting material and a second electrically conducting material in said device;
(c) contacting the macromolecule with a sample potentially comprising said analyte under conditions such that any said analyte in said sample binds to said macromolecule thereby forming a macromolecule/analyte complex that comprises said macromolecule and said analyte;
(d) attaching a second portion of any said macromolecule/analyte complex so formed to said second electrically conducting material in said device; and
(e) detecting any difference in said electromagnetic property between said first electrically conducting material and said second electrically conducting material.

311. The method of claim 237, 238, 239,240, 241, 242, 243, 244, 245, 246, 247, 248, 249, or 250 further comprising attaching said first portion of said macromolecule to said first electrically conducting material and said second protion of said macromolecule to said second electrically conducting material in said device in said plurality of devices prior to said detecting step (a).
